(19) United States
(12) Patent Application Publication
(10) Pub. No.: US 2016/0002204 A1
(43) Pub. Date: Jan. 7, 2016

(54) GLS1 INHIBITORS FOR TREATING DISEASE

(71) Applicant: Board of Regents, The University of Texas System, Austin, TX (US)

(72) Inventors: Maria Emilia Di Francesco, Houston, TX (US); Philip Jones, Houston, TX (US); Timothy Heffernan, Sugar Land, TX (US); Matthew M. Hamilton, Missouri City, TX (US); Zhijun Kang, Sugar Land, TX (US); Michael J. Soth, Sugar Land, TX (US); Jason P. Burke, Houston, TX (US); Christopher Lawrence Carroll, Houston, TX (US); Timothy McAfoos, Houston, TX (US); Wylie Palmer, Houston, TX (US); Zachary Herrera, Houston, TX (US)

(73) Assignee: Board of Regents, The University of Texas System, Austin, TX (US)

(21) Appl. No.: 14/791,284

(22) Filed: Jul. 3, 2015

Related U.S. Application Data
(60) Provisional application No. 62/020,519, filed on Jul. 3, 2014.

(51) Int. Cl. C07D 401/06; A61K 31/444; A61K 45/06; A61K 31/4439; C07D 401/14; A61K 31/501; C07D 403/06; A61K 31/513; C07D 417/06; C07D 487/04; A61K 31/519; A61K 31/4545; C07D 405/14; C07D 403/14; C07D 409/14; A61K 31/5377; A61K 31/541; A61N 5/10; A61N 7/00; C07D 417/14

(52) U.S. Cl. CPC C07D 401/06; C07D 417/14; A61K 31/444; A61K 45/06; A61K 31/4439; C07D 401/14; A61K 31/501; C07D 403/06; A61K 31/513; C07D 417/06; C07D 487/04; A61K 31/519; A61K 31/4545; C07D 405/14; C07D 403/14; C07D 409/14; A61K 31/5377; A61K 31/541; A61N 5/10; A61N 7/00

(57) ABSTRACT

Disclosed herein are compounds and compositions useful in the treatment of GLS1 mediated diseases, such as cancer, having the structure of Formula I:

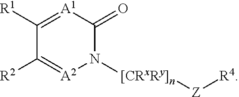

Methods of inhibition GLS1 activity in a human or animal subject are also provided.

This application claims the benefit of priority of U.S. provisional Application No. 62/020,519, filed Jul. 3, 2014, the disclosure of which is hereby incorporated by reference as if written herein in its entirety.

The present disclosure relates to new heterocyclic compounds and compositions, and their application as pharmaceuticals for the treatment of disease. Methods of inhibition of GLS1 activity in a human or animal subject are also provided for the treatment of diseases such as cancer.

Metabolic deregulation is a hallmark of cancer as tumors exhibit an increased demand for nutrients and macromolecules to fuel their rapid proliferation. Glutamine (Gln), the most abundant amino acid in circulation, plays an essential role in providing cancer cells with biosynthetic intermediates required to support proliferation and survival. Specifically, glutaminolysis, or the enzymatic conversion of glutamine to glutamate, provides proliferating cancer cells with a source of nitrogen for amino acid and nucleotide synthesis, and a carbon skeleton to fuel ATP and NADPH synthesis through the TCA cycle. In addition to supporting cell growth, glutamine metabolism plays a critical role in maintaining cellular redox homeostasis as glutamate can be converted into glutathione, the major intracellular antioxidant.

Glutaminolysis is regulated by mitochondrial glutaminase (GLS), the rate limiting enzyme that catalyzes the conversion of Gln to glutamate and ammonia. Mammalian cells contain 2 genes that encode glutaminase: the kidney-type (GLS1) and liver-type (GLS2) enzymes. Each has been detected in multiple tissue types, with GLS1 being widely distributed throughout the body. GLS1 is a phosphate-activated enzyme that exists in humans as two major splice variants, a long form (referred to as KGA) and a short form (GAC), which differ only in their C-terminal sequences. Both forms of GLS1 are thought to bind to the inner membrane of the mitochondrion in mammalian cells, although at least one report suggests that glutaminase may exist in the intramembrane space, dissociated from the membrane. GLS is frequently overexpressed in human tumors and has been shown to be positively regulated by oncogenes such as Myc. Consistent with the observed dependence of cancer cell lines on glutamine metabolism, pharmcological inhibition of GLS offers the potential to target Gln addicted tumors.

Thus, there is a need for glutaminase inhibitors that are specific and capable of being formulated for in vivo use.

### SUMMARY

Accordingly, the inventors herein disclose new compositions and methods for inhibiting glutaminase activity.

Provided is compound of structural Formula I

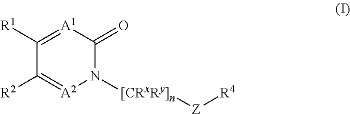

or a salt thereof, wherein: n is chosen from 3, 4, and 5; each R^{x} and R^{y} is independently chosen from alkyl, cyano, H, and halo, wherein two R^{x} groups together with the atoms to which they are attached optionally form a cycloalkyl ring; A¹ and A² are independently chosen from C—H, C—F, and N; R¹ and R⁴ are independently chosen from alkenyl, alkoxy, alkyl, aryl, arylalkyl, cyano, cycloalkyl, cycloalkylalkyl, H, halo, haloalkyl, heteroaryl, heteroarylalkyl, heterocycloalkyl, heterocycloalkylalkyl, hydroxyl, C(R³)₂C(O)R³, C(R³)₂C(O)N(R³)₂, C(R³)₂N(R³)₂, C(R³)₂NR³C(O)R³, C(R³)₂NR³C(O)OR³, C(R³)₂NR³C(O)N(R³)₂, C(R³)₂NR³S(O)R³, C(R³)₂NR³S(O)₂R³, N(R³)₂, NR³C(O)R³, NR³C(O)OR³, NR³C(O)N(R³)₂, NR³S(O)R³, NR³S(O)₂R³, C(O)N(R³)₂, S(O)N(R³)₂, S(O)₂N(R³)₂, C(O)R³, SR³, S(O)R³, and S(O)₂R³, wherein each R¹ and R⁴ may be optionally substituted with between 0 and 3 R^{z} groups; R² is chosen from alkyl, heterocycloalkyl, cyano, cycloalkyl, H, halo, and haloalkyl, wherein R¹ and R² together with the atoms to which they are attached optionally form an form an aryl, cycloalkyl, heteroaryl, or heterocycloalkyl ring, which may be optionally substituted with between 0 and 3 R^{z} groups; each R³ is independently chosen from alkenyl, alkoxy, alkyl, aryl, arylalkyl, cyano, cycloalkyl, cycloalkylalkyl, H, halo, haloalkyl, heteroaryl, heteroarylalkyl, heterocycloalkyl, heterocycloalkylalkyl, and hydroxyl, wherein each R³ may be optionally substituted with between 0 and 3 R^{z} groups, wherein two R³ groups together with the atoms to which they are attached optionally form an aryl, cycloalkyl, heteroaryl, or heterocycloalkyl ring, which may be optionally substituted with between 0 and 3 R^{z} groups; each R^{Z} group is independently chosen from alkenyl, alkoxy, alkoxyalkyl, alkoxyaryl, alkoxyarylalkyl, alkoxycycloalkyl, alkoxycycloalkylalkyl, alkoxyhaloalkyl, alkoxyheteroaryl, alkoxyheteroarylalkyl, alkoxyheterocycloalkyl, alkoxyheterocycloalkylalkyl, alkyl, alkylaryl, alkylarylalkyl, alkylcycloalkyl, alkylcycloalkylalkyl, alkylheteroaryl, alkylheteroarylalkyl, alkylheterocycloalkyl, alkylheterocycloalkylalkyl, aryl, arylalkyl, arylalkyloxy, arylhaloalkyl, aryloxy, cyano, cycloalkyl, cycloalkylalkyl, cycloalkylalkyloxy, cycloalkylhaloalkyl, cycloalkyloxy, H, halo, haloalkoxy, haloalkoxyalkyl, haloalkoxyaryl, haloalkoxyarylalkyl, haloalkoxycycloalkyl, haloalkoxycycloalkylalkyl, haloalkoxyheteroaryl, haloalkoxyheteroarylalkyl, haloalkoxyheterocycloalkyl, haloalkoxyheterocycloalkylalkyl, haloalkyl, haloalkylaryl, haloalkylarylalkyl, haloalkylcycloalkyl, haloalkylcycloalkylalkyl, haloalkylheteroaryl, haloalkylheteroarylalkyl, haloalkylheterocycloalkyl, haloalkylheterocycloalkylalkyl, haloaryl, haloarylalkyl, haloarylalkyloxy, haloaryloxy, halocycloalkyl, halocycloalkylalkyl, halocycloalkylalkyloxy, halocycloalkyloxy, haloheteroaryl, haloheteroarylalkyl, haloheteroarylalkyloxy, haloheteroaryloxy, haloheterocycloalkyl, haloheterocycloalkylalkyl, haloheterocycloalkylalkyloxy, haloheterocycloalkyloxy, heteroaryl, heteroarylalkyl, heteroarylalkyloxy, heteroarylhaloalkyl, heteroaryloxy, heterocycloalkyl, heterocycloalkylalkyl, heterocycloalkylalkyloxy, heterocycloalkylhaloalkyl, heterocycloalkyloxy, hydroxyl, oxo, N(R⁶)₂, NR⁶C(O)C(R⁶)₃, NR⁶C(O)OC(R⁶)₃, NR⁶C(O)N(R⁶)₂, NR⁶S(O)C(R⁶)₃, NR⁶S(O)₂C(R⁶)₃, C(O)N(R⁶)₂, S(O)N(R⁶)₂, S(O)₂N(R⁶)₂, C(O)C(R⁶)₃, SC(R⁶)₃, S(O)C(R⁶)₃, and S(O)₂C(R⁶)₃; each R⁶ is independently chosen from alkenyl, alkoxy, alkyl, aryl, arylalkyl, cyano, cycloalkyl, cycloalkylalkyl, H, halo, haloalkyl, heteroaryl, heteroarylalkyl, heterocycloalkyl, heterocycloalkylalkyl, and hydroxyl, wherein two R⁶ groups together with the atoms to which they are attached optionally form an aryl, cycloalkyl, heteroaryl, or heterocycloalkyl ring, which may be optionally substituted with between 0 and 3 R^{x} groups; and Z is heteroaryl, which may be optionally substituted.

Provided is a composition comprising a compound of Formula I and a pharmaceutically acceptable carrier, adjuvant, or vehicle.

Provided is a method of inhibiting GLS1 activity in a biological sample comprising contacting the biological sample with a compound of Formula I.

Provided is a method of treating a GLS1-mediated disorder in a subject in need thereof, comprising the step of administering to the subject a compound of Formula I.

Provided is a method of treating a GLS1-mediated disorder in a subject in need thereof, comprising the sequential or co-administration of a compound of Formula I or a pharmaceutically acceptable salt thereof, and another therapeutic agent.

Provided is a compound of any of Formula I for use in human therapy.

Provided is a compound of any of Formula I for use in treating a GLS1-mediated disease.

Provided is a use of a compound of Formula I for the manufacture of a medicament to treat a GLS1-mediated disease.

### DETAILED DESCRIPTION

### Abbreviations and Definitions

To facilitate understanding of the disclosure, a number of terms and abbreviations as used herein are defined below as follows:

When introducing elements of the present disclosure or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

The term “and/or” when used in a list of two or more items, means that any one of the listed items can be employed by itself or in combination with any one or more of the listed items.

For example, the expression “A and/or B” is intended to mean either or both of A and B, i.e. A alone, B alone or A and B in combination. The expression “A, B and/or C” is intended to mean A alone, B alone, C alone, A and B in combination, A and C in combination, B and C in combination or A, B, and C in combination.

When ranges of values are disclosed, and the notation “from n₁ . . . to n₂” or “between n₁ . . . and n₂” is used, where n₁ and n₂ are the numbers, then unless otherwise specified, this notation is intended to include the numbers themselves and the range between them. This range may be integral or continuous between and including the end values. By way of example, the range “from 2 to 6 carbons” is intended to include two, three, four, five, and six carbons, since carbons come in integer units. Compare, by way of example, the range “from 1 to 3 μM (micromolar),” which is intended to include 1 μM, 3 μM, and everything in between to any number of significant figures (e.g., 1.255 μM, 2.1 μM, 2.9999 μM, etc.).

The term “about,” as used herein, is intended to qualify the numerical values that it modifies, denoting such a value as variable within a margin of error. When no particular margin of error, such as a standard deviation to a mean value given in a chart or table of data, is recited, the term “about” should be understood to mean that range which would encompass the recited value and the range which would be included by rounding up or down to that figure as well, taking into account significant figures.

The term “acyl,” as used herein, alone or in combination, refers to a carbonyl attached to an alkenyl, alkyl, aryl, cycloalkyl, heteroaryl, heterocycle, or any other moiety were the atom attached to the carbonyl is carbon. An “acetyl” group refers to a —C(O)CH₃ group. An “alkylcarbonyl” or “alkanoyl” group refers to an alkyl group attached to the parent molecular moiety through a carbonyl group. Examples of such groups include methylcarbonyl and ethylcarbonyl. Examples of acyl groups include formyl, alkanoyl and aroyl.

The term “alkenyl,” as used herein, alone or in combination, refers to a straight-chain or branched-chain hydrocarbon radical having one or more double bonds and containing from 2 to 20 carbon atoms. In certain embodiments, the alkenyl will comprise from 2 to 6 carbon atoms. The term “alkenylene” refers to a carbon-carbon double bond system attached at two or more positions such as ethenylene [(—CH═CH—),(—C::C—)]. Examples of suitable alkenyl radicals include ethenyl, propenyl, 2-methylpropenyl, 1,4-butadienyl and the like. Unless otherwise specified, the term “alkenyl” may include “alkenylene” groups.

The term “alkoxy,” as used herein, alone or in combination, refers to an alkyl ether radical, wherein the term alkyl is as defined below. Examples of suitable alkyl ether radicals include methoxy, ethoxy, n-propoxy, isopropoxy, n-butoxy, iso-butoxy, sec-butoxy, tert-butoxy, and the like.

The term “alkyl,” as used herein, alone or in combination, refers to a straight-chain or branched-chain alkyl radical containing from 1 to 20 carbon atoms. In certain embodiments, the alkyl will comprise from 1 to 10 carbon atoms. In further embodiments, the alkyl will comprise from 1 to 6 carbon atoms. Alkyl groups may be optionally substituted as defined herein. Examples of alkyl radicals include methyl, ethyl, n-propyl, isopropyl, n-butyl, isobutyl, sec-butyl, tert-butyl, pentyl, iso-amyl, hexyl, octyl, noyl and the like. The term “alkylene,” as used herein, alone or in combination, refers to a saturated aliphatic group derived from a straight or branched chain saturated hydrocarbon attached at two or more positions, such as methylene (—CH₂—). Unless otherwise specified, the term “alkyl” may include “alkylene” groups.

The term “alkylamino,” as used herein, alone or in combination, refers to an alkyl group attached to the parent molecular moiety through an amino group. Suitable alkylamino groups may be mono- or dialkylated, forming groups such as, for example, N-methylamino, N-ethylamino, N,N-dimethylamino, N,N-ethylmethylamino and the like.

The term “alkylidene,” as used herein, alone or in combination, refers to an alkenyl group in which one carbon atom of the carbon-carbon double bond belongs to the moiety to which the alkenyl group is attached.

The term “alkylthio,” as used herein, alone or in combination, refers to an alkyl thioether (R—S—) radical wherein the term alkyl is as defined above and wherein the sulfur may be singly or doubly oxidized. Examples of suitable alkyl thioether radicals include methylthio, ethylthio, n-propylthio, isopropylthio, n-butylthio, iso-butylthio, sec-butylthio, tert-butylthio, methanesulfonyl, ethanesulfinyl, and the like.

The term “alkynyl,” as used herein, alone or in combination, refers to a straight-chain or branched chain hydrocarbon radical having one or more triple bonds and containing from 2 to 20 carbon atoms. In certain embodiments, the alkynyl comprises from 2 to 6 carbon atoms. In further embodiments, the alkynyl comprises from 2 to 4 carbon atoms. The term “alkynylene” refers to a carbon-carbon triple bond attached at two positions such as ethynylene (—C:::C—, —C≡C—). Examples of alkynyl radicals include ethynyl, propynyl, hydroxypropynyl, butyn-1-yl, butyn-2-yl, pentyn-1-yl, 3-methylbutyn-1-yl, hexyn-2-yl, and the like. Unless otherwise specified, the term “alkynyl” may include “alkynylene” groups.

The terms “amido” and “carbamoyl” as used herein, alone or in combination, refer to an amino group as described below attached to the parent molecular moiety through a carbonyl group, or vice versa. The term “C-amido” as used herein, alone or in combination, refers to a —C(O)N(RR′) group with R and R′ as defined herein or as defined by the specifically enumerated “R” groups designated. The term “N-amido” as used herein, alone or in combination, refers to a RC(O)N(R′)— group, with R and R′ as defined herein or as defined by the specifically enumerated “R” groups designated. The term “acylamino” as used herein, alone or in combination, embraces an acyl group attached to the parent moiety through an amino group. An example of an “acylamino” group is acetylamino (CH₃C(O)NH—).

The term “amino,” as used herein, alone or in combination, refers to —NRR′, wherein R and R′ are independently selected from the group consisting of hydrogen, alkyl, acyl, heteroalkyl, aryl, cycloalkyl, heteroaryl, and heterocycloalkyl, any of which may themselves be optionally substituted. Additionally, R and R′ may combine to form heterocycloalkyl, either of which may be optionally substituted.

The term “aryl,” as used herein, alone or in combination, means a carbocyclic aromatic system containing one, two or three rings wherein such polycyclic ring systems are fused together. The term “aryl” embraces aromatic groups such as phenyl, naphthyl, anthracenyl, and phenanthryl.

The term “arylalkenyl” or “aralkenyl,” as used herein, alone or in combination, refers to an aryl group attached to the parent molecular moiety through an alkenyl group.

The term “arylalkoxy” or “aralkoxy,” as used herein, alone or in combination, refers to an aryl group attached to the parent molecular moiety through an alkoxy group.

The term “arylalkyl” or “aralkyl,” as used herein, alone or in combination, refers to an aryl group attached to the parent molecular moiety through an alkyl group.

The term “arylalkynyl” or “aralkynyl,” as used herein, alone or in combination, refers to an aryl group attached to the parent molecular moiety through an alkynyl group.

The term “arylalkanoyl” or “aralkanoyl” or “aroyl,” as used herein, alone or in combination, refers to an acyl radical derived from an aryl-substituted alkanecarboxylic acid such as benzoyl, napthoyl, phenylacetyl, 3-phenylpropionyl (hydrocinnamoyl), 4-phenylbutyryl, (2-naphthyl)acetyl, 4-chlorohydrocinnamoyl, and the like.

The term aryloxy as used herein, alone or in combination, refers to an aryl group attached to the parent molecular moiety through an oxy.

The terms “benzo” and “benz,” as used herein, alone or in combination, refer to the divalent radical C₆H₄═ derived from benzene. Examples include benzothiophene and benzimidazole.

The term “carbamate,” as used herein, alone or in combination, refers to an ester of carbamic acid (—NHCOO—) which may be attached to the parent molecular moiety from either the nitrogen or acid end, and which may be optionally substituted as defined herein.

The term “O-carbamyl” as used herein, alone or in combination, refers to a —OC(O)NRR′, group-with R and R′ as defined herein.

The term “N-carbamyl” as used herein, alone or in combination, refers to a ROC(O)NR′— group, with R and R′ as defined herein.

The term “carbonyl,” as used herein, when alone includes formyl [—C(O)H] and in combination is a —C(O)— group.

The term “carboxyl” or “carboxy,” as used herein, refers to —C(O)OH or the corresponding “carboxylate” anion, such as is in a carboxylic acid salt. An “O-carboxy” group refers to a RC(O)O— group, where R is as defined herein. A “C-carboxy” group refers to a —C(O)OR groups where R is as defined herein.

The term “cyano,” as used herein, alone or in combination, refers to —CN.

The term “cycloalkyl,” or, alternatively, “carbocycle,” as used herein, alone or in combination, refers to a saturated or partially saturated monocyclic, bicyclic or tricyclic alkyl group wherein each cyclic moiety contains from 3 to 12 carbon atom ring members and which may optionally be a benzo fused ring system which is optionally substituted as defined herein. In certain embodiments, the cycloalkyl will comprise from 5 to 7 carbon atoms. Examples of such cycloalkyl groups include cyclopropyl, cyclobutyl, cyclopentyl, cyclohexyl, cycloheptyl, tetrahydronapthyl, indanyl, octahydronaphthyl, 2,3-dihydro-1H-indenyl, adamantyl and the like. “Bicyclic” and “tricyclic” as used herein are intended to include both fused ring systems, such as decahydronaphthalene, octahydronaphthalene as well as the multicyclic (multicentered) saturated or partially unsaturated type. The latter type of isomer is exemplified in general by, bicyclo[1,1,1]pentane, camphor, adamantane, and bicyclo[3,2,1]octane.

The term “ester,” as used herein, alone or in combination, refers to a carboxy group bridging two moieties linked at carbon atoms.

The term “ether,” as used herein, alone or in combination, refers to an oxy group bridging two moieties linked at carbon atoms.

The term “halo,” or “halogen,” as used herein, alone or in combination, refers to fluorine, chlorine, bromine, or iodine.

The term “haloalkoxy,” as used herein, alone or in combination, refers to a haloalkyl group attached to the parent molecular moiety through an oxygen atom.

The term “haloalkyl,” as used herein, alone or in combination, refers to an alkyl radical having the meaning as defined above wherein one or more hydrogens are replaced with a halogen. Specifically embraced are monohaloalkyl, dihaloalkyl and polyhaloalkyl radicals. A monohaloalkyl radical, for one example, may have an iodo, bromo, chloro or fluoro atom within the radical. Dihalo and polyhaloalkyl radicals may have two or more of the same halo atoms or a combination of different halo radicals. Examples of haloalkyl radicals include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. “Haloalkylene” refers to a haloalkyl group attached at two or more positions. Examples include fluoromethylene (—CFH—), difluoromethylene (—CF₂—), chloromethylene (—CHCl—) and the like.

The term “heteroalkyl,” as used herein, alone or in combination, refers to a stable straight or branched chain, or cyclic hydrocarbon radical, or combinations thereof, fully saturated or containing from 1 to 3 degrees of unsaturation, consisting of the stated number of carbon atoms and from one to three heteroatoms selected from the group consisting of O, N, and S, and wherein the nitrogen and sulfur atoms may optionally be oxidized and the nitrogen heteroatom may optionally be quaternized. The heteroatom(s) O, N and S may be placed at any interior position of the heteroalkyl group. Up to two heteroatoms may be consecutive, such as, for example, —CH₂—NH—OCH₃.

The term “heteroaryl,” as used herein, alone or in combination, refers to a 3 to 15 membered unsaturated heteromonocyclic ring, or a fused monocyclic, bicyclic, or tricyclic ring system in which at least one of the fused rings is aromatic, which contains at least one atom selected from the group consisting of O, S, and N. In certain embodiments, the heteroaryl will comprise from 5 to 7 carbon atoms. The term also embraces fused polycyclic groups wherein heterocyclic rings are fused with aryl rings, wherein heteroaryl rings are fused with other heteroaryl rings, wherein heteroaryl rings are fused with heterocycloalkyl rings, or wherein heteroaryl rings are fused with cycloalkyl rings. Examples of heteroaryl groups include pyrrolyl, pyrrolinyl, imidazolyl, pyrazolyl, pyridyl, pyrimidinyl, pyrazinyl, pyridazinyl, triazolyl, pyranyl, furyl, thienyl, oxazolyl, isoxazolyl, oxadiazolyl, thiazolyl, thiadiazolyl, isothiazolyl, indolyl, isoindolyl, indolizinyl, benzimidazolyl, quinolyl, isoquinolyl, quinoxalinyl, quinazolinyl, indazolyl, benzotriazolyl, benzodioxolyl, benzopyranyl, benzoxazolyl, benzoxadiazolyl, benzothiazolyl, benzothiadiazolyl, benzofuryl, benzothienyl, chromonyl, coumarinyl, benzopyranyl, tetrahydroquinolinyl, tetrazolopyridazinyl, tetrahydroisoquinolinyl, thienopyridinyl, furopyridinyl, pyrrolopyridinyl and the like. Exemplary tricyclic heterocyclic groups include carbazolyl, benzidolyl, phenanthrolinyl, dibenzofuranyl, acridinyl, phenanthridinyl, xanthenyl and the like.

The terms “heterocycloalkyl” and, interchangeably, “heterocycle,” as used herein, alone or in combination, each refer to a saturated, partially unsaturated, or fully unsaturated monocyclic, bicyclic, or tricyclic heterocyclic group containing at least one heteroatom as a ring member, wherein each the heteroatom may be independently selected from the group consisting of nitrogen, oxygen, and sulfur In certain embodiments, the hetercycloalkyl will comprise from 1 to 4 heteroatoms as ring members. In further embodiments, the hetercycloalkyl will comprise from 1 to 2 heteroatoms as ring members. In certain embodiments, the hetercycloalkyl will comprise from 3 to 8 ring members in each ring. In further embodiments, the hetercycloalkyl will comprise from 3 to 7 ring members in each ring. In yet further embodiments, the hetercycloalkyl will comprise from 5 to 6 ring members in each ring. “Heterocycloalkyl” and “heterocycle” are intended to include sulfones, sulfoxides, N-oxides of tertiary nitrogen ring members, and carbocyclic fused and benzo fused ring systems; additionally, both terms also include systems where a heterocycle ring is fused to an aryl group, as defined herein, or an additional heterocycle group. Examples of heterocycle groups include aziridinyl, azetidinyl, 1,3-benzodioxolyl, dihydroisoindolyl, dihydroisoquinolinyl, dihydrocinnolinyl, dihydrobenzodioxinyl, dihydro[1,3]oxazolo[4,5-b]pyridinyl, benzothiazolyl, dihydroindolyl, dihy-dropyridinyl, 1,3-dioxanyl, 1,4-dioxanyl, 1,3-dioxolanyl, isoindolinyl, morpholinyl, piperazinyl, pyrrolidinyl, tetrahydropyridinyl, piperidinyl, thiomorpholinyl, and the like. The heterocycle groups may be optionally substituted unless specifically prohibited.

The term “hydrazinyl” as used herein, alone or in combination, refers to two amino groups joined by a single bond, i.e., —N—N—.

The term “hydroxy,” as used herein, alone or in combination, refers to —OH.

The term “hydroxyalkyl,” as used herein, alone or in combination, refers to a hydroxy group attached to the parent molecular moiety through an alkyl group.

The term “imino,” as used herein, alone or in combination, refers to ═N—.

The term “iminohydroxy,” as used herein, alone or in combination, refers to ═N(OH) and ═N—O—.

The phrase “in the main chain” refers to the longest contiguous or adjacent chain of carbon atoms starting at the point of attachment of a group to the compounds of any one of the formulas disclosed herein.

The term “isocyanato” refers to a —NCO group.

The term “isothiocyanato” refers to a —NCS group.

The phrase “linear chain of atoms” refers to the longest straight chain of atoms independently selected from carbon, nitrogen, oxygen and sulfur.

The term “lower,” as used herein, alone or in a combination, where not otherwise specifically defined, means containing from 1 to and including 6 carbon atoms.

The term “lower aryl,” as used herein, alone or in combination, means phenyl or naphthyl, either of which may be optionally substituted as provided.

The term “lower heteroaryl,” as used herein, alone or in combination, means either 1) monocyclic heteroaryl comprising five or six ring members, of which between one and four the members may be heteroatoms selected from the group consisting of O, S, and N, or 2) bicyclic heteroaryl, wherein each of the fused rings comprises five or six ring members, comprising between them one to four heteroatoms selected from the group consisting of O, S, and N.

The term “lower cycloalkyl,” as used herein, alone or in combination, means a monocyclic cycloalkyl having between three and six ring members. Lower cycloalkyls may be unsaturated. Examples of lower cycloalkyl include cyclopropyl, cyclobutyl, cyclopentyl, and cyclohexyl.

The term “lower heterocycloalkyl,” as used herein, alone or in combination, means a monocyclic heterocycloalkyl having between three and six ring members, of which between one and four may be heteroatoms selected from the group consisting of O, S, and N. Examples of lower heterocycloalkyls include pyrrolidinyl, imidazolidinyl, pyrazolidinyl, piperidinyl, piperazinyl, and morpholinyl. Lower heterocycloalkyls may be unsaturated.

The term “lower amino,” as used herein, alone or in combination, refers to —NRR′, wherein R and R′ are independently selected from the group consisting of hydrogen, lower alkyl, and lower heteroalkyl, any of which may be optionally substituted. Additionally, the R and R′ of a lower amino group may combine to form a five- or six-membered heterocycloalkyl, either of which may be optionally substituted.

The term “mercaptyl” as used herein, alone or in combination, refers to an RS— group, where R is as defined herein.

The term “nitro,” as used herein, alone or in combination, refers to —NO₂.

The terms “oxy” or “oxa,” as used herein, alone or in combination, refer to —O—.

The term “oxo,” as used herein, alone or in combination, refers to ═O.

The term “perhaloalkoxy” refers to an alkoxy group where all of the hydrogen atoms are replaced by halogen atoms.

The term “perhaloalkyl” as used herein, alone or in combination, refers to an alkyl group where all of the hydrogen atoms are replaced by halogen atoms.

The terms “sulfonate,” “sulfonic acid,” and “sulfonic,” as used herein, alone or in combination, refer the —SO₃H group and its anion as the sulfonic acid is used in salt formation.

The term “sulfanyl,” as used herein, alone or in combination, refers to —S—.

The term “sulfinyl,” as used herein, alone or in combination, refers to —S(O)—.

The term “sulfonyl,” as used herein, alone or in combination, refers to —S(O)₂—.

The term “N-sulfonamido” refers to a RS(═O)₂NR′— group with R and R′ as defined herein.

The term “S-sulfonamido” refers to a —S(═O)₂NRR′, group, with R and R′ as defined herein.

The terms “thia” and “thio,” as used herein, alone or in combination, refer to a —S— group or an ether wherein the oxygen is replaced with sulfur. The oxidized derivatives of the thio group, namely sulfinyl and sulfonyl, are included in the definition of thia and thio.

The term “thiol,” as used herein, alone or in combination, refers to an —SH group.

The term “thiocarbonyl,” as used herein, when alone includes thioformyl —C(S)H and in combination is a —C(S)— group.

The term “N-thiocarbamyl” refers to an ROC(S)NR′— group, with R and R′ as defined herein.

The term “O-thiocarbamyl” refers to a —OC(S)NRR′, group with R and R′ as defined herein.

The term “thiocyanato” refers to a —CNS group.

The term “trihalomethanesulfonamido” refers to a X₃CS(O)₂NR— group with X is a halogen and R as defined herein.

The term “trihalomethanesulfonyl” refers to a X₃CS(O)₂— group where X is a halogen.

The term “trihalomethoxy” refers to a X₃CO— group where X is a halogen.

The term “trisubstituted silyl,” as used herein, alone or in combination, refers to a silicone group substituted at its three free valences with groups as listed herein under the definition of substituted amino. Examples include trimethysilyl, tert-butyldimethylsilyl, triphenylsilyl and the like.

Any definition herein may be used in combination with any other definition to describe a composite structural group. By convention, the trailing element of any such definition is that which attaches to the parent moiety. For example, the composite group alkylamido would represent an alkyl group attached to the parent molecule through an amido group, and the term alkoxyalkyl would represent an alkoxy group attached to the parent molecule through an alkyl group.

When a group is defined to be “null,” what is meant is that the group is absent.

The term “optionally substituted” means the anteceding group may be substituted or unsubstituted. When substituted, the substituents of an “optionally substituted” group may include, without limitation, one or more substituents independently selected from the following groups or a particular designated set of groups, alone or in combination: lower alkyl, lower alkenyl, lower alkynyl, lower alkanoyl, lower heteroalkyl, lower heterocycloalkyl, lower haloalkyl, lower haloalkenyl, lower haloalkynyl, lower perhaloalkyl, lower perhaloalkoxy, lower cycloalkyl, phenyl, aryl, aryloxy, lower alkoxy, lower haloalkoxy, oxo, lower acyloxy, carbonyl, carboxyl, lower alkylcarbonyl, lower carboxyester, lower carboxamido, cyano, hydrogen, halogen, hydroxy, amino, lower alkylamino, arylamino, amido, nitro, thiol, lower alkylthio, lower haloalkylthio, lower perhaloalkylthio, arylthio, sulfonate, sulfonic acid, trisubstituted silyl, N₃, SH, SCH₃, C(O)CH₃, CO₂CH₃, CO₂H, pyridinyl, thiophene, furanyl, lower carbamate, and lower urea. Two substituents may be joined together to form a fused five-, six-, or seven-membered carbocyclic or heterocyclic ring consisting of zero to three heteroatoms, for example forming methylenedioxy or ethylenedioxy. An optionally substituted group may be unsubstituted (e.g., —CH₂CH₃), fully substituted (e.g., —CF₂CF₃), monosubstituted (e.g., —CH₂CH₂F) or substituted at a level anywhere in-between fully substituted and monosubstituted (e.g., —CH₂CF₃). Where substituents are recited without qualification as to substitution, both substituted and unsubstituted forms are encompassed. Where a substituent is qualified as “substituted,” the substituted form is specifically intended. Additionally, different sets of optional substituents to a particular moiety may be defined as needed; in these cases, the optional substitution will be as defined, often immediately following the phrase, “optionally substituted with.”

The term R or the term R′, appearing by itself and without a number designation, unless otherwise defined, refers to a moiety selected from the group consisting of hydrogen, alkyl, cycloalkyl, heteroalkyl, aryl, heteroaryl and heterocycloalkyl, any of which may be optionally substituted. Such R and R′groups should be understood to be optionally substituted as defined herein. Whether an R group has a number designation or not, every R group, including R, R′ and Rn where n=(1, 2, 3, . . . n), every substituent, and every term should be understood to be independent of every other in terms of selection from a group. Should any variable, substituent, or term (e.g. aryl, heterocycle, R, etc.) occur more than one time in a formula or generic structure, its definition at each occurrence is independent of the definition at every other occurrence. Those of skill in the art will further recognize that certain groups may be attached to a parent molecule or may occupy a position in a chain of elements from either end as written. Thus, by way of example only, an unsymmetrical group such as —C(O)N(R)— may be attached to the parent moiety at either the carbon or the nitrogen.

Asymmetric centers exist in the compounds disclosed herein. These centers are designated by the symbols “R” or “S,” depending on the configuration of substituents around the chiral carbon atom. It should be understood that the disclosure encompasses all stereochemical isomeric forms, including diastereomeric, enantiomeric, and epimeric forms, as well as d-isomers and 1-isomers, and mixtures thereof. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds of particular stereochemistry are either commercially available or can be made and resolved by techniques known in the art. Additionally, the compounds disclosed herein may exist as geometric isomers. The present disclosure includes all cis, trans, syn, anti, entgegen (E), and zusammen (Z) isomers as well as the appropriate mixtures thereof. Additionally, compounds may exist as tautomers; all tautomeric isomers are provided by this disclosure. Additionally, the compounds disclosed herein can exist in unsolvated as well as solvated forms with pharmaceutically acceptable solvents such as water, ethanol, and the like. In general, the solvated forms are considered equivalent to the unsolvated forms.

The term “bond” refers to a covalent linkage between two atoms, or two moieties when the atoms joined by the bond are considered part of larger substructure. A bond may be single, double, or triple unless otherwise specified. A dashed line between two atoms in a drawing of a molecule indicates that an additional bond may be present or absent at that position.

The term “disease” as used herein is intended to be generally synonymous, and is used interchangeably with, the terms “disorder,” “syndrome,” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The term “combination therapy” means the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the conditions or disorders described herein.

GLS1 inhibitor is used herein to refer to a compound that exhibits an IC50 with respect to GLS1 activity of no more than about 100 μM and more typically not more than about 50 μM, as measured in the GLS1 enzyme assay described generally herein below. IC50 is that concentration of inhibitor that reduces the activity of an enzyme (e.g., GLS1) to half-maximal level. Certain compounds disclosed herein have been discovered to exhibit inhibition against GLS1. In certain embodiments, compounds will exhibit an IC50 with respect to GLS1 of no more than about 10 μM; in further embodiments, compounds will exhibit an IC50 with respect to GLS1 of no more than about 5 μM; in yet further embodiments, compounds will exhibit an IC50 with respect to GLS1 of not more than about 1 μM; in yet further embodiments, compounds will exhibit an IC50 with respect to GLS1 of not more than about 200 nM, as measured in the GLS1 binding assay described herein.

The phrase “therapeutically effective” is intended to qualify the amount of active ingredients used in the treatment of a disease or disorder or on the effecting of a clinical endpoint.

The term “therapeutically acceptable” refers to those compounds (or salts, prodrugs, tautomers, zwitterionic forms, etc.) which are suitable for use in contact with the tissues of patients without undue toxicity, irritation, and allergic response, are commensurate with a reasonable benefit/risk ratio, and are effective for their intended use.

As used herein, reference to “treatment” of a patient is intended to include prophylaxis. Treatment may also be preemptive in nature, i.e., it may include prevention of disease. Prevention of a disease may involve complete protection from disease, for example as in the case of prevention of infection with a pathogen, or may involve prevention of disease progression. For example, prevention of a disease may not mean complete foreclosure of any effect related to the diseases at any level, but instead may mean prevention of the symptoms of a disease to a clinically significant or detectable level. Prevention of diseases may also mean prevention of progression of a disease to a later stage of the disease.

The term “patient” is generally synonymous with the term “subject” and includes all mammals including humans. Examples of patients include humans, livestock such as cows, goats, sheep, pigs, and rabbits, and companion animals such as dogs, cats, rabbits, and horses. Preferably, the patient is a human.

The term “prodrug” refers to a compound that is made more active in vivo. Certain compounds disclosed herein may also exist as prodrugs, as described in Hydrolysis in Drug and Prodrug Metabolism: Chemistry, Biochemistry, and Enzymology (Testa, Bernard and Mayer, Joachim M. Wiley-VHCA, Zurich, Switzerland 2003). Prodrugs of the compounds described herein are structurally modified forms of the compound that readily undergo chemical changes under physiological conditions to provide the compound. Additionally, prodrugs can be converted to the compound by chemical or biochemical methods in an ex vivo environment. For example, prodrugs can be slowly converted to a compound when placed in a transdermal patch reservoir with a suitable enzyme or chemical reagent. Prodrugs are often useful because, in some situations, they may be easier to administer than the compound, or parent drug. They may, for instance, be bioavailable by oral administration whereas the parent drug is not. The prodrug may also have improved solubility in pharmaceutical compositions over the parent drug. A wide variety of prodrug derivatives are known in the art, such as those that rely on hydrolytic cleavage or oxidative activation of the prodrug. An example, without limitation, of a prodrug would be a compound which is administered as an ester (the “prodrug”), but then is metabolically hydrolyzed to the carboxylic acid, the active entity. Additional examples include peptidyl derivatives of a compound.

The compounds disclosed herein can exist as therapeutically acceptable salts. The present disclosure includes compounds listed above in the form of salts, including acid addition salts. Suitable salts include those formed with both organic and inorganic acids. Such acid addition salts will normally be pharmaceutically acceptable. However, salts of non-pharmaceutically acceptable salts may be of utility in the preparation and purification of the compound in question. Basic addition salts may also be formed and be pharmaceutically acceptable. For a more complete discussion of the preparation and selection of salts, refer to Pharmaceutical Salts: Properties, Selection, and Use (Stahl, P. Heinrich. Wiley-VCHA, Zurich, Switzerland, 2002).

The term “therapeutically acceptable salt,” as used herein, represents salts or zwitterionic forms of the compounds disclosed herein which are water or oil-soluble or dispersible and therapeutically acceptable as defined herein. The salts can be prepared during the final isolation and purification of the compounds or separately by reacting the appropriate compound in the form of the free base with a suitable acid. Representative acid addition salts include acetate, adipate, alginate, L-ascorbate, aspartate, benzoate, benzenesulfonate (besylate), bisulfate, butyrate, camphorate, camphorsulfonate, citrate, digluconate, formate, fumarate, gentisate, glutarate, glycerophosphate, glycolate, hemisulfate, heptanoate, hexanoate, hippurate, hydrochloride, hydrobromide, hydroiodide, 2-hydroxyethansulfonate (isethionate), lactate, maleate, malonate, DL-mandelate, mesitylenesulfonate, methanesulfonate, naphthylenesulfonate, nicotinate, 2-naphthalenesulfonate, oxalate, pamoate, pectinate, persulfate, 3-phenylproprionate, phosphonate, picrate, pivalate, propionate, pyroglutamate, succinate, sulfonate, tartrate, L-tartrate, trichloroacetate, trifluoroacetate, phosphate, glutamate, bicarbonate, para-toluenesulfonate (p-tosylate), and undecanoate. Also, basic groups in the compounds disclosed herein can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable addition salts include inorganic acids such as hydrochloric, hydrobromic, sulfuric, and phosphoric, and organic acids such as oxalic, maleic, succinic, and citric. Salts can also be formed by coordination of the compounds with an alkali metal or alkaline earth ion. Hence, the present disclosure contemplates sodium, potassium, magnesium, and calcium salts of the compounds disclosed herein, and the like.

Basic addition salts can be prepared during the final isolation and purification of the compounds by reacting a carboxy group with a suitable base such as the hydroxide, carbonate, or bicarbonate of a metal cation or with ammonia or an organic primary, secondary, or tertiary amine. The cations of therapeutically acceptable salts include lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N′-dibenzylethylenediamine. Other representative organic amines useful for the formation of base addition salts include ethylenediamine, ethanolamine, diethanolamine, piperidine, and piperazine.

A salt of a compound can be made by reacting the appropriate compound in the form of the free base with the appropriate acid.

### Compounds

The present disclosure provides a compound of structural Formula I

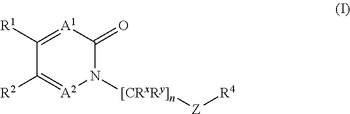

or a salt thereof, wherein: n is chosen from 3, 4, and 5; each R^{x} and R^{y} is independently chosen from alkyl, cyano, H, and halo, wherein two R^{x} groups together with the atoms to which they are attached optionally form a cycloalkyl ring; A¹ and A² are independently chosen from C—H, C—F, and N; R¹ and R⁴ are independently chosen from alkenyl, alkoxy, alkyl, aryl, arylalkyl, cyano, cycloalkyl, cycloalkylalkyl, H, halo, haloalkyl, heteroaryl, heteroarylalkyl, heterocycloalkyl, heterocycloalkylalkyl, hydroxyl, C(R³)₂C(O)R³, C(R³)₂C(O)N(R³)₂, C(R³)₂N(R³)₂, C(R³)₂NR³C(O)R³, C(R³)₂NR³C(O)OR³, C(R³)₂NR³C(O)N(R³)₂, C(R³)₂NR³S(O)R³, C(R³)₂NR³S(O)₂R³, N(R³)₂, NR³C(O)R³, NR³C(O)OR³, NR³C(O)N(R³)₂, NR³S(O)R³, NR³S(O)₂R³, C(O)N(R³)₂, S(O)N(R³)₂, S(O)₂N(R³)₂, C(O)R³, SR³, S(O)R³, and S(O)₂R³, wherein each R¹ and R⁴ may be optionally substituted with between 0 and 3 R^{z} groups; R² is chosen from alkyl, heterocycloalkyl, cyano, cycloalkyl, H, halo, and haloalkyl, wherein R¹ and R² together with the atoms to which they are attached optionally form an form an aryl, cycloalkyl, heteroaryl, or heterocycloalkyl ring, which may be optionally substituted with between 0 and 3 R^{z} groups; each R³ is independently chosen from alkenyl, alkoxy, alkyl, aryl, arylalkyl, cyano, cycloalkyl, cycloalkylalkyl, H, halo, haloalkyl, heteroaryl, heteroarylalkyl, heterocycloalkyl, heterocycloalkylalkyl, and hydroxyl, wherein each R³ may be optionally substituted with between 0 and 3 R^{z} groups, wherein two R³ groups together with the atoms to which they are attached optionally form an aryl, cycloalkyl, heteroaryl, or heterocycloalkyl ring, which may be optionally substituted with between 0 and 3 R^{z} groups; each R^{Z} group is independently chosen from alkenyl, alkoxy, alkoxyalkyl, alkoxyaryl, alkoxyarylalkyl, alkoxycycloalkyl, alkoxycycloalkylalkyl, alkoxyhaloalkyl, alkoxyheteroaryl, alkoxyheteroarylalkyl, alkoxyheterocycloalkyl, alkoxyheterocycloalkylalkyl, alkyl, alkylaryl, alkylarylalkyl, alkylcycloalkyl, alkylcycloalkylalkyl, alkylheteroaryl, alkylheteroarylalkyl, alkylheterocycloalkyl, alkylheterocycloalkylalkyl, aryl, arylalkyl, arylalkyloxy, arylhaloalkyl, aryloxy, cyano, cycloalkyl, cycloalkylalkyl, cycloalkylalkyloxy, cycloalkylhaloalkyl, cycloalkyloxy, H, halo, haloalkoxy, haloalkoxyalkyl, haloalkoxyaryl, haloalkoxyarylalkyl, haloalkoxycycloalkyl, haloalkoxycycloalkylalkyl, haloalkoxyheteroaryl, haloalkoxyheteroarylalkyl, haloalkoxyheterocycloalkyl, haloalkoxyheterocycloalkylalkyl, haloalkyl, haloalkylaryl, haloalkylarylalkyl, haloalkylcycloalkyl, haloalkylcycloalkylalkyl, haloalkylheteroaryl, haloalkylheteroarylalkyl, haloalkylheterocycloalkyl, haloalkylheterocycloalkylalkyl, haloaryl, haloarylalkyl, haloarylalkyloxy, haloaryloxy, halocycloalkyl, halocycloalkylalkyl, halocycloalkylalkyloxy, halocycloalkyloxy, haloheteroaryl, haloheteroarylalkyl, haloheteroarylalkyloxy, haloheteroaryloxy, haloheterocycloalkyl, haloheterocycloalkylalkyl, haloheterocycloalkylalkyloxy, haloheterocycloalkyloxy, heteroaryl, heteroarylalkyl, heteroarylalkyloxy, heteroarylhaloalkyl, heteroaryloxy, heterocycloalkyl, heterocycloalkylalkyl, heterocycloalkylalkyloxy, heterocycloalkylhaloalkyl, heterocycloalkyloxy, hydroxyl, oxo, N(R⁶)₂, NR⁶C(O)C(R⁶)₃, NR⁶C(O)OC(R⁶)₃, NR⁶C(O)N(R⁶)₂, NR⁶S(O)C(R⁶)₃, NR⁶S(O)₂C(R⁶)₃, C(O)N(R⁶)₂, S(O)N(R⁶)₂, S(O)₂N(R⁶)₂, C(O)C(R⁶)₃, SC(R⁶)₃, S(O)C(R⁶)₃, and S(O)₂C(R⁶)₃; each R⁶ is independently chosen from alkenyl, alkoxy, alkyl, aryl, arylalkyl, cyano, cycloalkyl, cycloalkylalkyl, H, halo, haloalkyl, heteroaryl, heteroarylalkyl, heterocycloalkyl, heterocycloalkylalkyl, and hydroxyl, wherein two R⁶ groups together with the atoms to which they are attached optionally form an aryl, cycloalkyl, heteroaryl, or heterocycloalkyl ring, which may be optionally substituted with between 0 and 3 R^{x} groups; and Z is heteroaryl, which may be optionally substituted.

In some embodiments, Z is a 5-6 membered monocyclic or 9-10 membered bicyclic heteroaryl, either of which contains one to four heteroatoms chosen from N, O, and S, and either of which may optionally be substituted by one to three substituents chosen from lower alkyl, halogen, CF₃, OCF₃, cyano, and hydroxyl.

In some embodiments, the compound, or a pharmaceutically acceptable salt thereof, has Formula II:

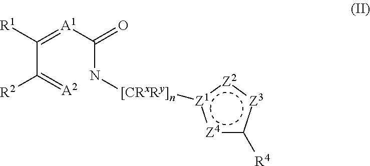

or a salt thereof, wherein; n is chosen from 3, 4, and 5; each R^{x} and R^{y} is independently chosen from alkyl, cyano, H, and halo, wherein two R^{x} groups together with the atoms to which they are attached optionally form a cycloalkyl ring; A¹ and A² are independently chosen from C—H, C—F, and N; Z¹ is chosen from C and N; Z², Z³, and Z⁴ are independently chosen from N, O, S, and CH, wherein at least one of Z¹, Z², Z³, and Z⁴ is chosen from N, O, and S; R¹ and R⁴ are independently chosen from alkenyl, alkoxy, alkyl, aryl, arylalkyl, cyano, cycloalkyl, cycloalkylalkyl, H, halo, haloalkyl, heteroaryl, heteroarylalkyl, heterocycloalkyl, heterocycloalkylalkyl, hydroxyl, C(R³)₂C(O)R³, C(R³)₂C(O)N(R³)₂, C(R³)₂N(R³)₂, C(R³)₂NR³C(O)R³, C(R³)₂NR³C(O)OR³, C(R³)₂NR³C(O)N(R³)₂, C(R³)₂NR³S(O)R³, C(R³)₂NR³S(O)₂R³, N(R³)₂, NR³C(O)R³, NR³C(O)OR³, NR³C(O)N(R³)₂, NR³S(O)R³, NR³S(O)₂R³, C(O)N(R³)₂, S(O)N(R³)₂, S(O)₂N(R³)₂, C(O)R³, SR³, S(O)R³, and S(O)₂R³, wherein each R¹ and R⁴ may be optionally substituted with between 0 and 3 R^{z} groups; R² is chosen from alkyl, heterocycloalkyl, cyano, cycloalkyl, H, halo, and haloalkyl, wherein R¹ and R² together with the atoms to which they are attached optionally form an form an aryl, cycloalkyl, heteroaryl, or heterocycloalkyl ring, which may be optionally substituted with between 0 and 3 R^{z} groups; each R³ is independently chosen from alkenyl, alkoxy, alkyl, aryl, arylalkyl, cyano, cycloalkyl, cycloalkylalkyl, H, halo, haloalkyl, heteroaryl, heteroarylalkyl, heterocycloalkyl, heterocycloalkylalkyl, and hydroxyl, wherein each R³ may be optionally substituted with between 0 and 3 R^{z} groups, wherein two R³ groups together with the atoms to which they are attached optionally form an aryl, cycloalkyl, heteroaryl, or heterocycloalkyl ring, which may be optionally substituted with between 0 and 3 R^{z} groups; each R^{Z} group is independently chosen from alkenyl, alkoxy, alkoxyalkyl, alkoxyaryl, alkoxyarylalkyl, alkoxycycloalkyl, alkoxycycloalkylalkyl, alkoxyhaloalkyl, alkoxyheteroaryl, alkoxyheteroarylalkyl, alkoxyheterocycloalkyl, alkoxyheterocycloalkylalkyl, alkyl, alkylaryl, alkylarylalkyl, alkylcycloalkyl, alkylcycloalkylalkyl, alkylheteroaryl, alkylheteroarylalkyl, alkylheterocycloalkyl, alkylheterocycloalkylalkyl, aryl, arylalkyl, arylalkyloxy, arylhaloalkyl, aryloxy, cyano, cycloalkyl, cycloalkylalkyl, cycloalkylalkyloxy, cycloalkylhaloalkyl, cycloalkyloxy, H, halo, haloalkoxy, haloalkoxyalkyl, haloalkoxyaryl, haloalkoxyarylalkyl, haloalkoxycycloalkyl, haloalkoxycycloalkylalkyl, haloalkoxyheteroaryl, haloalkoxyheteroarylalkyl, haloalkoxyheterocycloalkyl, haloalkoxyheterocycloalkylalkyl, haloalkyl, haloalkylaryl, haloalkylarylalkyl, haloalkylcycloalkyl, haloalkylcycloalkylalkyl, haloalkylheteroaryl, haloalkylheteroarylalkyl, haloalkylheterocycloalkyl, haloalkylheterocycloalkylalkyl, haloaryl, haloarylalkyl, haloarylalkyloxy, haloaryloxy, halocycloalkyl, halocycloalkylalkyl, halocycloalkylalkyloxy, halocycloalkyloxy, haloheteroaryl, haloheteroarylalkyl, haloheteroarylalkyloxy, haloheteroaryloxy, haloheterocycloalkyl, haloheterocycloalkylalkyl, haloheterocycloalkylalkyloxy, haloheterocycloalkyloxy, heteroaryl, heteroarylalkyl, heteroarylalkyloxy, heteroarylhaloalkyl, heteroaryloxy, heterocycloalkyl, heterocycloalkylalkyl, heterocycloalkylalkyloxy, heterocycloalkylhaloalkyl, heterocycloalkyloxy, hydroxyl, oxo,N(R⁶)₂, NR⁶C(O)C(R⁶)₃, NR⁶C(O)OC(R⁶)₃, NR⁶C(O)N(R⁶)₂, NR⁶S(O)C(R⁶)₃, NR⁶S(O)₂C(R⁶)₃, C(O)N(R⁶)₂, S(O)N(R⁶)₂, S(O)₂N(R⁶)₂, C(O)C(R⁶)₃, SC(R⁶)₃, S(O)C(R⁶)₃, and S(O)₂C(R⁶)₃; and each R⁶ is independently chosen from alkenyl, alkoxy, alkyl, aryl, arylalkyl, cyano, cycloalkyl, cycloalkylalkyl, H, halo, haloalkyl, heteroaryl, heteroarylalkyl, heterocycloalkyl, heterocycloalkylalkyl, and hydroxyl, wherein two R⁶ groups together with the atoms to which they are attached optionally form an aryl, cycloalkyl, heteroaryl, or heterocycloalkyl ring, which may be optionally substituted with between 0 and 3 R^{x} groups

In certain embodiments, n is 4; and A¹ and A² are CH.

In certain embodiments, n is 4; A¹ is N; and A² are CH.

In certain embodiments, Z₁ is C; Z₂ and Z₃ are N; Z₄ is S; and R⁴ is chosen from N(R³)₂, NR³C(O)C(R³)₃, NR³C(O)OC(R³)₃, and NR³C(O)N(R³)₂.

In certain embodiments, n is 4; A¹ and A² are CH; Z¹ is C; Z² and Z³ are N; Z⁴ is S; and R⁴ is chosen from N(R³)₂, NR³C(O)C(R³)₃, NR³C(O)OC(R³)₃, and NR³C(O)N(R³)₂.

In certain embodiments, Z¹ is C; Z² and Z³ are N; Z⁴ is S; and R⁴ is C(O)N(R³)₂.

In certain embodiments, n is 4; A and A² are CH; Z¹ is C; Z² and Z³ are N; Z⁴ is S; and R⁴ is C(O)N(R³)₂.

In certain embodiments, Z¹, Z², and Z³ are N; Z⁴ is CH; and R⁴ is chosen from N(R³)₂, NR³C(O)C(R³)₃, NR³C(O)OC(R³)₃, and NR³C(O)N(R³)₂.

In certain embodiments, n is 4; A and A² are CH; Z¹, Z², and Z³ are N; Z⁴ is CH; and R⁴ is chosen from N(R³)₂, NR³C(O)C(R³)₃, NR³C(O)OC(R³)₃, and NR³C(O)N(R³)₂.

In certain embodiments, Z¹, Z², and Z³ are N; Z⁴ is CH; and R⁴ is C(O)N(R³)₂.

In certain embodiments, n is 4; A and A² are CH; Z¹, Z², and Z³ are N; Z⁴ is CH; and R⁴ is C(O)N(R³)₂.

In some embodiments, the compound, or a pharmaceutically acceptable salt thereof, Formula III:

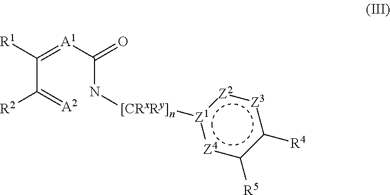

or a salt thereof, wherein: n is chosen from 3, 4, and 5; each R^{x} and R^{y} is independently chosen from alkyl, cyano, H, and halo, wherein two R^{x} groups together with the atoms to which they are attached optionally form a cycloalkyl ring; A¹ and A² are independently chosen from C—H, C—F, and N; Z¹ is chosen from C and N; Z² is chosen from N, CH, and C(O); Z³, and Z⁴ are independently chosen from N and CH, wherein at least one of Z¹, Z², Z³, and Z⁴ is N; R¹ and R⁴ are independently chosen from alkenyl, alkoxy, alkyl, aryl, arylalkyl, cyano, cycloalkyl, cycloalkylalkyl, H, halo, haloalkyl, heteroaryl, heteroarylalkyl, heterocycloalkyl, heterocycloalkylalkyl, hydroxyl, C(R³)₂C(O)R³, C(R³)₂C(O)N(R³)₂, C(R³)₂N(R³)₂, C(R³)₂NR³C(O)R³, C(R³)₂NR³C(O)OR³, C(R³)₂NR³C(O)N(R³)₂, C(R³)₂NR³S(O)R³, C(R³)₂NR³S(O)₂R³, N(R³)₂, NR³C(O)R³, NR³C(O)OR³, NR³C(O)N(R³)₂, NR³S(O)R³, NR³S(O)₂R³, C(O)N(R³)₂, S(O)N(R³)₂, S(O)₂N(R³)₂, C(O)R³, SR³, S(O)R³, and S(O)₂R³, wherein each R¹ and R⁴ may be optionally substituted with between 0 and 3 R^{z} groups; R² and R⁵ are chosen from alkyl, heterocycloalkyl, cyano, cycloalkyl, H, halo, and haloalkyl, wherein R¹ and R² together with the atoms to which they are attached optionally form an form an aryl, cycloalkyl, heteroaryl, or heterocycloalkyl ring, which may be optionally substituted with between 0 and 3 R^{z} groups, wherein R⁴ and R⁵ together with the atoms to which they are attached optionally form an aryl, cycloalkyl, heteroaryl, or heterocycloalkyl ring, which may be optionally substituted with between 0 and 3 R^{z} groups; each R³ is independently chosen from alkenyl, alkoxy, alkyl, aryl, arylalkyl, cyano, cycloalkyl, cycloalkylalkyl, H, halo, haloalkyl, heteroaryl, heteroarylalkyl, heterocycloalkyl, heterocycloalkylalkyl, and hydroxyl, wherein each R³ may be optionally substituted with between 0 and 3 R^{z} groups, wherein two R³ groups together with the atoms to which they are attached optionally form an aryl, cycloalkyl, heteroaryl, or heterocycloalkyl ring, which may be optionally substituted with between 0 and 3 R^{z} groups; each R^{Z} group is independently chosen from alkenyl, alkoxy, alkoxyalkyl, alkoxyaryl, alkoxyarylalkyl, alkoxycycloalkyl, alkoxycycloalkylalkyl, alkoxyhaloalkyl, alkoxyheteroaryl, alkoxyheteroarylalkyl, alkoxyheterocycloalkyl, alkoxyheterocycloalkylalkyl, alkyl, alkylaryl, alkylarylalkyl, alkylcycloalkyl, alkylcycloalkylalkyl, alkylheteroaryl, alkylheteroarylalkyl, alkylheterocycloalkyl, alkylheterocycloalkylalkyl, aryl, arylalkyl, arylalkyloxy, arylhaloalkyl, aryloxy, cyano, cycloalkyl, cycloalkylalkyl, cycloalkylalkyloxy, cycloalkylhaloalkyl, cycloalkyloxy, H, halo, haloalkoxy, haloalkoxyalkyl, haloalkoxyaryl, haloalkoxyarylalkyl, haloalkoxycycloalkyl, haloalkoxycycloalkylalkyl, haloalkoxyheteroaryl, haloalkoxyheteroarylalkyl, haloalkoxyheterocycloalkyl, haloalkoxyheterocycloalkylalkyl, haloalkyl, haloalkylaryl, haloalkylarylalkyl, haloalkylcycloalkyl, haloalkylcycloalkylalkyl, haloalkylheteroaryl, haloalkylheteroarylalkyl, haloalkylheterocycloalkyl, haloalkylheterocycloalkylalkyl, haloaryl, haloarylalkyl, haloarylalkyloxy, haloaryloxy, halocycloalkyl, halocycloalkylalkyl, halocycloalkylalkyloxy, halocycloalkyloxy, haloheteroaryl, haloheteroarylalkyl, haloheteroarylalkyloxy, haloheteroaryloxy, haloheterocycloalkyl, haloheterocycloalkylalkyl, haloheterocycloalkylalkyloxy, haloheterocycloalkyloxy, heteroaryl, heteroarylalkyl, heteroarylalkyloxy, heteroarylhaloalkyl, heteroaryloxy, heterocycloalkyl, heterocycloalkylalkyl, heterocycloalkylalkyloxy, heterocycloalkylhaloalkyl, heterocycloalkyloxy, hydroxyl, oxo, N(R⁶)₂, NR⁶C(O)C(R⁶)₃, NR⁶C(O)OC(R⁶)₃, NR⁶C(O)N(R⁶)₂, NR⁶S(O)C(R⁶)₃, NR⁶S(O)₂C(R⁶)₃, C(O)N(R⁶)₂, S(O)N(R⁶)₂, S(O)₂N(R⁶)₂, C(O)C(R⁶)₃, SC(R⁶)₃, S(O)C(R⁶)₃, and S(O)₂C(R⁶)₃; and each R⁶ is independently chosen from alkenyl, alkoxy, alkyl, aryl, arylalkyl, cyano, cycloalkyl, cycloalkylalkyl, H, halo, haloalkyl, heteroaryl, heteroarylalkyl, heterocycloalkyl, heterocycloalkylalkyl, and hydroxyl, wherein two R⁶ groups together with the atoms to which they are attached optionally form an aryl, cycloalkyl, heteroaryl, or heterocycloalkyl ring, which may be optionally substituted with between 0 and 3 R^{x} groups.

In certain embodiments, n is 4; and A¹ and A² are CH.

In certain embodiments, n is 4; A¹ is N; and A² are CH.

In certain embodiments, Z₁ is C; Z₂ and Z₃ are N; Z⁴ is CH; R⁴ is chosen from N(R³)₂, NR³C(O)C(R³)₃, NR³C(O)OC(R³)₃, and NR³C(O)N(R³)₂; and R⁵ is H.

In certain embodiments, n is 4; A¹ and A² are CH; Z¹ is C; Z² and Z³ are N; Z⁴ is CH; R⁴ is chosen from N(R³)₂, NR³C(O)C(R³)₃, NR³C(O)OC(R³)₃, and NR³C(O)N(R³)₂; and R⁵ is H.

In certain embodiments, Z¹ is C; Z² and Z³ are N; Z⁴ is CH; R⁴ is C(O)N(R³)₂; and R⁵ is H.

In certain embodiments, n is 4; A and A² are CH; Z¹ is C; Z² and Z³ are N; Z⁴ is CH; R⁴ is C(O)N(R³)₂; and R⁵ is H.

In certain embodiments, Z¹ is N; Z² is C(O); Z⁴ is CH; R⁴ is chosen from N(R³)₂, NR³C(O)C(R³)₃, NR³C(O)OC(R³)₃, and NR³C(O)N(R³)₂; and R⁵ is H.

In certain embodiments, n is 4; A and A² are CH; Z¹ is N; Z² is C(O); Z⁴ is CH; R⁴ is chosen from N(R³)₂, NR³C(O)C(R³)₃, NR³C(O)OC(R³)₃, and NR³C(O)N(R³)₂; and R⁵ is H.

In certain embodiments, Z¹ is N; Z² is C(O); Z⁴ is CH; R⁴ is C(O)N(R³)₂; and R⁵ is H.

In certain embodiments, n is 4; and A¹ and A² are CH; Z¹ is N; Z² is C(O); Z⁴ is CH; R⁴ is C(O)N(R³)₂; and R⁵ is H.

Also provided are embodiments wherein any of embodiment above in paragraphs [0006] and [00107]-[0131] above may be combined with any one or more of these embodiments, provided the combination is not mutually exclusive.

### Pharmaceutical Compositions

While it may be possible for the compounds of the subject disclosure to be administered as the raw chemical, it is also possible to present them as a pharmaceutical formulation. Accordingly, provided herein are pharmaceutical formulations which comprise one or more of certain compounds disclosed herein, or one or more pharmaceutically acceptable salts, esters, prodrugs, amides, or solvates thereof, together with one or more pharmaceutically acceptable carriers thereof and optionally one or more other therapeutic ingredients. The carrier(s) must be “acceptable” in the sense of being compatible with the other ingredients of the formulation and not deleterious to the recipient thereof. Proper formulation is dependent upon the route of administration chosen. Any of the well-known techniques, carriers, and excipients may be used as suitable and as understood in the art; e.g., in Remington's Pharmaceutical Sciences. The pharmaceutical compositions disclosed herein may be manufactured in any manner known in the art, e.g., by means of conventional mixing, dissolving, granulating, dragee-making, levigating, emulsifying, encapsulating, entrapping or compression processes.

The formulations include those suitable for oral, parenteral (including subcutaneous, intradermal, intramuscular, intravenous, intraarticular, and intramedullary), intraperitoneal, transmucosal, transdermal, rectal and topical (including dermal, buccal, sublingual and intraocular) administration although the most suitable route may depend upon for example the condition and disorder of the recipient. The formulations may conveniently be presented in unit dosage form and may be prepared by any of the methods well known in the art of pharmacy.

Typically, these methods include the step of bringing into association a compound of the subject disclosure or a pharmaceutically acceptable salt, ester, amide, prodrug or solvate thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

Compounds described herein can be administered as follows:

### Oral Administration

The compounds of the present invention may be administered orally, including swallowing, so the compound enters the gastrointestinal tract, or is absorbed into the blood stream directly from the mouth, including sublingual or buccal administration.

Suitable compositions for oral administration include solid formulations such as tablets, pills, cachets, lozenges and hard or soft capsules, which can contain liquids, gels, powders, or granules.

In a tablet or capsule dosage form the amount of drug present may be from about 0.05% to about 95% by weight, more typically from about 2% to about 50% by weight of the dosage form.

In addition, tablets or capsules may contain a disintegrant, comprising from about 0.5% to about 35% by weight, more typically from about 2% to about 25% of the dosage form. Examples of disintegrants include methyl cellulose, sodium or calcium carboxymethyl cellulose, croscarmellose sodium, polyvinylpyrrolidone, hydroxypropyl cellulose, starch and the like.

Suitable binders, for use in a tablet, include gelatin, polyethylene glycol, sugars, gums, starch, hydroxypropyl cellulose and the like. Suitable diluents, for use in a tablet, include mannitol, xylitol, lactose, dextrose, sucrose, sorbitol and starch.

Suitable surface active agents and glidants, for use in a tablet or capsule, may be present in amounts from about 0.1% to about 3% by weight, and include polysorbate 80, sodium dodecyl sulfate, talc and silicon dioxide.

Suitable lubricants, for use in a tablet or capsule, may be present in amounts from about 0.1% to about 5% by weight, and include calcium, zinc or magnesium stearate, sodium stearyl fumarate and the like.

Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with a liquid diluent. Dyes or pigments may be added to tablets for identification or to characterize different combinations of active compound doses.

Liquid formulations can include emulsions, solutions, syrups, elixirs and suspensions, which can be used in soft or hard capsules. Such formulations may include a pharmaceutically acceptable carrier, for example, water, ethanol, polyethylene glycol, cellulose, or an oil. The formulation may also include one or more emulsifying agents and/or suspending agents.

Compositions for oral administration may be formulated as immediate or modified release, including delayed or sustained release, optionally with enteric coating.

In another embodiment, a pharmaceutical composition comprises a therapeutically effective amount of a compound of Formula (I) or a pharmaceutically acceptable salt thereof, and a pharmaceutically acceptable carrier.

### Parenteral Administration

Compounds of the present invention may be administered directly into the blood stream, muscle, or internal organs by injection, e.g., by bolus injection or continuous infusion.

Suitable means for parenteral administration include intravenous, intra-muscular, subcutaneous intraarterial, intraperitoneal, intrathecal, intracranial, and the like. Suitable devices for parenteral administration include injectors (including needle and needle-free injectors) and infusion methods. The formulations may be presented in unit-dose or multi-dose containers, for example sealed ampoules and vials.

Most parenteral formulations are aqueous solutions containing excipients, including salts, buffering, suspending, stabilizing and/or dispersing agents, antioxidants, bacteriostats, preservatives, and solutes which render the formulation isotonic with the blood of the intended recipient, and carbohydrates.

Parenteral formulations may also be prepared in a dehydrated form (e.g., by lyophilization) or as sterile non-aqueous solutions. These formulations can be used with a suitable vehicle, such as sterile water. Solubility-enhancing agents may also be used in preparation of parenteral solutions.

Compositions for parenteral administration may be formulated as immediate or modified release, including delayed or sustained release. Compounds may also be formulated as depot preparations. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

### Topical Administration

Compounds of the present invention may be administered topically (for example to the skin, mucous membranes, ear, nose, or eye) or transdermally. Formulations for topical administration can include, but are not limited to, lotions, solutions, creams, gels, hydrogels, ointments, foams, implants, patches and the like. Carriers that are pharmaceutically acceptable for topical administration formulations can include water, alcohol, mineral oil, glycerin, polyethylene glycol and the like. Topical administration can also be performed by, for example, electroporation, iontophoresis, phonophoresis and the like.

Typically, the active ingredient for topical administration may comprise from 0.001% to 10% w/w (by weight) of the formulation. In certain embodiments, the active ingredient may comprise as much as 10% w/w; less than 5% w/w; from 2% w/w to 5% w/w; or from 0.1% to 1% w/w of the formulation.

Compositions for topical administration may be formulated as immediate or modified release, including delayed or sustained release.

### Rectal, Buccal, and Sublingual Administration

Suppositories for rectal administration of the compounds of the present invention can be prepared by mixing the active agent with a suitable non-irritating excipient such as cocoa butter, synthetic mono-, di-, or triglycerides, fatty acids, or polyethylene glycols which are solid at ordinary temperatures but liquid at the rectal temperature, and which will therefore melt in the rectum and release the drug.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

### Administration by Inhalation

For administration by inhalation, compounds may be conveniently delivered from an insufflator, nebulizer pressurized packs or other convenient means of delivering an aerosol spray or powder. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, the compounds according to the disclosure may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

Other carrier materials and modes of administration known in the pharmaceutical art may also be used. Pharmaceutical compositions of the invention may be prepared by any of the well-known techniques of pharmacy, such as effective formulation and administration procedures. Preferred unit dosage formulations are those containing an effective dose, as herein recited, or an appropriate fraction thereof, of the active ingredient. The precise amount of compound administered to a patient will be the responsibility of the attendant physician. The specific dose level for any particular patient will depend upon a variety of factors including the activity of the specific compound employed, the age, body weight, general health, sex, diets, time of administration, route of administration, rate of excretion, drug combination, the precise disorder being treated, and the severity of the indication or condition being treated. In addition, the route of administration may vary depending on the condition and its severity. The above considerations concerning effective formulations and administration procedures are well known in the art and are described in standard textbooks. Formulation of drugs is discussed in, for example, Hoover, John E., Remington's Pharmaceutical Sciences, Mack Publishing Co., Easton, Pa., 1975; Liberman, et al., Eds., Pharmaceutical Dosage Forms, Marcel Decker, New York, N. Y., 1980; and Kibbe, et al., Eds., Handbook of Pharmaceutical Excipients (3^{rd} Ed.), American Pharmaceutical Association, Washington, 1999.

### Methods of Treatment

The present disclosure provides compounds and pharmaceutical compositions that inhibit glutaminase activity, particularly GLS1 activity and are thus useful in the treatment or prevention of disorders associated with GLS1. Compounds and pharmaceutical compositions of the present disclosure selectively modulate GLS1 and are thus useful in the treatment or prevention of a range of disorders associated with GLS1 and include, but are not limited to, cancer, immunological or neurological diseases associated with GLS1.

### Neurological Disorders

In some embodiments, the compounds and pharmaceutical compositions of the present disclosure may be useful in the treatment or prevention of neurological diseases.

The most common neurotransmitter is glutamate, derived from the enzymatic conversion of glutamine via glutaminase. High levels of glutamate have been shown to be neurotoxic. Following traumatic insult to neuronal cells, there occurs a rise in neurotransmitter release, particularly glutamate. Accordingly, inhibition of glutaminase has been hypothesized as a means of treatment following an ischemic insult, such as stroke.

Huntington's disease is a progressive, fatal neurological condition. In genetic mouse models of Huntington's disease, it was observed that the early manifestation of the disease correlated with dysregulated glutamate release (Raymond et al., Neuroscience, 2011). In HIV-associated dementia, HIV infected macrophages exhibit upregulated glutaminase activity and increased glutamate release, leading to neuronal damage (Huang et al., J. Neurosci., 2011). Similarly, in another neurological disease, the activated microglia in Rett Syndrome release glutamate causing neuronal damage. The release of excess glutamate has been associated with the up-regulation of glutaminase (Maezawa et al., J. Neurosci, 2010). In mice bred to have reduced glutaminase levels, sensitivity to psychotic-stimulating drugs, such as amphetamines, was dramatically reduced, thus suggesting that glutaminase inhibition may be beneficial in the treatment of schizophrenia (Gaisler-Salomon et al., Neuropsychopharmacology, 2009). Bipolar disorder is a devastating illness that is marked by recurrent episodes of mania and depression. This disease is treated with mood stabilizers such as lithium and valproate; however, chronic use of these drugs appear to increase the abundance of glutamate receptors (Nanavati et al., J. Neurochem., 2011), which may lead to a decrease in the drug's effectiveness over time. Thus, an alternative treatment may be to reduce the amount of glutamate by inhibiting glutaminase. This may or may not be in conjunction with the mood stabilizers. Memantine, a partial antagonist of N-methyl-D-aspartate receptor (NMDAR), is an approved therapeutic in the treatment of Alzheimer's disease. Currently, research is being conducted looking at memantine as a means of treating vascular dementia and Parkinson's disease (Oliverares et al., Curr. Alzheimer Res., 2011). Since memantine has been shown to partially block the NMDA glutamate receptor also, it is not unresasonable to speculate that decreasing glutamate levels by inhibiting glutaminase could also treat Alzheimer's disease, vascular dementia and Parkinson's disease. Alzheimer's disease, bipolar disorder, HIV-associated dementia, Huntington's disease, ischemic insult, Parkinson's disease, schizophrenia, stroke, traumatic insult and vascular dementia are but a few of the neurological diseases that have been correlated to increased levels of glutamate. Thus, inhibiting glutaminase with a compound described herein can reduce or prevent neurological diseases. Therefore, in certain embodiments, the compounds may be used for the treatment or prevention of neurological diseases.

### Immunological Disorders

In some embodiments, the compounds and pharmaceutical compositions of the present disclosure may be useful in the treatment or prevention of immunological diseases.

Activation of T lymphocytes induces cell growth, proliferation, and cytokine production, thereby placing energetic and biosynthetic demands on the cell. Glutamine serves as an amine group donor for nucleotide synthesis, and glutamate, the first component in glutamine metabolism, plays a direct role in amino acid and glutathione synthesis, as well as being able to enter the Krebs cycle for energy production (Carr et al., J. Immunol., 2010). Mitogen-induced T cell proliferation and cytokine production require high levels of glutamine metabolism, thus inhibiting glutaminase may serve as a means of immune modulation. In multiple sclerosis, an inflammatory autoimmune disease, the activated microglia exhibit up-regulated glutaminase and release increased levels of extracellular glutamate. Glutamine levels are lowered by sepsis, injury, burns, surgery and endurance exercise (Calder et al., Amino Acids, 1999). These situations put the individual at risk of immunosuppression. In fact, in general, glutaminase gene expression and enzyme activity are both increased during T cell activity. Patients given glutamine following bone marrow transplantation resulted in a lower level of infection and reduced graft v. host disease (Crowther, Proc. Nutr. Soc., 2009). T cell proliferation and activiation is involved in many immunological diseases, such as inflammatory bowel disease, Crohn's disease, sepsis, psoriasis, arthritis (including rheumatoid arthritis), multiple sclerosis, graft v. host disease, infections, lupus and diabetes. In an embodiment of the invention, the compounds described herein can be used to treat or prevent immunological diseases.

### Cancer

In some embodiments, the compounds and pharmaceutical compositions of the present disclosure may be useful in the treatment or prevention of cancer.

In addition to serving as the basic building blocks of protein synthesis, amino acids have been shown to contribute to many processes critical for growing and dividing cells, and this is particularly true for cancer cells. Nearly all definitions of cancer include reference to dysregulated proliferation. Numerous studies on glutamine metabolism in cancer indicate that many tumors are avid glutamine consumers (Souba, Ann. Surg., 1993; Collins et al., J. Cell. Physiol., 1998; Medina, J. Nutr., 2001; Shanware et al., J. Mol. Med., 2011). An embodiment of the invention is the use of the compounds described herein for the treatment of cancer.

In some embodiments, the compounds of the present disclosure may be used to prevent or treat cancer, wherein the cancer is one or a variant of Acute Lymphoblastic Leukemia (ALL), Acute Myeloid Leukemia (AML), Adrenocortical Carcinoma, AIDS-Related Cancers (Kaposi Sarcoma and Lymphoma), Anal Cancer, Appendix Cancer, Atypical Teratoid/Rhabdoid Tumor, Basal Cell Carcinoma, Bile Duct Cancer (including Extrahepatic), Bladder Cancer, Bone Cancer (including Osteosarcoma and Malignant Fibrous Histiocytoma), Brain Tumor (such as Astrocytomas, Brain and Spinal Cord Tumors, Brain Stem Glioma, Central Nervous System Atypical Teratoid/Rhabdoid Tumor, Central Nervous System Embryonal Tumors, Craniopharyngioma, Ependymoblastoma, Ependymoma, Medulloblastoma, Medulloepithelioma, Pineal Parenchymal Tumors of Intermediate Differentiation, Supratentorial Primitive Neuroectodermal Tumors and Pineoblastoma), Breast Cancer, Bronchial Tumors, Burkitt Lymphoma, Basal Cell Carcinoma, Bile Duct Cancer (including Extrahepatic), Bladder Cancer, Bone Cancer (including Osteosarcoma and Malignant Fibrous Histiocytoma), Carcinoid Tumor, Carcinoma of Unknown Primary, Central Nervous System (such as Atypical Teratoid/Rhabdoid Tumor, Embryonal Tumors and Lymphoma), Cervical Cancer, Childhood Cancers, Chordoma, Chronic Lymphocytic Leukemia (CLL), Chronic Myelogenous Leukemia (CML), Chronic Myeloproliferative Disorders, Colon Cancer, Colorectal Cancer, Craniopharyngioma, Cutaneous T-Cell Lymphoma (Mycosis Fungoides and Sézary Syndrome), Duct, Bile (Extrahepatic), Ductal Carcinoma In Situ (DCIS), Embryonal Tumors (Central Nervous System), Endometrial Cancer, Ependymoblastoma, Ependymoma, Esophageal Cancer, Esthesioneuroblastoma, Ewing Sarcoma Family of Tumors, Extracranial Germ Cell Tumor, Extragonadal Germ Cell Tumor, Extrahepatic Bile Duct Cancer, Eye Cancer (like Intraocular Melanoma, Retinoblastoma), Fibrous Histiocytoma of Bone (including Malignant and Osteosarcoma) Gallbladder Cancer, Gastric (Stomach) Cancer, Gastrointestinal Carcinoid Tumor, Gastrointestinal Stromal Tumors (GIST), Germ Cell Tumor (Extracranial, Extragonadal, Ovarian), Gestational Trophoblastic Tumor, Glioma, Hairy Cell Leukemia, Head and Neck Cancer, Heart Cancer, Hepatocellular (Liver) Cancer, Histiocytosis, Langerhans Cell, Hodgkin Lymphoma, Hypopharyngeal Cancer, Intraocular Melanoma, Islet Cell Tumors (Endocrine, Pancreas), Kaposi Sarcoma, Kidney (including Renal Cell), Langerhans Cell Histiocytosis, Laryngeal Cancer, Leukemia (including Acute Lymphoblastic (ALL), Acute Myeloid (AML), Chronic Lymphocytic (CLL), Chronic Myelogenous (CML), Hairy Cell), Lip and Oral Cavity Cancer, Liver Cancer (Primary), Lobular Carcinoma In Situ (LCIS), Lung Cancer (Non-Small Cell and Small Cell), Lymphoma (AIDS-Related, Burkitt, Cutaneous T-Cell (Mycosis Fungoides and Sézary Syndrome), Hodgkin, Non-Hodgkin, Primary Central Nervous System (CNS), Macroglobulinemia, Waldenstrim, Male Breast Cancer, Malignant Fibrous Histiocytoma of Bone and Osteosarcoma, Medulloblastoma, Medulloepithelioma, Melanoma (including Intraocular (Eye)), Merkel Cell Carcinoma, Mesothelioma (Malignant), Metastatic Squamous Neck Cancer with Occult Primary, Midline Tract Carcinoma Involving NUT Gene, Mouth Cancer, Multiple Endocrine Neoplasia Syndromes, Multiple Myeloma/Plasma Cell Neoplasm, Mycosis Fungoides, Myelodysplastic Syndromes, Myelodysplastic/Myeloproliferative Neoplasms, Myelogenous Leukemia, Chronic (CML), Myeloid Leukemia, Acute (AML), Myeloma and Multiple Myeloma, Myeloproliferative Disorders (Chronic), Nasal Cavity and Paranasal Sinus Cancer, Nasopharyngeal Cancer, Neuroblastoma, Non-Hodgkin Lymphoma, Non-Small Cell Lung Cancer, Oral Cancer, Oral Cavity Cancer, Lip and, Oropharyngeal Cancer, Osteosarcoma and Malignant Fibrous Histiocytoma of Bone, Ovarian Cancer (such as Epithelial, Germ Cell Tumor, and Low Malignant Potential Tumor), Pancreatic Cancer (including Islet Cell Tumors), Papillomatosis, Paraganglioma, Paranasal Sinus and Nasal Cavity Cancer, Parathyroid Cancer, Penile Cancer, Pharyngeal Cancer, Pheochromocytoma, Pineal Parenchymal Tumors of Intermediate Differentiation, Pineoblastoma and Supraten 5 torial Primitive Neuroectodermal Tumors, Pituitary Tumor, Plasma Cell Neoplasm/Multiple Myeloma, Pleuropulmonary Blastoma, Pregnancy and Breast Cancer, Primary Central Nervous System (CNS) Lymphoma, Prostate Cancer, Rectal Cancer, Renal Cell (Kidney) Cancer, Renal Pelvis and Ureter, Transitional Cell Cancer, Retinoblastoma, Rhabdomyosarcoma, Salivary Gland Cancer, Sarcoma (like Ewing Sarcoma Family of Tumors, Kaposi, Soft Tissue, Uterine), Sézary Syndrome, Skin Cancer (such as Melanoma, Merkel Cell Carcinoma, Nonmelanoma), Small Cell Lung Cancer, Small Intestine Cancer, Soft Tissue Sarcoma, Squamous Cell Carcinoma, Squamous Neck Cancer with Occult Primary, Metastatic, Stomach (Gastric) Cancer, Supratentorial Primitive Neuroectodermal Tumors, T-Cell Lymphoma (Cutaneous, Mycosis Fungoides and Sézary Syndrome), Testicular Cancer, Throat Cancer, Thymoma and Thymic Carcinoma, Thyroid Cancer, Transitional Cell Cancer of the Renal Pelvis and Ureter, Trophoblastic Tumor (Gestational), Unknown Primary, Unusual Cancers of Childhood, Ureter and Renal Pelvis, Transitional Cell Cancer, Urethral Cancer, Uterine Cancer, Endometrial, Uterine Sarcoma, Waldenstrim Macroglobulinemia or Wilms Tumor.

In certain embodiments, the cancer to be treated is one specific to T-cells such as T-cell lymphomia and lymphblastic T-cell leukemia.

In some embodiments, methods described herein are used to treat a disease condition comprising administering to a subject in need thereof a therapeutically effective amount of a compound of Formula I or pharmaceutically acceptable salt thereof, wherein the condition is cancer which has developed resistance to chemotherapeutic drugs and/or ionizing radiation.

### Combinations and Combination Therapy

The compounds of the present invention can be used, alone or in combination with other pharmaceutically active compounds, to treat conditions such as those previously described hereinabove. The compound(s) of the present invention and other pharmaceutically active compound(s) can be administered simultaneously (either in the same dosage form or in separate dosage forms) or sequentially. Accordingly, in one embodiment, the present invention comprises methods for treating a condition by administering to the subject a therapeutically-effective amount of one or more compounds of the present invention and one or more additional pharmaceutically active compounds.

In another embodiment, there is provided a pharmaceutical composition comprising one or more compounds of the present invention, one or more additional pharmaceutically active compounds, and a pharmaceutically acceptable carrier.

In another embodiment, the one or more additional pharmaceutically active compounds is selected from the group consisting of anti-cancer drugs, anti-proliferative drugs, and anti-inflammatory drugs.

GLS1 inhibitor compositions described herein are also optionally used in combination with other therapeutic reagents that are selected for their therapeutic value for the condition to be treated. In general, the compounds described herein and, in embodiments where combination therapy is employed, other agents do not have to be administered in the same pharmaceutical composition and, because of different physical and chemical characteristics, are optionally administered by different routes. The initial administration is generally made according to established protocols and then, based upon the observed effects, the dosage, modes of administration and times of administration subsequently modified. In certain instances, it is appropriate to administer a GLS1 inhibitor compound, as described herein, in combination with another therapeutic agent. By way of example only, the therapeutic effectiveness of a GLS1 inhibitor is enhanced by administration of another therapeutic agent (which also includes a therapeutic regimen) that also has therapeutic benefit. Regardless of the disease, disorder or condition being treated, the overall benefit experienced by the patient is either simply additive of the two therapeutic agents or the patient experiences an enhanced (i.e., synergistic) benefit. Alternatively, if a compound disclosed herein has a side effect, it may be appropriate to administer an agent to reduce the side effect; or the therapeutic effectiveness of a compound described herein may be enhanced by administration of an adjuvant.

Therapeutically effective dosages vary when the drugs are used in treatment combinations. Methods for experimentally determining therapeutically effective dosages of drugs and other agents for use in combination treatment regimens are documented methodologies. Combination treatment further includes periodic treatments that start and stop at various times to assist with the clinical management of the patient. In any case, the multiple therapeutic agents (one of which is a GLS1 inhibitor as described herein) may be administered in any order, or simultaneously. If simultaneously, the multiple therapeutic agents are optionally provided in a single, unified form, or in multiple forms (by way of example only, either as a single pill or as two separate pills).

In some embodiments, one of the therapeutic agents is given in multiple doses, or both are given as multiple doses. If not simultaneous, the timing between the multiple doses optionally varies from more than zero weeks to less than twelve weeks.

In addition, the combination methods, compositions and formulations are not to be limited to the use of only two agents, the use of multiple therapeutic combinations are also envisioned. It is understood that the dosage regimen to treat, prevent, or ameliorate the condition(s) for which relief is sought, is optionally modified in accordance with a variety of factors. These factors include the disorder from which the subject suffers, as well as the age, weight, sex, diet, and medical condition of the subject. Thus, the dosage regimen actually employed varies widely, in some embodiments, and therefore deviates from the dosage regimens set forth herein.

The pharmaceutical agents which make up the combination therapy disclosed herein are optionally a combined dosage form or in separate dosage forms intended for substantially simultaneous administration. The pharmaceutical agents that make up the combination therapy are optionally also administered sequentially, with either agent being administered by a regimen calling for two-step administration. The two-step administration regimen optionally calls for sequential administration of the active agents or spaced-apart administration of the separate active agents. The time between the multiple administration steps ranges from a few minutes to several hours, depending upon the properties of each pharmaceutical agent, such as potency, solubility, bioavailability, plasma half-life and kinetic profile of the pharmaceutical agent.

In another embodiment, a GLS1 inhibitor is optionally used in combination with procedures that provide additional benefit to the patient. A GLS1 inhibitor and any additional therapies are optionally administered before, during or after the occurrence of a disease or condition, and the timing of administering the composition containing a GLS1 inhibitor varies in some embodiments. Thus, for example, a GLS1 inhibitor is used as a prophylactic and is administered continuously to subjects with a propensity to develop conditions or diseases in order to prevent the occurrence of the disease or condition. A GLS1 inhibitor and compositions are optionally administered to a subject during or as soon as possible after the onset of the symptoms. While embodiments of the present invention have been shown and described herein, it will be obvious to those skilled in the art that such embodiments are provided by way of example only. Numerous variations, changes, and substitutions will now occur to those skilled in the art without departing from the invention. It should be understood that in some embodiments of the invention various alternatives to the embodiments described herein are employed in practicing the invention.

A GLS1 inhibitor can be used in combination with anti-cancer drugs, including but not limited to the following classes: alkylating agents, anti-metabolites, plant alkaloids and terpenoids, topoisomerase inhibitors, cytotoxic antibiotics, angiogenesis inhibitors and tyrosine kinase inhibitors.

For use in cancer and neoplastic diseases a GLS1 inhibitor may be optimally used together with one or more of the following non-limiting examples of anti-cancer agents: (1) alkylating agents, including but not limited to cisplatin (PLATIN), carboplatin (PARAPLATIN), oxaliplatin (ELOXATIN), streptozocin (ZANOSAR), busulfan (MYLERAN) and cyclophosphamide (ENDOXAN); (2) anti-metabolites, including but not limited to mercaptopurine (PURINETHOL), thioguanine, pentostatin (NIPENT), cytosine arabinoside (ARA-C), gemcitabine (GEMZAR), fluorouracil (CARAC), leucovorin (FUSILEV) and methotrexate (RHEUMATREX); (3) plant alkaloids and terpenoids, including but not limited to vincristine (ONCOVIN), vinblastine and paclitaxel (TAXOL); (4) topoisomerase inhibitors, including but not limited to irinotecan (CAMPTOSAR), topotecan (HYCAMTIN) and etoposide (EPOSIN); (5) cytotoxic antibiotics, including but not limited to actinomycin D (COSMEGEN), doxorubicin (ADRIAMYCIN), bleomycin (BLENOXANE) and mitomycin (MITOSOL); (6) angiogenesis inhibitors, including but not limited to sunitinib (SUTENT) and bevacizumab (AVASTIN); and (7) tyrosine kinase inhibitors, including but not limited to imatinib (GLEEVEC), erlotinib (TARCEVA), lapatininb (TYKERB) and axitinib (INLYTA).

Where a subject is suffering from or at risk of suffering from an inflammatory condition, a GLS1 inhibitor compound described herein is optionally used together with one or more agents or methods for treating an inflammatory condition in any combination. Therapeutic agents/treatments for treating an autoimmune and/or inflammatory condition include, but are not limited to any of the following examples: (1) corticosteroids, including but not limited to cortisone, dexamethasone, and methylprednisolone; (2) nonsteroidal anti-inflammatory drugs (NSAIDs), including but not limited to ibuprofen, naproxen, acetaminophen, aspirin, fenoprofen (NALFON), flurbiprofen (ANSAID), ketoprofen, oxaprozin (DAYPRO), diclofenac sodium (VOLTAREN), diclofenac potassium (CATAFLAM), etodolac (LODINE), indomethacin (INDOCIN), ketorolac (TORADOL), sulindac (CLINORIL), tolmetin (TOLECTIN), meclofenamate (MECLOMEN), mefenamic acid (PONSTEL), nabumetone (RELAFEN) and piroxicam (FELDENE); (3) immunosuppressants, including but not limited to methotrexate (RHEUMATREX), leflunomide (ARAVA), azathioprine (IMURAN), cyclosporine (NEORAL, SANDIMMUNE), tacrolimus and cyclophosphamide (CYTOXAN); (4) CD20 blockers, including but not limited to rituximab (RITUXAN); (5) Tumor Necrosis Factor (TNF) blockers, including but not limited to etanercept (ENBREL), infliximab (REMICADE) and adalimumab (HUMIRA); (6) interleukin-1 receptor antagonists, including but not limited to anakinra (KINERET); (7) interleukin-6 inhibitors, including but not limited to tocilizumab (ACTEMRA); (8) interleukin-17 inhibitors, including but not limited to AIN457; (9) Janus kinase inhibitors, including but not limited to tasocitinib; and (10) syk inhibitors, including but not limited to fostamatinib.

### Compound Synthesis

Compounds of the present invention can be prepared using methods illustrated in general synthetic schemes and experimental procedures detailed below. General synthetic schemes and experimental procedures are presented for purposes of illustration and are not intended to be limiting. Starting materials used to prepare compounds of the present invention are commercially available or can be prepared using routine methods known in the art.

### LIST OF ABBREVIATIONS

Ac₂O=acetic anhydride; AcCl=acetyl chloride; AcOH=acetic acid; AIBN=azobisisobutyronitrile; aq.=aqueous; Bu₃SnH=tributyltin hydride; CD₃OD=deuterated methanol; CDCl₃=deuterated chloroform; CDI=1,1′-Carbonyldiimidazole; DBU=1,8-diazabicyclo[5.4.0]undec-7-ene; DCM=dichloromethane; DEAD=diethyl azodicarboxylate; DIBAL-H=di-iso-butyl aluminium hydride; DIEA=DIPEA=N,N-diisopropylethylamine; DMAP=4-dimethylaminopyridine; DMF=N,N-dimethylformamide; DMSO-d₆=deuterated dimethyl sulfoxide; DMSO=dimethyl sulfoxide; DPPA=diphenylphosphoryl azide; EDC.HCl=EDCI.HCl=1-ethyl-3-(3-dimethylaminopropyl)carbodiimide hydrochloride; Et₂O=diethyl ether; EtOAc=ethyl acetate; EtOH=ethanol; h=hour; HATU=2-(1H-7-azabenzotriazol-1-yl)-1,1,3,3-tetramethyl uronium hexafluorophosphate methanaminium; HMDS=hexamethyldisilazane; HOBT=1-hydroxybenzotriazole; i-PrOH=isopropanol; LAH=lithium aluminiumhydride; LiHMDS=Lithium bis(trimethylsilyl)amide; MeCN=acetonitrile; MeOH=methanol; MP-carbonate resin=macroporous triethylammonium methylpolystyrene carbonate resin; MsCl=mesyl chloride; MTBE=methyl tertiary butyl ether; n-BuLi=n-butyllithium; NaHMDS=Sodium bis(trimethylsilyl)amide; NaOMe=sodium methoxide; NaOtBu=sodium t-butoxide; NBS=N-bromosuccinimide; NCS=N-chlorosuccinimide; NMP=N-Methyl-2-pyrrolidone; Pd(Ph₃)₄=tetrakis(triphenylphosphine)palladium(0); Pd₂(dba)₃=tris(dibenzylideneacetone)dipalladium(0); PdCl₂(PPh₃)₂=bis(triphenylphosphine)palladium(II) dichloride; PG=protecting group; prep-HPLC=preparative high-performance liquid chromatography; PyBop=(benzotriazol-1-yloxy)tripyrrolidinophosphonium hexafluorophosphate; Pyr=pyridine; RT=room temperature; RuPhos=2-dicyclohexylphosphino-2′,6′-diisopropoxybiphenyl; sat.=saturated; ss=saturated solution; t-BuOH=tert-butanol; T3P=Propylphosphonic Anhydride; TEA=Et₃N=triethylamine; TFA=trifluoroacetic acid; TFAA=trifluoroacetic anhydride; THF=tetrahydrofuran; Tol=toluene; TsC1=tosyl chloride; XPhos=2-dicyclohexylphosphino-2′,4′,6′-triisopropylbiphenyl.

### General Methods for Preparing Compounds

The following schemes can be used to practice the present invention. Additional structural groups, including but not limited to those defined elsewhere in the specification and not shown in the compounds described in the schemes can be incorporated to give various compounds disclosed herein, or intermediate compounds which can, after further manipulations using techniques known to those skilled in the art, be converted to compounds of the present invention. For example in certain embodiments the A and B rings in the structures described in the schemes—wherein A and B are heteroaromatic rings—can be substituted with various groups as defined herein.

One route for preparation of compounds of the present invention is described in Scheme 1. A substituted functionalized heteroaromatic ring containing a pyridone-like moiety can be functionalized by reaction with an alkyl nitrile containing a suitable leaving group such as halogen, mesylate or tosylate, in the presence of a base such as K₂CO₃ or Cs₂CO₃, in a polar solvent such as DMF or NMP, with or without heating. Typically mixtures of O- and N-alkylated products are obtained, and the two regio-isomeric products can be separated by means of SiO₂ gel or reverse phase chromatography. The addition of lithium salts, for example LiC1, to the reaction mixture can be employed to favor N- vs. O-alkylation. The obtained alkyl nitrile can be further converted to the corresponding 5-alkyl-2-amino thiadazole by heating in the presence of TFA and hydrazinecarbothioamide. Functionalization of the 2-amino group is then possible by employing standard methodologies known to those skilled in the art. For example, acylation with an acyl chloride in the presence of a base such as DIEA, pyridine, TEA and in a solvent such as DCM can afford the corresponding 2-carboxamides. Alternatively, the same transformation can be achieved by employing a carboxylic acid and coupling reagents such as EDC.HCl/HOBT, PyBOP, HATU or T3P in the presence of a base such as TEA or DIPEA, in a solvent such as DCM or DMF, with or without heating.

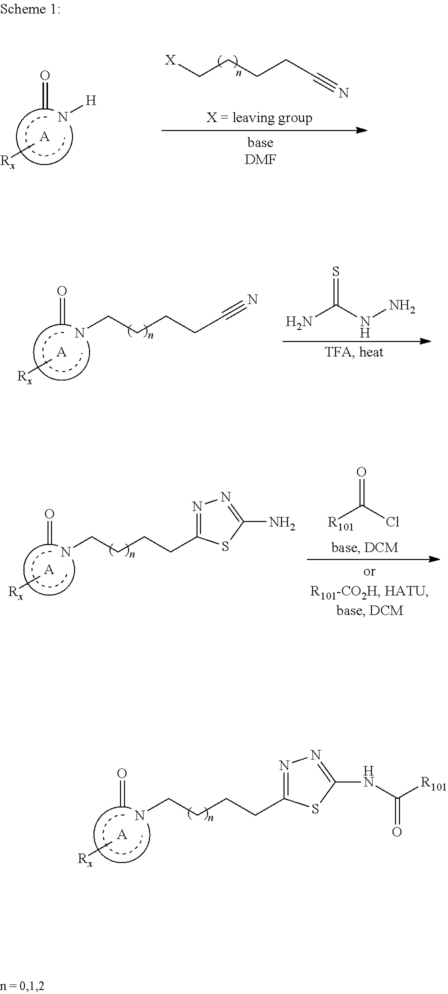

A further route of preparation of the compounds described in this invention is depicted in Scheme 2a and 2b. A substituted functionalized heteroaromatic ring containing a pyridone-like moiety can be functionalized by reaction with an alkyl compound containing two suitable leaving groups such as halogen, mesylate or tosylate, in the presence of a base such K₂CO₃ or Cs₂CO₃, in a polar solvent such as DMF or NMP, with or without heating. Controlled displacement of one of the two leaving groups can be achieved by adjusting the stoichiometry of the reagents. Similarly to what already described for Scheme 1, mixtures of N- and O-alkylated products can be expected, and the two region-isomeric products can typically be separated by chromatography. The obtained alkyl compound can be further converted into the corresponding azide by displacement of the remaining leaving group with NaN₃ in a solvent such as DMF or THF/H₂O, with or without heating. The obtained azide derivative can then be progressed to the corresponding triazole 4-carboxylic ester by copper-mediated azide-alkyne cycloaddition with a suitable alkyl propriolate in the presence of a base (e.g. TEA or DIEA), and a copper (I) salt such as CuI, or a copper (II) salt such as CuSO₄ in the presence of a reducing agent such as sodium ascorbate, in a solvent such as THF, DMSO, tBuOH or H₂O (H. C. Kolb, M. G. Finn and K. B. Sharpless, Angewandte Chemie International Edition, 2001, 40 (11): 2004-2021). The triazole 4-carboxylic ester can then be converted into the corresponding 4-carboxamide by direct displacement with a suitable amine heating in a polar solvent such as DMF. Alternatively, the same transformation could be achieved with a two-step sequence involving the base-mediated hydrolysis of the carboxylic ester followed by coupling of the resulting carboxylic acid with an amine, using standard coupling reagents such as HATU, PyBOP or EDCI.HCl/HOBT, T3P in the presence of a suitable base such as TEA or DIEA, in a polar solvent such as DMF, with or without heating. An alternative way to obtain the derivatives described in this invention is described in Scheme 2b, whereby the functionalized alkyl-azide described in Scheme 2a can be be progressed directly to the corresponding triazole 4-carboxamides by copper-mediated azide-alkyne cycloaddition with a suitable propiolamide in similar conditions to those described for Scheme 2a.

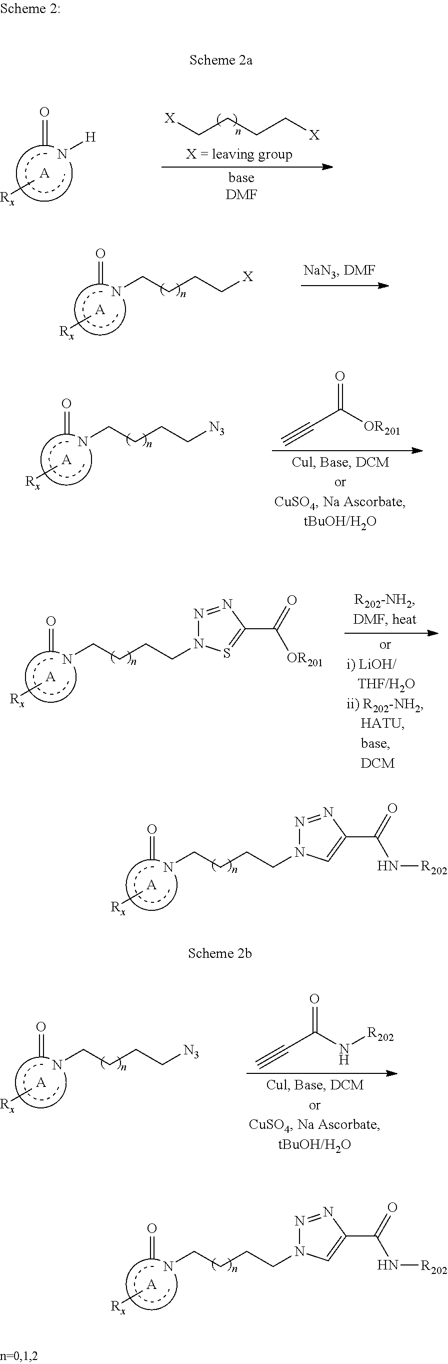

An additional route for the preparation of the compounds described in this invention is described in Scheme 3a. A substituted functionalized heteroaromatic ring containing a pyridone-like moiety can be functionalized by reaction with an alkyl compound containing an alkyne, in an analogous manner and using the same conditions to those described in Schemes 1 and 2. Like in those previously described routes, the N-alkylated product can be isolated from the O-alkylated regio-isomeric compound by means of chromatographic separation. The obtained alkyne can be further functionalized for example by Sonogashira cross-coupling reaction with a suitably functionalized heteroaromatic halide (Tetrahedron Lett. 16: 4467-4470). Typically, the above transformation is performed in the presence of a suitable Pd catalyst such as PdCl₂(PPh₃)₂ or Pd(PPh₃)₄, of a copper co-catalyst, typically a halide salt of copper (I), such as CuI or CuBr, and a base such as DIEA or TEA. The transformation can be run at RT or with mild heating in a variety of solvents, including DMF, toluene and EtOAc. Further functional group manipulations could include hydrogenation of the resulting heteroaromatic alkyne derivative in the presence of a suitable Pd catalyst (such as Pd/C or Pd(OH₂)) in a solvent such as EtOH.

An additional route to prepare the derivatives described in Scheme 3a is depicted in Scheme 3b. In this case, the functionalized heteroaromatic ring containing a pyridone-like moiety can be alkylated by reaction with suitably functionalized heteroalkyl compound containing a suitable leaving group, in an analogous manner and using the same conditions to those described in Schemes 1-3. As in the previous examples, the reaction results in the formation of a mixture of O- and N-alkylated products, which can be separated by chromatography.

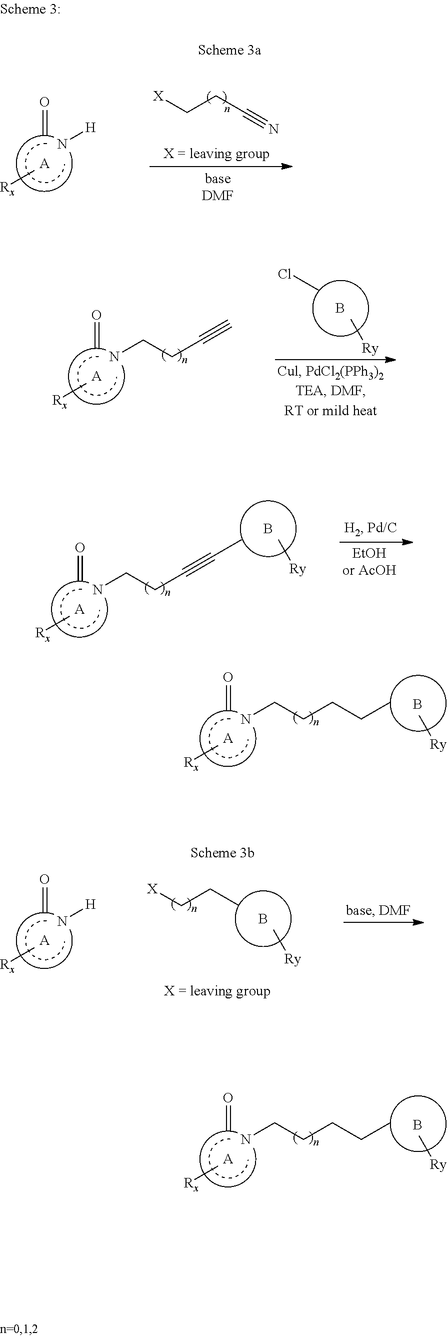

Standard functional group manipulations known to those skilled in the art can be performed with the R^{x} and R^{y} substituents groups of the structures reported in Schemes 1-3. A non-limiting number of examples of such transformations are exemplified in Scheme 4. For simplicity these transformations are described for the R^{x} group only, although they can be applied independently to one or both of the R^{x} and R^{y} groups.

In Scheme 4a, where R^{x}=carboxylic ester, the corresponding carboxamides can be obtained either by direct displacement with a suitable amine heating in a polar solvent such as DMF, or with a two-step sequence involving the base-mediated hydrolysis of the carboxylic ester followed by coupling of the resulting carboxylic acid with an amine. The coupling reaction can be performed using standard coupling reagents such as HATU, PyBOP or EDCI.HCl/HOBT or T3P in the presence of a suitable base such as TEA or DIEA, in a polar solvent such as DMF, with or without heating.

In Scheme 4b the Rx group is a protected amine group. Suitable protecting groups for the amine moiety can be chosen amongst substituted carbamates, amides and amines (e.g. benzyl amine, 3,4-dimethoxy-benzylamine, t-butyl carbamate, trifluoroacetamide) or amongst other suitable functional groups known to those skilled in the art (see also: P.G.M. Wutz, T.W. Greene, “Greene's protective Groups in Organic Synthesis”, Fourth Edition, John Wiley & Sons). The free amino group can be obtained by removal of the amine protecting group with techniques known to those skilled in the art (for example: reductive removal of a benzyl group; acid-mediated removal of the tert-butyl carbamoyl group and other conditions reported by P.G.M. Wutz, T.W. Greene in the reference cited above). The obtained amino derivatives can be further functionalized according to the non-limiting reaction examples reported in Scheme 4c. Thus, the corresponding carboxamides can be prepared by acylation with a carboxylic chloride in the presence of a base such as DIEA, pyridine or TEA in a solvent such as DCM, or employing a carboxylic acid and coupling reagents such as EDC.HCl/HOBT, PyBOP, HATU or T3P in the presence of a base such as TEA or DIPEA, in a solvent such as DCM or DMF, with or without heating. Similarly, carbamate and sulfamides derivatives can be obtained by reaction of the amine compounds with a suitable carbamoyl- or sulforyl chloride respectively, in the presence of a base such as TEA or DIPEA in a solvent such as DCM. Urea derivatives can be prepared by reaction of the amine moiety with a suitable isocyanate, in a solvent such as THF or DCM. Alkylation of the amino group can be achieved by treatment with a suitable alkylating agent, for example an alkyl, benzyl or heterobenzyl bromide in the presence of a base of a suitable strength, for example NaH, in a solvent such as THF.

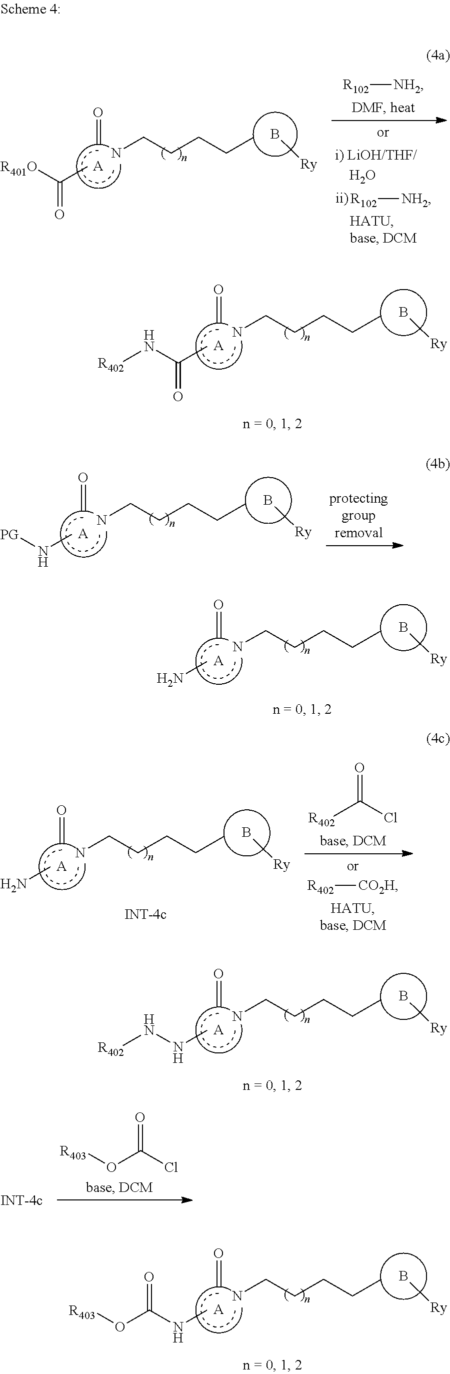

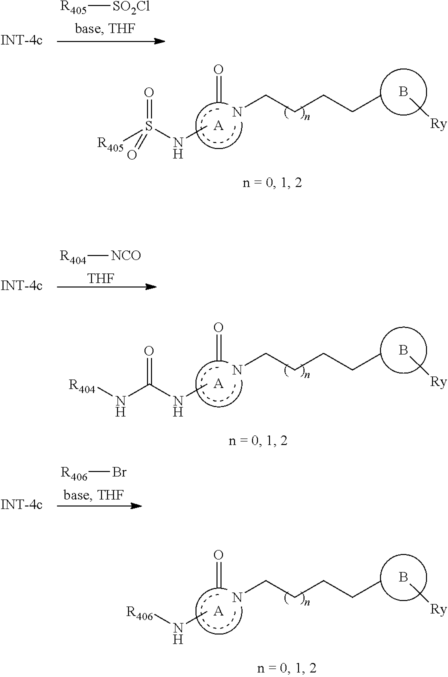

Non-limiting examples include the following compounds and pharmaceutically acceptable salts thereof.

### Example 1

### N-(2-oxo-1-(4-(5-(2-(pyridin-2-yl)acetamido)-1,3,4-thiadiazol-2-yl)butyl)-1,2-dihydropyridin-4-yl)-2-phenylacetamide

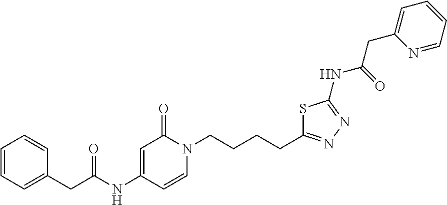 [SMILES: O=C(Cc1ccccc1)Nc1ccn(CCCCc2nnc(NC(=O)Cc3ccccn3)s2)c(=O)c1]

### Steps 1 to 3

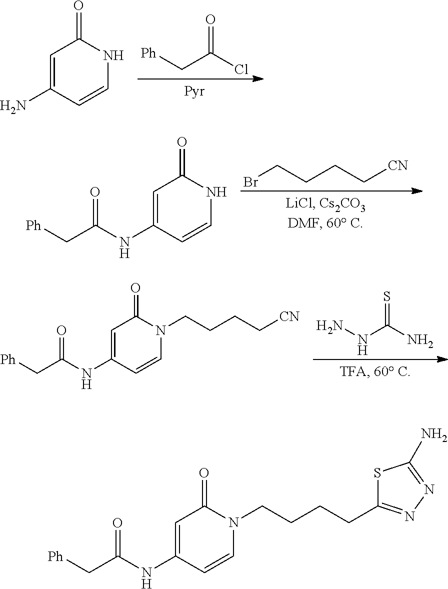

### Step 1: N-(2-oxo-1,2-dihydropyridin-4-yl)-2-phenylacetamide

To a mixture of pyridine (0.220 ml, 2.72 mmol) and 4-aminopyridin-2(1H)-one (0.20 g, 1.8 mmol) in DMF (2 ml) was added 2-phenylacetyl chloride (0.290 ml, 2.36 mmol). The resulting mixture was allowed to stir at RT for 24 h, diluted with water (20 mL), stirred at 0° C. for 10 minutes and filtered. The solid was washed with cold water (5 mL) followed by Et₂O (5 mL) to give the title compound as a white solid (261 mg, 63%). MS (ES⁺) C₁₃H₁₂N₂O₂ requires: 228. found: 229 [M+H]+.

### Step 2: N-(1-(4-cyanobutyl)-2-oxo-1,2-dihydropyridin-4-yl)-2-phenylacetamide

A vial was charged with LiCl (0.037 g, 0.88 mmol), Cs₂CO₃ (0.171 g, 0.526 mmol), N-(2-oxo-1,2-dihydropyridin-4-yl)-2-phenylacetamide (0.10 g, 0.44 mmol), 5-bromopentanenitrile (0.061 ml, 0.526 mmol) and DMF (1.7 ml). The resulting mixture was heated at 60° C. for 15 h, concentrated under reduced pressure and the residue was purified by mass-triggered preparative HPLC (Mobile phase: A=0.1% TFA/H₂O, B=0.1% TFA/MeCN; Gradient: B=30-70%; 12 minutes; Column: C18) to give the title compound as an off-white solid (49 mg, 36% yield). MS (ES⁺) C₁₈H₁₉N₃O₂ requires: 309. found: 310 [M+H]⁺.

### Step 3: N-(1-(4-(5-amino-1,3,4-thiadiazol-2-yl)butyl)-2-oxo-1,2-dihydropyridin-4-yl)-2-phenylacetamide

To a solution of N-(1-(4-cyanobutyl)-2-oxo-1,2-dihydropyridin-4-yl)-2-phenylacetamide (49 mg, 0.16 mmol) in trifluoroacetic acid (122 al, 1.58 mmol) was added hydrazinecarbothioamide (16 mg, 0.17 mmol) and the resulting mixture was stirred at 60° C. for 4 h. The reaction was then diluted with toluene (5 mL) and concentrated under reduced pressure. The residue was purified by mass-triggered preparative HPLC (Mobile phase: A=0.1% TFA/H₂O, B=0.1% TFA/MeCN; Gradient: B=20-60%; 12 minutes; Column: C18) to give the title compound as a thick oil (54 mg, 68%). MS (ES⁺) C₁₉H₂₁N₅O₂S requires: 383. found: 384 [M+H]⁺; ¹H NMR (600 MHz, Methanol-d₄) δ 7.57 (d, J=7.4 Hz, 1H), 7.34-7.28 (m, 4H), 7.25 (m, 1H), 7.01 (d, J=2.3 Hz, 1H), 6.66 (dd, J=7.4, 2.4 Hz, 1H), 3.98 (t, J=7.0 Hz, 2H), 3.68 (s, 2H), 2.94 (t, J=7.3 Hz, 2H), 1.84-1.70 (m, 4H).

### Step 4

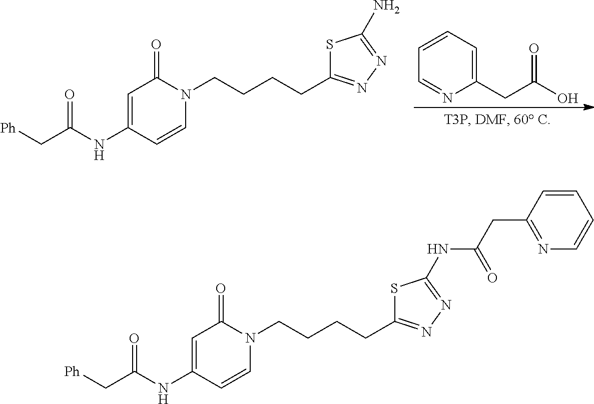

### Step 4: N-(2-oxo-1-(4-(5-(2-(pyridin-2-yl)acetamido)-1,3,4-thiadiazol-2-yl)butyl)-1,2-dihydropyridin-4-yl)-2-phenylacetamide

To a suspension of 2-(pyridin-2-yl)acetic acid hydrochloride (10 mg, 0.058 mmol) and N-(1-(4-(5-amino-1,3,4-thiadiazol-2-yl)butyl)-2-oxo-1,2-dihydropyridin-4-yl)-2-phenylacetamide (17 mg, 0.044 mmol) in DMF (222 μl) was added T3P (36.7 mg, 0.058 mmol, 50% solution in DMF) and the resulting mixture was stirred at 60° C. for 3 h. The reaction was quenched with MeOH (1 mL) and concentrated under reduced pressure. The residue was purified by mass-triggered preparative HPLC (Mobile phase: A=0.1% TFA/H₂O, B=0.1% TFA/MeCN; Gradient: B=20-50%; 16 minutes; Column: C18) to give the title compound as a pale yellow solid. MS (ES⁺) C₂₆H₂₆N₆O₃S requires: 502. found: 503 [M+H]⁺; ¹H NMR (600 MHz, DMSO-d₆) δ 12.61 (s, 1H), 10.25 (s, 1H), 8.51 (m, 1H), 7.76 (m, 1H), 7.56 (d, J=7.4 Hz, 1H), 7.39 (d, J=7.8 Hz, 1H), 7.35-7.21 (m, 6H), 6.72 (d, J=2.3 Hz, 1H), 6.36 (dd, J=7.4, 2.4 Hz, 1H), 3.98 (s, 2H), 3.85-3.75 (m, 2H), 3.65 (s, 2H), 3.02-2.93 (m, 2H), 1.69-1.58 (m, 4H).

### Example 2

### N-benzyl-1-(4-(2-oxo-4-(2-phenylacetamido)pyridin-1(2H)-yl)butyl)-1H-1,2,3-triazole-4-carboxamide

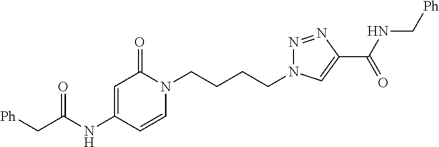 [SMILES: O=C(Cc1ccccc1)Nc1ccn(CCCCn2cc(C(=O)NCc3ccccc3)nn2)c(=O)c1]

### Steps 1 to 4

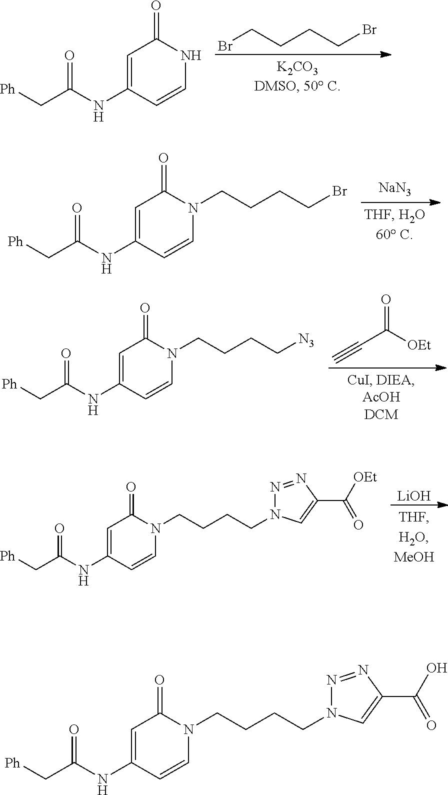

### Step 1: N-(1-(4-bromobutyl)-2-oxo-1,2-dihydropyridin-4-yl)-2-phenylacetamide

To a suspension of K₂CO₃ (0.945 g, 6.83 mmol) and N-(2-oxo-1,2-dihydropyridin-4-yl)-2-phenylacetamide (0.78 g, 3.42 mmol) in DMSO (6.83 ml) was added 1,4-dibromobutane (0.816 ml, 6.83 mmol) and the resulting mixture was stirred at 50 OC for 6 h. The mixture was diluted with EtOAc (50 mL) and washed with water (2×20 mL) and brine (2×10 mL). The organic layer was dried (MgSO₄) and concentrated under reduced pressure. The residue was purified via SiO₂ gel chromatography (0-20% MeOH in DCM) to give the title compound as a yellow thick oil that solidified upon standing (0.67 mg, 43%). MS (ES⁺) C₁₇H₁₉BrN₂O₂ requires: 362. found: 363 [M+H]⁺; ¹H NMR (600 MHz, DMSO-d₆) δ 10.27 (s, 1H), 7.57 (d, J=7.4 Hz, 1H), 7.33-7.26 (m, 4H), 7.28-7.23 (m, 1H), 6.74 (d, J=2.3 Hz, 1H), 6.38 (dd, J=7.4, 2.3 Hz, 1H), 3.83 (t, J=6.7 Hz, 2H), 3.65 (s, 2H), 3.54 (t, J=6.4 Hz, 2H), 1.79-1.66 (m, 4H).

### Step 2: N-(1-(4-azidobutyl)-2-oxo-1,2-dihydropyridin-4-yl)-2-phenylacetamide

To a solution of N-(1-(4-bromobutyl)-2-oxo-1,2-dihydropyridin-4-yl)-2-phenylacetamide (0.665 g, 1.83 mmol) in THF (7.32 ml) and water (1.83 ml) was added NaN₃ (0.131 g, 2.01 mmol) and the resulting mixture was stirred at 60° C. for 6 h. The reaction mixture was concentrated under reduced pressure, the residue was taken up in EtOAc (50 mL), and washed with water (2×20 mL) and brine (20 mL). The organic layers were dried (MgSO₄) and concentrated under reduced pressure to give the title compound as an off-white solid (0.58 g, 98%). MS (ES⁺) C₁₇H₁₉N₅O₂ requires: 325. found: 326 [M+H]⁺.

### Step 3: Ethyl 1-(4-(2-oxo-4-(2-phenylacetamido)pyridin-1(2H)-yl)butyl)-1H-1,2,3-triazole-4-carboxylate

To a solution of acetic acid (2.9 al, 0.051 mmol), DIEA (8.9 al, 0.051 mmol), ethyl propiolate (0.078 ml, 0.77 mmol), and N-(1-(4-azidobutyl)-2-oxo-1,2-dihydropyridin-4-yl)-2-phenylacetamide (0.167 g, 0.513 mmol) in DCM (2.57 ml) was added copper(I) iodide (4.9 mg, 0.026 mmol) and the resulting mixture was stirred at RT for 3 h. The volatiles were removed under reduced pressure and the residue was purified via silica gel chromatography (0-15% MeOH in DCM with 1% NH₄OH) to give the title compound as a white solid (123 mg, 54%). MS (ES+) C₂₂H₂₅N₅O₄ requires: 423. found: 424 [M+H]⁺.

### Step 4: 1-(4-(2-oxo-4-(2-phenylacetamido)pyridin-1(2H)-yl)butyl)-1H-1,2,3-triazole-4-carboxylic acid

To a solution of ethyl 1-(4-(2-oxo-4-(2-phenylacetamido)pyridin-1(2H)-yl)butyl)-1H-1,2,3-triazole-4-carboxylate (0.117 g, 0.276 mmol) in THF (1.10 ml) and MeOH (0.276 ml) was added LiOH (2M solution in water, 0.152 ml, 0.304 mmol,) and the resulting mixture was stirred at RT for 3 h. The reaction was concentrated under reduced pressure to give the title compound as an off-white solid (109 mg, 100%, Li salt). MS (ES⁺) C₂₀H₂₁N₅O₄ requires: 395. found: 396 [M+H]⁺; ¹H NMR (500 MHz, DMSO-d₆) δ 10.64 (s, 1H), 8.04 (s, 1H), 7.54 (d, J=7.4 Hz, 1H), 7.33-7.28 (m, 4H), 7.28-7.21 (m, 1H), 6.78 (d, J=2.3 Hz, 1H), 6.40 (dd, J=7.5, 2.3 Hz, 1H), 4.32 (t, J=7.0 Hz, 2H), 3.81 (t, J=7.6 Hz, 2H), 3.67 (s, 2H), 3.17 (d, J=4.8 Hz, 1H), 1.77-1.74 (m, 2H), 1.57-1.48 (m, 2H).

### Step 5

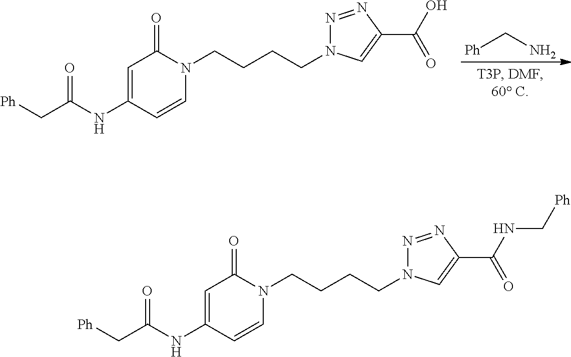 [SMILES: O=C(Cc1ccccc1)Nc1ccn(CCCCn2cc(C(=O)O)nn2)c(=O)c1 ; NCc1ccccc1 ; O=C(Cc1ccccc1)Nc1ccn(CCCCn2cc(C(=O)NCc3ccccc3)nn2)c(=O)c1]

### N-benzyl-1-(4-(2-oxo-4-(2-phenylacetamido)pyridin-1(2H)-yl)butyl)-1H-1,2,3-triazole-4-carboxamide

To a suspension of 1-(4-(2-oxo-4-(2-phenylacetamido)pyridin-1 (2H)-yl)butyl)-1H-1,2,3-triazole-4-carboxylic acid lithium salt (20 mg, 0.051 mmol) and benzylamine (6.6 μl, 0.061 mmol) in DMF (253 μl) was T3P (39 mg, 0.061 mmol, 50% solution in DMF) and the resulting mixture was stirred at 60° C. for 3 h. The mixture was concentrated under reduced pressure and the residue was purified by mass-triggered preparative HPLC (Mobile phase: A=0.1% TFA/H₂O, B=0.1% TFA/MeCN; Gradient: B=30-70%; 12 minutes; Column: C18) to give the title compound as an off-white solid (3.5 mg, 12%). MS (ES⁺) C₂₇H₂₈N₆O₃ requires: 484. found: 485 [M+H]⁺; ¹H NMR (600 MHz, DMSO-d₆) δ 10.29 (s, 1H), 9.06 (t, J=6.3 Hz, 1H), 8.57 (s, 1H), 7.56 (d, J=7.4 Hz, 1H), 7.31-7.27 (m, 8H), 7.26-7.24 (m, 2H), 6.75 (d, J=2.3 Hz, 1H), 6.37 (dd, J=7.4, 2.3 Hz, 1H), 4.45-4.40 (m, 4H), 3.82 (t, J=7.1 Hz, 2H), 3.65 (s, 2H), 1.81-1.79 (m, 2H), 1.57-1.48 (m, 2H).

### Example 3

### Tert-butyl(2-oxo-1-(4-(4-((3-(trifluoromethoxy)benzyl)carbamoyl)-1H-1,2,3-triazol-1-yl)butyl)-1,2-dihydropyridin-4-yl)carbamate

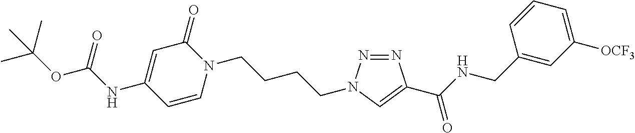 [SMILES: CC(C)(C)OC(=O)Nc1ccn(CCCCn2cc(C(=O)NCc3cccc(OC(F)(F)F)c3)nn2)c(=O)c1]

To a solution of tert-butyl(1-(4-azidobutyl)-2-oxo-1,2-dihydropyridin-4-yl)carbamate (Example 2, Step 2, 80 mg, 0.26 mmol) in DCM (3 ml) were added DIEA (4.5 μl, 0.026 mmol), AcOH (1.5 μl, 0.026 mmol), N-(3-(trifluoromethoxy)benzyl)propiolamide (63.3 mg, 0.260 mmol), and CuI (2.5 mg, 0.013 mmol) and the resulting mixture was stirred at RT for 2.5 h. The reaction mixture was directly loaded on SiO₂ and was purified via silica gel chromatography (0-100% EtOAc in hexane with 10% MeOH) to give the title compound as a white solid (112 mg, 78%). MS (ES+) C₂₅H₂₉F₃N₆O₅ requires: 550.2. found: 551 [M+H]+. 1H NMR (600 MHz, DMSO-d₆) δ: 9.56 (s, 1H), 9.16 (t, J=6.2 Hz, 1H), 8.58 (s, 1H), 7.49 (d, J=7.6 Hz, 1H), 7.44 (m, 1H), 7.34 (d, J=7.6 Hz, 1H), 7.29 (s, 1H), 7.23 (d, J=8.3 Hz, 1H), 6.49 (d, J=2.3 Hz, 1H), 6.31 (dd, J=7.2, 2.3 Hz, 1H), 4.48 (d, J=6.4 Hz, 2H), 4.43 (t, J=7.0 Hz, 2H), 3.80 (t, J=7.0 Hz, 2H), 1.76-1.83 (m, 2H), 1.57-1.53 (m, 2H), 1.46 (s, 9H).

### Example 4

### 1-(4-(4-Amino-2-oxopyridin-1(2H)-yl)butyl)-N-(3-(trifluoromethoxy)benzyl)-1H-1,2,3-triazole-4-carboxamide

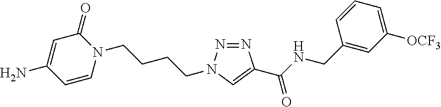 [SMILES: Nc1ccn(CCCCn2cc(C(=O)NCc3cccc(OC(F)(F)F)c3)nn2)c(=O)c1]

To a solution of tert-butyl(2-oxo-1-(4-(4-((3-(trifluoromethoxy)benzyl)carbamoyl)-1H-1,2,3-triazol-1-yl)butyl)-1,2-dihydropyridin-4-yl)carbamate (Example 3, 95 mg, 0.178 mmol) in DCM (3 mL) was added TFA (1 mL) and the reaction stirred at RT for 45 minutes. The reaction mixture was then concentrated under reduced pressure, the residue was dissolved in DCM/MeOH (1:1 v:v, 8 mL) and stirred with 1 g of MP-carbonate resin (3 mmol/g) at RT for 15 minutes. The mixture was filtered through a cotton plug and a syringe filter (13 mm PP 0.45 μm), the filtrate was concentrated under reduced pressure to give the title compound as a white solid (78 mg, 97%). MS (ES⁺) C₂₀H₂₁F₃N₆O₃ requires: 450.2. found: 451 [M+H]⁺. ¹H NMR (600 MHz, DMSO-d₆) δ 9.16 (t, J=6.2 Hz, 1H), 8.58 (s, 1H), 7.45 (m, 1H), 7.34 (d, J=7.6 Hz, 1H), 7.29 (s, 1H), 7.21-7.25 (m, 2H), 5.93 (s, 2H), 5.63 (dd, J=7.2, 2.3 Hz, 1H), 5.21 (d, J=2.3 Hz, 1H), 4.48 (d, J=6.0 Hz, 2H), 4.43 (t, J=7.2 Hz, 2H), 3.70 (t, J=7.0 Hz, 2H), 1.81-1.76 (m, 2H), 1.52-1.48 (m, 2H).

### Example 5

### 1-(4-(4-(3-methylbutanamido)-2-oxopyridin-1(2H)-yl)butyl)-N-(3-(trifluoromethoxy)benzyl)-1H-1,2,3-triazole-4-carboxamide

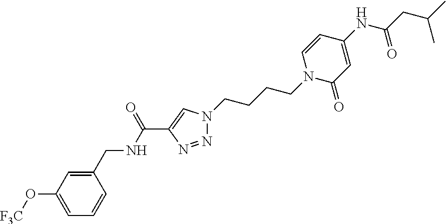 [SMILES: CC(C)CC(=O)Nc1ccn(CCCCn2cc(C(=O)NCc3cccc(OC(F)(F)F)c3)nn2)c(=O)c1]

To a solution of 1-(4-(4-amino-2-oxopyridin-(2H)-yl)butyl)-N-(3-(trifluoromethoxy)benzyl)-1H-1,2,3-triazole-4-carboxamide (Example 4, 15 mg, 0.033 mmol) and DIEA (7.6 uL, 0.043 mmol) in DMF (0.5 mL) at 0° C. was added 3-methylbutanoyl chloride (4.8 mg, 0.040 mmol). The resulting mixture was stirred at RT for 16 h, diluted with DCM and the pH was adjusted to ˜7-8 by addition of 1 N aqueous NaOH. The organic layer was washed with water, dried over Na₂SO₄, filtered, concentrated under reduced pressure and purified by silica gel column chromatography (DCM:MeOH=10:0 to 10:1) to give the title compound (2.2 mg, 12%). MS (ES⁺) C₂₅H₂₉F₃N₆O₄ requires: 534. found: 535 [M+H]⁺; ¹H NMR (600 MHz, DMSO-d₆) δ 9.95 (s, 1H), 9.16 (t, J=6.0 Hz, 1H), 8.59 (s, 1H), 7.54 (d, J=7.8 Hz, 1H), 7.45 (m, 1H), 7.34 (d, J=7.8 Hz, 1H), 7.29 (s, 1H), 7.23 (d, J=7.8 Hz, 1H), 6.75 (d, J=2.4 Hz, 1H), 6.35 (dd, J=7.8, 2.4 Hz, 1H), 4.48 (d, J=6.0 Hz, 2H), 4.47-4.42 (m, 2H), 3.84-3.86 (m, 2H), 2.18 (d, J=7.2 Hz, 2H), 2.04 (m, 1H), 1.83-1.79 (m, 2H), 1.57-1.54 (m, 2H), 0.91 (d, J=7.2 Hz, 6H).

### Example 6

### 1-(4-(2-oxo-4-(2-(pyridin-2-yl)acetamido)pyridin-1 (2H)-yl)butyl)-N-(3-(trifluoromethoxy)benzyl)-1H-1,2,3-triazole-4-carboxamide

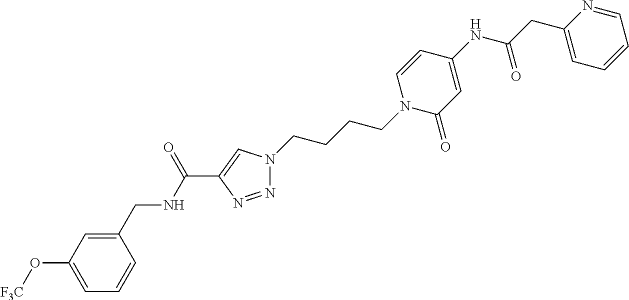 [SMILES: O=C(Cc1ccccn1)Nc1ccn(CCCCn2cc(C(=O)NCc3cccc(OC(F)(F)F)c3)nn2)c(=O)c1]

To a solution of 1-(4-(4-amino-2-oxopyridin-1(2H)-yl)butyl)-N-(3-(trifluoromethoxy)benzyl)-1H-1,2,3-triazole-4-carboxamide (Example 4, 15 mg, 0.033 mmol) and 2-(pyridin-2-yl)acetic acid hydrochloride (7.5 mg, 0.043 mmol) in DMF (0.3 mL) were added HATU (19 mg, 0.050 mmol) and DIEA (15 uL, 0.083 mmol). The resulting mixture was stirred at RT for 16 h, diluted with DCM, and the pH was adjusted to ˜7-8 by addition of 1 N aqueous NaOH. The organic layer was washed with water, dried over Na₂SO₄, filtered, concentrated under reduced pressure and purified by silica gel column chromatography (DCM:MeOH=10:0 to 10:1) to give the title compound (5.7 mg, 30%). MS (ES⁺) C₂₇H₂₆F₃N₇O₄ requires: 569. found: 570 [M+H]⁺; ¹H NMR (600 MHz, DMSO-d₆) δ 10.48 (s, 1H), 9.16 (t, J=6.0 Hz, 1H), 8.71 (s, 1H), 8.57 (s, 1H), 8.17 (m, 1H), 7.69 (d, J=7.8 Hz, 1H), 7.63 (m, 1H), 7.58 (d, J=7.8 Hz, 1H), 7.45 (m, 1H), 7.34 (d, J=7.8 Hz, 1H), 7.29 (s, 1H), 7.23 (d, J=7.8 Hz, 1H), 6.73 (d, J=2.4 Hz, 1H), 6.37 (dd, J=7.8, 2.4 Hz, 1H), 4.48 (d, J=6.0 Hz, 2H), 4.44 (t, J=7.2 Hz, 2H), 4.06 (s, 2H), 3.84 (d, J=7.2 Hz, 2H), 1.84-1.80 (m, 2H), 1.59-1.55 (m, 2H).

### Example 7

### Isobutyl(2-oxo-1-(4-(4-((3-(trifluoromethoxy)benzyl)carbamoyl)-1H-1,2,3-triazol-1-yl)butyl)-1,2-dihydropyridin-4-yl)carbamate

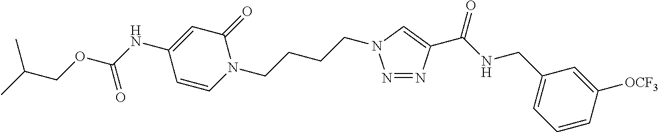 [SMILES: CC(C)COC(=O)Nc1ccn(CCCCn2cc(C(=O)NCc3cccc(OC(F)(F)F)c3)nn2)c(=O)c1]

To a solution of 1-(4-(4-amino-2-oxopyridin-1(2H)-yl)butyl)-N-(3-(trifluoromethoxy)benzyl)-1H-1,2,3-triazole-4-carboxamide (Example 4, 12.2 mg, 0.027 mmol) in pyridine (0.25 ml) was added isobutyl carbonochloridate (7.0 μl, 0.054 mmol) and the resulting mixture was stirred at RT for 16 h. The reaction mixture was then concentrated under reduced pressure, the residue was taken up in DMSO and purified by mass-triggered preparative HPLC (Mobile phase: A=0.1% TFA/H₂O, B=0.1% TFA/MeCN; Gradient: B=40-80%; 12 min; Column: C18) to give the title compound as a white solid (7.6 mg, 51%). MS (ES⁺) C₂₅H₂₉F₃N₆O₅ requires: 550.2. found: 551 [M+H]⁺. ¹H NMR (600 MHz Methanol-d₄) δ: 8.38 (s, 1H), 7.55 (d, J=7.6 Hz, 1H), 7.39-7.44 (m, 1H), 7.36 (d, J=7.9 Hz, 1H), 7.27 (s, 1H), 7.16 (d, J=8.3 Hz, 1H), 6.83 (d, J=2.3 Hz, 1H), 6.62 (dd, J=7.2, 2.3 Hz, 1H), 4.60 (s, 2H), 4.50 (t, J=7.0 Hz, 2H), 3.99 (t, J=7.2 Hz, 2H), 3.96-3.92 (m, 2H), 1.90-2.01 (m, 2H), 1.76-1.72 (m, 2H), 0.97 (d, J=6.8 Hz, 6H).

### Example 8

### N-Benzyl-2-oxo-1-(4-(4-((3-(trifluoromethoxy)benzyl)carbamoyl)-1H-1,2,3-triazol-1-yl)butyl)-1,2-dihydropyridine-4-carboxamide

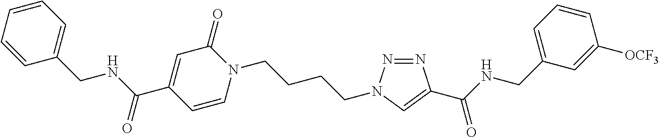 [SMILES: O=C(NCc1ccccc1)c1ccn(CCCCn2cc(C(=O)NCc3cccc(OC(F)(F)F)c3)nn2)c(=O)c1]

### Steps 1 to 5

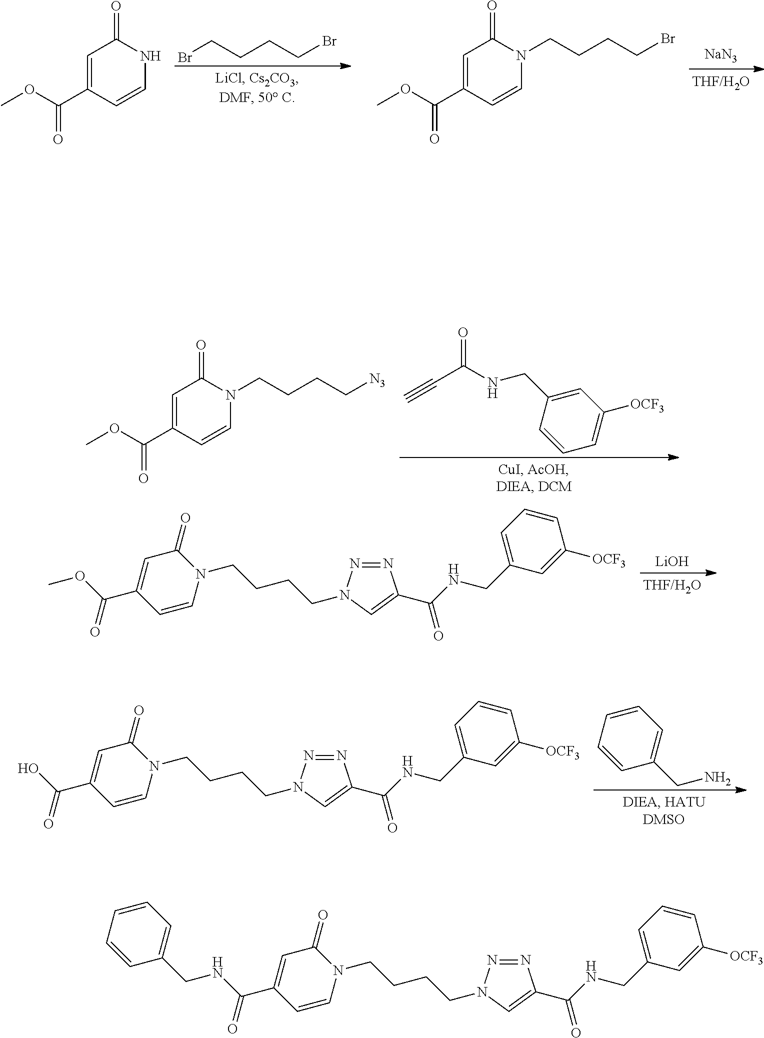

### Step 1: methyl 1-(4-bromobutyl)-2-oxo-1,2-dihydropyridine-4-carboxylate

To a solution of methyl 2-oxo-1,2-dihydropyridine-4-carboxylate (500 mg, 3.27 mmol) in DMF (10 ml) were added Cs₂CO₃ (2.13 g, 6.53 mmol), LiCl (138 mg, 3.27 mmol) and 1,4-dibromobutane (1.17 ml, 9.80 mmol), and the resulting mixture was stirred at 50° C. for 3.5 h. The reaction mixture was then concentrated under reduced pressure, diluted with DCM and filtered. The solid was washed sequentially with DCM, DCM/MeOH, DCM, and the filtrate was concentrated under reduced pressure. The residue was taken up in DCM and adsorbed onto Celite® and purified via SiO₂ gel chromatography (0-75% EtOAc in hexane with 10% MeOH) to give the title compound as a pale yellow solid (498 mg, 53%). MS (ES⁺) C₁₁H₁₄BrNO₃ requires: 287.0. found: 288 [M+H]⁺. ¹H NMR (600 MHz, DMSO-d₆) 7.84 (d, J=7.2 Hz, 1H), 6.86 (s, 1H), 6.57 (dd, J=7.0, 1.3 Hz, 1H), 3.95 (t, J=6.6 Hz, 2H), 3.84 (s, 3H), 3.58-3.52 (m, 2H), 1.83-1.73 (m, 4H).

### Step 2: methyl 1-(4-azidobutyl)-2-oxo-1,2-dihydropyridine-4-carboxylate

To a solution of methyl 1-(4-bromobutyl)-2-oxo-1,2-dihydropyridine-4-carboxylate (494 mg, 1.71 mmol) in THF (4 ml) and water (1.00 ml) was added NaN₃ (223 mg, 3.43 mmol) and the resulting mixture was stirred in at 60° C. for 17 h. The reaction mixture was then diluted with water (30 mL) and extracted with EtOAc (2×25 mL). The combined organic layers were washed with brine, dried over MgSO₄, filtered and concentrated under reduced pressure to give the title compound (415 mg, 97%). MS (ES⁺) C₁₁H₁₄N₄O₃ requires: 250.1. found: 251 [M+H]⁺. ¹H NMR (600 MHz, DMSO-d₆) 7.84 (d, J=6.8 Hz, 1H), 6.86 (s, 1H), 6.57 (dd, J=7.0, 1.3 Hz, 1H), 3.94 (t, J=7.2 Hz, 2H), 3.84 (s, 3H), 3.36 (t, J=7.0 Hz, 2H), 1.72-1.66 (m, 2H), 1.56-1.47 (m, 2H).

### Step 3: methyl 2-oxo-1-(4-(4-((3-(trifluoromethoxy)benzyl)carbamoyl)-1H-1,2,3-triazol-1-yl)butyl)-1,2-dihydropyridine-4-carboxylate

To a solution of N-(3-(trifluoromethoxy)benzyl)propiolamide (401 mg, 1.65 mmol) in DCM (8 ml) was added to methyl 1-(4-azidobutyl)-2-oxo-1,2-dihydropyridine-4-carboxylate (413 mg, 1.65 mmol) were added AcOH (9.45 μl, 0.165 mmol), DIEA (0.029 ml, 0.165 mmol), and CuI (15.7 mg, 0.0830 mmol). The resulting mixture was stirred at RT for 24 h. The reaction mixture was diluted with MeOH, loaded on Celite® and purified by SiO₂ gel chromatography (0 to 100% EtOAc in hexane with 10% MeOH) to give the title compound as an off white solid (481 mg, 59%). MS (ES⁺) C₂₂H₂₂F₃N₅O₅ requires: 493.2. found: 494 [M+H]⁺. ¹H NMR (600 MHz, DMSO-d₆) δ 9.16 (t, J=6.2 Hz, 1H), 8.59 (s, 1H), 7.81 (d, J=7.2 Hz, 1H), 7.45 (m, 1H), 7.34 (d, J=7.9 Hz, 1H), 7.28 (s, 1H), 7.23 (d, J=8.3 Hz, 1H), 6.85 (d, J=1.1 Hz, 1H), 6.55 (dd, J=6.8, 1.5 Hz, 1H), 4.51-4.40 (m, 4H), 3.94 (t, J=7.0 Hz, 2H), 3.84 (s, 3H), 1.86-1.81 (m, 2H), 1.64-1.59 (m, 2H).

### Step 4: 2-oxo-1-(4-(4-((3-(trifluoromethoxy)benzyl)carbamoyl)-1H-1,2,3-triazol-1-yl)butyl)-1,2-dihydropyridine-4-carboxylic acid

To a suspension of methyl 2-oxo-1-(4-(4-((3-(trifluoromethoxy)benzyl)carbamoyl)-1H-1,2,3-triazol-1-yl)butyl)-1,2-dihydropyridine-4-carboxylate (476 mg, 0.965 mmol) in THF (3 ml) was added MeOH (3.00 ml), water (3.00 ml) and lithium hydroxide hydrate (46.5 mg, 1.11 mmol) and the resulting mixture was stirred at RT for 2.5 h. The reaction mixture was then concentrated, diluted with water and brought to pH ˜2-3 with 1 N aq. HCl. The resulting precipitate was filtered and washed sequentially with water, hexanes, ether/hexanes (1:1 v/v), hexanes, and dried under reduced pressure to give the title compound as a white solid (420 mg, 91%). MS (ES⁺) C₂₁H₂₀F₃N₅O₅ requires: 479.1. found: 480 [M+H]⁺. ¹H NMR (600 MHz, DMSO-d₆) 9.16 (t, J=6.2 Hz, 1H), 8.59 (s, 1H), 7.77 (d, J=6.8 Hz, 1H), 7.45 (m, 1H), 7.34 (d, J=7.9 Hz, 1H), 7.29 (s, 1H), 7.23 (d, J=7.9 Hz, 1H), 6.82 (s, 1H), 6.54 (dd, J=7.0, 1.3 Hz, 1H), 4.40-4.52 (m, 4H), 3.93 (t, J=7.2 Hz, 2H), 1.87-1.82 (m, 2H), 1.63-1.60 (m, 2H).

### Step 5: N-benzyl-2-oxo-1-(4-(4-((3-(trifluoromethoxy)benzyl)carbamoyl)-1H-1,2,3-triazol-1-yl)butyl)-1,2-dihydropyridine-4-carboxamide

To a solution of 2-oxo-1-(4-(4-((3-(trifluoromethoxy)benzyl)carbamoyl)-1H-1,2,3-triazol-1-yl)butyl)-1,2-dihydropyridine-4-carboxylic acid (10.4 mg, 0.022 mmol) in DMSO (0.1 ml) were added phenylmethanamine (4.65 mg, 0.043 mmol), DIEA (0.015 ml, 0.087 mmol), and HATU (24.7 mg, 0.065 mmol) and the resulting mixture was stirred at RT for 13 h. The reaction mixture was then diluted with water (4 mL) and the resulting precipitate was filtered, washed with water, hexanes and dried under reduced pressure to give the title compound as a white solid (12.6 mg, 100%). MS (ES⁺) C₂₈H₂₇F₃N₆O₄ requires: 568.2. found: 569 [M+H]⁺. ¹H NMR (600 MHz, DMSO-d₆): 9.21-9.09 (m, 2H), 8.59 (s, 1H), 7.76 (d, J=7.2 Hz, 1H), 7.46 (m, 1H), 7.37-7.31 (m, 3H), 7.29 (m, 3H), 7.27-7.20 (m, 2H), 6.83 (s, 1H), 6.52-6.57 (m, 1H), 4.38-4.51 (m, 6H), 3.93 (t, J=7.0 Hz, 2H), 1.86-1.80 (m, 2H), 1.64-1.61 (m, 2H).

### Example 9

### tert-Butyl N-[(1-{4-[2-oxo-4-(2-phenylacetamido)-1,2-dihydropyridin-1-yl]butyl}-1H-1,2,3-triazol-4-yl)methyl]carbamate

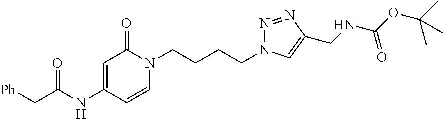 [SMILES: CC(C)(C)OC(=O)NCc1cn(CCCCn2ccc(NC(=O)Cc3ccccc3)cc2=O)nn1]

To a mixture of tert-butyl prop-2-yn-1-ylcarbamate (21.5 mg, 0.138 mmol), DIEA (1.61 μl, 9.22 μmol), AcOH (0.53 μl, 9.2 μmol), and N-(1-(4-azidobutyl)-2-oxo-1,2-dihydropyridin-4-yl)-2-phenylacetamide (Example 2, Step 2; 30 mg, 0.092 mmol) in DCM (922 μl) was added copper(I) iodide (0.88 mg, 4.6 mol). The resulting reaction mixture was stirred at RT for 6 h, then loaded directly onto SiO₂ and purified via silica gel chromatography (0-15% MeOH in DCM with 1% NH₄OH) to give the title compound as a white solid (25 mg, 56%). MS (ES+) C₂₅H₃₂N₆O₄ requires: 480. found: 481 [M+H]⁺; ¹H NMR (600 MHz, DMSO-d₆) δ 10.25 (s, 1H), 7.84 (s, 1H), 7.54 (d, J=7.4 Hz, 1H), 7.34-7.22 (m, 6H), 6.73 (d, J=2.3 Hz, 1H), 6.36 (dd, J=7.5, 2.3 Hz, 1H), 4.34 (t, J=7.1 Hz, 2H), 4.14 (d, J=5.9 Hz, 2H), 3.80 (t, J=7.1 Hz, 2H), 3.64 (s, 2H), 1.75-1.72 (m, 2H), 1.56-1.54 (m, 2H), 1.37 (s, 9H).

### Example 10

### 1-(4-(2-oxo-4-(2-(3-(trifluoromethoxy)phenyl)acetamido)pyridin-1(2H)-yl)butyl)-N-(pyridin-2-ylmethyl)-1H-1,2,3-triazole-4-carboxamide

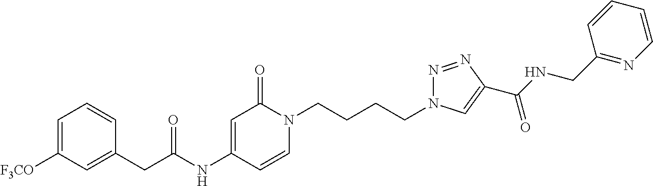 [SMILES: O=C(Cc1cccc(OC(F)(F)F)c1)Nc1ccn(CCCCn2cc(C(=O)NCc3ccccn3)nn2)c(=O)c1]

### Steps 1 to 5

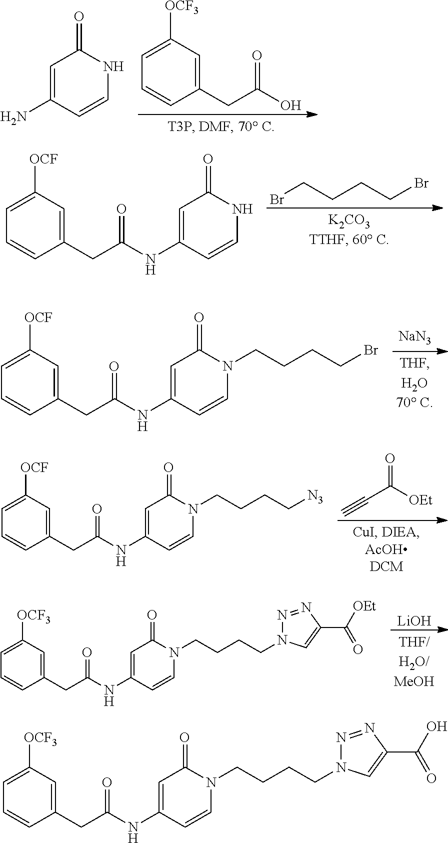

### Step 1: N-(2-oxo-1,2-dihydropyridin-4-yl)-2-(3-(trifluoromethoxy)phenyl)acetamide

T3P in DMF (1.39 g, 2.18 mmol, 50% wt solution) was added to 4-aminopyridin-2(1H)-one (0.20 g, 1.8 mmol) and 2-(3-(trifluoromethoxy)phenyl)acetic acid (0.400 g, 1.82 mmol) and the mixture stirred at 70° C. for 3 h. The reaction mixture was cooled to RT, diluted with EtOAc (20 mL) and washed with sat. NaHCO₃ (2×10 mL) and brine (10 mL). The organic layer was dried over MgSO₄ and concentrated under reduced pressure to give the title compound as a light brown solid (0.35 g, 62%). MS (ES⁺) C₁₄H₁₁F₃N₂O₃ requires: 312. found: 313 [M+H]⁺.

### Step 2: N-(1-(4-bromobutyl)-2-oxo-1,2-dihydropyridin-4-yl)-2-(3-(trifluoromethoxy)phenyl)acetamide

To a suspension of K₂CO₃ (0.155 g, 1.12 mmol) and N-(2-oxo-1,2-dihydropyridin-4-yl)-2-(3-(trifluoromethoxy)phenyl)acetamide (0.35 g, 1.1 mmol) in THF (2.24 ml) was added 1,4-dibromobutane (0.268 ml, 2.24 mmol) and the resulting mixture was stirred at 70° C. for 12 h. The reaction mixture was cooled to RT and concentrated under reduced pressure. The residue was purified via silica gel chromatography (0-10% MeOH in DCM) to give the title compound as an orange solid (72 mg, 14%). MS (ES⁺) C₁₈H₁₈BrF₃N₂O₃ requires: 446. found: 447 [M+H]⁺. Step 3: N-(1-(4-azidobutyl)-2-oxo-1,2-dihydropyridin-4-yl)-2-(3-(trifluoromethoxy)phenyl)acetamide.

To a solution of N-(1-(4-bromobutyl)-2-oxo-1,2-dihydropyridin-4-yl)-2-(3-(trifluoromethoxy)phenyl)acetamide (72 mg, 0.16 mmol) in THF (644 μl) and water (161 μl) was added NaN₃ (12.6 mg, 0.190 mmol) and the resulting mixture was stirred at 70° C. for 4 h. The reaction mixture was cooled to RT, concentrated under reduced pressure. The residue was taken up in EtOH and concentrated again to give the title compound as a light yellow solid (66 mg, 100% yield). MS (ES⁺) C₁₈H₁₈F₃N₅O₃ requires: 409. found: 410 [M+H]⁺.

### Step 4: ethyl 1-(4-(2-oxo-4-(2-(3-(trifluoromethoxy)phenyl)acetamido)pyridin-1(2H)-yl)butyl)-1H-1,2,3-triazole-4-carboxylate

To a mixture of AcOH (0.92 μl, 0.016 mmol), DIEA (2.8 μl, 0.016 mmol), ethyl propiolate (0.025 ml, 0.24 mmol), and N-(1-(4-azidobutyl)-2-oxo-1,2-dihydropyridin-4-yl)-2-(3-(trifluoromethoxy)phenyl)acetamide (0.066 g, 0.16 mmol) in DCM (0.8 ml) was added copper(I) iodide (1.5 mg, 8.1 μmol) and the resulting mixture was stirred at RT for 3 h. The reaction mixture was then concentrated under reduced pressure and purified via silica gel chromatography (0-15% MeOH in DCM with 1% NH₄OH) to give the title compound as a yellow solid (72 mg, 88%). MS (ES⁺) C₂₃H₂₄F₃N₅O₅ requires: 507. found: 508 [M+H]⁺.

### Step 5: 1-(4-(2-oxo-4-(2-(3-(trifluoromethoxy)phenyl)acetamido)pyridin-1(2H)-yl)butyl)-1H-1,2,3-triazole-4-carboxylic acid

To a solution of ethyl 1-(4-(2-oxo-4-(2-(3-(trifluoromethoxy)phenyl)acetamido)pyridin-1(2H)-yl)butyl)-1H-1,2,3-triazole-4-carboxylate (72 mg, 0.14 mmol) in THF (568 μl) and MeOH (142 μl) was added aq. LiOH (2M, 78 μl, 0.15 mmol) and the resulting mixture was stirred at RT for 4 h. The reaction mixture was then concentrated under reduced pressure to give the title compound as a light green solid (70 mg, 100%, Li salt). MS (ES⁺) C₂₁H₂₀F₃N₅O₃ requires: 479. found: 480 [M+H]⁺.

### Step 6

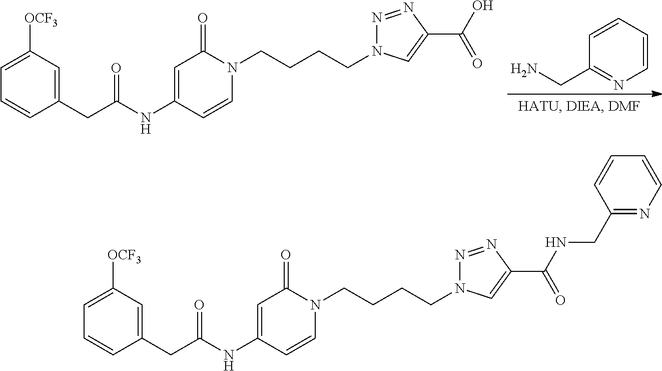 [SMILES: O=C(Cc1cccc(OC(F)(F)F)c1)Nc1ccn(CCCCn2cc(C(=O)O)nn2)c(=O)c1 ; NCc1ccccn1 ; O=C(Cc1cccc(OC(F)(F)F)c1)Nc1ccn(CCCCn2cc(C(=O)NCc3ccccn3)nn2)c(=O)c1]

### 1-(4-(2-oxo-4-(2-(3-(trifluoromethoxy)phenyl)acetamido)pyridin-1(2H)-yl)butyl)-N-(pyridin-2-ylmethyl)-1H-1,2,3-triazole-4-carboxamide

To a mixture of 1-(4-(2-oxo-4-(2-(3-(trifluoromethoxy)phenyl)acetamido)pyridin-1(2H)-yl)butyl)-1H-1,2,3-triazole-4-carboxylic acid (20 mg, 0.042 mmol), pyridin-2-ylmethanamine (5.2 μl, 0.050 mmol) and HATU (24 mg, 0.063 mmol) in DMF (209 μl) was added DIEA (14.6 μl, 0.0830 mmol) and the resulting mixture was stirred at RT for 2 h. The reaction was then concentrated under reduced pressure and the residue was purified via silica gel chromatography (0-15% MeOH in DCM with 1% NH₄OH) to give the title compound as a pink solid (12 mg, 51%). MS (ES⁺) C₂₇H₂₆N₇O₄ requires: 569. found: 570 [M+H]⁺; ¹H NMR (500 MHz, DMSO-d₆) δ 10.31 (s, 1H), 9.04 (t, J=6.1 Hz, 1H), 8.61 (s, 1H), 8.50 (m, 1H), 7.75 (m, 1H), 7.57 (d, J=7.4 Hz, 1H), 7.47 (m, 1H), 7.37-7.20 (m, 4H), 6.72 (d, J=2.2 Hz, 1H), 6.36 (dd, J=7.5, 2.4 Hz, 1H), 4.55 (d, J=6.0 Hz, 1H), 4.44 (t, J=7.0 Hz, 2H), 3.83 (t, J=7.1 Hz, 2H), 3.75 (s, 2H), 1.85-1.78 (m, 2H), 1.60-1.52 (m, 2H).

### Example 11

### 1-(4-(4-methyl-2-oxopyridin-1(2H)-yl)butyl)-N-(3-(trifluoromethoxy)benzyl)-1H-1,2,3-triazole-4-carboxamide

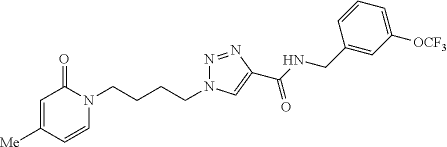 [SMILES: Cc1ccn(CCCCn2cc(C(=O)NCc3cccc(OC(F)(F)F)c3)nn2)c(=O)c1]

### Steps 1 to 2

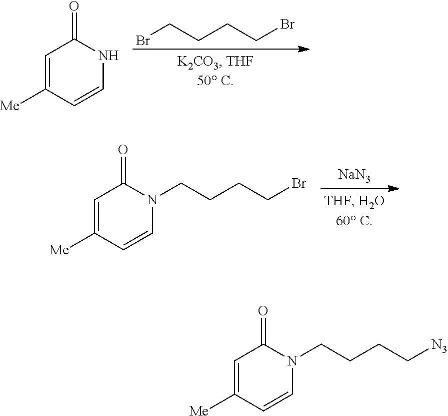

### Step 1: 1-(4-bromobut)-4-methylpyridin-2(H)-one

To a suspension of K₂CO₃ (0.127 g, 0.916 mmol) and 4-methylpyridin-2(1H)-one (0.10 g, 0.92 mmol) in THF (1.83 ml) was added 1,4-dibromobutane (0.164 ml, 1.38 mmol) and the resulting mixture was stirred at 50° C. for 12 h. The reaction mixture was then cooled to RT and concentrated under reduced pressure. The residue was purified via silica gel chromatography (0-10% MeOH in DCM) to give the title compound as a pale yellow liquid (116 mg, 47%). MS (ES⁺) C₁₀H₁₄BrNO requires: 243. found: 244 [M+H]+.

### Step 2: 1-(4-azidobutyl)-4-methylpyridin-2(1H)-one

To a solution of 1-(4-bromobutyl)-4-methylpyridin-2(1H)-one (115 mg, 0.471 mmol) in THF (1884 μl) and water (471 μl) were added NaN₃ (33.7 mg, 0.518 mmol) and the resulting mixture was stirred at 60° C. for 12 h. The reaction mixture was then concentrated under reduced pressure, the residue was taken up in EtOH and concentrated again. The residue was purified via silica gel chromatography (0-10% MeOH in DCM) to give the title compound as a pale yellow liquid (68 mg, 70%). MS (ES⁺) C₁₀H₁₄N₄O requires: 206. found: 207 [M+H]⁺.

### Step 3: N-(3-(trifluoromethoxy)benzyl)propiolamide

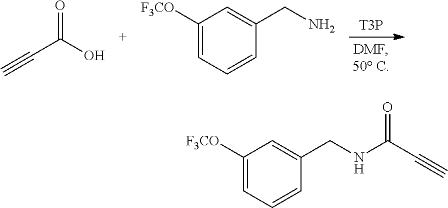

A vial was charged with T3P in DMF (3.72 g, 5.84 mmol, 50 wt % solution), propiolic acid (0.264 ml, 4.28 mmol), and (3-(trifluoromethoxy)phenyl)methanamine (0.744 ml, 3.89 mmol) and allowed to stir at 50° C. for 12 h. The orange viscous solution was concentrated under reduced pressure, and the residue was purified via silica gel chromatography (0-100% EtOAc in hexanes) to give the title compound as a pale yellow liquid (506 mg, 53%). MS (ES⁺) C₁₁H₈F₃NO₂ requires: 243. found: 244 [M+H]⁺; ¹H NMR (600 MHz, DMSO-d₆) δ 9.30 (t, J=5.8 Hz, 1H), 7.47 (t, J=7.9 Hz, 1H), 7.28 (d, J=7.7 Hz, 1H), 7.26 (m, 1H), 7.22 (s, 1H), 4.34 (d, J=6.2 Hz, 2H), 4.20 (s, 1H).

### Step 4: 1-(4-(4-methyl-2-oxopyridin-1 (2H)-yl)butyl)-N-(3-(trifluoromethoxy)benzyl)-1H-1,2,3-triazole-4-carboxamide

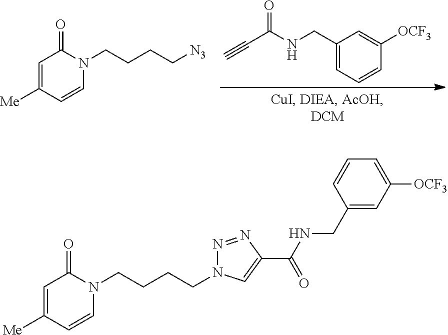

To a mixture of AcOH (0.471 μl, 8.22 μmol), DIEA (1.43 μl, 8.22 μmol), N-(3-(trifluoromethoxy)benzyl)propiolamide (20 mg, 0.082 mmol), and 1-(4-azidobutyl)-4-methylpyridin-2(1H)-one (17 mg, 0.082 mmol) in DCM (411 μl) was added copper(I) iodide (0.78 mg, 4.1 μmol) and the resulting mixture was stirred at RT for 3 h. The reaction mixture was concentrated under reduced pressure and the residue was purified via silica gel chromatography (0-10% MeOH in DCM with 1% NH₄OH) to give the title compound as a tan solid (32 mg, 74%). MS (ES⁺) C₂₁H₂₂F₃N₅O₃ requires: 449. found: 450 [M+H]⁺; ¹H NMR (600 MHz, Methanol-d₄) δ 8.38 (s, 1H), 7.47 (d, J=6.9 Hz, 1H), 7.41 (t, J=7.9 Hz, 1H), 7.35 (d, J=7.7 Hz, 1H), 7.27 (s, 1H), 7.15 (m, 1H), 6.34 (s, 1H), 6.24 (dd, J=6.9, 1.8 Hz, 1H), 4.60 (s, 2H), 4.50 (t, J=7.0 Hz, 2H), 3.98 (t, J=7.2 Hz, 2H), 2.20 (s, 3H), 1.99-1.90 (m, 2H), 1.77-1.69 (m, 2H).

### Example 12

### 1-(2-fluoro-4-(2-oxo-4-(2-phenylacetamido)pyridin-1(2H)-yl)butyl)-N-(2-fluoro-5-(trifluoromethoxy)benzyl)-1H-1,2,3-triazole-4-carboxamide

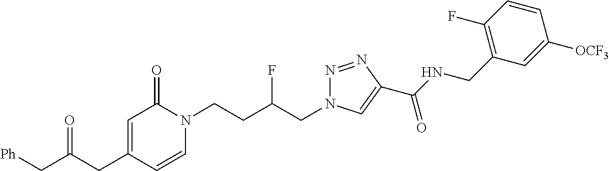 [SMILES: O=C(Cc1ccccc1)Cc1ccn(CCC(F)Cn2cc(C(=O)NCc3cc(OC(F)(F)F)ccc3F)nn2)c(=O)c1]

### Steps 1 to 5

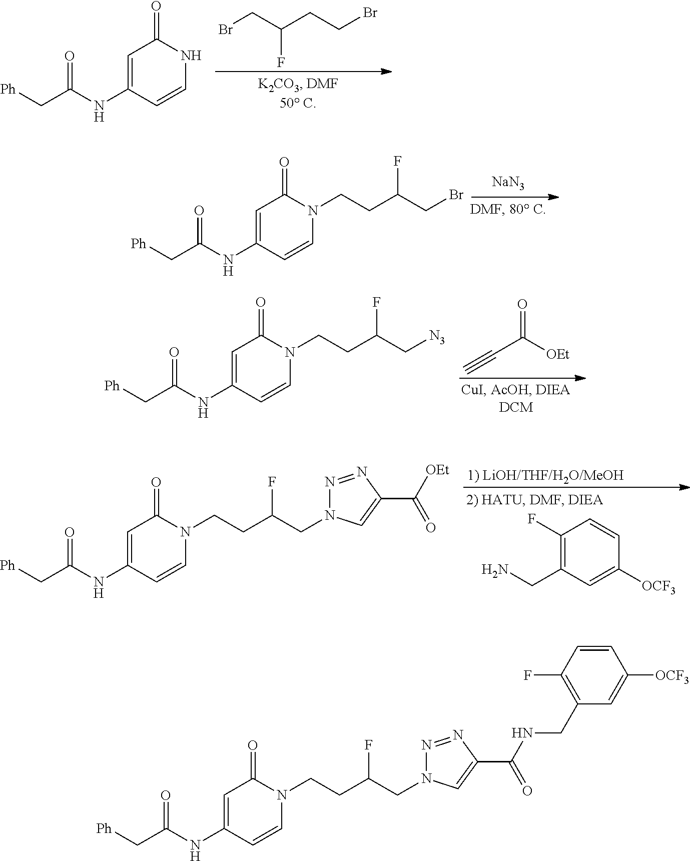

### Step 1: N-(1-(4-bromo-3-fluorobutyl)-2-oxo-1,2-dihydropyridin-4-yl)-2-phenylacetamide

To a suspension of K₂CO₃ (0.36 g, 2.6 mmol) and N-(2-oxo-1,2-dihydropyridin-4-yl)-2-phenylacetamide (0.30 g, 1.3 mmol) in DMF (2.6 ml) was added 1,4-dibromo-2-fluorobutane (0.62 g, 2.6 mmol, prepared according to WO2013/020993 A1, p. 156) and the resulting mixture was stirred at 50° C. for 5 h. The mixture was then concentrated under reduced pressure and the residue was purified via silica gel chromatography (0-10% MeOH in DCM) to give the title compound as a pale yellow, viscous liquid (0.45 g, 90%). MS (ES⁺) C₁₇H₁₈BrFN₂O₂ requires: 380. found: 381 [M+H]⁺.

### Step 2: N-(1-(4-azido-3-fluorobutyl)-2-oxo-1,2-dihydropyridin-4-yl)-2-phenylacetamide

To a suspension of N-(1-(4-bromo-3-fluorobutyl)-2-oxo-1,2-dihydropyridin-4-yl)-2-phenylacetamide (0.45 g, 1.2 mmol) in DMF (5.0 mL) was added NaN₃ (0.084 g, 1.3 mmol) and the resulting mixture was stirred at 80° C. for 3 h. The reaction was concentrated under reduced pressure and the residue was purified via silica gel chromatography (0-10% MeOH in DCM) to give the title compound as an off-white solid (0.33 g, 82%). MS (ES⁺) C₁₇H₁₈FN₅O₂ requires: 343. found: 344 [M+H]⁺.

### Step 3: Ethyl 1-(2-fluoro-4-(2-oxo-4-(2-phenylacetamido)pyridin-1(2H)-vyl)butyl)-1H-1,2,3-triazole-4-carboxylate

To a suspension of AcOH (10.0 μl, 0.175 mmol), DIEA (0.031 mL, 0.18 mmol), ethyl propiolate (0.103 g, 1.05 mmol), and N-(1-(4-azido-3-fluorobutyl)-2-oxo-1,2-dihydropyridin-4-yl)-2-phenylacetamide (0.30 g, 0.87 mmol) in DCM (4.37 ml) was added copper(I) iodide (8.3 mg, 0.044 mmol) and the resulting mixture was stirred at RT for 6 h. The reaction mixture was loaded onto a SiO₂ column and purified via silica gel chromatography (0-15% MeOH in DCM with 1% NH₄OH) to give the title compound as a white solid (0.23 g, 60%). MS (ES⁺) C₂₂H₂₄FN₅O₄ requires: 441. found: 442 [M+H]⁺.

### Step 4: 1-(2-Fluoro-4-(2-oxo-4-(2-phenylacetamido)pyridin-1(2H)-yl)butyl)-1H-1,2,3-triazole-4-carboxylic acid

To a suspension of ethyl 1-(2-fluoro-4-(2-oxo-4-(2-phenylacetamido)pyridin-1(2H)-yl)butyl)-1H-1,2,3-triazole-4-carboxylate (0.23 g, 0.52 mmol) in THF (2.1 mL) and MeOH (0.52 mL) was added aq. LiOH (0.29 mL, 0.57 mmol, 2M) and the resulting mixture was stirred at RT for 2 h. The mixture was concentrated under reduced pressure to give the title compound as an off-white solid (0.22 g, >100%, Li salt). MS (ES⁺) C₂₀H₂₀FN₅O₄ requires: 413. found: 414 [M+H]⁺.

### Step 5: 1-(2-fluoro-4-(2-oxo-4-(2-phenylacetamido)pyridin-1(2H)-yl)butyl)-N-(2-fluoro-5-(trifluoromethoxy)benzyl)-1H-1,2,3-triazole-4-carboxamide

To a suspension of 1-(2-fluoro-4-(2-oxo-4-(2-phenylacetamido)pyridin-1(2H)-yl)butyl)-1H-1,2,3-triazole-4-carboxylic acid (20 mg, 0.048 mmol), (2-fluoro-5-(trifluoromethoxy)phenyl)methanamine (10 mg, 0.048 mmol) and HATU (24 mg, 0.063 mmol) in DMF (242 μl) was added DIEA (17 μl, 0.097 mmol) and the resulting mixture was stirred at RT for 2 h. The reaction mixture was concentrated under reduced pressure. The residue was purified by mass-triggered preparative HPLC (Mobile phase: A=0.1% TFA/H₂O, B=0.1% TFA/MeCN; Gradient: B=40-80%; 12 min; Column: C18) to give the title compound as a white solid (22 mg, 75%). MS (ES⁺) C₂₈H₂₅F₅N₆O₄ requires: 604. found: 605 [M+H]⁺; ¹H NMR (600 MHz, DMSO-d₆) δ 10.28 (s, 1H), 9.18 (t, J=6.2 Hz, 1H), 8.58 (s, 1H), 7.56 (d, J=7.4 Hz, 1H), 7.37-7.28 (m, 7H), 7.25 (m, 1H), 6.77 (d, J=2.0 Hz, 1H), 6.37 (dd, J=7.6, 2.2 Hz, 1H), 5.04-4.88 (m, 1H), 4.84-4.66 (m, 2H), 4.51 (d, J=6.0 Hz, 2H), 3.99-3.91 (m, 2H), 3.65-3.50 (m, 2H), 2.12-1.83 (m, 2H).

### Example 13

### 1-(3-fluoro-4-(2-oxo-4-(2-phenylacetamido)pyridin-1(2H)-yl)butyl)-N-(2-fluoro-5-(trifluoromethoxy)benzyl)-1H-1,2,3-triazole-4-carboxamide

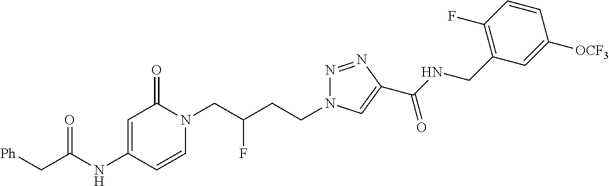 [SMILES: O=C(Cc1ccccc1)Nc1ccn(CC(F)CCn2cc(C(=O)NCc3cc(OC(F)(F)F)ccc3F)nn2)c(=O)c1]

### Steps 1 to 4

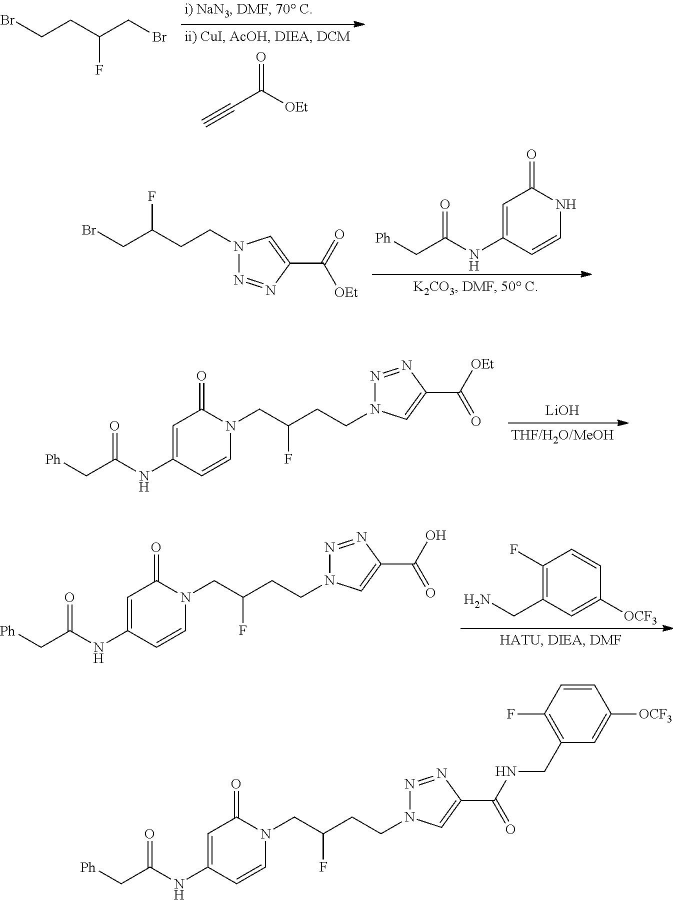

### Step 1: Ethyl 1-(4-bromo-3-fluorobutyl)-1H-1,2,3-triazole-4-carboxylate

To a solution of 1,4-dibromo-2-fluorobutane (1.0 g, 4.3 mmol; prepared according to WO2013/020993 A1, p. 156) in DMF (1.94 mL) was added NaN₃ (0.28 g, 4.3 mmol) and the resulting mixture was stirred at 70° C. for 1 h. The reaction was then cooled to RT and diluted with DCM (19 mL) before adding AcOH (0.049 mL, 0.86 mmol), DIEA (0.15 mL, 0.86 mmol), and ethyl propiolate (0.84 mL, 8.6 mmol). Finally, copper(I) iodide (0.041 g, 0.21 mmol) was added and the resulting mixture was stirred at RT for 5 h. The mixture was loaded directly onto SiO₂ and purified via silica gel chromatography (0-100% EtOAc in hexanes) to give the title compound as a pale yellow solid (0.49 g, 39%). MS (ES⁺) C₉H₁₃BrFN₃O₂ requires: 293. found: 294 [M+H]⁺.

### Step 2: Ethyl 1-(3-fluoro-4-(2-oxo-4-(2-phenylacetamido)pyridin-1(2H)-yl)butyl)-1H-1,2,3-triazole-4-carboxylate

To a suspension of K₂CO₃ (0.24 g, 1.8 mmol) and N-(2-oxo-1,2-dihydropyridin-4-yl)-2-phenylacetamide (0.20 g, 0.88 mmol) in DMF (1.8 mL) was added ethyl 1-(4-bromo-3-fluorobutyl)-1H-1,2,3-triazole-4-carboxylate (0.26 g, 0.88 mmol) and the resulting mixture was stirred at 50° C. for 5 h. The mixture was concentrated under reduced pressure and the residue was purified via silica gel chromatography (0-10% MeOH in DCM) to give the title compound as a white solid (0.17 g, 43%). MS (ES⁺) C₂₂H₂₄FN₅O₄ requires: 441. found: 442 [M+H]⁺.

### Step 3: 1-(3-Fluoro-4-(2-oxo-4-(2-phenylacetamido)pyridin-1(2H)-yl)butyl)-1H-1,2,3-triazole-4-carboxylic acid

To a suspension of ethyl 1-(3-fluoro-4-(2-oxo-4-(2-phenylacetamido)pyridin-1(2H)-yl)butyl)-1H-1,2,3-triazole-4-carboxylate (0.17 g, 0.37 mmol) in THF (1.5 mL) and MeOH (0.37 mL) was added aq. LiOH (2M, 0.21 mL, 0.41 mmol,) and the resulting mixture was stirred at RT for 2 h. The mixture was concentrated under reduced pressure to give the title compound as an off-white solid (160 mg, >100%, Li salt). MS (ES⁺) C₂₀H₂₀FN₅O₄ requires: 413. found: 414 [M+H]⁺.

### Step 4: 1-(3-fluoro-4-(2-oxo-4-(2-phenylacetamido)pyridin-1(2H)-yl)butyl)-N-(2-fluoro-5-(trifluoromethoxy)benzyl)-1H-1,2,3-triazole-4-carboxamide

To a suspension of 1-(3-fluoro-4-(2-oxo-4-(2-phenylacetamido)pyridin-1(2H)-yl)butyl)-1H-1,2,3-triazole-4-carboxylic acid (20 mg, 0.048 mmol), (2-fluoro-5-(trifluoromethoxy)phenyl)methanamine (10 mg, 0.048 mmol) and HATU (24 mg, 0.063 mmol) in DMF (242 μl) was added DIEA (17 μl, 0.097 mmol) and the resulting mixture was stirred at RT for 2 h. The reaction mixture was concentrated under reduced pressure and the residue was purified by mass-triggered preparative HPLC (Mobile phase: A=0.1% TFA/H₂O, B=0.1% TFA/MeCN; Gradient: B=40-80%; 12 min; Column: C18) to give the title compound as an off-white solid (16 mg, 55%). MS (ES⁺) C₂₈H₂₅F₅N₆O₄ requires: 604. found: 605 [M+H]⁺; ¹H NMR (500 MHz, DMSO-d₆) δ 10.32 (s, 1H), 9.17 (t, J=6.1 Hz, 1H), 8.64 (s, 1H), 7.50 (d, J=7.5 Hz, 1H), 7.40-7.18 (m, 8H), 6.79 (d, J=2.3 Hz, 1H), 6.37 (dd, J=7.5, 2.3 Hz, 1H), 4.88-4.69 (m, 1H), 4.58 (t, J=7.1 Hz, 2H), 4.50 (d, J=6.1 Hz, 2H), 4.17 (ddd, J=29.6, 14.2, 2.8 Hz, 1H), 4.01 (ddd, J=18.3, 14.2, 7.7 Hz, 1H), 3.65-3.50 (m, 2H), 2.35-2.08 (m, 2H).

### Example 14

### N-(2-oxo-1-(4-(6-(2-(3-(trifluoromethoxy)phenyl)acetamido)pyridazin-3-yl)butyl)-1,2-dihydropyridin-4-yl)-2-phenylacetamide

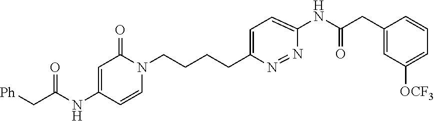 [SMILES: O=C(Cc1ccccc1)Nc1ccn(CCCCc2ccc(NC(=O)Cc3cccc(OC(F)(F)F)c3)nn2)c(=O)c1]

### Steps 1 to 3

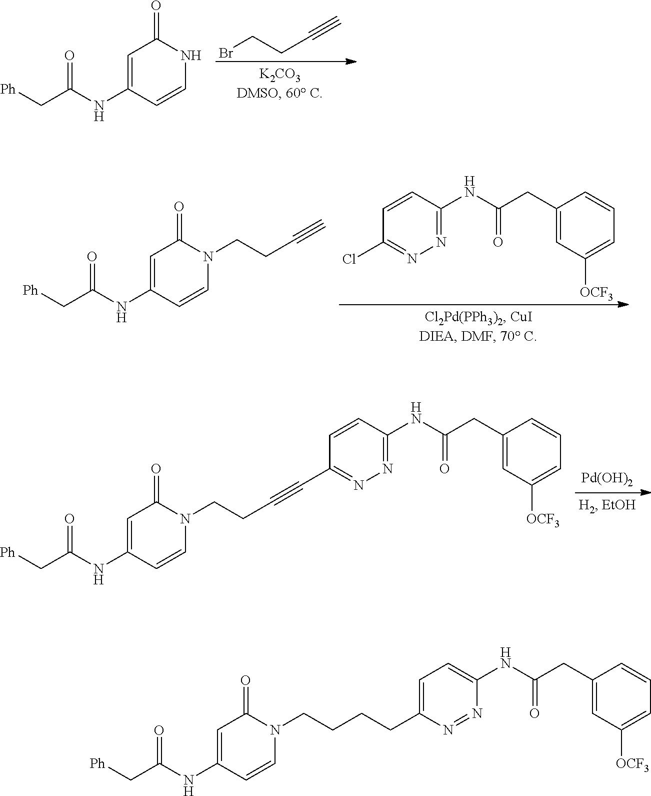

### Step 1: N-(1-(but-3-yn-1-yl)-2-oxo-1,2-dihydropyridin-4-yl)-2-phenylacetamide

To a mixture of N-(2-oxo-1,2-dihydropyridin-4-yl)-2-phenylacetamide (Example 1, Step 1; 1.2 g, 5.3 mmol) and K₂CO₃ (1.45 g, 10.5 mmol) in DMSO (10.5 ml) at 60° C. was added 4-bromobut-1-yne (0.987 ml, 10.5 mmol) in four equal portions over 4 h. The reaction mixture was then diluted with EtOAc (100 mL) and washed with water (3×10 mL) and brine (2×10 mL). The combined organic layers were concentrated under reduced pressure and the residue was adsorbed onto SiO₂ and purified via SiO₂ gel chromatography (0-20% MeOH in DCM) to give the title compound as a brown solid (407 mg, 17%). MS (ES⁺) C₁₇H₁₆N₂O₂ requires: 280. found: 281 [M+H]⁺; ¹H NMR (600 MHz, DMSO-d₆) δ 10.30 (s, 1H), 7.59 (d, J=7.4 Hz, 1H), 7.35-7.28 (m, 4H), 7.25 (m, 1H), 6.75 (d, J=2.3 Hz, 1H), 6.36 (dd, J=7.4, 2.4 Hz, 1H), 3.90 (t, J=6.7 Hz, 2H), 3.66 (s, 2H), 2.88 (t, J=2.6 Hz, 1H), 2.53 (td, J=6.8, 2.7 Hz, 2H).

### Step 2: N-(2-oxo-1-(4-(6-(2-(3-(trifluoromethoxy)phenyl)acetamido)pyridazin-3-yl)but-3-yn-1-yl)-1,2-dihydropyridin-4-yl)-2-phenylacetamide

A mixture of bis(triphenylphosphine)palladium(II) chloride (15 mg, 0.021 mmol), DIEA (748 μl, 4.28 mmol), CuI (8.1 mg, 0.043 mmol), N-(1-(but-3-yn-1-yl)-2-oxo-1,2-dihydropyridin-4-yl)-2-phenylacetamide (120 mg, 0.428 mmol) and N-(6-chloropyridazin-3-yl)-2-(3-(trifluoromethoxy)phenyl)acetamide (170 mg, 0.514 mmol) in DMF (1.43 mL) was degassed with N₂ for 5 minutes. The reaction mixture was then heated to 70° C. and stirred for 2 h, concentrated under reduced pressure and the residue was purified via silica gel chromatography (0-15% MeOH in DCM) to give the title compound as a tan solid (35 mg, 14%). MS (ES⁺) C₃₀H₂₄F₃N₅O₄ requires: 575. found: 576 [M+H]⁺; ¹H NMR (600 MHz, DMSO-d₆) δ 11.55 (s, 1H), 10.28 (s, 1H), 8.26 (d, J=9.3 Hz, 1H), 7.68 (m, 2H), 7.47 (m, 1H), 7.37 (m, 2H), 7.34-7.22 (m, 6H), 6.78 (d, J=2.3 Hz, 1H), 6.38 (dd, J=7.5, 2.3 Hz, 1H), 4.05 (t, J=6.7 Hz, 2H), 3.88 (s, 2H), 3.65 (s, 2H), 2.88 (t, J=6.7 Hz, 2H).

### Step 3: N-(2-oxo-1-(4-(6-(2-(3-(trifluoromethoxy)phenyl)acetamido)pyridazin-3-yl)butyl)-1,2-dihydropyridin-4-yl)-2-phenylacetamide

A reaction vessel was charged with palladium hydroxide on carbon (85 mg, 0.030 mmol, 5% w/w), N-(2-oxo-1-(4-(6-(2-(3-(trifluoromethoxy)phenyl)acetamido)pyridazin-3-yl)but-3-yn-1-yl)-1,2-dihydropyridin-4-yl)-2-phenylacetamide (35 mg, 0.061 mmol) and EtOH (608 μl) under an atmosphere of N₂. The suspension was degassed with N₂ for 5 minutes and purged with H₂ for 10 minutes. The reaction mixture was stirred under an H₂ balloon for 2 h at RT, then purged with N₂, filtered through a pad of Celite®, and concentrated under reduced pressure. The residue was purified via silica gel chromatography (0-15% MeOH in DCM) to give the title compound as a white solid (16 mg, 45%). MS (ES⁺) C₃₀H₂₈F₃N₅O₄ requires: 579. found: 580 [M+H]⁺; ¹H NMR (600 MHz, DMSO-d₆) δ 11.28 (s, 1H), 10.24 (s, 1H), 8.18 (d, J=9.1 Hz, 1H), 7.58-7.51 (m, 2H), 7.47 (m, 1H), 7.39-7.22 (m, 8H), 6.72 (d, J=2.3 Hz, 1H), 6.36 (dd, J=7.4, 2.4 Hz, 1H), 3.88-3.77 (m, 4H), 3.64 (s, 2H), 2.89-2.82 (m, 2H), 1.67-1.59 (m, 4H).

### Example 15

### N,N′-(butane-1,4-diylbis(2-oxo-1,2-dihydropyrimidine-1,4-diyl))bis(2-phenylacetamide)

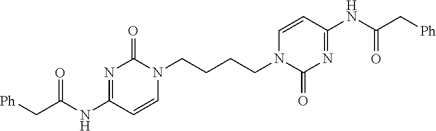 [SMILES: O=C(Cc1ccccc1)Nc1ccn(CCCCn2ccc(NC(=O)Cc3ccccc3)nc2=O)c(=O)n1]

### Steps 1 to 2

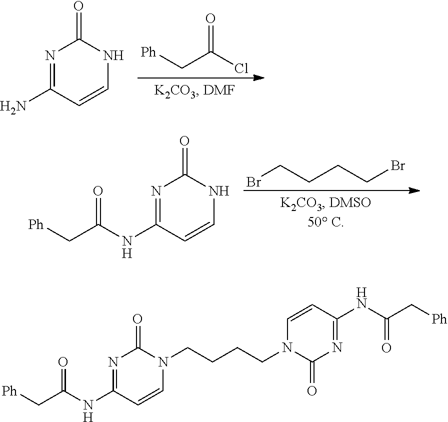

### Step 1: N-(2-oxo-1,2-dihydropyrimidin-4-yl)-2-phenylacetamide

To a suspension of 4-aminopyrimidin-2(1H)-one (1.0 g, 9.0 mmol) and K₂CO₃ (1.24 g, 9.00 mmol) in DMF (36.0 ml) at 0° C. was added 2-phenylacetyl chloride (2.21 ml, 18.0 mmol). The resulting mixture was stirred at RT for 48 h, diluted with water (200 mL) and allowed to stir at RT for 5 minutes. The mixture was then filtered through a Buchner funnel and the filter cake was washed with water (10 mL) to give the title compound as a light yellow solid (1.12 g, 54%). MS (ES⁺) C₁₂H₁₁N₃O₂ requires: 229. found: 230 [M+H]⁺.

### Step 2: N,N′-(butane-1,4-diylbis(2-oxo-1,2-dihydropyrimidine-1,4-diyl))bis(2-phenylacetamide)

To a suspension of N-(2-oxo-1,2-dihydropyrimidin-4-yl)-2-phenylacetamide (21 mg, 0.093 mmol) and K₂CO₃ (13 mg, 0.093 mmol) in DMSO (0.18 mL) was added 1,4-dibromobutane (0.0055 mL, 0.046 mmol), and the resulting mixture was heated at 50° C. for 1 h. The reaction mixture was then filtered and purified by mass-triggered preparative HPLC (Mobile phase: A=0.1% TFA/H₂O, B=0.1% TFA/MeCN; Gradient: B=30-70%; 20 minutes; Column: C18) to give the title compound as an off-white solid (8 mg, 34% yield). MS (ES⁺) C₂₈H₂₈N₆O₄ requires: 512. found: 513 [M+H]⁺; ¹H NMR (500 MHz, DMSO-d₆) δ 11.04 (s, 2H), 8.08 (d, J=7.2 Hz, 2H), 7.36-7.28 (m, 8H), 7.28-7.22 (m, 2H), 7.10 (d, J=7.2 Hz, 2H), 3.84-3.77 (m, 4H), 3.71 (s, 4H), 1.66-1.53 (m, 4H).

### Example 16

### N-(2-oxo-1-(4-(5-(2-(pyridin-3-yl)acetamido)-1,3,4-thiadiazol-2-yl)butyl)-1,2-dihydropyrimidin-4-yl)-2-phenylacetamide

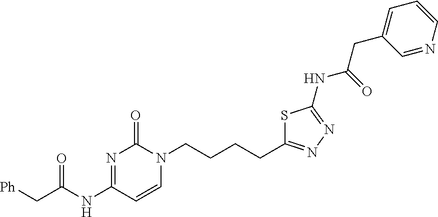 [SMILES: O=C(Cc1ccccc1)Nc1ccn(CCCCc2nnc(NC(=O)Cc3cccnc3)s2)c(=O)n1]

### Steps 1 to 3

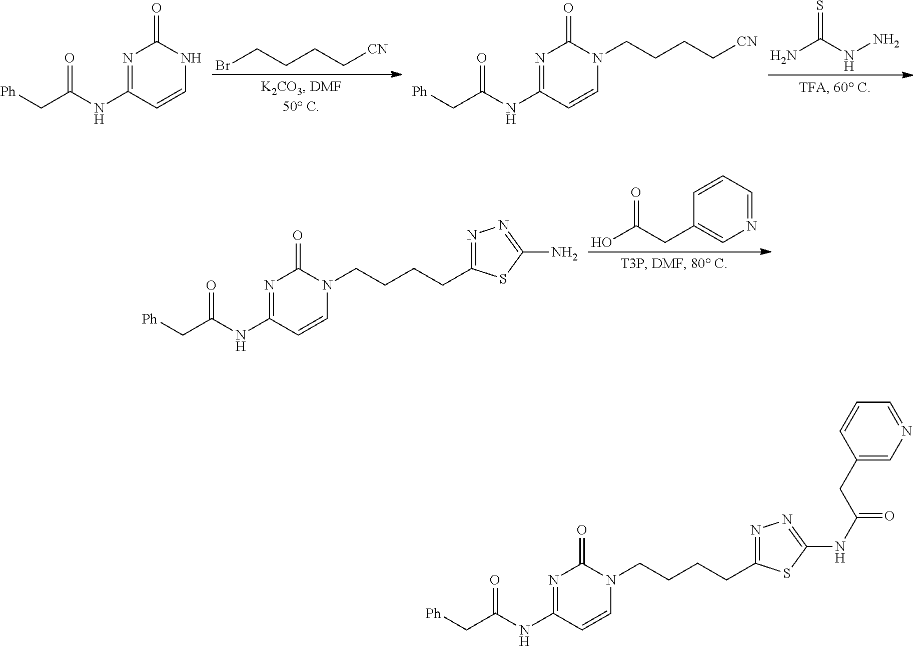

### Step 1: N-(1-(4-cyanobutyl)-2-oxo-1,2-dihydropyrimidin-4-yl)-2-phenylacetamide

To a suspension of N-(2-oxo-1,2-dihydropyrimidin-4-yl)-2-phenylacetamide (Example 15, Step 1; 0.480 g, 2.09 mmol) and K₂CO₃ (0.289 g, 2.09 mmol) in DMF (10.5 ml) was added 5-bromopentanenitrile (0.293 ml, 2.51 mmol). The resulting mixture was heated at 50° C. for 1 h. The reaction mixture was filtered, concentrated under reduced pressure, and the residue purified by mass-triggered preparative HPLC (Mobile phase: A=0.1% TFA/H₂O, B=0.1% TFA/MeCN; Gradient: B=30-70%; 12 minutes; Column: C18) to give the title compound (0.241 g, 37%) as a pale yellow solid. MS (ES⁺) C₁₇H₁₈N₄O₂ requires: 310. found: 311 [M+H]⁺; ¹H NMR (600 MHz, DMSO-d₆) δ 11.04 (s, 1H), 8.09 (d, J=7.2 Hz, 1H), 7.34-7.28 (m, 4H), 7.28-7.21 (m, 1H), 7.12 (d, J=7.2 Hz, 1H), 3.81 (t, J=7.0 Hz, 2H), 3.72 (s, 2H), 3.69 (s, 2H), 2.53 (t, J=7.1 Hz, 2H), 1.77-1.67 (m, 2H), 1.58-1.49 (m, 2H).

### Step 2: N-(1-(4-(5-amino-1,3,4-thiadiazol-2-yl)butyl)-2-oxo-1,2-dihydropyrimidin-4-yl)-2-phenylacetamide

To a solution of N-(1-(4-cyanobutyl)-2-oxo-1,2-dihydropyrimidin-4-yl)-2-phenylacetamide (240 mg, 0.773 mmol) in trifluoroacetic acid (596 al, 7.73 mmol) was added hydrazinecarbothioamide (70.5 mg, 0.773 mmol) and the resulting mixture was stirred at 60° C. for 12 h. The reaction was diluted with toluene (5 mL) and concentrated under reduced pressure. The residue was purified via silica gel chromatography (0-20% MeOH in DCM) to give the title compound as a light yellow solid (72 mg, 24% yield). MS (ES⁺) C₁₈H₂₀N₆O₂S requires: 384. found: 385 [M+H]⁺; ¹H NMR (600 MHz, Methanol-d₄) δ 8.00 (d, J=7.3 Hz, 1H), 7.37 (d, J=7.2 Hz, 1H), 7.33-7.30 (m, 4H), 7.28-7.24 (m, 1H), 3.94 (t, J=7.0 Hz, 2H), 3.75 (s, 2H), 2.96 (t, J=7.3 Hz, 2H), 1.88-1.72 (m, 4H).

### Step 3: N-(2-oxo-1-(4-(5-(2-(pyridin-3-yl)acetamido)-1,3,4-thiadiazol-2-yl)butyl)-1,2-dihydropyrimidin-4-yl)-2-phenylacetamide

To a suspension of N-(1-(4-(5-amino-1,3,4-thiadiazol-2-yl)butyl)-2-oxo-1,2-dihydropyrimidin-4-yl)-2-phenylacetamide (35 mg, 0.091 mmol) and 2-(pyridin-3-yl)acetic acid hydrochloride (17.4 mg, 0.100 mmol) in DMF (910 μl) was added T3P (63.7 μl, 0.100 mmol, 50% solution in DMF) and the resulting mixture was stirred at 80° C. for 8 h. The reaction mixture was then concentrated under reduced pressure and the residue purified by mass-triggered preparative HPLC (Mobile phase: A=0.1% TFA/H₂O, B=0.1% TFA/MeCN; Gradient: B=20-60%; 12 minutes; Column: C18) to give the title compound as a pale yellow solid (5 mg, 9%). MS (ES⁺) C₂₅H₂₅N₇O₃ requires: 503. found: 504 [M+H]⁺; ¹H NMR (600 MHz, DMSO-d₆) δ 12.78 (s, 1H), 11.03 (s, 1H), 8.83-8.73 (m, 2H), 8.32 (dd, J=8.0, 1.9 Hz, 1H), 8.08 (d, J=7.2 Hz, 1H), 7.88 (dd, J=8.0, 5.4 Hz, 1H), 7.33-7.29 (m, 4H), 7.27-7.24 (m, 1H), 7.10 (d, J=7.2 Hz, 1H), 4.08 (s, 2H), 3.82 (t, J=6.6 Hz, 2H), 3.71 (s, 2H), 3.00 (t, J=7.0 Hz, 2H), 1.73-1.61 (m, 4H).

### Example 17

### (S)-2-hydroxy-N-(5-(4-(2-oxo-4-(2-phenylacetamido)pyrimidin-1(2H)-yl)butyl)-1,3,4-thiadiazol-2-yl)-2-phenylacetamide

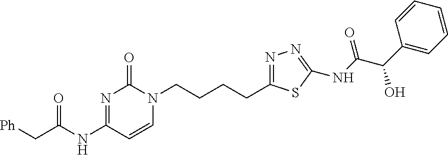 [SMILES: O=C(Cc1ccccc1)Nc1ccn(CCCCc2nnc(NC(=O)[C@@H](O)c3ccccc3)s2)c(=O)n1]

A vial was charged with N-methylmorpholine (14.6 μl, 0.133 mmol), N-(1-(4-(5-amino-1,3,4-thiadiazol-2-yl)butyl)-2-oxo-1,2-dihydropyrimidin-4-yl)-2-phenylacetamide (Step 3, Example 16; 51 mg, 0.13 mmol), (S)-2-hydroxy-2-phenylacetic acid (26.2 mg, 0.172 mmol), EDC (30.5 mg, 0.159 mmol), HOBT (24.4 mg, 0.159 mmol), and DMF (663 μl). The resulting mixture was stirred at RT for 15 h. The reaction was concentrated under reduced pressure and the residue was purified by mass-triggered preparative HPLC (Mobile phase: A=0.1% TFA/H₂O, B=0.1% TFA/MeCN; Gradient: B=30-70%; 12 minutes; Column: C18). The residual TFA was neutralized using Agilent StratoSpheres PL-HCO3 MP resin before concentrating to give the title compound (11 mg, 13% yield) as a colorless thick oil. MS (ES+) C₂₆H₂₆N₆O₄S requires: 518. found: 519 [M+H]⁺; ¹H NMR (600 MHz, Methanol-d₄) δ 7.55-7.48 (m, 2H), 7.37-7.26 (m, 10H), 5.32 (d, J=4.1 Hz, 1H), 3.93 (t, J=6.6 Hz, 2H), 3.91-3.84 (m, 1H), 3.75 (s, 1H), 3.59 (s, 1H), 3.09-3.03 (m, 2H), 1.84-1.76 (m, 4H).

### Example 18

### N-(2-oxo-1-{4-[5-(2-phenylacetamido)-1,3,4-thiadiazol-2-yl]butyl}-1,2-dihydropyrimidin-4-yl)-2-[3-(trifluoromethoxy)phenyl]acetamide

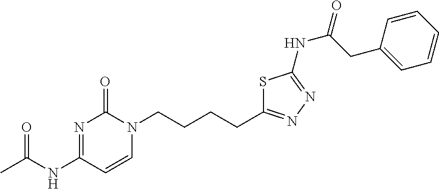 [SMILES: CC(=O)Nc1ccn(CCCCc2nnc(NC(=O)Cc3ccccc3)s2)c(=O)n1]

Prepared as Example 16 (405 mg, 95%); MS (ES⁺) C₂₀H₂₂N₆O₃S requires: 426. found: 427 [M+H]⁺. ¹H NMR (600 MHz, DMSO-d₆) δ 12.65 (s, 1H), 10.78 (s, 1H), 8.07 (d, J=7.2 Hz, 1H), 7.35-7.24 (m, 5H), 7.11 (d, J=7.2 Hz, 1H), 3.83-3.78 (m, 4H), 2.99 (t, J=7.2 Hz, 2H), 2.08 (s, 3H), 1.72-1.62 (m, 4H).

### Example 19

### N-(2-oxo-1-{4-[5-(2-phenylacetamido)-1,3,4-thiadiazol-2-yl]butyl}-1,2dihydropyrimidin-4-yl)-2-[3-(trifluoromethoxy)phenyl]acetamide

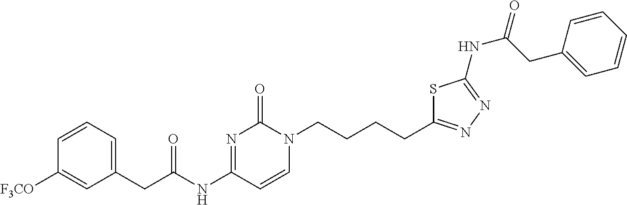 [SMILES: O=C(Cc1cccc(OC(F)(F)F)c1)Nc1ccn(CCCCc2nnc(NC(=O)Cc3ccccc3)s2)c(=O)n1]

### Steps 1 and 2

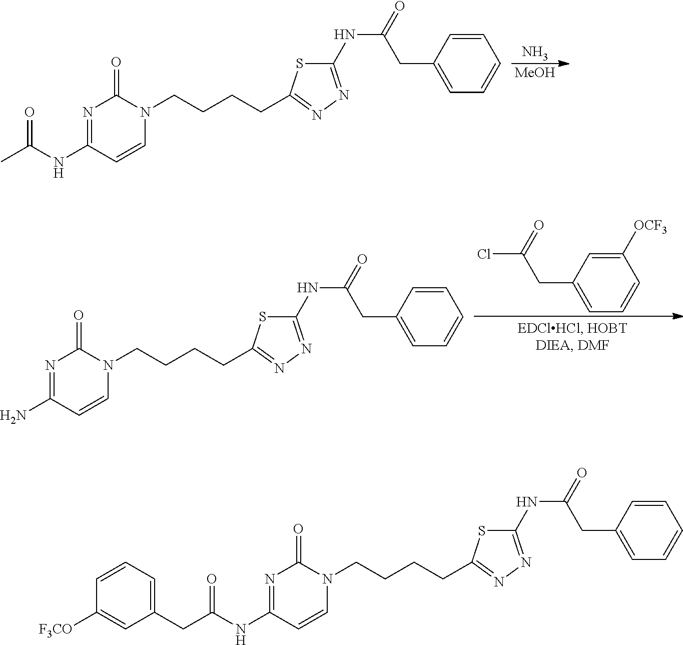

### Step 1: N-(5-(4-(4-amino-2-oxopyrimidin-1(2H)-vyl)butyl)-1,3,4-thiadiazol-2-yl)-2-phenylacetamide

A suspension of N-(5-(4-(4-acetamido-2-oxopyrimidin-1(2H)-yl)butyl)-1,3,4-thiadiazol-2-yl)-2-phenylacetamide (Example 18; 85 mg, 0.20 mmol) in ammonia in MeOH (7N, 3 ml) was stirred at 20° C. for 16 h. The precipitate was filtered to give N-(5-(4-(4-amino-2-oxopyrimidin-1(2H)-yl)butyl)-1,3,4-thiadiazol-2-yl)-2-phenylacetamide (52 mg, 68%) as a white solid. MS (ES⁺) C₁₈H₂₀N₆O₂S requires: 384. found: 385 [M+H]⁺.

### Step 2: N-(2-oxo-1-{4-[5-(2-phenylacetamido)-1,3,4-thiadiazol-2-yl]butyl}-1,2-dihydropyrimidin-4-yl)-2-[3-(trifluoromethoxy)phenyl]acetamide

To a suspension of N-(5-(4-(4-amino-2-oxopyrimidin-1(2H)-yl)butyl)-1,3,4-thiadiazol-2-yl)-2-phenylacetamide (40 mg, 0.10 mmol) in DMF (1 ml) were added 2-(3-(trifluoromethoxy)phenyl)acetic acid (27.5 mg, 0.125 mmol), HOBT (19.1 mg, 0.125 mmol), DIEA (0.035 mL, 0.19 mmol) and EDC.HCl (23.9 mg, 0.125 mmol) and the resulting mixture was stirred at 20° C. for 16 h. The volatiles were removed under reduced pressure. The crude residue was taken up in DMSO and purified by mass-triggered preparative HPLC (Mobile phase: A=0.1% TFA/H₂O, B=0.1% TFA/MeCN; Gradient: B=40-80%; 12 minutes; Column: C18) to give the title compound (30 mg, 49%) as a white solid. MS (ES⁺) C₂₇H₂₅F₃N₆O₄S requires: 586. found: 587 [M+H]⁺. ¹H NMR (600 MHz, DMSO-d₆) δ 12.67 (s, 1H), 11.08 (s, 1H), 8.09 (d, J=7.2 Hz, 1H), 7.46 (t, J=8.4 Hz, 1H), 7.35-7.24 (m, 8H), 7.09 (d, J=7.2 Hz, 1H), 3.83-3.78 (m, 6H), 2.99 (t, J=7.2 Hz, 2H), 1.72-1.62 (m, 4H).

### Example 20

### N-(2-oxo-1-(4-(6-(2-phenylacetamido)pyridazin-3-yl)butyl)-1,2-dihydropyrimidin-4-yl)-2-phenylacetamide

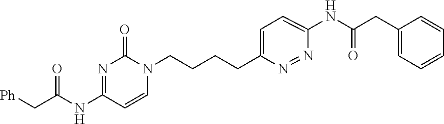 [SMILES: O=C(Cc1ccccc1)Nc1ccc(CCCCn2ccc(NC(=O)Cc3ccccc3)nc2=O)nn1]

Prepared as Example 1; MS (ES⁺) C₂₈H₂₈F₃N₆O₃ requires: 496. found: 497 [M+H]⁺; ¹H NMR (500 MHz, DMSO-d₆) δ 11.16 (m, 2H), 8.18 (d, J=9.1 Hz, 1H), 8.06 (d, J=7.2 Hz, 1H), 7.55 (d, J=9.1 Hz, 1H), 7.39-7.18 (m, 10H), 7.08 (d, J=7.2 Hz, 1H), 3.85-3.78 (m, 2H), 3.76 (s, 2H), 3.70 (s, 2H), 2.90-2.82 (m, 2H), 1.71-1.58 (m, 4H).

### Example 21

### N-(6-(4-(4-amino-2-oxopyrimidin-1(2H)-yl)butyl)pyridazin-3-yl)-2-phenylacetamide

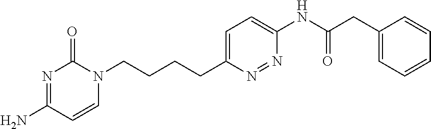 [SMILES: Nc1ccn(CCCCc2ccc(NC(=O)Cc3ccccc3)nn2)c(=O)n1]

### Steps 1 to 4

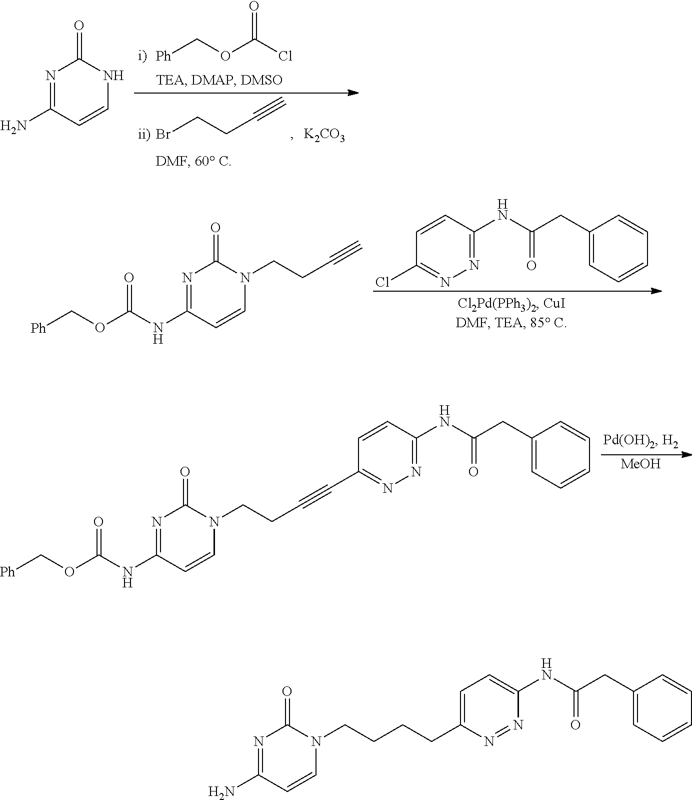

### Step 1: Benzyl(2-oxo-1,2-dihydropyrimidin-4-yl)carbamate

To a suspension of 4-aminopyrimidin-2(1H)-one (1.0 g, 9.0 mmol), TEA (1.88 mL, 13.5 mmol), and DMAP (110 mg, 0.90 mmol) in DMSO (30 mL) at 0° C. was added dropwise Cbz-C1 (1.93 mL, 13.5 mmol) and the mixture was stirred at RT for 5 days. The reaction mixture was then poured into ice-water (100 mL) and cooled to 0° C. for 10 minutes. The precipitate was filtered, washed with water (50 mL) and Et₂O (50 mL) to give the title compound as a white solid (1.6 g, 72%). MS (ES⁺) C₁₂H₁₁N₃O₃ requires: 245. found: 246 [M+H]⁺.

### Step 2: Benzyl(1-(but-3-yn-1-yl)-2-oxo-1,2-dihydropyrimidin-4-yl)carbamate

A mixture of benzyl(2-oxo-1,2-dihydropyrimidin-4-yl)carbamate (3.0 g, 12 mmol), 4-bromobut-1-yne (1.7 mL, 18 mmol) and K₂CO₃ (1.7 g, 12 mmol) in DMF (50 mL) was heated at 60° C. for 16 h. The mixture was concentrated under reduced pressure and the residue was purified by SiO₂ gel column chromatography (0 to 10% MeOH in DCM) to give the title compound (2.3 g, 80%). MS (ES⁺) C₁₆H₁₅N₃O₃ requires: 297. found: 298 [M+H]⁺.

### Step 3: Benzyl(2-oxo-1-(4-(6-(2-phenylacetamido)pyridazin-3-yl)but-3-yn-1-v)-1,2-dihydropyrimidin-4-yl)carbamate

To a solution of benzyl(1-(but-3-yn-1-yl)-2-oxo-1,2-dihydropyrimidin-4-yl)carbamate (2.2 g, 5.9 mmol)) in DMF (30 mL) was added TEA (8.25 mL, 59.2 mmol) and the mixture was degassed with a stream of N₂ for 2 minutes. CuI (56 mg, 0.30 mmol), N-(6-chloropyridazin-3-yl)-2-phenylacetamide (1.47 g, 5.92 mmol), and bis(triphenylphosphine)palladium(II) chloride (208 mg, 0.300 mmol) were then added, and the resulting mixture was heated at 85° C. for 4 h under N₂. The mixture was concentrated under reduced pressure at 60° C., and the residue was purified by silica gel column chromatography (DCM:MeOH=10:0 to 10:1) to give the title compound (1.68 g, 56%). MS (ES⁺) C₂₈H₂₄N₆O₄ requires: 508. found: 509 [M+H]⁺.

### Step 4: N-(6-(4-(4-amino-2-oxopyrimidin-1 (2H)-yl)butyl)pyridazin-3-yl)-2-phenylacetamide

A reaction vessel was charged with palladium hydroxide on carbon (20% w/w, 1.16 g, 1.65 mmol), benzyl(2-oxo-1-(4-(6-(2-phenylacetamido)pyridazin-3-yl)but-3-yn-1-yl)-1,2-dihydropyrimidin-4-yl)carbamate (1.68 g, 3.30 mmol) and MeOH (50 mL) under an atmosphere of N₂. The suspension was degassed with N₂ for 5 minutes, purged with H₂ and shaken under H₂ (40 PSI) at RT for 10 h in a Parr apparatus. The mixture was then purged with N₂, filtered through a pad of Celite® and washed with MeOH. The filtrate was concentrated under reduced pressure, the residue was taken up in MeOH/DCM/Hex (1:1:10), stirred with for 30 minutes, filtered and washed with hexanes to give the title compound (950 mg, 76%). MS (ES⁺) C₂₀H₂₂N₆O₂ requires: 378. found: 379 [M+H]⁺. ¹H NMR (600 MHz, Methanol-d₄) δ 8.39 (d, J=9.2 Hz, 1H), 7.92 (d, J=7.7 Hz, 1H), 7.65 (d, J=9.2 Hz, 1H), 7.38-7.24 (m, 5H), 6.02 (d, J=7.7 Hz, 1H), 3.88 (t, J=6.4 Hz, 2H), 3.79 (s, 2H), 3.00-2.93 (m, 2H), 1.83-1.74 (m, 4H).

### Example 22

### (S)-2-hydroxy-N-(2-oxo-1-(4-(6-(2-phenylacetamido)pyridazin-3-yl)butyl)-1,2-dihydropyrimidin-4-yl)-2-phenylacetamide

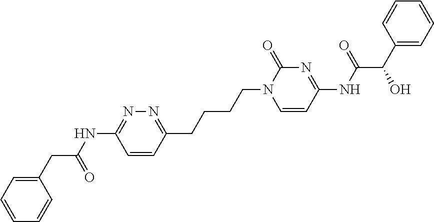 [SMILES: O=C(Cc1ccccc1)Nc1ccc(CCCCn2ccc(NC(=O)[C@@H](O)c3ccccc3)nc2=O)nn1]

To a mixture of N-(6-(4-(4-amino-2-oxopyrimidin-1(2H)-yl)butyl)pyridazin-3-yl)-2-phenylacetamide (Example 21; 24 mg, 0.063 mmol) and (S)-2-hydroxy-2-phenylacetic acid (12 mg, 0.082 mmol) and DIEA (0.020 mL, 0.19 mmol) in DMF (0.5 mL) were added EDC.HCl (15 mg, 0.076 mmol) and HOBT (12 mg, 0.11 mmol). The mixture was stirred at RT for 6 h, then diluted with DCM. The pH was adjusted to ˜7-8 by addition of aq. 1 N NaOH. The two layers were separated, the organic layer was washed with water, dried over Na₂SO₄, filtered, and concentrated under reduced pressure. The residue was purified by SiO₂ gel column chromatography (DCM:MeOH=10:0 to 10:1) to give the title compound (8.5 mg, 26%). MS (ES⁺) C₂₈H₂₈N₆O₄ requires: 512. found: 513 [M+H]⁺; ¹H NMR (600 MHz, DMSO-d₆) δ 11.23 (s, 1H), 10.34 (s, 1H), 8.17 (d, J=9.0 Hz, 1H), 8.10 (d, J=7.2 Hz, 1H), 7.54 (d, J=9.0 Hz, 1H), 7.48 (d, J=7.2 Hz, 2H), 7.37-7.30 (m, 7H), 7.25 (d, J=7.2 Hz, 1H), 7.08 (d, J=7.2 Hz, 1H), 6.40 (bs, 1H), 5.22 (bs, 1H), 3.82-3.81 (m, 2H), 3.76 (s, 2H), 2.87-2.85 (m, 2H), 1.66-1.64 (m, 4H).

### Example 23

### N-(6-(4-(4-(3-benzylureido)-2-oxopyrimidin-1(2H)-yl)butyl)pyridazin-3-yl)-2-(3-(trifluoromethoxy)phenyl)acetamide

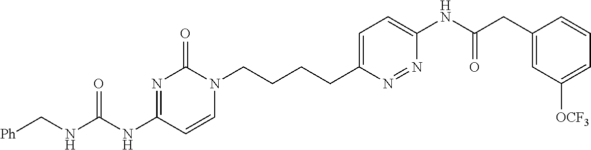 [SMILES: O=C(Cc1cccc(OC(F)(F)F)c1)Nc1ccc(CCCCn2ccc(NC(=O)NCc3ccccc3)nc2=O)nn1]

To a solution of N-(6-(4-(4-amino-2-oxopyrimidin-1(2H)-yl)butyl)pyridazin-3-yl)-2-(3-(trifluoromethoxy)phenyl)acetamide (Example 21; 34 mg, 0.074 mmol) in THF (294 μl) was added (isocyanatomethyl)benzene (12 μl, 0.096 mmol) and the resulting mixture was stirred at RT for 12 h. The mixture was concentrated under reduced pressure and the residue was purified by mass-triggered preparative HPLC (Mobile phase: A=0.1% TFA/H₂O, B=0.1% TFA/MeCN; Gradient: B=40-80%; 12 minutes; Column: C18) to give the title compound as a white solid (3 mg, 7%). MS (ES⁺) C₂₉H₂₈F₃N₇O₄ requires: 595. found: 596 [M+H]⁺; ¹H NMR (600 MHz, DMSO-d₆) δ 11.31 (s, 1H), 9.90 (s, 1H), 9.36 (s, 1H), 8.19 (d, J=9.1 Hz, 1H), 7.93 (d, J=7.1 Hz, 1H), 7.56 (d, J=9.2 Hz, 1H), 7.47 (t, J=7.9 Hz, 1H), 7.39-7.23 (m, 8H), 6.20 (s, 1H), 4.43 (s, 2H), 3.85 (s, 2H), 3.79-3.73 (m, 2H), 2.91-2.82 (m, 2H), 1.69-1.58 (m, 4H).

### Example 24

### Benzyl(2-oxo-1-(4-(6-(2-(3-(trifluoromethoxy)phenyl)acetamido)pyridazin-3-yl)butyl)-1,2-dihydropyrimidin-4-yl)carbamate

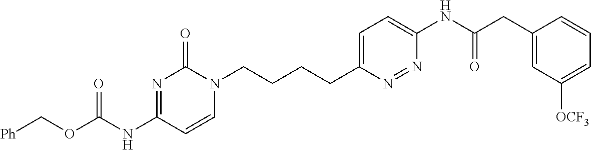 [SMILES: O=C(Cc1cccc(OC(F)(F)F)c1)Nc1ccc(CCCCn2ccc(NC(=O)OCc3ccccc3)nc2=O)nn1]

To a mixture of N-(6-(4-(4-amino-2-oxopyrimidin-1(2H)-yl)butyl)pyridazin-3-yl)-2-(3-(trifluoromethoxy)phenyl)acetamide (Example 21; 20 mg, 0.043 mmol) and pyridine (7.0 μl, 0.087 mmol) in DMF (216 μl) was added benzyl carbonochloridate (9.2 μl, 0.065 mmol). The orange solution was allowed to stir at RT for 24 h. The reaction was concentrated under reduced pressure and the residue was purified via silica gel chromatography (0-20% MeOH in DCM) to give the title compound as an off-white solid (10 mg, 39%). MS (ES+) C₂₉H₂₇F₃N₆O₅ requires: 596. found: 597 [M+H]⁺; ¹H NMR (600 MHz, Methanol-d₄) δ 8.34 (d, J=9.1 Hz, 1H), 7.95 (d, J=7.3 Hz, 1H), 7.57 (d, J=9.2 Hz, 1H), 7.46-7.28 (m, 8H), 7.24 (d, J=7.3 Hz, 1H), 7.21-7.14 (m, 1H), 5.21 (s, 2H), 3.92 (t, J=6.6 Hz, 2H), 3.85 (s, 2H), 2.95 (t, J=7.0 Hz, 2H), 1.82-1.72 (m, 4H).

### Example 25

### N-(6-(4-(4-((3-methoxypropyl)amino)-2-oxopyrimidin-1(2H)-yl)butyl)pyridazin-3-yl)-2-phenylacetamide

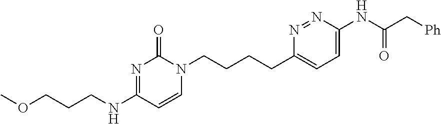 [SMILES: COCCCNc1ccn(CCCCc2ccc(NC(=O)Cc3ccccc3)nn2)c(=O)n1]

To a suspension of NaH (3.80 mg, 0.095 mmol) in DMF (793 μl) was added dropwise N-(6-(4-(4-amino-2-oxopyrimidin-1(2H)-yl)butyl)pyridazin-3-yl)-2-phenylacetamide (Example 21; 30 mg, 0.079 mmol) and the resulting mixture was stirred at RT for 30 minutes. A solution of 1-bromo-3-methoxypropane (11 L, 0.095 mmol) in DMF (793 μl) was added dropwise, and the resulting mixture was stirred at RT for 2 h. The reaction mixture was then diluted with EtOAc (10 mL) and washed with H₂O (10 mL). The layers were separated, and the organic layer was washed with brine (10 mL), dried over Na₂SO₄, filtered and concentrated under reduced pressure. The residue was purified by mass-triggered preparative HPLC (Mobile phase: A=0.1% TFA/H₂O, B=0.1% TFA/MeCN; Gradient: B=10-40%; 12 minutes; Column: C18) to give the title compound as a white solid (5 mg, 11%); MS (ES⁺) C₂₄H₃₀N₆O₃ requires: 450. found: 451 [M+H]⁺; ¹H NMR (600 MHz, CD₃OD) δ 8.70 (d, J=9.4 Hz, 1H), 8.11 (d, J=9.3 Hz, 1H), 7.93 (d, J=7.6 Hz, 1H), 7.40-7.32 (m, 3H), 7.34-7.26 (m, 2H), 6.04 (d, J=7.6 Hz, 1H), 4.72 (t, J=6.8 Hz, 2H), 3.96 (s, 2H), 3.91-3.89 (m, 2H), 3.43 (t, J=5.7 Hz, 2H), 3.25 (s, 3H), 3.02 (t, J=6.9 Hz, 2H), 2.22 (p, J=6.3 Hz, 2H), 1.86-1.80 (m, 4H).

### Example 26

### N-(6-(4-(4-(2-methylpropylsulfonamido)-2-oxopyrimidin-1(2H)-yl)butyl)pyridazin-3-yl)-2-phenylacetamide

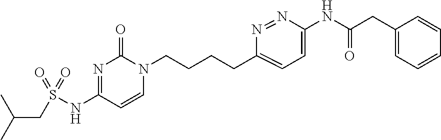 [SMILES: CC(C)CS(=O)(=O)Nc1ccn(CCCCc2ccc(NC(=O)Cc3ccccc3)nn2)c(=O)n1]

To a suspension of N-(6-(4-(4-amino-2-oxopyrimidin-1(2H)-yl)butyl)pyridazin-3-yl)-2-phenylacetamide (Example 21; 30 mg, 0.079 mmol) in THF (0.4 ml) was added NaH (60% mineral oil dispersion; 8.4 mg, 0.21 mmol). The resulting mixture was stirred at RT for 1 h, 2-methylpropane-1-sulfonyl chloride (10 μl, 0.079 mmol) was added and the resulting mixture was stirred at RT for another 16 h. The reaction mixture was then diluted with DMSO (1 mL), filtered, and the filtrate was purified by mass-triggered preparative HPLC (Mobile phase: A=0.1% TFA/H₂O, B=0.1% TFA/MeCN; Gradient: B=30-70%; 12 minutes; Column: C18) to give the title compound as an off-white solid (5 mg, 13%). MS (ES⁺) C₂₄H₃₀N₆O₄S requires: 498.2. found: 499 [M+H]+. ¹H NMR (DMSO-d₆) δ: 11.87 (br s, 1H), 11.26 (s, 1H), 8.21 (d, J=9.4 Hz, 1H), 7.81 (d, J=7.9 Hz, 1H), 7.58 (d, J=9.1 Hz, 1H), 7.29-7.38 (m, 4H), 7.20-7.28 (m, 1H), 6.40 (d, J=7.9 Hz, 1H), 3.68-3.81 (m, 4H), 2.82-2.95 (m, 4H), 2.08-2.21 (m, 1H), 1.57-1.73 (m, 4H), 0.97-1.07 (m, 6H).

### Example 27

### N-benzyl-1-(4-(2-oxo-4-(2-(3-(trifluoromethoxy)phenyl)acetamido)pyrimidin-1(2H)-yl)butyl)-1H-1,2,3-triazole-4-carboxamide

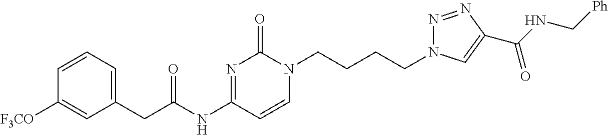 [SMILES: O=C(Cc1cccc(OC(F)(F)F)c1)Nc1ccn(CCCCn2cc(C(=O)NCc3ccccc3)nn2)c(=O)n1]

### Steps 1 to 5

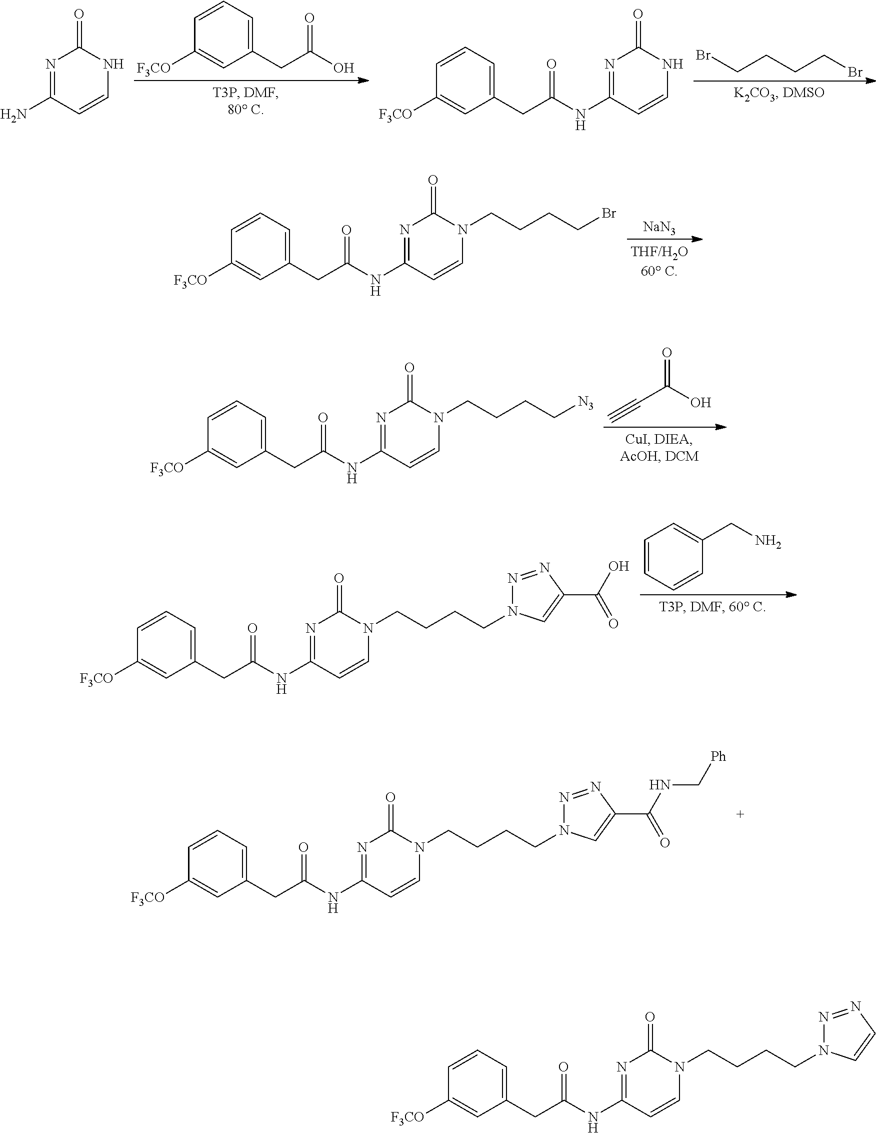

### Step 1: N-(2-oxo-1,2-dihydropyrimidin-4-yl)-2-(3-(trifluoromethoxy)phenyl)acetamide

T3P in DMF (6.87 g, 10.8 mmol, 50% solution) was added to 4-aminopyrimidin-2(1H)-one (1.0 g, 9.0 mmol) and the mixture stirred at 80° C. for 4 h. The orange solution was cooled to 0° C. and diluted with water (70 mL). After stirring at 0° C. for 10 minutes the mixture was filtered on a Buchner funnel. The solid was washed with Et₂O (10 mL), and the title compound as collected as a yellow-orange solid (1.64 g, 55%). MS (ES⁺) C₁₃H₁₀F₃N₃O₃ requires: 313. found: 314 [M+H]⁺. Step 2: N-(1-(4-bromobutyl)-2-oxo-1,2-dihydropyrimidin-4-yl)-2-(3-(trifluoromethoxy)phenyl)acetamide.

To a mixture of K₂CO₃ (0.35 g, 2.5 mmol) and N-(2-oxo-1,2-dihydropyrimidin-4-yl)-2-(3-(trifluoromethoxy)phenyl)acetamide (0.80 g, 2.5 mmol) in DMSO (5.1 mL) was added 1,4-dibromobutane (0.30 mL, 0.64 mmol) and the resulting mixture was stirred at RT for 4 h. The reaction mixture was then filtered, diluted with EtOAc (50 mL) and washed with brine (5×10 mL) before concentrating under reduced pressure. The residue was purified via silica gel chromatography (0-20% MeOH in DCM) to give the title compound as a colorless oil (249 mg, 22%). MS (ES⁺) C₁₇H₁₇BrF₃N₃O₃ requires: 447. found: 448 [M+H]⁺. Step 3: N-(1-(4-azidobutyl)-2-oxo-1,2-dihydropyrimidin-4-yl)-2-(3-(trifluoromethoxy)phenyl)acetamide.

To a solution of N-(1-(4-bromobutyl)-2-oxo-1,2-dihydropyrimidin-4-yl)-2-(3-(trifluoromethoxy)phenyl)acetamide (0.25 g, 0.56 mmol) in THF (2.2 ml) and water (0.56 ml) was added sodium azide (0.040 g, 0.61 mmol) and the resulting mixture was stirred at 60° C. for 12 h. The reaction mixture was concentrated under reduced pressure to give the title compound as a light yellow solid (0.25 g, >100%). MS (ES⁺) C₁₇H₁₇F₃N₆O₃ requires: 410. found: 411 [M+H]⁺.

### Step 4: 1-(4-(2-oxo-4-(2-(3-(trifluoromethoxy)phenyl)acetamido)pyrimidin-1(2H)-yl)butyl)-1H-1,2,3-triazole-4-carboxylic acid

To a mixture of DIEA (98 μl, 0.56 mmol), AcOH (6.4 μl, 0.11 mmol), and N-(1-(4-azidobutyl)-2-oxo-1,2-dihydropyrimidin-4-yl)-2-(3-(trifluoromethoxy)phenyl)acetamide (230 mg, 0.560 mmol) in DCM (2.2 mL) were added CuI (11 mg, 0.056 mmol) and propiolic acid (44.8 μl, 0.729 mmol). The resulting mixture was stirred at RT for 15 h. The reaction mixture was concentrated under reduced pressure and the residue purified by mass-triggered preparative HPLC (Mobile phase: A=0.1% TFA/H₂O, B=0.1% TFA/MeCN; Gradient: B=30-70%; 20 minutes; Column: C18) to give the title compound as an off-white solid (62 mg, 23%). MS (ES⁺) C₂₀H₁₉F₃N₆O₅ requires: 480. found: 481 [M+H]⁺.

### Step 5: N-benzyl-1-(4-(2-oxo-4-(2-(3-(trifluoromethoxy)phenyl)acetamido)pyrimidin-1(2H)-yl)butyl)-1H-1,2,3-triazole-4-carboxamide

To a suspension of 1-(4-(2-oxo-4-(2-(3-(trifluoromethoxy)phenyl)acetamido)pyrimidin-1(2H)-yl)butyl)-1H-1,2,3-triazole-4-carboxylic acid (20 mg, 0.042 mmol) and phenylmethanamine (5.5 μl, 0.050 mmol) in DMF (208 μl) was added T3P (26 mg, 0.042 mmol, 50% wt. solution) and the resulting mixture was heated at 60° C. for 3 h. The mixture was concentrated under reduced pressure and the residue was purified by mass-triggered preparative HPLC (Mobile phase: A=0.1% TFA/H₂O, B=0.1% TFA/MeCN; Gradient: B=30-70%; 20 minutes; Column: C18) to give the title compound as a white solid (3.1 mg, 13%). MS (ES⁺) C₂₇H₂₆F₃N₇O₄ requires: 569. found: 570 [M+H]⁺; ¹H NMR (600 MHz, DMSO-d₆) δ 11.06 (s, 1H), 9.03 (m, 1H), 8.57 (s, 1H), 8.06 (d, J=7.2 Hz, 1H), 7.46 (m, 1H), 7.34-7.29 (m, 6H), 7.27-7.20 (m, 2H), 7.08 (d, J=7.2 Hz, 1H), 4.47-4.39 (m, 4H), 3.86-3.75 (m, 4H), 1.87-1.77 (m, 2H), 1.65-1.52 (m, 2H).

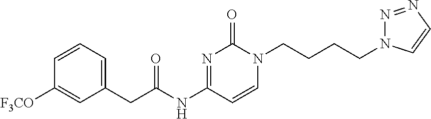 [SMILES: O=C(Cc1cccc(OC(F)(F)F)c1)Nc1ccn(CCCCn2ccnn2)c(=O)n1]

### N-(1-(4-(1H-1,2,3-triazol-1-yl)butyl)-2-oxo-1,2-dihydropyrimidin-4-yl)-2-(3-(trifluoromethoxy)phenyl)acetamide

The title compound was also isolated from the above reaction as an off-white solid (3 mg, 17%). MS (ES⁺) C₁₉H₁₉F₃N₆O₃ requires: 436. found: 437 [M+H]⁺; ¹H NMR (600 MHz, Methanol-d₄) δ 7.99-7.95 (m, 2H), 7.71 (d, J=1.0 Hz, 1H), 7.42 (m, 1H), 7.37 (d, J=7.3 Hz, 1H), 7.32 (d, J=7.8 Hz, 1H), 7.27 (s, 1H), 7.19 (d, J=8.2 Hz, 1H), 4.49 (t, J=7.0 Hz, 2H), 3.93 (t, J=7.2 Hz, 2H), 3.81 (s, 2H), 1.99-1.89 (m, 2H), 1.78-1.70 (m, 2H).

### Example 28

### Benzyl 2-oxo-1-(4-(5-(3-(trifluoromethoxy)benzylcarbamoyl)-1,3,4-thiadiazol-2-yl)butyl)-1,2-dihydropyrimidin-4-ylcarbamate

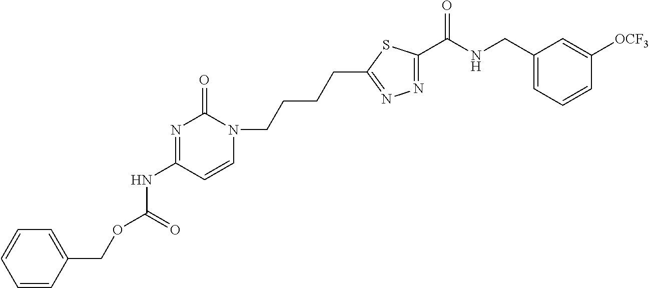 [SMILES: O=C(Nc1ccn(CCCCc2nnc(C(=O)NCc3cccc(OC(F)(F)F)c3)s2)c(=O)n1)OCc1ccccc1]

### Steps 1 to 6

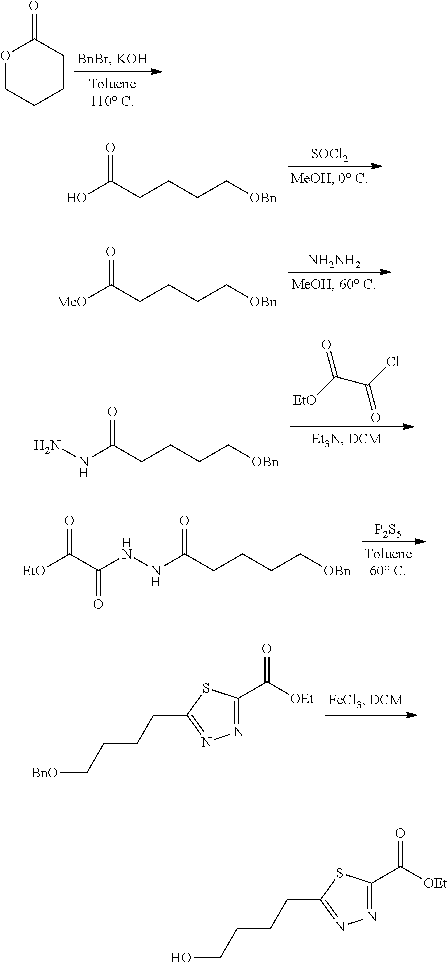

### Step 1: 5-(benzyloxy)pentanoic acid

To a solution of tetrahydro-2H-pyran-2-one (5.0 g, 50 mmol) in toluene (50 mL) were added KOH (15.8 g, 28.2 mmol) and benzylbromide (17.8 mL, 150 mmol), and the mixture was stirred at 110° C. for 16 h. The solution was cooled to RT and diluted with ice-cold H₂O (70 mL). The organic layer was separated and the aqueous layer was washed with MTBE (3×30 mL). The aqueous layer was then cooled to 0° C. and the pH was adjusted to 3-4 by addition of concentrated aq. HCl (15 mL) and 6 N aq. HCl (8 mL). The mixture was extracted with EtOAc (4×50 mL) and the combined organic layers were washed with brine (20 mL), dried (Na₂SO₄) and concentrated under reduced pressure to give the title compound as a light yellow oil (10 g, 96%). MS (ES⁺) C₁₂H₁₆O₃ requires: 208. found: 209[M+H]⁺.

### Step 2: methyl 5-(benzyloxy)pentanoate

To a solution of 5-(benzyloxy)pentanoic acid (1.0 g, 4.8 mmol) in MeOH (40 mL) at 0° C. was slowly added SOCl₂ (0.39 mL, 5.3 mmol) and the mixture was stirred at RT for 1 h. The mixture was then treated with saturated aq. NaHCO₃ (5 mL), the volatiles were removed under reduced pressure, and the aqueous layer was extracted with DCM (3×10 mL). The combined organic layers were washed with brine (5 mL), dried (Na₂SO₄) and concentrated under reduced pressure to afford the title compound as a light yellow oil (1.0 g, 94%). MS (ES⁺) C₁₃H₁₈O₃ requires: 222. found: 223[M+H]⁺.

### Step 3: 5-(benzyloxy)pentanehydrazide

A mixture of methyl 5-(benzyloxy)pentanoate (1.0 g, 4.5 mmol) and NH₂NH₂ (35% wt. solution in H₂O, 1.3 mL, 13 mmol) in MeOH (15 mL) was heated at reflux in a pressure safe vial for 18 h. The mixture was cooled to RT and the volatiles were removed under reduced pressure. The residue was taken up in toluene (2×5 mL), concentrated again, and the resulting solid was triturated with hexane (2×5 mL) and filtered to give the title compound as a white solid (840 mg, 84%). MS (ES⁺) C₁₂H₁₈N₂O₂ requires: 222. found: 223 [M+H]⁺.

### Step 4: ethyl 2-(2-(5-(benzyloxy)pentanoyl)hydrazinyl)-2-oxoacetate

To a solution of 5-(benzyloxy)pentanehydrazide (500 mg, 2.25 mmol) and TEA (0.63 mL, 4.5 mmol) in DCM (5.0 mL) at 0° C. was added ethyl 2-chloro-2-oxoacetate (0.45 mL, 4.0 mmol), and the resulting mixture was stirred at RT for 30 minutes. The volatiles were removed under reduced pressure to give the title compound as a white solid (800 mg, 100%). MS (ES⁺) C₁₆H₂₂N₂O₅ requires: 322. found: 323 [M+H]⁺.

### Step 5: ethyl 5-(4-(benzyloxy)butyl)-1,3,4-thiadiazole-2-carboxylate

To a mixture of ethyl 2-(2-(5-(benzyloxy)pentanoyl)hydrazinyl)-2-oxoacetate (400 mg, 2.3 mmol) in toluene (5 mL) at 60° C. was added P₂S₅ (500 mg, 2.3 mmol). The reaction mixture was stirred at 60° C. for 15 minutes, cooled to RT, and partitioned between saturated aq. NaHCO₃ (30 mL) and EtOAc (50 mL). The organic layer was washed with brine (5 mL), dried (Na₂SO₄) and concentrated under reduced pressure. The residue was purified by SiO₂ gel chromatography (5-20% EtOAc in hexanes) to give the title compound as a yellow oil (1.3 g, 48%). MS (ES⁺) C₁₆H₂₀N₂O₃S requires: 320. found: 321 [M+H]⁺.

### Step 6: ethyl 5-(4-hydroxybutyl)-1,3,4-thiadiazole-2-carboxylate

To a solution of ethyl 5-(4-(benzyloxy)butyl)-1,3,4-thiadiazole-2-carboxylate (1.0 g, 3.1 mmol) in DCM (30 mL) was added anhydrous FeCl₃ (2.0 g, 12 mmol) portionwise. The reaction mixture was stirred at RT for 25 minutes, a further aliquot of anhydrous FeCl₃ (1.0 g, 6.3 mmol) was added, and the mixture was stirred for an additional 10 minutes. The mixture was then treated with saturated aq. NH₄Cl (10 mL), the layers were separated, and the aqueous phase was extracted with DCM/MeOH=10/1 v/v (3×50 mL). The combined the organic layers were washed with brine (10 mL), dried (Na₂SO₄) and concentrated under reduced pressure. The residue was purified by SiO₂ gel chromatography (0-2% MeOH in DCM) to give the title compound as a brown oil (730 mg, 100%). MS (ES⁺) C₉H₁₄N₂O₃S requires: 230. found: 231 [M+H]⁺.

### Steps 7 to 9

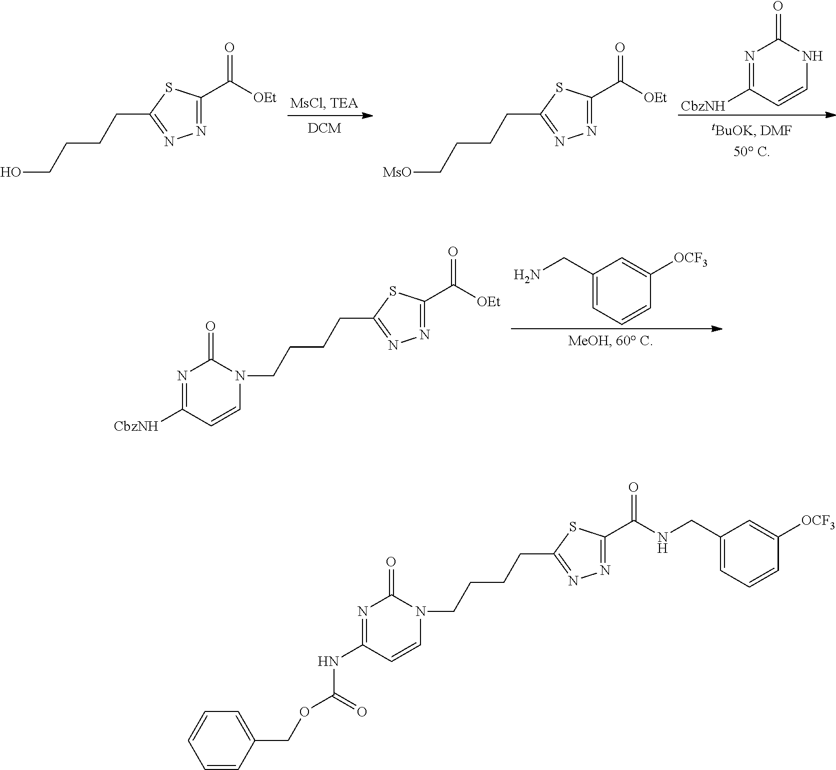

Step 7: ethyl 5-(4-(methylsulfonyloxy)butyl)-1,3,4-thiadiazole-2-carboxylate.

To a mixture of ethyl 5-(4-hydroxybutyl)-1,3,4-thiadiazole-2-carboxylate (100 mg, 0.43 mmol) and TEA (131 mg, 1.30 mmol) in DCM (1.5 mL) at 0° C. was added dropwise MsCl (75 mg, 0.65 mmol) as a solution in DCM (0.5 mL). The resulting mixture was stirred for 10 minutes at 0° C. and for a further 10 minutes at RT, then washed diluted with DCM (5 mL) and washed with brine (3 mL). The organic layer was dried (Na₂SO₄) and concentrated under reduced pressure to give the title compound as a brown oil (130 mg, 97%). MS (ES⁺) C₁₀H₁₆N₂O₅S₂ requires: 308. found: 309 [M+H]⁺. Step 8: ethyl 5-(4-(4-(benzyloxycarbonylamino)-2-oxopyrimidin-1(2H)-yl)butyl)-1,3,4-thiadiazole-2-carboxylate.

A mixture of ethyl 5-(4-(methylsulfonyloxy)butyl)-1,3,4-thiadiazole-2-carboxylate (640 mg, 2.08 mmol), benzyl 2-oxo-1,2-dihydropyrimidin-4-ylcarbamate (764 mg, 3.11 mmol) and t-BuOK (349 mg, 3.11 mmol) in DMF (8.0 mL) was stirred at 50° C. for 2 h. The mixture was filtered, and the filtrate was purified by preparative HPLC (Mobile phase: A=0.1% TFA/H₂O, B=0.1% TFA/MeCN; Gradient: B=30-70%; 12 min; Column: C18) to give the title compound as a white solid (155 mg, 27%). MS (ES⁺) C₂₁H₂₃N₅O₅S requires: 457. found: 458 [M+H]⁺.

### Step 9: benzyl 2-oxo-1-(4-(5-(3-(trifluoromethoxy)benzylcarbamoyl)-1,3,4-thiadiazol-2-yl)butyl)-1,2-dihydropyrimidin-4-ylcarbamate

A mixture of ethyl 5-(4-(4-(benzyloxycarbonylamino)-2-oxopyrimidin-1(2H)-yl)butyl)-1,3,4-thiadiazole-2-carboxylate (30 mg, 0.060 mmol) and (3-(trifluoromethoxy)phenyl)methanamine (75 mg, 0.40 mmol) in MeOH (0.5 mL) was stirred at 60° C. in a sealed vial for 5 h. The mixture was then cooled to RT, the volatiles were removed under reduced pressure, and the residue was purified by preparative HPLC (Mobile phase: A=0.1% TFA/H₂O, B=0.1% TFA/MeCN; Gradient: B=30-70%; 12 min; Column:

C18) to give the title compound as a white solid (6.8 mg, 17%). MS (ES⁺) C₂₇H₂₅F₃N₆O₅S requires: 602. found: 603 [M+H]⁺; ¹H NMR (400 MHz, DMSO-d₆) δ 10.75 (bs, 1H), 9.85 (t, J=6.2 Hz, 1H), 8.08 (d, J=7.2 Hz, 1H), 7.48 (m, 1H), 7.48-7.20 (m, 8H), 6.98 (d, J=7.6 Hz, 1H), 5.18 (s, 2H), 4.51 (d, J=6.0 Hz, 2H), 3.86-3.80 (m, 2H), 3.25-3.15 (m, 2H), 1.78-1.70 (m, 4H).

### Example 29

### N-(5-(4-(6-benzyl-2-oxo-2,7-dihydro-3H-pyrrolo[2,3-d]pyrimidin-3-yl)butyl)-1,3,4-thiadiazol-2-yl)-7-phenylacetamide

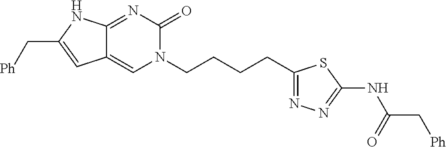 [SMILES: O=C(Cc1ccccc1)Nc1nnc(CCCCn2cc3cc(Cc4ccccc4)[nH]c3nc2=O)s1]

### Steps 1 to 4

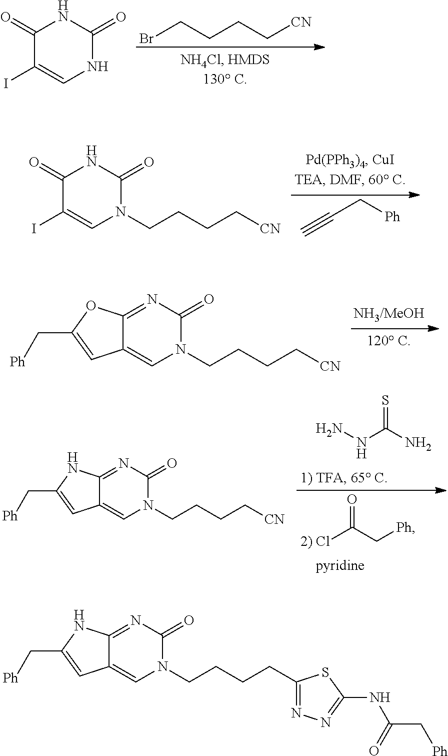

### Step 1: 5-(5-iodo-2,4-dioxo-3,4-dihydropyrimidin-1(2H)-yl)pentanenitrile

A mixture of NH₄Cl (0.45 g, 4.40 mmol), HMDS (17.9 ml, 84.0 mmol), and 5-iodopyrimidine-2,4(1H, 3H)-dione (2.0 g, 8.4 mmol) was heated at 130° C. for 4 h. The resulting clear solution was cooled to RT and concentrated under reduced pressure. 5-Bromopentanenitrile (0.98 ml, 8.4 mmol) was then added and the mixture was heated at 130° C. for 2 h. The reaction mixture was cooled to RT, diluted with EtOAc (20 mL) and i-PrOH (10 mL), stirred for 10 minutes at RT and filtered through a pad of Celite®. The filtrate was concentrated under reduced pressure and purified via SiO₂ gel chromatography (0-10% MeOH in DCM) to give the title compound as a light yellow solid (2.02 g, 75%). MS (ES⁺) C₉H₁₀IN₃O₂ requires: 319. found: 320 [M+H]⁺.

### Step 2: 5-(6-benzyl-2-oxofuro[2,3-d]pyrimidin-3(2H)-yl)pentanenitrile

A mixture of Pd(PPh₃)₄ (0.362 g, 0.313 mmol), triethylamine (5.0 mL, 36 mmol), CuI (1.19 g, 6.27 mmol), prop-2-yn-1-ylbenzene (1.01 mL, 8.15 mmol), and 5-(5-iodo-2,4-dioxo-3,4-dihydropyrimidin-1(2H)-yl)pentanenitrile (2.0 g, 6.3 mmol) in DMF (100 mL) was degassed with a stream of N₂ for 10 minutes. The reaction mixture was heated to 70° C. and stirred for 6 h, then cooled to RT and concentrated under reduced pressure. The residue was purified via silica gel chromatography (0-15% MeOH in DCM) to give a brown oil (1.25 g). The residue was further purified by mass-triggered preparative HPLC (Mobile phase: A=0.1% TFA/H2O, B=0.1% TFA/MeCN; Gradient: B=30-70%; 12 minutes; Column: C18) to give the title compound as a brown solid (170 mg, 9%). MS (ES⁺) C₁₈H₁₇N₃O₂ requires: 307. found: 308 [M+H]⁺.

### Step 3: 5-(6-benzyl-2-oxo-2,7-dihydro-3H-pyrrolo[2,3-d]pyrimidin-3-yl)pentanenitrile

Ammonia in MeOH (7N, 7.7 mL, 54 mmol) was added to 5-(6-benzyl-2-oxofuro[2,3-d]pyrimidin-3(2H)-yl)pentanenitrile (165 mg, 0.537 mmol) and the resulting solution was heated at 120° C. for 4 h in a pressure safe vial. The reaction mixture was concentrated under reduced pressure and the residue was purified by mass-triggered preparative HPLC (Mobile phase: A=0.1% TFA/H2O, B=0.1% TFA/MeCN; Gradient: B=20-50%; 16 minutes; Column: C18) to give the title compound as a brown thick oil (72 mg, 44%). MS (ES⁺) C₁₈H₁₈N₄O requires: 306. found: 307 [M+H]⁺.

### Step 4: 3-(4-(5-amino-1,3,4-thiadiazol-2-yl)butyl)-6-benzyl-3,7-dihydro-2H-pyrrolo[2,3-d]pyrimidin-2-one

A mixture of TFA (0.063 mL, 0.082 mmol), hydrazinecarbothioamide (8.2 mg, 0.090 mmol) and 5-(6-benzyl-2-oxo-2,7-dihydro-3H-pyrrolo[2,3-d]pyrimidin-3-yl)pentanenitrile (25 mg, 0.082 mmol) was heated at 60° C. for 2 h. The reaction was concentrated under reduced pressure and the residue was purified by mass-triggered preparative HPLC (Mobile phase: A=0.1% TFA/H₂O, B=0.1% TFA/MeCN; Gradient: B=20-50%; 12 minutes; Column: C18) to give the title compound as a pale yellow solid (38 mg, 94%). MS (ES⁺) C₁₉H₂₀N₆OS requires: 380. found: 381 [M+H]⁺.

### Step 5: N-(5-(4-(6-benzyl-2-oxo-2,7-dihydro-3H-pyrrolo[2,3-d]pyrimidin-3-yl)butyl)-1,3,4-thiadiazol-2-yl)-2-phenylacetamide

To a mixture of pyridine (14.0 μl, 0.173 mmol) and 3-(4-(5-amino-1,3,4-thiadiazol-2-yl)butyl)-6-benzyl-3,7-dihydro-2H-pyrrolo[2,3-d]pyrimidin-2-one (33 mg, 0.087 mmol) in DMF (434 μl) was added 2-phenylacetyl chloride (16 μl, 0.13 mmol). The resulting mixture was stirred at RT for 12 h and concentrated under reduced pressure. The residue was purified by mass-triggered preparative HPLC (Mobile phase: A=0.1% TFA/H2O, B=0.1% TFA/MeCN; Gradient: B=40-80%; 12 minutes; Column: C18) to give the title compound as an off-white solid after neutralizing residual TFA with Agilent StratoSpheres PL-HCO3 MP resin (3 mg, 7%). MS (ES⁺) C₂₇H₂₆N₆O₂S requires: 498. found: 499 [M+H]⁺. 1H NMR (600 MHz, DMSO-d₆) δ 12.67 (s, 1H), 11.17 (s, 1H), 8.28 (s, 1H), 7.35-7.21 (m, 10H), 5.83 (s, 1H), 3.90 (t, J=6.8 Hz, 2H), 3.86 (s, 2H), 3.78 (s, 2H), 2.97 (t, J=7.2 Hz, 2H), 1.74-1.61 (m, 4H).

Additional non-limiting examples include the following compounds and pharmaceutically acceptable salts thereof.

### Example 180

### 1-(2-fluoro-4-(3-fluoro-2-oxo-4-(2-phenylacetamido)pyridin-1(2H)-yl)butyl)-N-(3-(trifluoromethoxy)benzyl)-1H-1,2,3-triazole-4-carboxamide

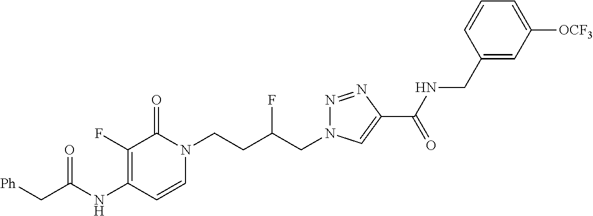 [SMILES: O=C(Cc1ccccc1)Nc1ccn(CCC(F)Cn2cc(C(=O)NCc3cccc(OC(F)(F)F)c3)nn2)c(=O)c1F]

### Steps 1 to 4

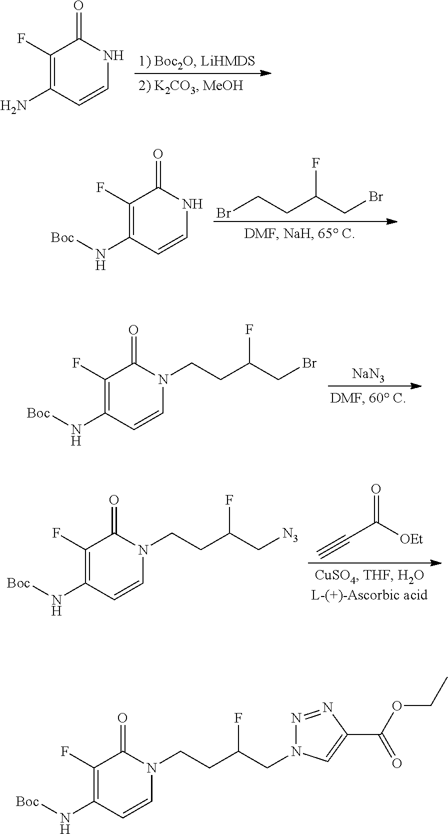

### Step 1: tert-butyl(3-fluoro-2-oxo-1,2-dihydropyridin-4-yl)carbamate

To a suspension of 4-amino-3-fluoropyridin-2(1H)-one (4.43 g, 34.6 mmol, prepared according to J. Med. Chem. 1987, 30, 340-347) and di-tert-butyl dicarbonate (16.6 g, 76 mmol) in THF (50 mL) was added LiHMDS in THF (76 mL, 76 mmol, 1.0 M) and the resulting mixture was stirred at 50° C. for 4 h. The reaction was concentrated, diluted with DCM (100 mL), and washed with ice-cold 1 M HCl (100 mL). The organic layer was concentrated to give a yellow oil (11.1 g, 99%). The crude oil was diluted with MeOH (60 mL) and K₂CO₃ (5.13 g, 37.1 mmol) was added. The resulting mixture was stirred at 50° C. for 45 min and then allowed to warm to RT overnight. The reaction was filtered and the filtrate was adsorbed on Celite® and purified via silica gel chromatography (0-30% MeOH in DCM) to give the title compound as a yellow solid (5.92 g, 77%). MS (ES⁺) C₁₀H₁₃FN₂O₃ requires: 228. found: 229 [M+H]⁺.

### Step 2: tert-butyl 1-(4-bromo-3-fluorobutyl)-3-fluoro-2-oxo-1,2-dihydropyridin-4-ylcarbamate

NaH (5.26 g, 131 mmol) was added to a solution of ethyl tert-butyl 3-fluoro-2-oxo-1,2-dihydropyridin-4-ylcarbamate (20.0 g, 87.7 mmol) in DMF (400 mL) at RT and stirred for 1 h, then 1,4-dibromo-2-fluorobutane (30.5 g, 131 mmol) was added. The mixture was stirred for 15 h at RT. The reaction solution was poured into water (2000 mL), extracted with EtOAc (500 mL×3), and the combined organics were washed with water (500 mL). The volitiles were concentrated and the residue purified via silica gel chromatography (Pet. Ether:EtOAc=from 10:1 to 1:2, v/v) to give the title compound as a solid (16.8 g, 50%). MS (ES⁺) C₁₄H₁₉BrF₂N₂O₃ requires: 380. found: 381 [M+H]⁺.

### Step 3: tert-butyl 1-(4-azido-3-fluorobutyl)-3-fluoro-2-oxo-1,2-dihydropyridin-4-ylcarbamate

NaN₃ (1.10 g, 19.7 mmol) was added to a solution of tert-butyl 1-(4-bromo-3-fluorobutyl)-3-fluoro-2-oxo-1,2-dihydropyridin-4-ylcarbamate (5.0 g, 13.1 mmol) in DMF (100 mL) and heated at 60-65° C. for 15 h. The mixture was cooled to RT, poured into water (800 mL), and extracted with EtOAc (100 mL×3). The combined organics were washed with water (100 mL×2) and brine (100 mL), dried over Na₂SO₄, and concentrated to give the title compound as an oil (4.2 g, 93%). MS (ES⁺) C₁₄H₁₉F₂N₅O₃ requires: 343. found: 344 [M+H]⁺.

### Step 4: ethyl 1-(4-(4-(tert-butoxycarbonylamino)-3-fluoro-2-oxopyridin-1(2H)-yl)-2-fluorobutyl)-1H-1,2,3-triazole-4-carboxylate

Tert-butyl 1-(4-azido-3-fluorobutyl)-3-fluoro-2-oxo-1,2-dihydropyridin-4-ylcarbamate (20 g, 58 mmol), CuSO₄.5H₂O (4.0 g, 16 mmol), L(+)-ascorbic acid (8.0 g, 45 mmol) and water (300 mL) were added to t-BuOH (300 mL). The reaction mixture was stirred for 5 min, then ethyl propiolate (6.85 g, 69.9 mmol) was added at RT. The mixture was stirred for 15 h at RT. The reaction mixture was concentrated in vacuo at 40-50° C., the residue was diluted with water (1000 mL) and extracted with EtOAc (200 mL×3). The combined organics were washed with water (300 mL×2), dried over Na₂SO₄, and concentrated in vacuo. The residue was purified via silica gel chromatography (Pet. Ether:EtOAc=from 1:1 to 1:2, v/v) to give the title compound as a white solid (18.5 g, 72%). MS (ES⁺) C₁₉H₂₅F₂N₅O₅ requires: 441. found: 442 [M+H]⁺; ¹H NMR (600 MHz, DMSO-d₆) δ 9.48 (s, 1H), 8.76 (s, 1H), 7.45 (dd, J=7.8, 1.5 Hz, 1H), 6.87 (dd, J=7.7, 6.5 Hz, 1H), 5.06-4.90 (m, 1H), 4.84-4.66 (m, 2H), 4.31 (q, J=7.1 Hz, 2H), 4.11-3.94 (m, 2H), 2.15-1.82 (m, 2H), 1.47 (s, 9H), 1.30 (t, J=7.1 Hz, 3H). The regioisomer ethyl 1-(4-(4-((tert-butoxycarbonyl)amino)-3-fluoro-2-oxopyridin-1(2H)-yl)-3-fluorobutyl)-1H-1,2,3-triazole-4-carboxylate was also isolated as a minor side product (435 mg, 2%). MS (ES⁺) C₁₉H₂₅F₂N₅O₅ requires: 441. found: 442 [M+H]⁺; ¹H NMR (600 MHz, DMSO-d₆) δ 9.47 (s, 1H), 8.92 (s, 1H), 7.32 (dd, J=7.8, 1.6 Hz, 1H), 6.84 (dd, J=7.7, 6.4 Hz, 1H), 5.03 (ddt, J=15.7, 10.6, 5.2 Hz, 1H), 4.87-4.71 (m, 2H), 4.32 (q, J=7.2 Hz, 2H), 3.90-3.75 (m, 2H), 2.41-2.23 (m, 2H), 1.46 (s, 9H), 1.32 (t, J=7.1 Hz, 3H).

### Steps 5 to 8

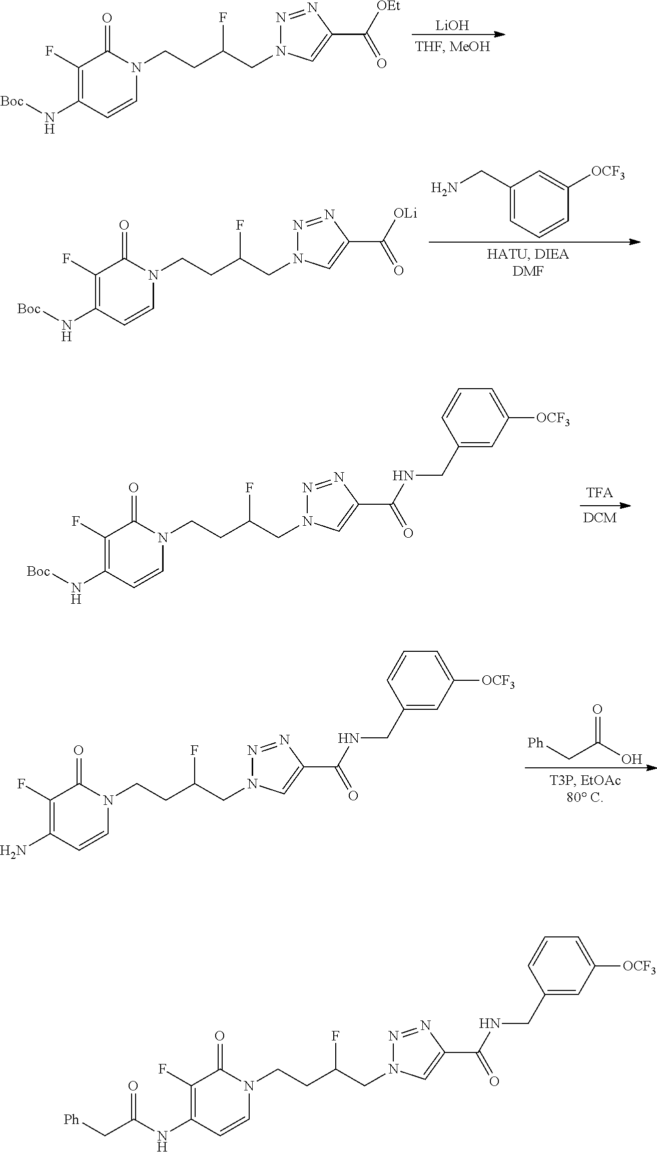

### Step 5: lithium 1-(4-(4-((tert-butoxycarbonyl)amino)-3-fluoro-2-oxopyridin-1(2H)-yl)-2-fluorobutyl)-1H-1,2,3-triazole-4-carboxylate

To a suspension of ethyl 1-(4-(4-((tert-butoxycarbonyl)amino)-3-fluoro-2-oxopyridin-1(2H)-yl)-2-fluorobutyl)-1H-1,2,3-triazole-4-carboxylate (1.0 g, 2.26 mmol) in THF (9.1 mL) and MeOH (2.3 mL) was added aqueous LiOH (1.25 mL, 2.49 mmol, 2.0 M) and the resulting mixture was stirred at RT for 2 h. The reaction was concentrated to give the title compound as a green amorphous material (0.896 g, 100%, Li salt). MS (ES⁺) C₁₇H₂₁F₂N₅O₅ requires: 413. found: 414 [M+H]⁺.

### Step 6: tert-butyl(3-fluoro-1-(3-fluoro-4-(4-((3-(trifluoromethoxy)benzyl)carbamoyl)-1H-1,2,3-triazol-1-yl)butyl)-2-oxo-1,2-dihydropyridin-4-yl)carbamate

To a suspension of 1-(4-(4-((tert-butoxycarbonyl)amino)-3-fluoro-2-oxopyridin-1(2H)-yl)-2-fluorobutyl)-1H-1,2,3-triazole-4-carboxylic acid (0.896 g, 2.17 mmol), (3-(trifluoromethoxy)phenyl)methanamine (0.456 g, 2.38 mmol) and HATU (0.989 g, 2.60 mmol) in DMF (10.8 mL) at 0° C. was added DIEA (0.757 mL, 4.34 mmol) and the resulting mixture was stirred at RT for 2 h. The volatiles were removed under reduced pressure. The residue was purified via silica gel chromatography (0-15% MeOH in DCM with 1% NH₄OH) to give the title compound as an off-white solid (1.8 g, >100%, 70% purity). MS (ES⁺) C₂₅H₂₇F₅N₆O₅ requires: 586. found: 587 [M+H]⁺.

### Step 7: 1-(4-(4-amino-3-fluoro-2-oxopyridin-1 (2H)-yl)-2-fluorobutyl)-N-(3-(trifluoromethoxy)benzyl)-1H-1,2,3-triazole-4-carboxamide

To a suspension of tert-butyl(3-fluoro-1-(3-fluoro-4-(4-((3-(trifluoromethoxy)benzyl)carbamoyl)-1H-1,2,3-triazol-1-yl)butyl)-2-oxo-1,2-dihydropyridin-4-yl)carbamate (1.27 g, 2.16 mmol) in DCM (10.8 mL) was added trifluoroacetic acid (3.34 mL, 43.3 mmol) and the resulting mixture was stirred at RT for 4 h. The reaction was concentrated and the residue was purified via silica gel chromatography (0-15% MeOH in DCM with 1% NH₄OH) to give the title compound as an off-white solid (880 mg, 84%). MS (ES⁺) C₂₀H₁₉F₅N₆O₃ requires: 486. found: 487 [M+H]⁺.

### Step 8: 1-(2-fluoro-4-(3-fluoro-2-oxo-4-(2-phenylacetamido)pyridin-1 (2H)-yl)butyl)-N-(3-(trifluoromethoxy)benzyl)-1H-1,2,3-triazole-4-carboxamide

To a suspension of 1-(4-(4-amino-3-fluoro-2-oxopyridin-1(2H)-yl)-2-fluorobutyl)-N-(3-(trifluoromethoxy)benzyl)-1H-1,2,3-triazole-4-carboxamide (50 mg, 0.10 mmol) and 2-phenylacetic acid (28.0 mg, 0.206 mmol) was added 2,4,6-tripropyl-1,3,5,2,4,6-trioxatriphosphinane 2,4,6-trioxide (327 mg, 0.514 mmol, 50 wt. % in EtOAc) and the resulting mixture was stirred at 80° C. for 2 h. The reaction was concentrated briefly and purified by mass-triggered preparative HPLC (Mobile phase: A=0.1% TFA/H₂O, B=0.1% TFA/MeCN; Gradient: B=40-80%; 12 min; Column: C18) to give the title compound as a white solid (20 mg, 32%). MS (ES⁺) C₂₈H₂₅F₅N₆O₄ requires: 604. found: 605 [M+H]⁺; ¹H NMR (600 MHz, DMSO-d₆) δ 10.21 (s, 1H), 9.20 (t, J=6.4 Hz, 1H), 8.56 (s, 1H), 7.49-7.37 (m, 2H), 7.36-7.17 (m, 8H), 7.05 (dd, J=7.7, 6.3 Hz, 1H), 4.99 (dddt, J=49.6, 9.9, 6.7, 3.0 Hz, 1H), 4.84-4.66 (m, 2H), 4.48 (d, J=6.2 Hz, 2H), 4.15-3.95 (m, 2H), 3.78 (s, 2H), 2.16-1.87 (m, 2H).

### Example 365

### tert-butyl 4-(2-((3-fluoro-1-(2-fluoro-4-(4-((3-(trifluoromethoxy)benzyl)carbamoyl)-1H-1,2,3-triazol-1-yl)butyl)-2-oxo-1,2-dihydropyridin-4-yl)amino)-2-oxoethyl)piperidine-1-carboxylate

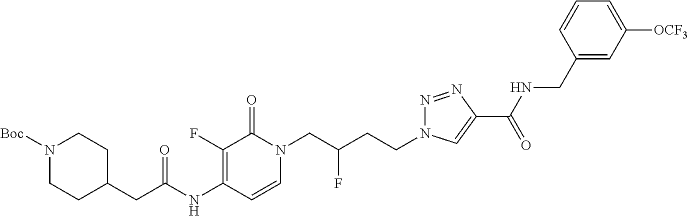 [SMILES: CC(C)(C)OC(=O)N1CCC(CC(=O)Nc2ccn(CC(F)CCn3cc(C(=O)NCc4cccc(OC(F)(F)F)c4)nn3)c(=O)c2F)CC1]

### Steps 1 to 3

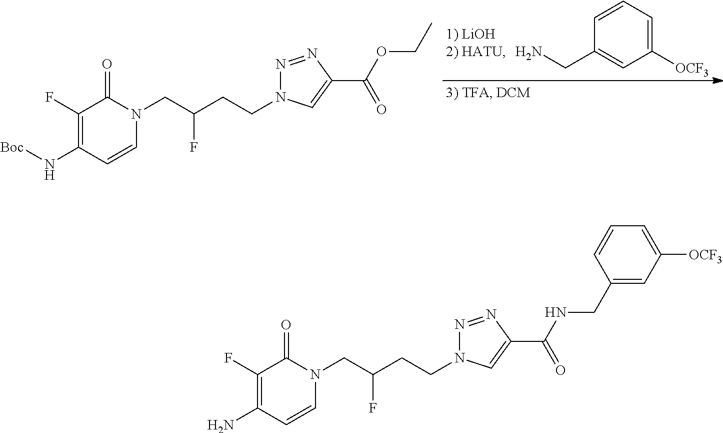 [SMILES: CCOC(=O)c1cn(CCC(F)Cn2ccc(NC(=O)OC(C)(C)C)c(F)c2=O)nn1 ; NCc1cccc(OC(F)(F)F)c1 ; Nc1ccn(CC(F)CCn2cc(C(=O)NCc3cccc(OC(F)(F)F)c3)nn2)c(=O)c1F]

### Steps 1 to 3: 1-(4-(4-amino-3-fluoro-2-oxopyridin-1 (2H)-yl)-3-fluorobutyl)-N-(3-(trifluoromethoxy)benzyl)-1H-1,2,3-triazole-4-carboxamide

Using steps 5 to 7 of EXAMPLE 180, the title compound was obtained as a tan solid (98 mg, 20% overall). MS (ES⁺) C₂₀H₁₉F₅N₆O₃ requires: 486. found: 487 [M+H]⁺.

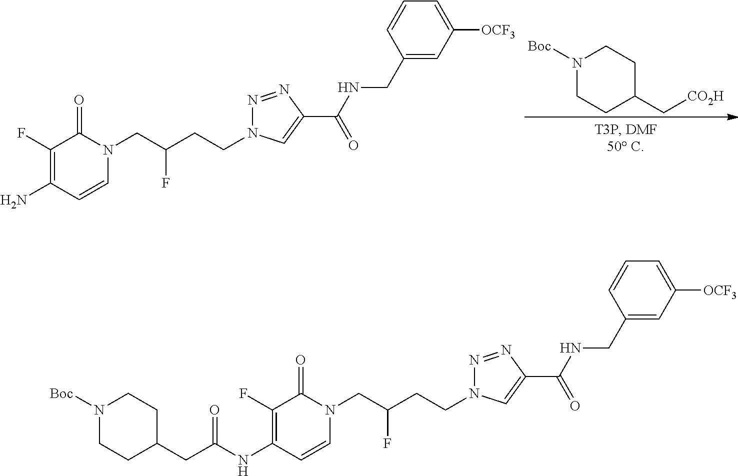 [SMILES: Nc1ccn(CC(F)CCn2cc(C(=O)NCc3cccc(OC(F)(F)F)c3)nn2)c(=O)c1F ; CC(C)(C)OC(=O)N1CCC(CC(=O)O)CC1 ; CC(C)(C)OC(=O)N1CCC(CC(=O)Nc2ccn(CC(F)CCn3cc(C(=O)NCc4cccc(OC(F)(F)F)c4)nn3)c(=O)c2F)CC1]

### Step 4: tert-butyl 4-(2-((3-fluoro-1-(2-fluoro-4-(4-((3-(trifluoromethoxy)benzyl)carbamoyl)-1H-1,2,3-triazol-1-yl)butyl)-2-oxo-1,2-dihydropyridin-4-yl)amino)-2-oxoethyl)piperidine-1-carboxylate

To a solution of 1-(4-(4-amino-3-fluoro-2-oxopyridin-1(2H)-yl)-3-fluorobutyl)-N-(3-(trifluoromethoxy)benzyl)-1H-1,2,3-triazole-4-carboxamide (20 mg, 0.041 mmol), pyridine (16.56 μl, 0.206 mmol), and T3P® in DMF (122 μl, 0.206 mmol, 50% wt) in DMF (411 μl) was added 2-(1-(tert-butoxycarbonyl)piperidin-4-yl)acetic acid (20 mg, 0.082 mmol) and the resulting mixture was stirred at 50° C. overnight. The reaction mixture was concentrated and the residue was purified by mass-triggered preparative HPLC (Mobile phase: A=0.1% TFA/H₂O, B=0.1% TFA/MeCN; Gradient: B=40-80%; 12 min; Column: C18) to give the title compound as a white solid (2.2 mg, 7.5%). MS (ES⁺) C₃₂H₃₈F₅N₇O₆ requires: 711. found: 612 [M-Boc+H]+; ¹H NMR (600 MHz, DMSO-d₆) δ 10.73 (s, 1H), 9.69 (s, 1H), 9.21 (t, J=6.3 Hz, 1H), 8.56 (s, 1H), 7.56 (d, J=7.7 Hz, 0H), 7.46 (t, J=7.9 Hz, 1H), 7.35 (d, J=8.4 Hz, 1H), 7.29 (s, 1H), 7.23 (d, J=8.0 Hz, 0H), 6.97 (t, J=6.5 Hz, 1H), 5.09-4.87 (m, 1H), 4.85-4.68 (m, 2H), 4.48 (d, J=6.2 Hz, 2H), 4.27-4.20 (m, 1H), 4.17-4.11 (m, 1H), 4.09-4.03 (m, 1H), 3.55 (d, J=13.0 Hz, 1H), 3.36 (d, J=9.7 Hz, 1H), 3.09-2.91 (m, 2H), 2.18-2.02 (m, 1H), 2.01-1.88 (m, 1H), 1.86-1.74 (m, 3H), 1.73-1.61 (m, 2H), 1.53 (d, J=6.8 Hz, 3H), 1.47-1.39 (m, 1H).

### Example 337

### 1-(2-fluoro-4-(3-fluoro-2-oxo-4-(2-(piperidin-1-yl)propanamido)pyridin-1(2H)-yl)butyl)-N-(3-(trifluoromethoxy)benzyl)-1H-1,2,3-triazole-4-carboxamide

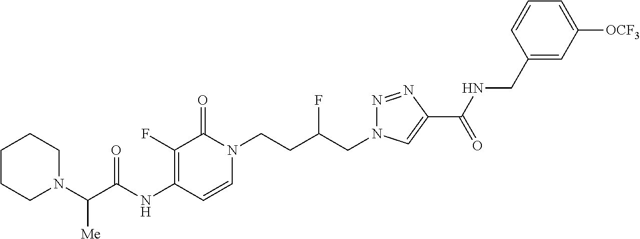 [SMILES: CC(C(=O)Nc1ccn(CCC(F)Cn2cc(C(=O)NCc3cccc(OC(F)(F)F)c3)nn2)c(=O)c1F)N1CCCCC1]

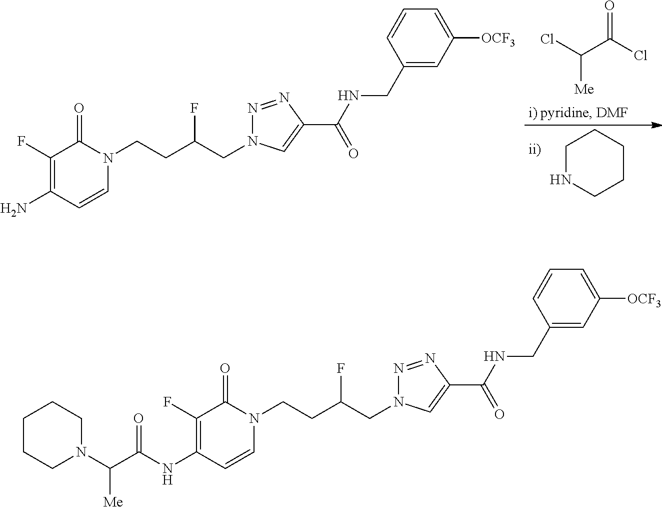

### Step 1: 1-(2-fluoro-4-(3-fluoro-2-oxo-4-(2-(piperidin-1-yl)propanamido)pyridin-1(2H)-yl)butyl)-N-(3-(trifluoromethoxy)benzyl)-1H-1,2,3-triazole-4-carboxamide

To a solution of 1-(4-(4-amino-3-fluoro-2-oxopyridin-1(2H)-yl)-2-fluorobutyl)-N-(3-(trifluoromethoxy)benzyl)-1H-1,2,3-triazole-4-carboxamide (30 mg, 0.062 mmol) and pyridine (15 μl, 0.18 mmol) in DMF (308 μl) at 0° C. was added 2-chloropropanoyl chloride (15 mg, 0.12 mmol). The resulting mixture was stirred at RT for 1 h. The reaction was cooled to 0° C. and piperidine (61 al, 0.62 mmol) was added before warming to RT and stirring for 12 h. The reaction was purified by mass-triggered preparative HPLC (Mobile phase: A=0.1% TFA/H₂O, B=0.1% TFA/MeCN; Gradient: B=30-70%; 12 min; Column: C18) to give the title compound as a pale yellow solid (35 mg, 91% yield, TFA salt). MS (ES⁺) C₂₈H₃₂F₅N₇O₄ requires: 625 found: 626 [M+H]⁺; ¹H NMR (600 MHz, DMSO-d₆) δ 10.73 (s, 1H), 9.69 (s, 1H), 9.21 (t, J=6.3 Hz, 1H), 8.56 (s, 1H), 7.56 (d, J=7.7 Hz, 0H), 7.46 (t, J=7.9 Hz, 1H), 7.35 (d, J=8.4 Hz, 1H), 7.29 (s, 1H), 7.23 (d, J=8.0 Hz, 0H), 6.97 (t, J=6.5 Hz, 1H), 5.09-4.87 (m, 1H), 4.85-4.68 (m, 2H), 4.48 (d, J=6.2 Hz, 2H), 4.27-4.20 (m, 1H), 4.17-4.11 (m, 1H), 4.09-4.03 (m, 1H), 3.55 (d, J=13.0 Hz, 1H), 3.36 (d, J=9.7 Hz, 1H), 3.09-2.91 (m, 2H), 2.18-2.02 (m, 1H), 2.01-1.88 (m, 1H), 1.86-1.74 (m, 3H), 1.73-1.61 (m, 2H), 1.53 (d, J=6.8 Hz, 3H), 1.47-1.39 (m, 1H).

### Example 414

### 1-(2-fluoro-4-(2-oxo-4-(phenoxymethyl)pyridin-1(2H)-yl)butyl)-N-(3-(trifluoromethoxy)benzyl)-1H-1,2,3-triazole-4-carboxamide

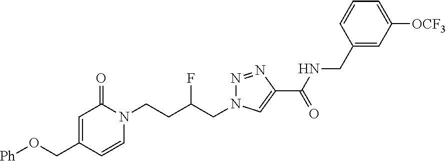 [SMILES: O=C(NCc1cccc(OC(F)(F)F)c1)c1cn(CC(F)CCn2ccc(COc3ccccc3)cc2=O)nn1]

### Steps 1 to 5

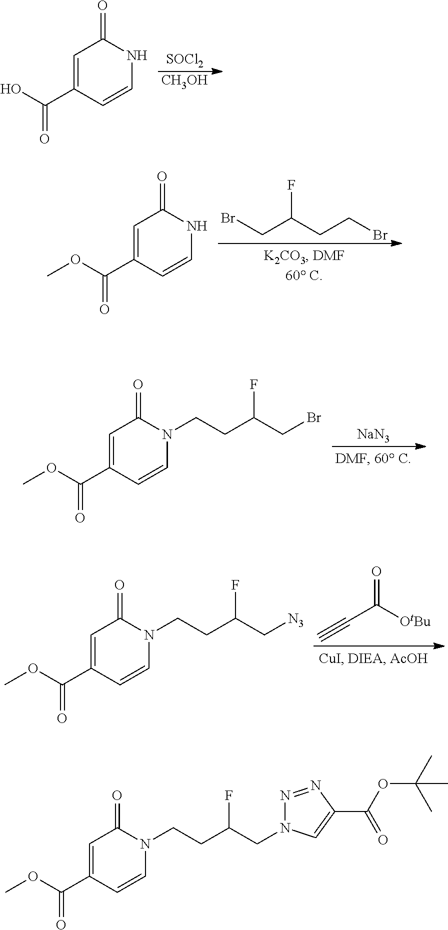

### Step 1: 1,4-dibromo-2-fluorobutane

To a solution of 1,4-dibromobutan-2-ol (15 g, 65.5 mmol) in DCM (100 mL) at 0° C. was added DAST (20 mL, 131 mmol). The reaction mixture was stirred at 0° C. for 10 min, then at RT for 12 h. The reaction was quenched with sat. NaHCO₃ (30 mL) and extracted with the EtOAc (3×50 mL). The combined organics were dried over Na₂SO₄ and concentrated to gave the title compound as an orange oil (14.7 g, 98%). ¹F NMR (500 MHz, MeOH-d₄) δ 192.3.

### Step 2: methyl 2-oxo-1,2-dihydropyridine-4-carboxylate

To a solution of 2-oxo-1,2-dihydropyridine-4-carboxylic acid (10 g, 72 mmol) in CH₃OH (100 mL) was added SOCl₂ (21 mL, 180 mmol) slowly at 0° C. The reaction mixture was stirred at 0° C. for 10 min, then at 65° C. for 12 h. The reaction mixture was concentrated, then washed with water (30 mL), and the solid filtered to give the title compound as an orange solid (9.0 g, 78%). MS (ES⁺) C₇H₇NO₃ requires: 153 found: 154 [M+H]⁺.

### Step 3: methyl 1-(4-bromo-3-fluorobutyl)-2-oxo-1,2-dihydropyridine-4-carboxylate

A mixture of methyl 2-oxo-1,2-dihydropyridine-4-carboxylate (8.0 g, 52 mmol), 1,4-dibromo-2-fluorobutane (24 g, 104 mmol), and K₂CO₃ (14 g, 104 mmol) in DMF (30 mL) was stirred at 60° C. for 12 h. The mixture was filtered and poured into water (100 mL), extracted with EtOAc (3×50 mL), washed with brine (3×50 mL), and dried over Na₂SO₄. Concentration gave the title compound as an orange solid (9.1 g, 50%). MS (ES⁺) C₁₁H₁₃BrFNO₃ requires: 305 found: 306 [M+H]⁺.

### Step 4: methyl 1-(4-azido-3-fluorobutyl)-2-oxo-1,2-dihydropyridine-4-carboxylate

A mixture of methyl 1-(4-bromo-3-fluorobutyl)-2-oxo-1,2-dihydropyridine-4-carboxylate (9.0 g, 29.5 mmol) and NaN₃ (3.8 g, 59 mmol) in DMF (40 mL) was stirred at 60° C. for 12 h. The mixture was filtered off and poured into water (100 mL), extracted with EtOAc (3×50 mL), washed with brine (3×50 mL), then dried by Na₂SO₄, filtered off and concentrated to give the title compound as an orange solid (7.9 g, 98%). MS (ES⁺) C₁₁H₁₃FN₄O₃ requires: 268 found: 269 [M+H]⁺.

### Step 5: methyl 1-(4-(4-(tert-butoxycarbonyl)-1H-1,2,3-triazol-1-yl)-3-fluorobutyl)-2-oxo-1,2-dihydropyridine-4-carboxylate

A mixture of methyl 1-(4-azido-3-fluorobutyl)-2-oxo-1,2-dihydropyridine-4-carboxylate (7.9 g, 29 mmol), tert-butyl propiolate (7.5 g, 59.7 mmol), CuSO₄ (1.6 g, 10 mmol) and L-sodium ascrobate (3.2 mg, 16 mmol) in t-BuOH (20 mL) and water (20 mL) was stirred at RT for 1 h. The mixture was filtered off and poured into water, extracted with EtOAc (3×50 mL), dried over Na₂SO₄, and concentrated. The resulting residue was purified via the silica gel chromatography to give the title compound as a white solid (9 g, 85%). MS (ES⁺) C₁₈H₂₃FN₄O₅ requires: 394. found: 395 [M+H]⁺. ¹H NMR (500 MHz, DMSO-d₆) δ 8.63 (s, 1H), 7.79 (s, 1H), 6.87 (s, 1H), 6.57 (s, 1H), 5.65-4.93 (m, 1H), 4.82-4.73 (m, 2H), 4.13-4.02 (m, 2H), 3.85 (s, 3H), 1.67 (s, 2H), 1.53 (s, 9H).

### Steps 6 to 9

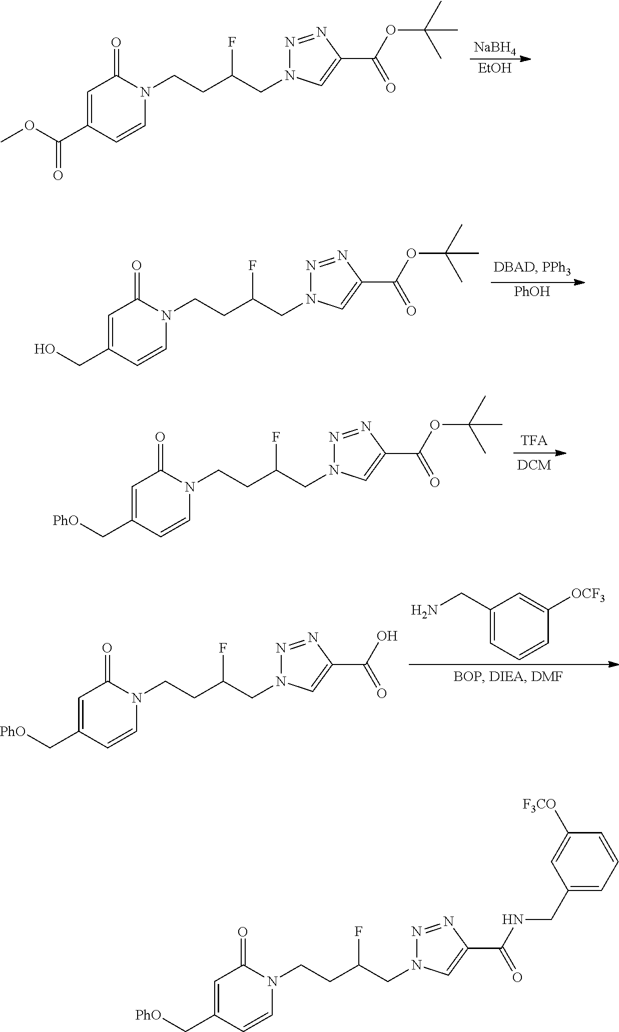

### Step 6: tert-butyl 1-(2-fluoro-4-(4-(hydroxymethyl)-2-oxopyridin-1(2H)-yl)butyl)-1H-1,2,3-triazole-4-carboxylate

To a suspension of methyl 1-(4-(4-(tert-butoxycarbonyl)-1H-1,2,3-triazol-1-yl)-3-fluorobutyl)-2-oxo-1,2-dihydropyridine-4-carboxylate (1.0 g, 2.5 mmol) in EtOH (12.7 mL) was added sodium borohydride (0.288 g, 7.61 mmol) and the resulting mixture was stirred at RT for 24 h. At that time an additional amount of NaBH₄ was added (0.288 g) and allowed to stir at RT for another 24 h. The reaction mixture was concentrated. The residue was adsorbed onto Celite® and purified via silica gel chromatography (0-15% MeOH in DCM with 1% NH₄OH) to give the title compound as a colorless amorphous material (710 mg, 76%). MS (ES⁺) C₁₇H₂₃FN₄O₄ requires: 366 found: 367 [M+H]⁺.

### Step 7: tert-butyl 1-(2-fluoro-4-(2-oxo-4-(phenoxymethyl)pyridin-1(2H)-yl)butyl)-1H-1,2,3-triazole-4-carboxylate

To a solution of phenol (19.3 mg, 0.205 mmol), tert-butyl 1-(2-fluoro-4-(4-(hydroxymethyl)-2-oxopyridin-1(2H)-yl)butyl)-1H-1,2,3-triazole-4-carboxylate (50 mg, 0.14 mmol), and di-tert-butyl hydrazine-1,2-dicarboxylate (63 mg, 0.27 mmol) in THF (682 μl) was added triphenylphosphine (72 mg, 0.27 mmol) and the resulting mixture was stirred at RT for 2 h. The reaction was concentrated. The residue was purified via silica gel chromatography (0-10% MeOH in DCM with 1% NH₄OH) to give the title compound as a colorless amorphous material (117 mg, >100%, ˜50% purity). MS (ES⁺) C₂₃H₂₇FN₄O₄ requires: 442 found: 443 [M+H]⁺.

### Step 8: 1-(2-fluoro-4-(2-oxo-4-(phenoxymethyl)pyridin-1(2H)-yl)butyl)-1H-1,2,3-triazole-4-carboxylic acid

A reaction vessel was charged with tert-butyl 1-(2-fluoro-4-(2-oxo-4-(phenoxymethyl)pyridin-1(2H)-yl)butyl)-1H-1,2,3-triazole-4-carboxylate (117 mg, 0.264 mmol), trifluoroacetic acid (1.0 mL, 13 mmol) and DCM (1 mL) under an atmosphere of N₂. The resulting solution was allowed to stir at RT for 1 h. The solution was concentrated to give the title compound as an amorphous material (102 mg, 100%). MS (ES⁺) C₁₉H₁₉FN₄O₄ requires: 386 found: 387 [M+H]⁺.

### Step 9: 1-(2-fluoro-4-(2-oxo-4-(phenoxymethyl)pyridin-1 (2H)-yl)butyl)-N-(3-(trifluoromethoxy)benzyl)-1H-1,2,3-triazole-4-carboxamide

To a suspension of 1-(2-fluoro-4-(2-oxo-4-(phenoxymethyl)pyridin-1(2H)-yl)butyl)-1H-1,2,3-triazole-4-carboxylic acid (50 mg, 0.13 mmol), (3-(trifluoromethoxy)phenyl)methanamine (27 mg, 0.14 mmol) and BOP (74 mg, 0.17 mmol) in DMF (647 μl) was added DIEA (45 al, 0.26 mmol) and the resulting mixture was stirred at RT for 1 h. The reaction mixture was purified by mass-triggered preparative HPLC (Mobile phase: A=0.1% TFA/H₂O, B=0.1% TFA/MeCN; Gradient: B=40-80%; 12 min; Column: C18) to give the title compound as a white solid (3.2 mg, 3.7%). MS (ES⁺) C₂₇H₂₅F₄N₅O₄ requires: 559. found: 560 [M+H]⁺; ¹H NMR (500 MHz, Methanol-d₄) δ 9.11 (t, J=6.4 Hz, 1H), 8.41 (d, J=1.2 Hz, 1H), 7.45-7.39 (m, 1H), 7.36 (d, J=7.8 Hz, 1H), 7.31-7.23 (m, 3H), 7.16 (d, J=8.2 Hz, 1H), 6.96 (dd, J=13.7, 7.5 Hz, 3H), 6.63 (s, 1H), 6.44 (d, J=6.9 Hz, 1H), 5.01 (s, 2H), 4.85 (s, 1H), 4.66 (t, J=7.0 Hz, 2H), 4.60 (d, J=5.5 Hz, 2H), 4.37 (dd, J=29.5, 14.4 Hz, 1H), 4.17-4.03 (m, 1H), 2.44-2.19 (m, 2H).

### Example 491

### 3-(trifluoromethoxy)benzyl(1-(3-fluoro-4-(4-((3-(trifluoromethoxy)benzyl)carbamoyl)-1H-1,2,3-triazol-1-yl)butyl)-2-oxo-1,2-dihydropyrimidin-4-yl)carbamate

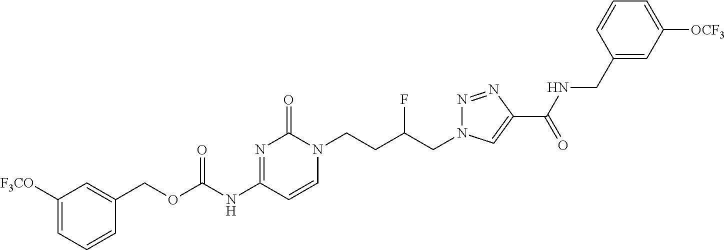 [SMILES: O=C(Nc1ccn(CCC(F)Cn2cc(C(=O)NCc3cccc(OC(F)(F)F)c3)nn2)c(=O)n1)OCc1cccc(OC(F)(F)F)c1]

### Steps 1 to 4

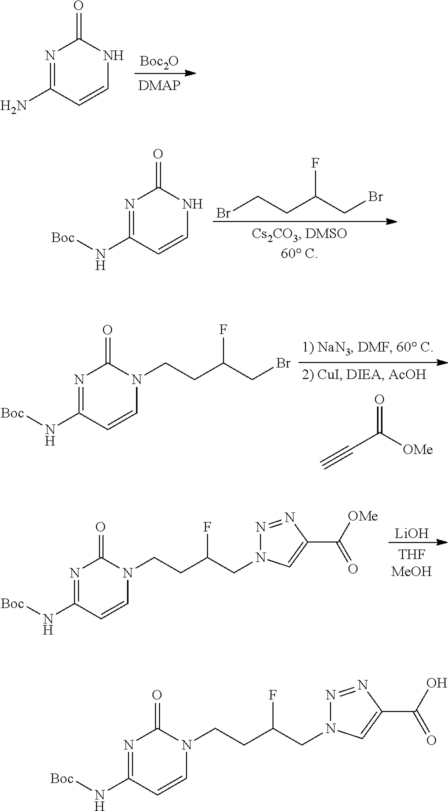

### Step 1: tert-butyl 2-oxo-1,2-dihydropyrimidin-4-ylcarbamate

To a solution of 4-aminopyrimidin-2(1H)-one (10 g, 90 mmol) in DMF (100 mL), DMAP (1.1 g, 9.0 mmol) and Boc₂O (29.5 g, 135 mmol) were added. The resulted mixture was stirred at RT for 15 h. To the mixture was added water (200 mL) and the solid filtered off to obtain the title compound as a white solid (7.0 g, 37%). MS (ES+) C₉H₁₃N₃O₃ requires: 211. found: 212 [M+H]⁺.

### Step 2: tert-butyl 1-(4-bromo-3-fluorobutyl)-2-oxo-1,2-dihydropyrimidin-4-ylcarbamate

To a solution of tert-butyl 2-oxo-1,2-dihydropyrimidin-4-ylcarbamate (5 g, 24 mmol) in DMSO (100 mL), 1,4-dibromo-2-fluorobutane (6.65 g, 28.4 mmol) and Cs₂CO₃ (15.4 g, 47.4 mmol) were added. The resulting mixture was stirred at 60° C. for 15 h. The resulted mixture was poured into water (500 mL), extracted with EtOAc (200 mL×2), concentrated, and purified by silica gel chromatography (DCM: MeOH=19:1) to give the title compound as a yellow solid (1.5 g, 17%). MS (ES+) C₁₃H₁₉BrFN₃03 requires: 363. found: 364 [M+H]⁺.

### Step 3: tert-butyl 1-(4-azido-3-fluorobutyl)-2-oxo-1,2-dihydropyrimidin-4-ylcarbamate

To a solution of tert-butyl 1-(4-bromo-3-fluorobutyl)-2-oxo-1,2-dihydropyrimidin-4-ylcarbamate (1.5 g, 4.13 mmol) in DMF (15 mL), NaN₃ (270 mg, 4.13 mmol) was added. The resulting mixture was stirred at 60° C. overnight. The resulting mixture was cooled to RT, and DCM (20 mL), methyl propiolate (347 mg, 4.13 mmol), AcOH (51 mg, 0.83 mmol), DIEA (51 mg, 0.83 mmol) and CuI (39 mg, 0.21 mmol) were added. The resulting mixture was stirred at RT for 2 h. The mixture was concentrated, then water (20 mL) was added, and the solid was filtered off to give the title compound as a white solid (1.6 g, 95% overall). MS (ES+) C₁₇H₂₃FN₆O₅ requires: 410. found: 411 [M+H]⁺.

### Step 4: 1-(4-(4-(tert-butoxycarbonylamino)-2-oxopyrimidin-1(2H)-yl)-2-fluorobutyl)-1H-1,2,3-triazole-4-carboxylic acid

To a solution of methyl 1-(4-(4-(tert-butoxycarbonylamino)-2-oxopyrimidin-1(2H)-yl)-2-fluorobutyl)-1H-1,2,3-triazole-4-carboxylate (1.6 g, 3.9 mmol) in THF (4 mL) and MeOH (4 mL), a solution of aqueous LiOH (94 mg, 3.9 mmol) was added. The resulting mixture was stirred at RT for 3 h. The mixture was concentrated, the pH adjusted with 1 N HCl, and the precipitate was filtered off to give the title compound as a yellow solid (1.25 g, 81%). MS (ES+) C₁₆H₂₁FN₆O₅ requires: 396. found: 397 [M+H]⁺.

### Steps 5 to 7

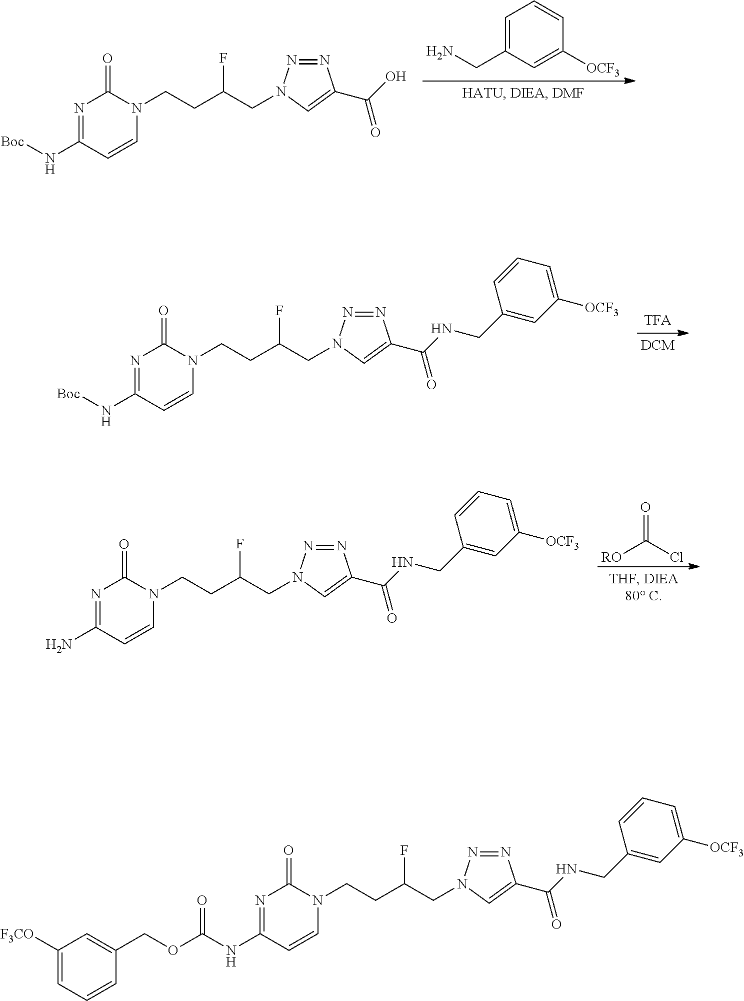

### Step 5: tert-butyl 1-(3-fluoro-4-(4-(3-(trifluoromethoxy)benzylcarbamoyl)-1H-1,2,3-triazol-1-yl)butyl)-2-oxo-1,2-dihydropyrimidin-4-ylcarbamate

To a solution of 1-(4-(4-(tert-butoxycarbonylamino)-2-oxopyrimidin-1(2H)-yl)-2-fluorobutyl)-1H-1,2,3-triazole-4-carboxylic acid (600 mg, 1.52 mmol) and (3-(trifluoromethoxy)phenyl)methanamine (345 mg, 1.82 mmol) in DMF (4 mL), HATU (691 mg, 1.82 mmol) and DIEA (489 mg, 3.8 mmol) were added. The resulting mixture was stirred at RT for 2 h. The reaction mixture was diluted with water (20 mL), and the precipitate was filtered off to give the title compound as a yellow solid (800 mg, 90%). MS (ES+) C₂₄H₂₇F₄N₇O₅ requires: 569. found: 570 [M+H]+.

### Step 6: 1-(4-(4-amino-2-oxopyrimidin-1(2H)-yl)-2-fluorobutyl)-N-(3-(trifluoromethoxy)benzyl)-1H-1,2,3-triazole-4-carboxamide

To a solution of tert-butyl 1-(3-fluoro-4-(4-(3-(trifluoromethoxy)benzylcarbamoyl)-1H-1,2,3-triazol-1-yl)butyl)-2-oxo-1,2-dihydropyrimidin-4-ylcarbamate (800 mg, 1.41 mmol) in DCM (2 mL), TFA (10 mL) was added. The resulting mixture was stirred at RT for 3 h. The mixture was concentrated, diluted with sat. NaHCO₃, extracted with EtOAc (150 mL x 2), and concentrated to give the title compound as a yellow solid (550 mg, 83%). MS (ES+) C₁₉H₁₉F₄N₇O₃ requires: 469. found: 470 [M+H]+.

### Step 7: 3-(trifluoromethoxy)benzyl 1-(3-fluoro-4-(4-(3-(trifluoromethoxy)benzylcarbamoyl)-1H-1,2,3-triazol-1-yl)butyl)-2-oxo-1,2-dihydropyrimidin-4-ylcarbamate

To a solution of tert (3-(trifluoromethoxy)phenyl)methanol (123 mg, 0.64 mmol) and DIEA (1.37 g, 10.7 mmol) in THF (3 mL), triphosgene (127 mg, 0.43 mmol) was added. The resulting mixture was stirred at RT for 3 h. Then 1-(4-(4-amino-2-oxopyrimidin-1(2H)-yl)-2-fluorobutyl)-N-(3-(trifluoromethoxy)benzyl)-1H-1,2,3-triazole-4-carboxamide (51 mg, 0.11 mmol) was added. The mixture was stirred at 80° C. overnight. The resulting mixture was concentrated and the residue purified by silica gel chromatography (0-100% EtOAc/Pet. Ether) to give the crude product which was further purified by prep-HPLC (Mobile phase: A=+0.1% ammonium hydroxide/H₂O, B=acetonitrile; Gradient: B=5%-95% in 18 min; Column: XBridge C18, 5 um, 30 mm×150 mm) to give the title compound as a white solid (20 mg, 26%). MS (ES+) C₂₈H₂₄F₇N₇O₆ requires: 687. found: 688 [M+H]⁺; ¹H NMR (500 MHz, DMSO-d6) δ 9.26 (t, J=6.3 Hz, 1H), 8.58 (s, 1H), 8.07 (d, J=7.3 Hz, 1H), 7.54 (t, J=8.1 Hz, 1H), 7.46 (dd, J=9.7, 6.1 Hz, 3H), 7.34 (d, J=7.6 Hz, 2H), 7.29 (s, 1H), 7.24 (d, J=7.8 Hz, 1H), 6.97 (d, J=7.3 Hz, 1H), 5.23 (s, 2H), 5.07-4.97 (m, 1H), 4.88-4.58 (m, 2H), 4.48 (d, J=6.2 Hz, 2H), 3.95 (ddd, J=20.5, 13.1, 6.5 Hz, 2H), 2.19-1.92 (m, 2H).

### Example 386

### 1-(4-(4-(2-cyclopentylacetamido)-6-oxopyridazin-1(6H)-yl)-2-fluorobutyl)-N-(3-(trifluoromethoxy)benzyl)-1H-1,2,3-triazole-4-carboxamide

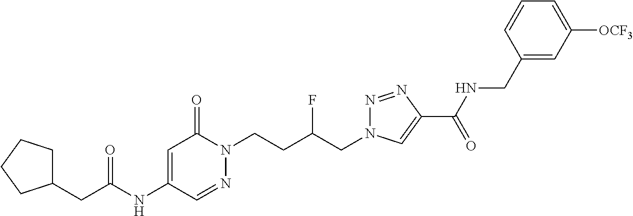 [SMILES: O=C(CC1CCCC1)Nc1cnn(CCC(F)Cn2cc(C(=O)NCc3cccc(OC(F)(F)F)c3)nn2)c(=O)c1]

### Steps 1 to 7

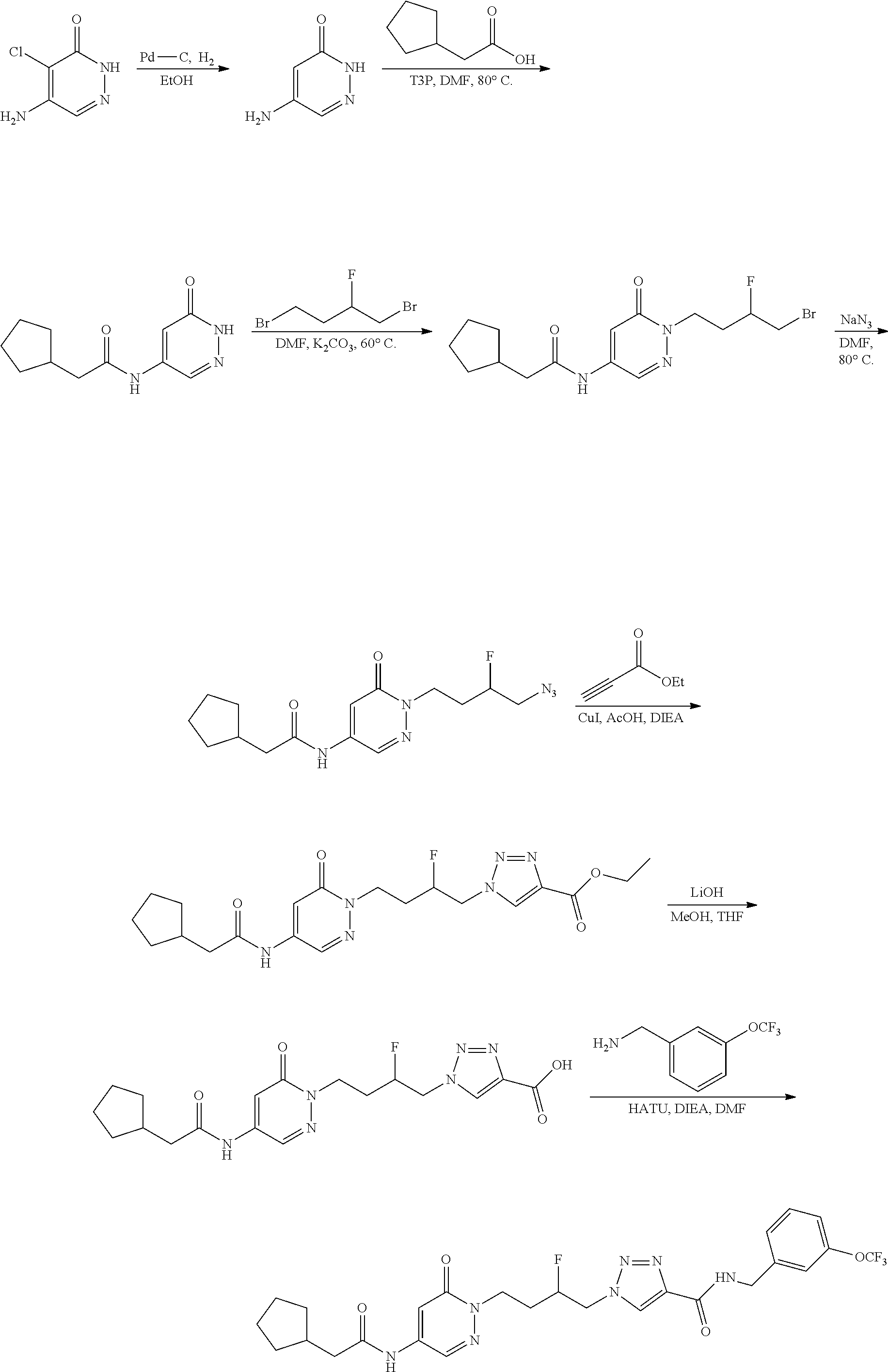

### Step 1: 5-aminopyridazin-3(2H)-one

A reaction vessel was charged with 5-amino-4-chloropyridazin-3(2H)-one (0.60 g, 4.1 mmol), palladium on carbon (2.19 g, 2.061 mmol, 10 wt. %) and EtOH (21 mL) under an atmosphere of N₂. The solution was degassed with N₂ for 5 min and purged with H₂ for 5 min.

The reaction mixture was then heated at 50° C. for 6 h under 1 atm of H₂. The reaction mixture was purged with N₂, filtered through Celite®, and concentrated under reduced pressure to give the title compound as a light gray solid (460 mg, 100). MS (ES⁺) C₄H₅N₃O requires: 111. found: 112 [M+H]⁺; ¹H NMR (600 MHz, DMSO-d₆) δ 13.10 (s, 1H), 7.75 (s, 1H), 7.57-6.74 (m, 2H), 5.97 (s, 1H).

### Step 2: 2-cyclopentyl-N-(6-oxo-1,6-dihydropyridazin-4-yl)acetamide

To a suspension of 2-cyclopentylacetic acid (0.124 mL, 0.990 mmol) and 5-aminopyridazin-3(2H)-one (0.10 g, 0.90 mmol) in pyridine (0.080 mL, 0.99 mmol) was added T3P® in DMF (0.687 g, 1.08 mmol, 50 wt. %) and the resulting mixture was stirred at 80° C. for 1 h. The reaction was cooled and diluted with EtOAc (15 mL), allowed to stir at 0° C. for 10 min, and filtered. The solid was washed with cold EtOAc (10 mL). The filtrate was concentrated and the residue was purified via silica gel chromatography (0-15% MeOH in DCM with 1% NH₄OH) to give the title compound as a yellow amorphous material (130 mg, 65%). MS (ES⁺) C₁₁H₁₅N₃O₂ requires: 221. found: 222 [M+H]⁺.

### Step 3: N-(1-(4-bromo-3-fluorobutyl)-6-oxo-1,6-dihydropyridazin-4-yl)-2-cyclopentylacetamide

To a suspension of 2-cyclopentyl-N-(6-oxo-1,6-dihydropyridazin-4-yl)acetamide (0.130 g, 0.588 mmol) and potassium carbonate (0.162 g, 1.17 mmol) in DMF (1.2 mL) was added 1,4-dibromo-2-fluorobutane (0.275 g, 1.17 mmol) and the resulting mixture was stirred at 60° C. for 5 h. The reaction was concentrated, and the residue was purified via silica gel chromatography (0-15% MeOH in DCM) to give the title compound as a tan solid (101 mg, 46%). MS (ES⁺) C₁₅H₂₁BrFN₃O₂ requires: 373. found: 374 [M+H]⁺.

### Step 4: N-(1-(4-azido-3-fluorobutyl)-6-oxo-1,6-dihydropyridazin-4-yl)-2-cyclopentylacetamide

To a solution of N-(1-(4-bromo-3-fluorobutyl)-6-oxo-1,6-dihydropyridazin-4-yl)-2-cyclopentylacetamide (101 mg, 0.270 mmol) in DMF (540 μl) was added sodium azide (21 mg, 0.32 mmol) and the resulting mixture was stirred at 80° C. for 1 h. The reaction was concentrated (bath temperature <40° C.) to give the title compound as a off-white residue (91 mg, 100%). MS (ES⁺) C₁₅H₂₁FN₆O₄ requires: 336. found: 337 [M+H]⁺.

### Step 5: ethyl 1-(4-(4-(2-cyclopentylacetamido)-6-oxopyridazin-1(6H)-yl)-2-fluorobutyl)-1H-1,2,3-triazole-4-carboxylate

To a suspension of acetic acid (3.1 μl, 0.054 mmol), DIEA (9.4 μl, 0.054 mmol), ethyl propiolate (53 mg, 0.54 mmol), and N-(1-(4-azido-3-fluorobutyl)-6-oxo-1,6-dihydropyridazin-4-yl)-2-cyclopentylacetamide (91 mg, 0.27 mmol) in DCM (1.3 mL) was added copper(I) iodide (2.6 mg, 0.014 mmol) and the resulting mixture was stirred at RT for 12 h. The reaction mixture was adsorbed onto silica gel and purified via silica gel chromatography (0-15% MeOH in DCM with 1% NH₄OH) to give the title compound as a yellow solid (35 mg, 30%). MS (ES⁺) C₂₀H₂₇FN₆O₄ requires: 434. found: 435 [M+H]⁺.

### Step 6: 1-(4-(4-(2-cyclopentylacetamido)-6-oxopyridazin-1(6H)-yl)-2-fluorobutyl)-1H-1,2,3-triazole-4-carboxylic acid

To a suspension of ethyl 1-(4-(4-(2-cyclopentylacetamido)-6-oxopyridazin-1(6H)-yl)-2-fluorobutyl)-1H-1,2,3-triazole-4-carboxylate (35 mg, 0.081 mmol) in THF (322 μl) and MeOH (81 μl) was added aqueous LiOH (44 al, 0.089 mmol, 2.0 M) and the resulting mixture was stirred at RT for 2 h. The reaction was concentrated to give the title compound as a yellow amorphous material (33 mg, 100%, Li salt). MS (ES⁺) C18H₂₃FN₆O₄ requires: 406. found: 407 [M+H]⁺.

### Step 7: 1-(4-(4-(2-cyclopentylacetamido)-6-oxopyridazin-1(6H)-yl)-2-fluorobutyl)-N-(3-(trifluoromethoxy)benzyl)-1H-1,2,3-triazole-4-carboxamide

To a suspension of 1-(4-(4-(2-cyclopentylacetamido)-6-oxopyridazin-1(6H)-yl)-2-fluorobutyl)-1H-1,2,3-triazole-4-carboxylic acid (33 mg, 0.081 mmol), (3-(trifluoromethoxy)phenyl)methanamine (15 mg, 0.081 mmol) and HATU (46 mg, 0.12 mmol) in DMF (406 μl) was added DIEA (28 μl, 0.16 mmol) and the resulting mixture was stirred at RT for 1 h. The reaction mixture was purified by mass-triggered preparative HPLC (Mobile phase: A=0.1% TFA/H₂O, B=0.1% TFA/MeCN; Gradient: B=40-80%; 12 min; Column: C18) to give the title compound as a off-white solid (8.0 mg, 17%). MS (ES⁺) C₂₆H₂₉F₄N₇O₄ requires: 579. found: 580 [M+H]⁺; ¹H NMR (600 MHz, DMSO-d₆) δ 10.34 (s, 1H), 9.20 (t, J=6.3 Hz, 1H), 8.56 (s, 1H), 7.90 (d, J=2.4 Hz, 1H), 7.46 (t, J=7.9 Hz, 1H), 7.35 (dt, J=7.8, 1.2 Hz, 1H), 7.29 (d, J=2.0 Hz, 1H), 7.26-7.20 (m, 2H), 5.01 (dddt, J=49.4, 10.1, 6.7, 3.0 Hz, 1H), 4.84-4.67 (m, 2H), 4.48 (d, J=6.2 Hz, 2H), 4.21-4.07 (m, 2H), 2.36 (d, J=7.4 Hz, 2H), 2.24-2.03 (m, 2H), 1.95 (dddd, J=21.7, 15.0, 9.3, 6.9 Hz, 1H), 1.78-1.70 (m, 2H), 1.64-1.45 (m, 4H), 1.21-1.11 (m, 2H).

### Example 127

### (S)-1-(2-fluoro-4-(2-oxo-4-(2-phenylacetamido)pyridin-1(2H)-yl)butyl)-N-(3-(trifluoromethoxy)benzyl)-1H-1,2,3-triazole-4-carboxamide

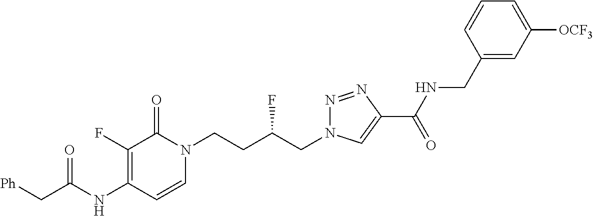 [SMILES: O=C(Cc1ccccc1)Nc1ccn(CC[C@H](F)Cn2cc(C(=O)NCc3cccc(OC(F)(F)F)c3)nn2)c(=O)c1F]

### Steps 1 to 6

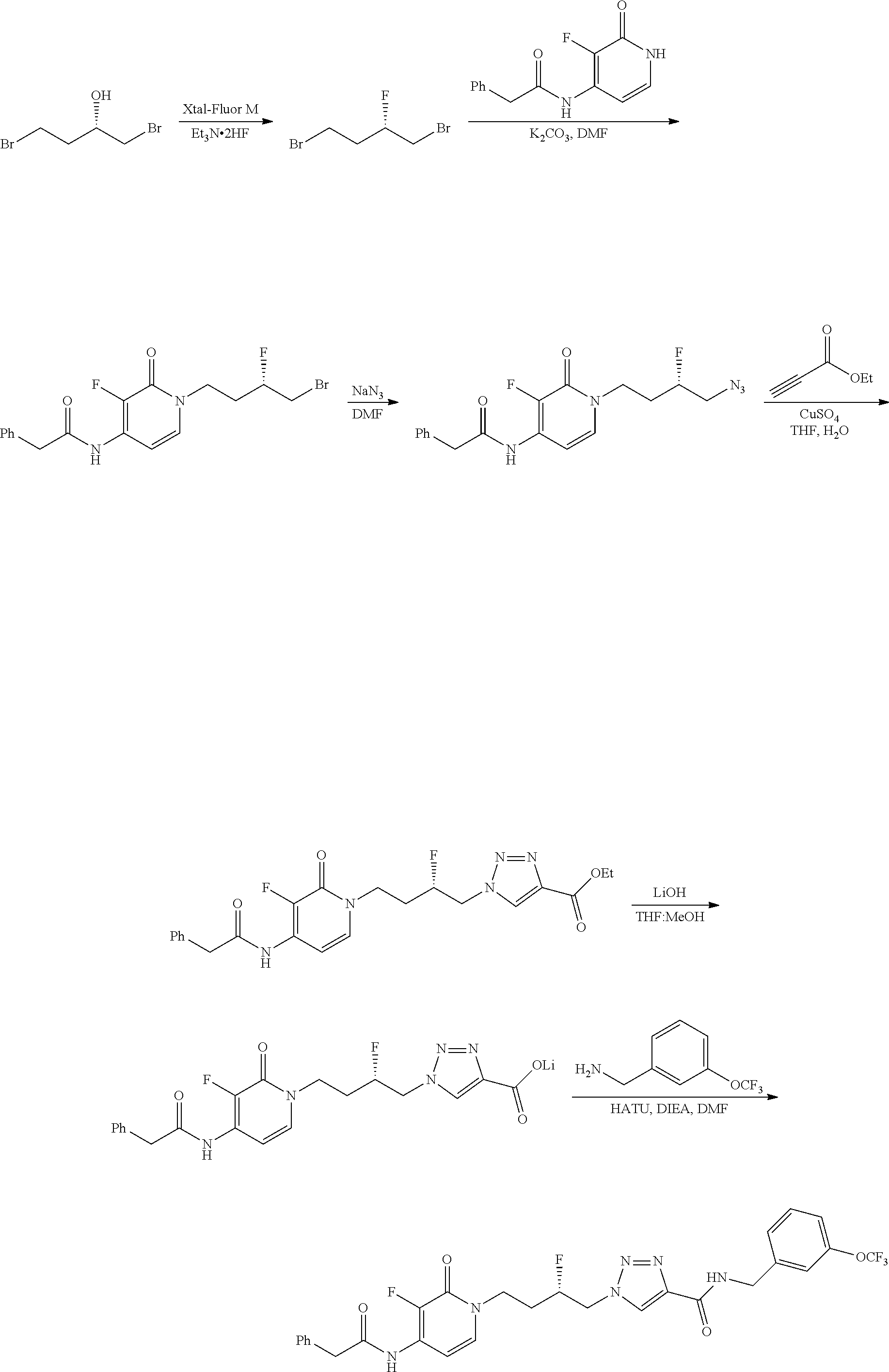

### Step 1: (S)-1,4-dibromo-2-fluorobutane

To a cooled solution of triethylamine trihydrofluoride (2.81 mL, 17.2 mmol) in DCM (43.1 mL) at 0° C. was added triethylamine (1.20 mL, 8.62 mmol). After 5 min, XtalFluor-M (3.14 g, 12.9 mmol) was added. After an additional 5 min (S)-1,4-dibromobutan-2-ol (1.00 mL, 8.62 mmol) was added and the resulting mixture was stirred as it warmed to RT over 7 h. The reaction was quenched with 5% aqueous NaHCO₃ (20 mL), the layers were separated, and the aqueous layer was extracted with DCM (2×20 mL). The organic layers were combined, dried over Na₂SO₄, and the volatiles were removed under reduced pressure to give the title compound as a light yellow oil (1.35 g, 67%). ¹H NMR (600 MHz, Chloroform-d) δ 4.90 (ddtd, J=47.6, 9.5, 4.9, 2.9 Hz, 1H), 3.59-3.46 (m, 4H), 2.40-2.28 (m, 1H), 2.27-2.13 (m, 1H).

To determine absolute stereochemistry and enantiopurity, an analytical sample was derivatized as follows:

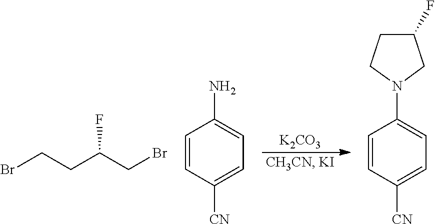

A microwave vial was charged with K₂CO₃ (591 mg, 4.28 mmol), KI (71 mg, 0.43 mmol), 4-aminobenzonitrile (303 mg, 2.57 mmol), (S)-1,4-dibromo-2-fluorobutane (400 mg, 1.71 mmol) and MeCN (1710 μl). The vial was sealed and the reaction mixture was heated to 170° C. in the microwave reactor for 3 h. The volatiles were removed under reduced pressure and the residue was purified via mass-triggered preparative HPLC (Mobile phase: A=0.1% TFA/H₂O, B=0.1% TFA/MeCN; Gradient: B=20-60%; 12 min; Column: C18) to give (S)-4-(3-fluoropyrrolidin-1-yl)benzonitrile (45 mg, 14%, TFA salt) as a white crystalline solid. MS (ES⁺) C₁₁H₁₁FN₂ requires: 190. found: 191 [M+H]⁺; ¹H NMR (600 MHz, CDCl3) δ 7.49 (d, 2H, J=8.7 Hz), 6.53 (d, 2H, J=8.7 Hz), 5.40 (dm, 1H, J=53 Hz), 3.63 (m, 1H), 3.56-3.60 (m, 1H), 3.53-3.56 (m, 1H), 3.52 (m, 1H), 2.43 (m, 1H), 2.10-2.25 (m, 1H). Analytical chiral HPLC (Phenomenex Lux-3 250×4.6 mm @1.0 mL/min, 20% EtOH in Hexane with 0.1% TFA) determined the sample to be 85% ee. Racemic and enantiopure standards were synthesized from 3-fluoropyrrolidine hydrochloride, 4-fluorobenzonitrile, and potassium carbonate in MeCN. The standard derived from commerically available (S)-3-fluoropyrrolidine hydrochloride matched the sample prepared above, which allowed assignment of the (S)-configuration.

### Step 2 to 6: (S)-1-(2-fluoro-4-(2-oxo-4-(2-phenylacetamido)pyridin-1(2H)-vyl)butyl)-N-(3-(trifluoromethoxy)benzyl)-1H-1,2,3-triazole-4-carboxamide

Following EXAMPLE 180, steps 2 to 8, gave the title compound as a white solid. MS (ES⁺) C₂₈H₂₅F₅N₆O₄ requires: 604. found: 605 [M+H]⁺

### Example 304

### 1-(4-(4-(2-cyclobutylacetamido)-3-fluoro-2-oxopyridin-1(2H)-yl)-2-fluorobutyl)-N-((6-methylpyridin-3-yl)methyl)-1H-1,2,3-triazole-4-carboxamide

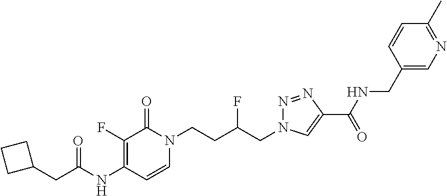 [SMILES: Cc1ccc(CNC(=O)c2cn(CC(F)CCn3ccc(NC(=O)CC4CCC4)c(F)c3=O)nn2)cn1]

### Step 1: 1-(4-(4-(2-cyclobutylacetamido)-3-fluoro-2-oxopyridin-1(2H)-yl)-2-fluorobutyl)-N-((6-methylpyridin-3-yl)methyl)-1H-1,2,3-triazole-4-carboxamide

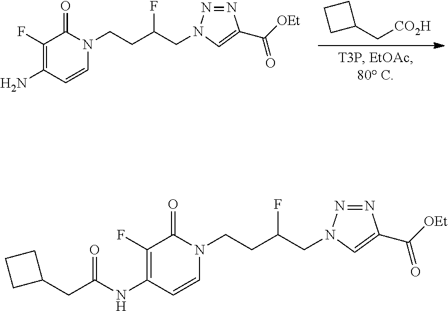 [SMILES: CCOC(=O)c1cn(CC(F)CCn2ccc(N)c(F)c2=O)nn1 ; O=C(O)CC1CCC1 ; CCOC(=O)c1cn(CC(F)CCn2ccc(NC(=O)CC3CCC3)c(F)c2=O)nn1]

To a solution of ethyl 1-(4-(4-amino-3-fluoro-2-oxopyridin-1(2H)-yl)-2-fluorobutyl)-1H-1,2,3-triazole-4-carboxylate (0.30 g, 0.88 mmol), and 2-cyclobutylacetic acid (0.15 g, 1.3 mmol) was added 2,4,6-tripropyl-1,3,5,2,4,6-trioxatriphosphinane 2,4,6-trioxide (T3P®) in EtOAc (2.80 g, 4.39 mmol, 50 wt. %) and pyridine (0.213 mL, 2.64 mmol). The resulting mixture was stirred for 1 h at 80° C. The reaction was cooled to RT and the resulting thick white suspension was diluted with EtOAc (10 mL) and filtered. The collected solids were washed with EtOAc and dried to give the title compound as a white solid (326 mg, 76%). MS(ES⁺) C₂₀H₂₅F₂N₅O₄ requires: 437. found: 438 [M+H]⁺.

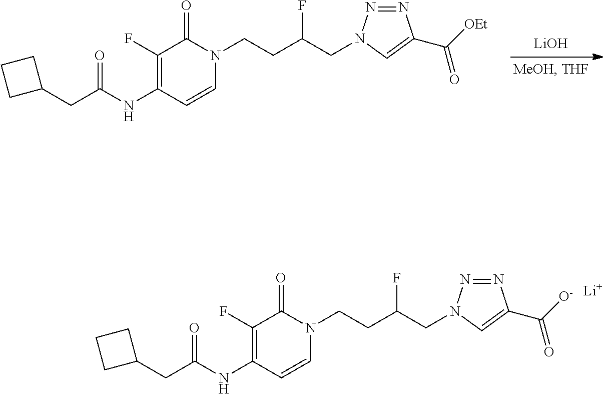 [SMILES: CCOC(=O)c1cn(CC(F)CCn2ccc(NC(=O)CC3CCC3)c(F)c2=O)nn1 ; "LiOH" ; O=C(CC1CCC1)Nc1ccn(CCC(F)Cn2cc(C(=O)[O-])nn2)c(=O)c1F ; [Li+]]

### Step 2: lithium 1-(4-(4-(2-cyclobutylacetamido)-3-fluoro-2-oxopyridin-1(2H)-yl)-2-fluorobutyl)-1H-1,2,3-triazole-4-carboxylate

To a suspension of ethyl 1-(4-(4-(2-cyclobutylacetamido)-3-fluoro-2-oxopyridin-1(2H)-yl)-2-fluorobutyl)-1H-1,2,3-triazole-4-carboxylate (0.325 g, 0.743 mmol) in THF (2.97 mL) and MeOH (0.74 mL) was added aq. lithium hydroxide (0.409 mL, 0.817 mmol, 2.0 M) and the resulting mixture was stirred at RT for 2 h. The reaction was concentrated, azeotroping with EtOH, to give the title compound (0.304 g, 100%) as a green foam. MS(ES⁺) C₁₈H₂₁F₂N₅O₄ requires: 409. found: 410 [M+H]⁺.

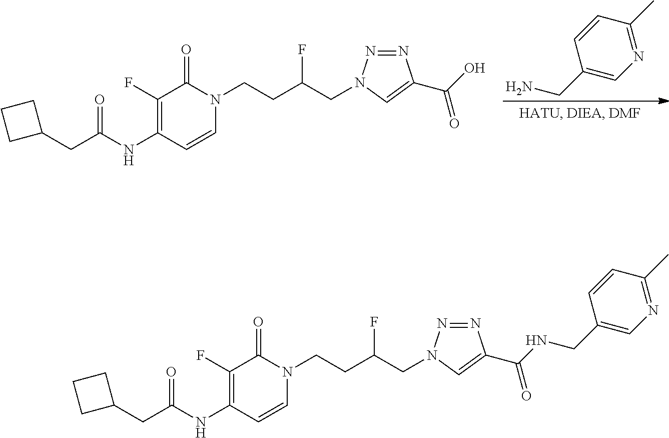

### Step 3: 1-(4-(4-(2-cyclobutylacetamido)-3-fluoro-2-oxopyridin-1(2H)-yl)-2-fluorobutyl)-N-((6-methylpyridin-3-yl)methyl)-1H-1,2,3-triazole-4-carboxamide

To a suspension of (6-methylpyridin-3-yl)methanamine (28.6 mg, 0.234 mmol), 1-(4-(4-(2-cyclobutylacetamido)-3-fluoro-2-oxopyridin-1(2H)-yl)-2-fluorobutyl)-1H-1,2,3-triazole-4-carboxylic acid (80 mg, 0.195 mmol) and 2-(3H-[1,2,3]triazolo[4,5-b]pyridin-3-yl)-1,1,3,3-tetramethylisouronium hexafluorophosphate(V) (111 mg, 0.293 mmol) in DMF (977 μl) was added N-ethyl-N-isopropylpropan-2-amine (68 μl, 0.39 mmol) and the resulting mixture was stirred at RT for 1 h. The reaction mixture was concentrated and the residue was purified via silica gel chromatography (0-15% MeOH in DCM with 1% NH₄OH) to give the title compound as an off-white solid (22 mg, 21%). MS(ES⁺) C₂₅H₂₉F₂N₇O₃ requires: 513. found: 514 [M+H]⁺. ¹H NMR (500 MHz, DMSO-d₆) δ 9.91 (s, 1H), 9.16 (t, J=6.2 Hz, 1H), 8.55 (s, 1H), 8.39 (d, J=2.2 Hz, 1H), 7.60 (dd, J=8.0, 2.3 Hz, 1H), 7.44 (dd, J=7.7, 1.6 Hz, 1H), 7.19 (d, J=8.0 Hz, 1H), 7.05 (dd, J=7.7, 6.4 Hz, 1H), 5.09-4.88 (m, 1H), 4.85-4.66 (m, 2H), 4.41 (d, J=6.1 Hz, 2H), 4.04 (ddt, J=44.4, 13.5, 7.2 Hz, 2H), 2.69-2.56 (m, 1H), 2.53 (d, J=7.5 Hz, 3H), 2.16-1.63 (m, 8H).

### Example 358

### 1-(2-fluoro-4-(3-fluoro-2-oxo-4-(2-oxo-2-(piperidin-1-yl)acetamido)pyridin-1(2H)-yl)butyl)-N-(3-(trifluoromethoxy)benzyl)-1H-1,2,3-triazole-4-carboxamide

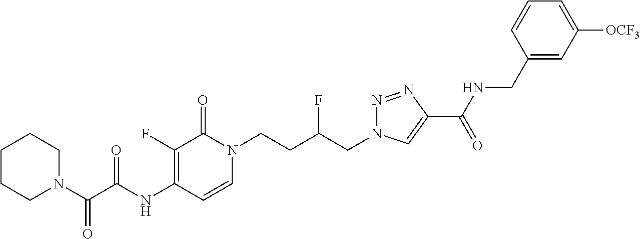 [SMILES: O=C(Nc1ccn(CCC(F)Cn2cc(C(=O)NCc3cccc(OC(F)(F)F)c3)nn2)c(=O)c1F)C(=O)N1CCCCC1]

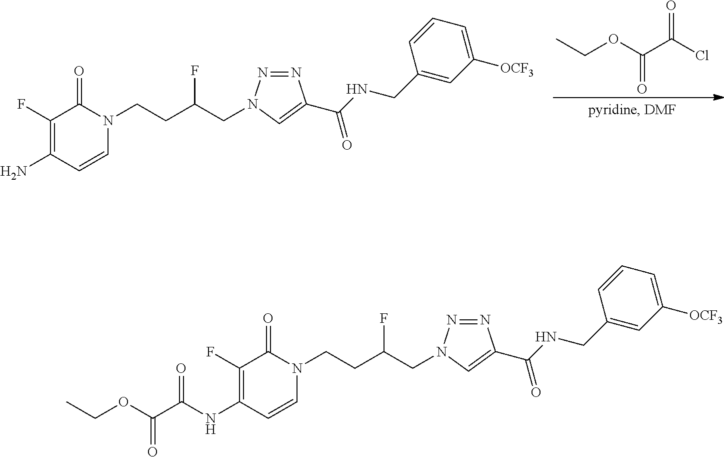

### Step 1: ethyl 2-((3-fluoro-1-(3-fluoro-4-(4-((3-(trifluoromethoxy)benzyl)carbamoyl)-1H-1,2,3-triazol-1-yl)butyl)-2-oxo-1,2-dihydropyridin-4-yl)amino)-2-oxoacetate

To a solution of 1-(4-(4-amino-3-fluoro-2-oxopyridin-1(2H)-yl)-2-fluorobutyl)-N-(3-(trifluoromethoxy)benzyl)-1H-,2,3-triazole-4-carboxamide (100 mg, 0.206 mmol) in DMF (0.2 mL) was added pyridine (0.067 mL, 0.822 mmol). The reaction mixture was cooled to 0° C. in an ice bath, and ethyl 2-chloro-2-oxoacetate (33.7 mg, 0.247 mmol) added. The resulting mixture was removed from the bath and stirred at RT for 2 h. An additional amount of ethyl 2-chloro-2-oxoacetate (33.7 mg, 0.247 mmol) was added and the reaction mixture was stirred at RT for an additional 2 h. The reaction mixture was quenched with MeOH, concentrated, and the resulting solids were taken up into a minimal amount of MeOH, the suspension was filtered, and the collected solids were washed with a minimal amount of MeOH, then hexanes to give the title compound as a white solid (99 mg, 82%). MS (ES⁺) C₂₄H₂₃F₅N₆O₆ requires: 586. found: 587 [M+H]⁺.

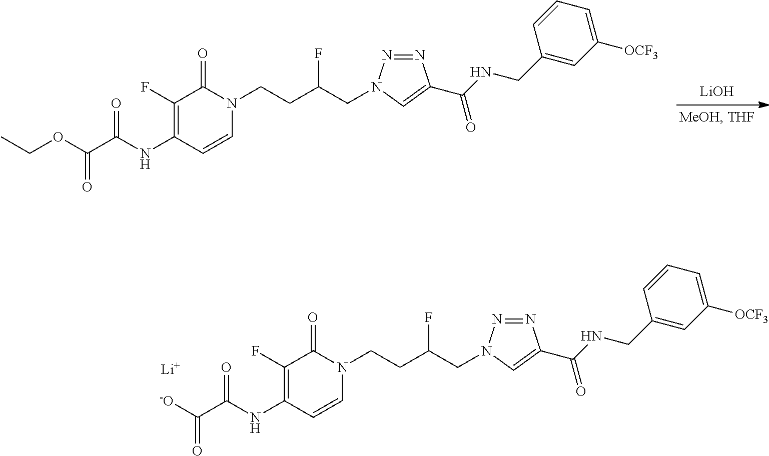

### Step 2: Lithium 2-((3-fluoro-1-(3-fluoro-4-(4-((3-(trifluoromethoxy)benzyl)carbamoyl)-1H-1,2,3-triazol-1-yl)butyl)-2-oxo-1,2-dihydropyridin-4-yl)amino)-2-oxoacetate

To a suspension of ethyl 2-((3-fluoro-1-(3-fluoro-4-(4-((3-(trifluoromethoxy)benzyl)carbamoyl)-1H-1,2,3-triazol-1-yl)butyl)-2-oxo-1,2-dihydropyridin-4-yl)amino)-2-oxoacetate (38 mg, 0.065 mmol) in THF (2.6 mL) and MeOH (65 μl) was added aq. lithium hydroxide (35.6 μl, 0.071 mmol, 2.0 M) and the resulting mixture was stirred at RT for 1 h. The reaction mixture was concentrated to give the title compound as a white solid (36 mg, 100%). MS (ES⁺) C₂₂H₁₉F₅N₆06 requires: 558. found: 559 [M+H]⁺.

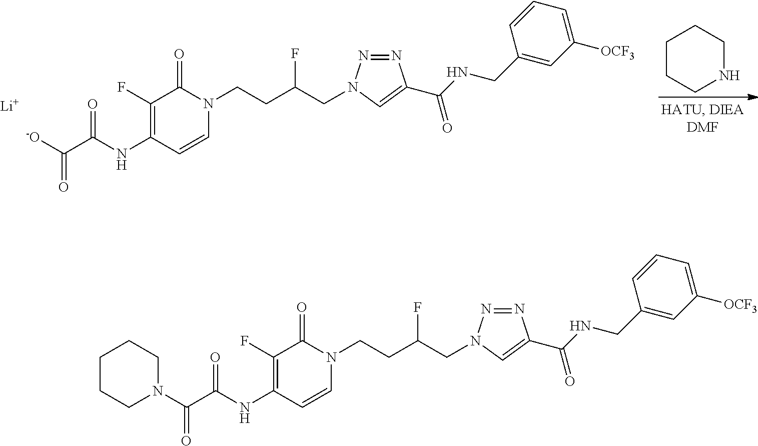

### Step 3: 1-(2-fluoro-4-(3-fluoro-2-oxo-4-(2-oxo-2-(piperidin-1-yl)acetamido)pyridin-1(2H)-yl)butyl)-N-(3-(trifluoromethoxy)benzyl)-1H-1,2,3-triazole-4-carboxamide

To a suspension of piperidine (7.66 μl, 0.077 mmol), lithium 2-((3-fluoro-1-(3-fluoro-4-(4-((3-(trifluoromethoxy)benzyl)carbamoyl)-1H-1,2,3-triazol-1-yl)butyl)-2-oxo-1,2-dihydropyridin-4-yl)amino)-2-oxoacetate (36 mg, 0.064 mmol) and 2-(3H-[1,2,3]triazolo[4,5-b]pyridin-3-yl)-1,1,3,3-tetramethylisouronium hexafluorophosphate(V) (37 mg, 0.097 mmol) in DMF (0.65 mL) was added DIEA (23 μl, 0.13 mmol) and the resulting mixture was stirred at RT for 1 h. The reaction mixture was purified by mass-triggered preparative HPLC (Mobile phase: A=0.1% TFA/H₂O, B=0.1% TFA/MeCN; Gradient: B=40-80%; 12 min; Column: C18) to give the title compound as a white solid (10 mg, 25%). MS (ES⁺) C₂₇H₂₈F₅N₇O₅ requires: 625. found: 626 [M+H]⁺. ¹H NMR (600 MHz, DMSO-d₆) δ 10.92 (s, 1H), 9.20 (t, J=6.2 Hz, 1H), 8.57 (s, 1H), 7.54 (dd, J=7.6, 1.6 Hz, 1H), 7.46 (t, J=7.9 Hz, 1H), 7.35 (d, J=7.7 Hz, 1H), 7.29 (s, 1H), 7.26-7.18 (m, 1H), 6.92 (t, J=6.9 Hz, 1H), 5.01 (dddt, J=49.8, 10.0, 6.7, 3.0 Hz, 1H), 4.86-4.67 (m, 2H), 4.48 (d, J=6.2 Hz, 2H), 4.17-4.01 (m, 2H), 3.41 (dt, J=69.8, 5.5 Hz, 4H), 2.19-1.89 (m, 2H), 1.68-1.50 (m, 6H).

### Example 422

### 11-(2-fluoro-4-(3-fluoro-2-oxo-4-(2-oxo-2-(piperidin-1-yl)acetamido)pyridin-1(2H)-yl)butyl)-N-(2-fluoro-5-(trifluoromethoxy)benzyl)-1H-1,2,3-triazole-4-carboxamide

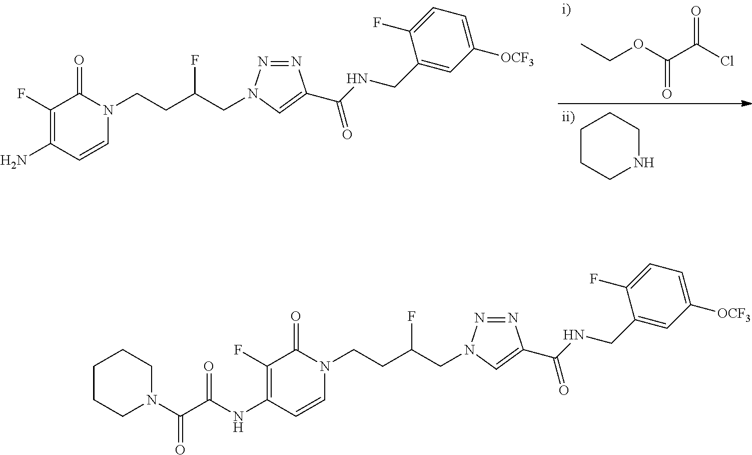

To a suspension of 1-(4-(4-amino-3-fluoro-2-oxopyridin-1(2H)-yl)-2-fluorobutyl)-N-(2-fluoro-5-(trifluoromethoxy)benzyl)-1H-1,2,3-triazole-4-carboxamide (122 mg, 0.242 mmol) and DIEA (211 μl, 1.21 mmol) in THF (4.8 mL) at 0° C. was added oxalyl chloride (151 μl, 0.302 mmol). The resulting mixture was stirred as it warmed to RT for 25 min. The reaction was cooled to 0° C. again and piperidine (48 μl, 0.48 mmol) was added. The resulting mixture was stirred as it warmed to RT for 1 h. The volatiles were removed under reduced pressure and the residue was purified via silica gel chromatography (0-10% MeOH in DCM with 1% NH₄OH) to give the title compound (67 mg, 43%) as an off-white crystalline solid. MS (ES⁺) C₂₇H₂₇F₆N₇O₅ requires: 643. found: 644 [M+H]⁺; ¹H NMR (600 MHz, DMSO-d₆) δ 10.93 (s, 1H), 9.20 (t, J=6.1 Hz, 1H), 8.59 (s, 1H), 7.56-7.51 (m, 1H), 7.37-7.31 (m, 3H), 6.93 (t, J=6.8 Hz, 1H), 5.02 (dt, J=49.4, 8.5 Hz, 1H), 4.85-4.69 (m, 2H), 4.52 (d, J=6.1 Hz, 2H), 4.10 (ddt, J=50.8, 13.7, 6.9 Hz, 2H), 3.51-3.37 (m, 4H), 2.19-1.89 (m, 2H), 1.67-1.50 (m, 6H).

### Example 435

### 1-(2-fluoro-4-(3-fluoro-2-oxo-4-(2-oxo-2-(piperidin-1-yl)acetamido)pyridin-1(2H)-yl)butyl)-N-(3-(trifluoromethyl)benzyl)-1H-1,2,3-triazole-4-carboxamide

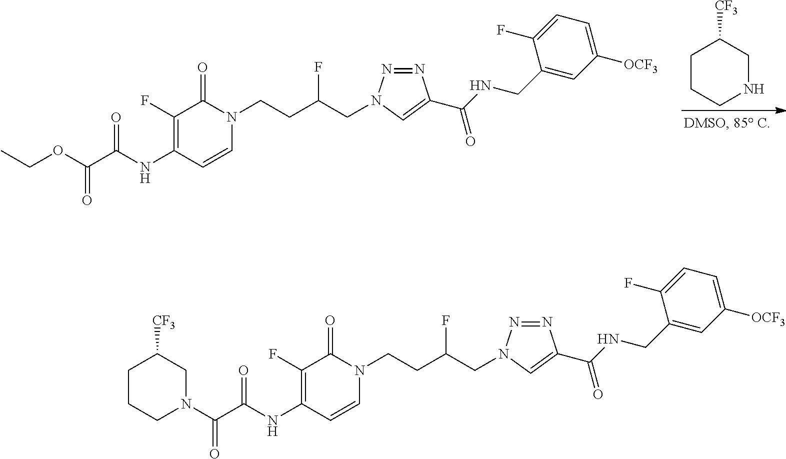 [SMILES: CCOC(=O)C(=O)Nc1ccn(CCC(F)Cn2cc(C(=O)NCc3cc(OC(F)(F)F)ccc3F)nn2)c(=O)c1F ; FC(F)(F)[C@H]1CCCNC1 ; O=C(Nc1ccn(CCC(F)Cn2cc(C(=O)NCc3cc(OC(F)(F)F)ccc3F)nn2)c(=O)c1F)C(=O)N1CCC[C@H](C(F)(F)F)C1]

To a solution of ethyl 2-((3-fluoro-1-(3-fluoro-4-(4-((2-fluoro-5-(trifluoromethoxy)benzyl)carbamoyl)-1H-1,2,3-triazol-1-yl)butyl)-2-oxo-1,2-dihydropyridin-4-yl)amino)-2-oxoacetate (33 mg, 0.055 mmol) in DMSO (1 mL) was added (S)-3-(trifluoromethyl)piperidine (52 mg, 0.340 mmol) and the resulting mixture was stirred at 85° C. for 5 h. The residue was purified by mass-triggered preparative HPLC (Mobile phase: A=0.1% TFA/H₂O, B=0.1% TFA/MeCN; Gradient: B=40-80%; 12 min; Column: C18) and lyophilized to give to the title compound as a white powder (26 mg, 67% yield, 1:1 mixture of diastereomers). MS(ES⁺) C₂₈H₂₆F₉N₇O₅ requires: 711. found: 712 [M+H]⁺. ¹H NMR (600 MHz, DMSO-d₆) δ ppm 10.94 (s, 2H), 9.20 (m, 2H), 8.59 (s, 2H), 7.55 (d, J=7.6 Hz, 2H), 7.36-7.31 (m, 6H), 6.95 (m, 1H), 6.83 (m, 1H), 5.05 (m, 1H), 4.97 (m, 1H), 4.84-4.69 (m, 4H), 4.50 (d, J=6.1 Hz, 4H), 4.36 (m, 1H), 4.22-4.00 (m, 5H), 3.83 (d, J=13 Hz, 1H), 3.63 (d, J=13.4 Hz, 1H), 3.27 (dd, J=11.3 Hz, 1H), 3.18 (t, J=12.6 Hz, 1H), 2.91-2.81 (m, 2H), 2.18-1.91 (m, 6H), 1.78 (m, 2H), 1.66-1.46 (m, 4H).

### Example 464

### 5-(4-(4-(2-cyclobutylacetamido)-3-fluoro-2-oxopyridin-1(2H)-yl)butyl)-N-((6-methylpyridin-3-yl)methyl)-1,3,4-thiadiazole-2-carboxamide

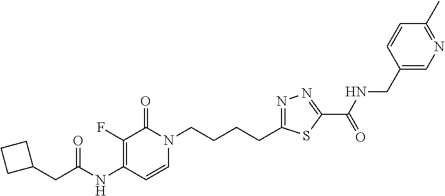 [SMILES: Cc1ccc(CNC(=O)c2nnc(CCCCn3ccc(NC(=O)CC4CCC4)c(F)c3=O)s2)cn1]

### Steps 1 to 4

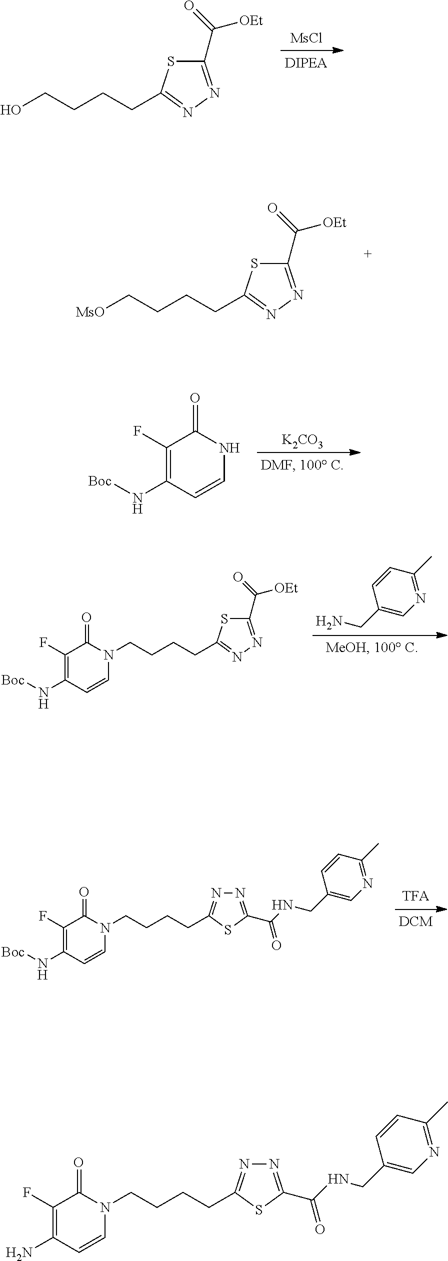

### Step 1: ethyl 5-(4-(methylsulfonyloxy)butyl)-1,3,4-thiadiazole-2-carboxylate

To a solution of ethyl 5-(4-hydroxybutyl)-1,3,4-thiadiazole-2-carboxylate (4.2 g, 18 mmol, see EXAMPLE 28) and DIEA (7.1 g, 54.8 mmol) in DCM (50 mL), MsCl (3.1 g, 27.4 mmol) was slowly added. The mixture was stirred at RT for 3 h. The solution was diluted with water (100 mL) and extracted with DCM (2×100 mL). The combined organic layers was dried over sodium sulfate, filtered, and concentrated under reduced pressure to obtain the title compound as a brown oil (5.2 g, 95%). MS (ES+) C₁₀H₁₆N₂O₅S₂ requires: 308. found: 309 [M+H]⁺.

### Step 2: ethyl 5-(4-(4-(tert-butoxycarbonylamino)-3-fluoro-2-oxopyridin-1(2H)-yl)butyl)-1,3,4-thiadiazole-2-carboxylate

A mixture of ethyl 5-(4-(methylsulfonyloxy)butyl)-1,3,4-thiadiazole-2-carboxylate (5.2 g, 17.1 mmol), tert-butyl 3-fluoro-2-oxo-1,2-dihydropyridin-4-ylcarbamate (3.12 g, 13.7 mmol), and K₂CO₃ (5.6 g, 41.1 mmol) in DMF (20 mL) was stirred at 100° C. for 1 h. The mixture was concentrated and the residue was purified by silica gel chromatography (Pet. Ether: EtOAc=1:1-1:2) to obtain the title compound as a brown solid (1.7 g, 30%). MS (ES+) C₁₉H₂₅FN₄O₅S requires: 440. found: 441 [M+H]⁺.

### Step 3: tert-butyl 3-fluoro-1-(4-(5-((6-methylpyridin-3-yl)methylcarbamoyl)-1,3,4-thiadiazol-2-yl)butyl)-2-oxo-1,2-dihydropyridin-4-ylcarbamate

A solution of ethyl 5-(4-(4-(tert-butoxycarbonylamino)-3-fluoro-2-oxopyridin-1(2H)-yl)butyl)-1,3,4-thiadiazole-2-carboxylate (500 mg, 1.14 mmol) and (6-methylpyridin-3-yl)methanamine (416 mg, 3.4 mmol) in MeOH (5 mL) was stirred at 100° C. for 4 h. The reaction mixture was concentrated and the residue was purified by eluting through a short plug of silica gel (eluting with EtOAc) to obtain the title compound as white solid (540 mg, 70%). MS (ES+) C₂₄H₂₉FN₆O₄S requires: 516. found: 517 [M+H]⁺.

### Step 5: 5-(4-(4-amino-3-fluoro-2-oxopyridin-1(2H)-yl)butyl)-N-((6-methylpyridin-3-yl)methyl)-1,3,4-thiadiazole-2-carboxamide

To a solution of tert-butyl 3-fluoro-1-(4-(5-((6-methylpyridin-3-yl)methylcarbamoyl)-1,3,4-thiadiazol-2-yl)butyl)-2-oxo-1,2-dihydropyridin-4-ylcarbamate (540 mg, 0.97 mmol) in DCM (5 mL) was added TFA (5 mL) and the resulting solution was stirred at RT for 3 h. The mixture was concentrated to give the title compound as a brown oil (600 mg, >100%, TFA salt). MS (ES+) C₁₉H₂₁FN₆02 S requires: 416. found: 417 [M+H]⁺.

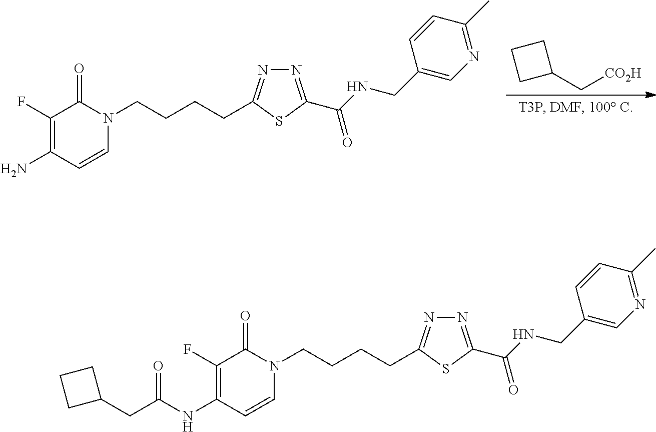

### Step 5: 5-(4-(4-(2-cyclobutylacetamido)-3-fluoro-2-oxopyridin-1(2H)-yl)butyl)-N-((6-methylpyridin-3-yl)methyl)-1,3,4-thiadiazole-2-carboxamide

To a solution of 5-(4-(4-amino-3-fluoro-2-oxopyridin-1(2H)-yl)butyl)-N-((6-methylpyridin-3-yl)methyl)-1,3,4-thiadiazole-2-carboxamide (100 mg, 0.24 mmol, TFA salt) and 2-cyclobutylacetic acid (72 mg, 0.72 mmol) in DMF (5 mL) was added T3P® in EtOAc (759 mg, 1.20 mmol, 50 wt. %). The resulting mixture was stirred at 100° C. overnight. The mixture was purified by Prep-HPLC (Mobile phase: A=+0.1% NH₄OH/H₂O, B=acetonitrile; Gradient: B=5%-95% in 18 min; Column: C18) to obtain the title compound as a white solid (20 mg, 27%). MS (ES+) C₂₅H₂₉FN₆O₃S requires: 512. found: 513 [M+H]⁺. ¹H NMR (500 MHz, DMSO-d6) δ 9.90 (s, 1H), 9.85 (s, 1H), 8.62 (s, 1H), 8.09 (s, 1H), 7.61 (s, 1H), 7.47 (d, J=7.4 Hz, 1H), 7.05 (s, 1H), 4.54 (d, J=5.3 Hz, 2H), 3.93 (s, 2H), 3.18 (s, 2H), 2.58 (t, J=21.9 Hz, 6H), 2.03 (s, 2H), 1.82 (d, J=8.5 Hz, 2H), 1.71 (s, 6H).

### Example 468

### Cyclobutyl(3-fluoro-1-(4-(5-(((6-methylpyridin-3-yl)methyl)carbamoyl)-1,3,4-thiadiazol-2-yl)butyl)-2-oxo-1,2-dihydropyridin-4-yl)carbamate

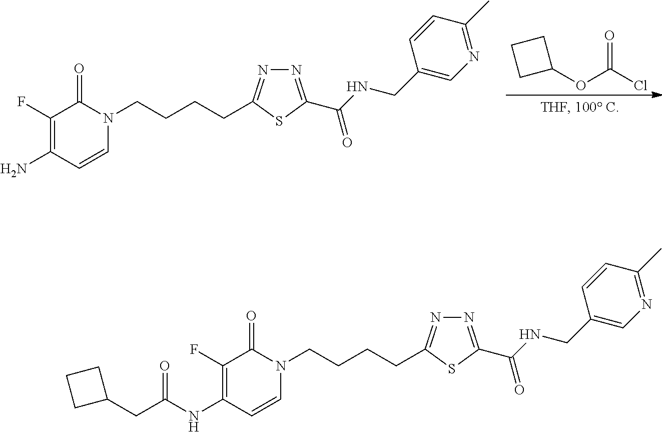

To a solution of cyclobutanol (52 mg, 0.43 mmol) and DIEA (155 mg, 1.202 mmol) in THF (10 mL) was added bis(trichloromethyl) carbonate (128 mg, 0.721 mmol) at 0° C. The reaction was stirred at RT for 6 h. The mixture was then added to a solution of 5-(4-(4-amino-3-fluoro-2-oxopyridin-1(2H)-yl)butyl)-N-((6-methylpyridin-3-yl)methyl)-1,3,4-thiadiazole-2-carboxamide (100 mg, 0.24 mmol) in THF (10 mL) and stirred at 100° C. for 24 h. The mixture was concentrated and purified by Prep-HPLC (Mobile phase: A=+0.1% NH₄OH/H₂O, B=acetonitrile; Gradient: B=5%-95% in 18 min; Column: C18) to obtain the title compound as a white solid (12 mg, 10%). MS (ES+) C₂₄H₂₇FN₆O₄S requires: 514. found: 515 [M+H]⁺. ¹H NMR (500 MHz, DMSO-d6) δ 9.86 (t, J=6.1 Hz, 1H), 9.82 (s, 1H), 8.66 (s, 1H), 8.18 (d, J=6.7 Hz, 1H), 7.69 (d, J=8.3 Hz, 1H), 7.48 (d, J=6.9 Hz, 1H), 6.96-6.63 (m, 1H), 4.93 (p, J=7.3 Hz, 1H), 4.56 (d, J=6.1 Hz, 2H), 3.92 (s, 2H), 3.18 (s, 2H), 2.62 (s, 3H), 2.29 (dd, J=16.2, 8.2 Hz, 2H), 2.11-1.97 (m, 2H), 1.80-1.50 (m, 6H).

Non-limiting examples include the following compounds and pharmaceutically acceptable salts thereof:

TABLE 1
Synthesized Compounds

| Ex. | Structure | IUPAC Name |
|---|---|---|
| 30 | 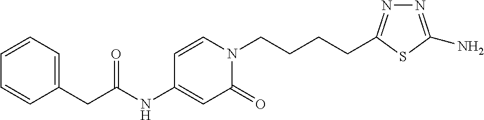 [SMILES: Nc1nnc(CCCCn2ccc(NC(=O)Cc3ccccc3)cc2=O)s1] | N-{1-[4-(5-amino-1,3,4-thiadiazol-2- yl)butyl]-2-oxo-1,2-dihydropyridin-4- yl}-2-phenylacetamide |
| 31 | 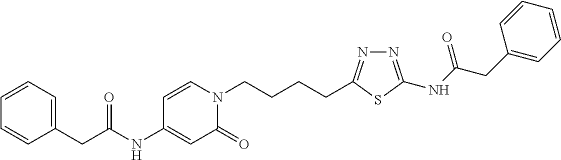 [SMILES: O=C(Cc1ccccc1)Nc1ccn(CCCCc2nnc(NC(=O)Cc3ccccc3)s2)c(=O)c1] | N-(2-oxo-1-{4-[5-(2-phenylacetamido)- 1,3,4-thiadiazol-2-yl]butyl}-1,2- dihydropyridin-4-yl)-2-phenylacetamide |
| 32 | 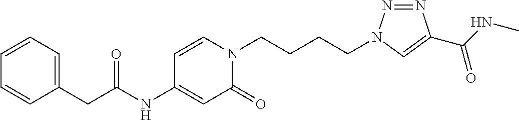 [SMILES: CNC(=O)c1cn(CCCCn2ccc(NC(=O)Cc3ccccc3)cc2=O)nn1] | N-methyl-1-{4-[2-oxo-4-(2- phenylacetamido)-1,2-dihydropyridin-1- yl]butyl}-1H-1,2,3-triazole-4- carboxamide |
| 33 | 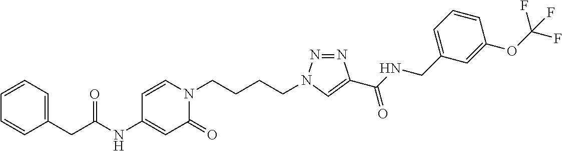 [SMILES: O=C(Cc1ccccc1)Nc1ccn(CCCCn2cc(C(=O)NCc3cccc(OC(F)(F)F)c3)nn2)c(=O)c1] | 1-{4-[2-oxo-4-(2-phenylacetamido)-1,2- dihydropyridin-1-yl]butyl}-N-{[3- (trifluoromethoxy)phenyl]methyl}-1H- 1,2,3-triazole-4-carboxamide |
| 34 | 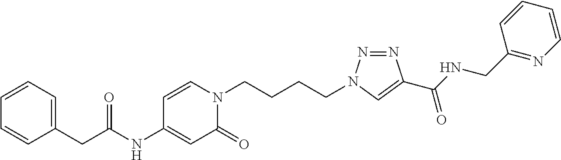 [SMILES: O=C(Cc1ccccc1)Nc1ccn(CCCCn2cc(C(=O)NCc3ccccn3)nn2)c(=O)c1] | 1-{4-[2-oxo-4-(2-phenylacetamido)-1,2- dihydropyridin-1-yl]butyl}-N-(pyridin- 2-ylmethyl)-1H-1,2,3-triazole-4- carboxamide |
| 35 | 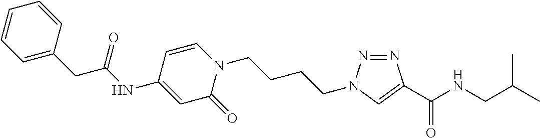 [SMILES: CC(C)CNC(=O)c1cn(CCCCn2ccc(NC(=O)Cc3ccccc3)cc2=O)nn1] | N-(2-methylpropyl)-1-{4-[2-oxo-4-(2- phenylacetamido)-1,2-dihydropyridin-1- yl]butyl}-1H-1,2,3-triazole-4- carboxamide |
| 36 | 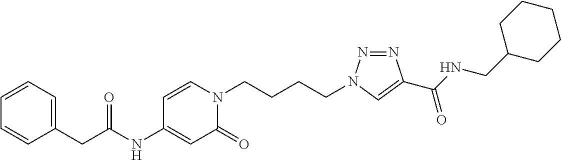 [SMILES: O=C(Cc1ccccc1)Nc1ccn(CCCCn2cc(C(=O)NCC3CCCCC3)nn2)c(=O)c1] | N-(cyclohexylmethyl)-1-{4-[2-oxo-4- (2-phenylacetamido)-1,2- dihydropyridin-1-yl]butyl}-1H-1,2,3- triazole-4-carboxamide |
| 37 | 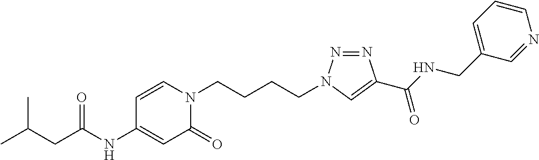 [SMILES: CC(C)CC(=O)Nc1ccn(CCCCn2cc(C(=O)NCc3cccnc3)nn2)c(=O)c1] | 1-{4-[4-(3-methylbutanamido)-2-oxo- 1,2-dihydropyridin-1-yl]butyl}-N- (pyridin-3-ylmethyl)-1H-1,2,3-triazole- 4-carboxamide |
| 38 | 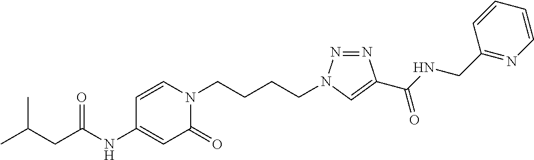 [SMILES: CC(C)CC(=O)Nc1ccn(CCCCn2cc(C(=O)NCc3ccccn3)nn2)c(=O)c1] | 1-{4-[4-(3-methylbutanamido)-2-oxo- 1,2-dihydropyridin-1-yl]butyl}-N- (pyridin-2-ylmethyl)-1H-1,2,3-triazole- 4-carboxamide |
| 39 | 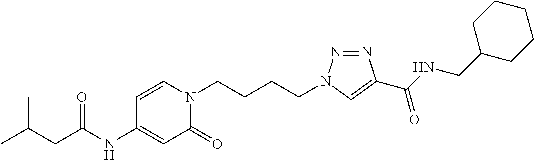 [SMILES: CC(C)CC(=O)Nc1ccn(CCCCn2cc(C(=O)NCC3CCCCC3)nn2)c(=O)c1] | N-(cyclohexylmethyl)-1-{4-[4-(3- methylbutanamido)-2-oxo-1,2- dihydropyridin-1-yl]butyl}-1H-1,2,3- triazole-4-carboxamide |
| 40 | 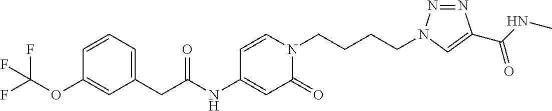 [SMILES: CNC(=O)c1cn(CCCCn2ccc(NC(=O)Cc3cccc(OC(F)(F)F)c3)cc2=O)nn1] | N-methyl-1-[4-(2-oxo-4-{2-[3- (trifluoromethoxy)phenyl]acetamido}- 1,2-dihydropyridin-1-yl)butyl]-1H- 1,2,3-triazole-4-carboxamide |
| 41 | 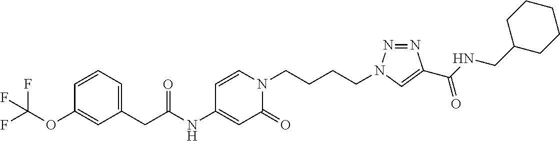 [SMILES: O=C(Cc1cccc(OC(F)(F)F)c1)Nc1ccn(CCCCn2cc(C(=O)NCC3CCCCC3)nn2)c(=O)c1] | N-(cyclohexylmethyl)-1-[4-(2-oxo-4- {2-[3-(trifluoro- methoxy)phenyl]acetamido}- 1,2-dihydropyridin-1-yl)butyl]-1H- 1,2,3-triazole-4-carboxamide |
| 42 | 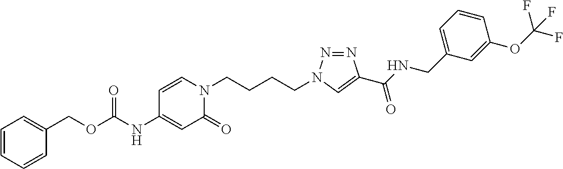 [SMILES: O=C(Nc1ccn(CCCCn2cc(C(=O)NCc3cccc(OC(F)(F)F)c3)nn2)c(=O)c1)OCc1ccccc1] | benzyl N-(2-oxo-1-{4-[4-({[3- (trifluoromethoxy)phenyl]methyl} carbamoyl)-1H-1,2,3-triazol-1-yl]butyl}- 1,2-dihydropyridin-4-yl)carbamate |
| 43 | 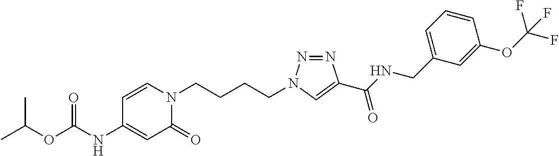 [SMILES: CC(C)OC(=O)Nc1ccn(CCCCn2cc(C(=O)NCc3cccc(OC(F)(F)F)c3)nn2)c(=O)c1] | propan-2-yl N-(2-oxo-1-{4-[4-({[3- (trifluoromethoxy)phenyl]methyl} carbamoyl)-1H-1,2,3-triazol-1- yl]butyl}-1,2- dihydropyridin-4-yl)carbamate |
| 44 | 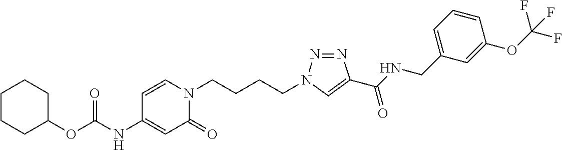 [SMILES: O=C(Nc1ccn(CCCCn2cc(C(=O)NCc3cccc(OC(F)(F)F)c3)nn2)c(=O)c1)OC1CCCCC1] | cyclohexyl N-(2-oxo-1-{4-[4-({[3- (trifluoromethoxy)phenyl]methyl} carbamoyl)-1H-1,2,3-triazol-1- yl]butyl}-1,2- dihydropyridin-4-yl)carbamate |
| 45 | 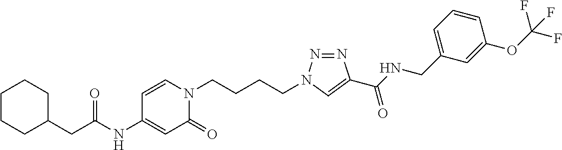 [SMILES: O=C(CC1CCCCC1)Nc1ccn(CCCCn2cc(C(=O)NCc3cccc(OC(F)(F)F)c3)nn2)c(=O)c1] | 1-{4-[4-(2-cyclohexylacetamido)-2- oxo-1,2-dihydropyridin-1-yl]butyl}-N- {[3-(trifluoromethoxy)phenyl]methyl}- 1H-1,2,3-triazole-4-carboxamide |
| 46 | 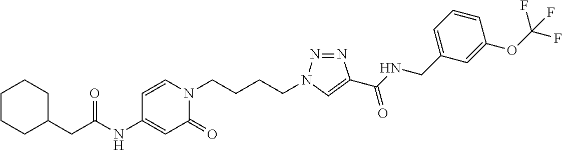 [SMILES: O=C(CC1CCCCC1)Nc1ccn(CCCCn2cc(C(=O)NCc3cccc(OC(F)(F)F)c3)nn2)c(=O)c1] | 1-(4-{4-[2-(1-methyl-1H-pyrazol-4- yl)acetamido]-2-oxo-1,2- dihydropyridin-1-yl}butyl)-N-{[3- (trifluoromethoxy)phenyl]methyl}-1H- 1,2,3-triazole-4-carboxamide |
| 47 | 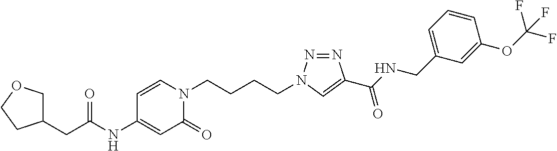 [SMILES: O=C(CC1CCOC1)Nc1ccn(CCCCn2cc(C(=O)NCc3cccc(OC(F)(F)F)c3)nn2)c(=O)c1] | 1-(4-{2-oxo-4-[2-(oxolan-3- yl)acetamido]-1,2-dihydropyridin-1- yl}butyl)-N-{[3- (trifluoromethoxy)phenyl]methyl}-1H- 1,2,3-triazole-4-carboxamide |
| 48 | 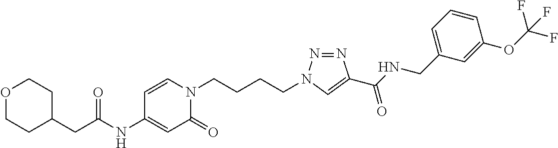 [SMILES: O=C(CC1CCOCC1)Nc1ccn(CCCCn2cc(C(=O)NCc3cccc(OC(F)(F)F)c3)nn2)c(=O)c1] | 1-(4-{4-[2-(oxan-4-yl)acetamido]-2- oxo-1,2-dihydropyridin-1-yl}butyl)-N- {[3-(trifluoromethoxy)phenyl]methyl}- 1H-1,2,3-triazole-4-carboxamide |
| 49 | 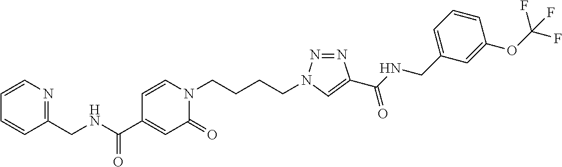 [SMILES: O=C(NCc1ccccn1)c1ccn(CCCCn2cc(C(=O)NCc3cccc(OC(F)(F)F)c3)nn2)c(=O)c1] | 2-oxo-N-(pyridin-2-ylmethyl)-1-{4- [4-({[3-(trifluoro- methoxy)phenyl]methyl}carbamoyl)- 1H-1,2,3-triazol-1-yl]butyl}-1,2- dihydropyridine-4-carboxamide |
| 50 | 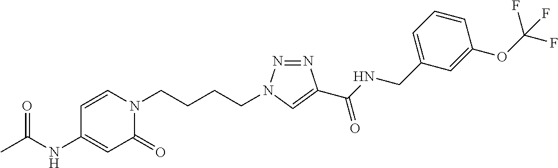 [SMILES: CC(=O)Nc1ccn(CCCCn2cc(C(=O)NCc3cccc(OC(F)(F)F)c3)nn2)c(=O)c1] | 1-[4-(4-acetamido-2-oxo-1,2- dihydropyridin-1-yl)butyl]-N-{[3- (trifluoromethoxy)phenyl]methyl}-1H- 1,2,3-triazole-4-carboxamide |
| 51 | 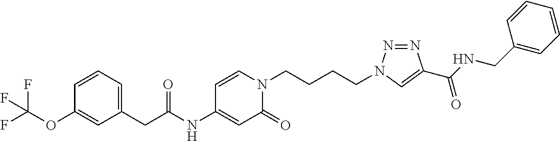 [SMILES: O=C(Cc1cccc(OC(F)(F)F)c1)Nc1ccn(CCCCn2cc(C(=O)NCc3ccccc3)nn2)c(=O)c1] | N-benzyl-1-[4-(2-oxo-4-{2-[3- (trifluoromethoxy)phenyl]acetamido}- 1,2-dihydropyridin-1-yl)butyl]-1H- 1,2,3-triazole-4-carboxamide |
| 52 | 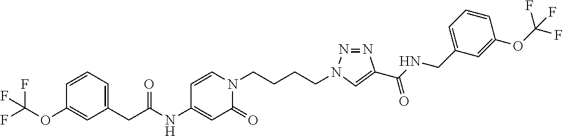 [SMILES: O=C(Cc1cccc(OC(F)(F)F)c1)Nc1ccn(CCCCn2cc(C(=O)NCc3cccc(OC(F)(F)F)c3)nn2)c(=O)c1] | 1-[4-(2-oxo-4-{2-[3- (trifluoromethoxy)phenyl]acetamido}- 1,2-dihydropyridin-1-yl)butyl]-N-{[3- (trifluoromethoxy)phenyl]methyl}-1H- 1,2,3-triazole-4-carboxamide |
| 53 | 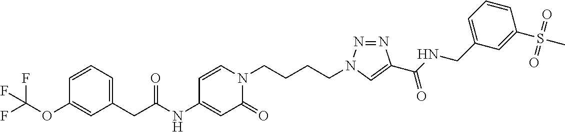 [SMILES: CS(=O)(=O)c1cccc(CNC(=O)c2cn(CCCCn3ccc(NC(=O)Cc4cccc(OC(F)(F)F)c4)cc3=O)nn2)c1] | N-[(3-methanesulfonylphenyl)methyl]- 1-[4-(2-oxo-4-{2-[3- (trifluoromethoxy)phenyl]acetamido}- 1,2-dihydropyridin-1-yl)butyl]-1H- 1,2,3-triazole-4-carboxamide |
| 54 | 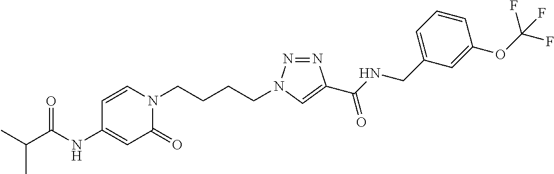 [SMILES: CC(C)C(=O)Nc1ccn(CCCCn2cc(C(=O)NCc3cccc(OC(F)(F)F)c3)nn2)c(=O)c1] | 1-{4-[4-(2-methylpropanamido)-2-oxo- 1,2-dihydropyridin-1-yl]butyl}-N-{[3- (trifluoromethoxy)phenyl]methyl}-1H- 1,2,3-triazole-4-carboxamide |
| 55 | 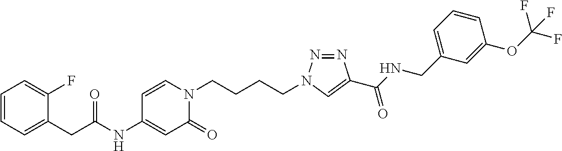 [SMILES: O=C(Cc1ccccc1F)Nc1ccn(CCCCn2cc(C(=O)NCc3cccc(OC(F)(F)F)c3)nn2)c(=O)c1] | 1-(4-{4-[2-(2-fluorophenyl)acetamido]- 2-oxo-1,2-dihydropyridin-1-yl}butyl)- N-{[3- (trifluoromethoxy)phenyl]methyl}-1H- 1,2,3-triazole-4-carboxamide |
| 56 | 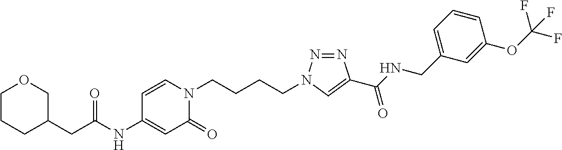 [SMILES: O=C(CC1CCCOC1)Nc1ccn(CCCCn2cc(C(=O)NCc3cccc(OC(F)(F)F)c3)nn2)c(=O)c1] | 1-(4-{4-[2-(oxan-3-yl)acetamido]-2- oxo-1,2-dihydropyridin-1-yl}butyl)-N- {[3-(trifluoromethoxy)phenyl]methyl}- 1H-1,2,3-triazole-4-carboxamide |
| 57 | 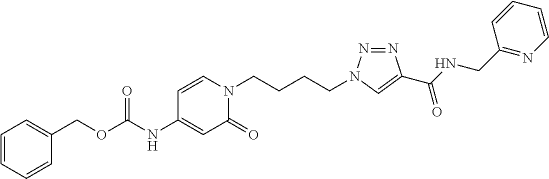 [SMILES: O=C(Nc1ccn(CCCCn2cc(C(=O)NCc3ccccn3)nn2)c(=O)c1)OCc1ccccc1] | benzyl N-[2-oxo-1-(4-{4-[(pyridin-2- ylmethyl)carbamoyl]-1H-1,2,3-triazol- 1-yl}butyl)-1,2-dihydropyridin-4- yl]carbamate |
| 58 | 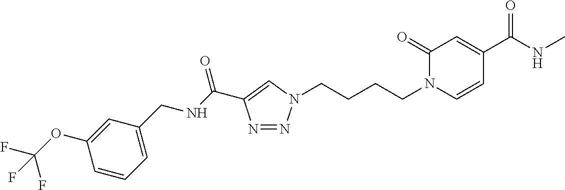 [SMILES: CNC(=O)c1ccn(CCCCn2cc(C(=O)NCc3cccc(OC(F)(F)F)c3)nn2)c(=O)c1] | N-methyl-2-oxo-1-{4-[4-({[3- (trifluoromethoxy)phenyl]methyl} carbamoyl)-1H-1,2,3-triazol- 1-yl]butyl}-1,2- dihydropyridine-4-carboxamide |
| 59 | 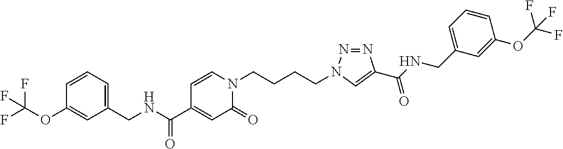 [SMILES: O=C(NCc1cccc(OC(F)(F)F)c1)c1ccn(CCCCn2cc(C(=O)NCc3cccc(OC(F)(F)F)c3)nn2)c(=O)c1] | 2-oxo-N-{[3- (trifluoromethoxy)phenyl]methyl}-1- {4-[4-({[3-(trifluoro- methoxy)phenyl]methyl}carbamoyl)- 1H-1,2,3-triazol-1-yl]butyl}-1,2- dihydropyridine-4-carboxamide |
| 60 | 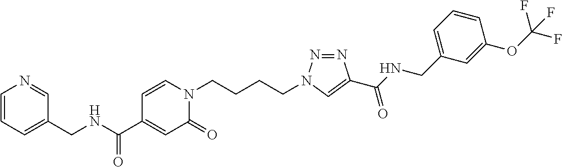 [SMILES: O=C(NCc1cccnc1)c1ccn(CCCCn2cc(C(=O)NCc3cccc(OC(F)(F)F)c3)nn2)c(=O)c1] | 2-oxo-N-(pyridin-3-ylmethyl)-1-{4-[4- ({[3-(trifluoro- methoxy)phenyl]methyl}carbamoyl)- 1H-1,2,3-triazol-1-yl]butyl}-1,2- dihydropyridine-4-carboxamide |
| 61 | 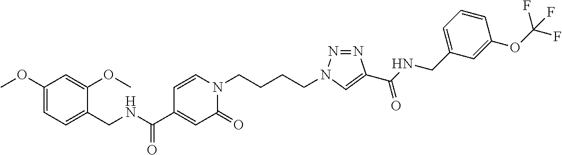 [SMILES: COc1ccc(CNC(=O)c2ccn(CCCCn3cc(C(=O)NCc4cccc(OC(F)(F)F)c4)nn3)c(=O)c2)c(OC)c1] | N-[(2,4-dimethoxyphenyl)methyl]-2- oxo-1-{4-[4-({[3-(trifluoro- methoxy)phenyl]methyl}carbamoyl)- 1H-1,2,3-triazol-1-yl]butyl}-1,2- dihydropyridine-4-carboxamide |
| 62 | 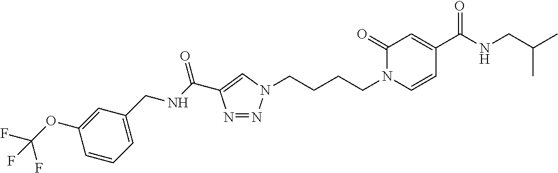 [SMILES: CC(C)CNC(=O)c1ccn(CCCCn2cc(C(=O)NCc3cccc(OC(F)(F)F)c3)nn2)c(=O)c1] | N-(2-methylpropyl)-2-oxo-1-{4-[4-({[3- (trifluoromethoxy)phenyl]methyl} carbamoyl)-1H-1,2,3-triazol- 1-yl]butyl}-1,2- dihydropyridine-4-carboxamide |
| 63 | 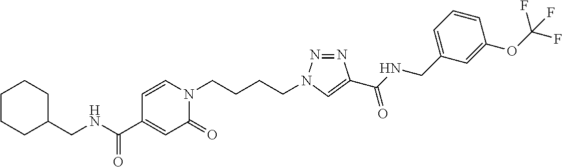 [SMILES: O=C(NCC1CCCCC1)c1ccn(CCCCn2cc(C(=O)NCc3cccc(OC(F)(F)F)c3)nn2)c(=O)c1] | N-(cyclohexylmethyl)-2-oxo-1-{4-[4- ({[3-(trifluoro- methoxy)phenyl]methyl}carbamoyl)- 1H-1,2,3-triazol-1-yl]butyl}-1,2- dihydropyridine-4-carboxamide |
| 64 | 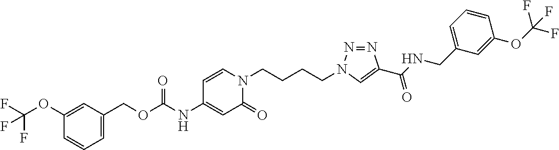 [SMILES: O=C(Nc1ccn(CCCCn2cc(C(=O)NCc3cccc(OC(F)(F)F)c3)nn2)c(=O)c1)OCc1cccc(OC(F)(F)F)c1] | [3-(trifluoromethoxy)phenyl]methyl N- (2-oxo-1-{4-[4-({[3-(trifluoro- methoxy)phenyl]methyl}carbamoyl)- 1H-1,2,3-triazol-1-yl]butyl}-1,2- dihydropyridin-4-yl)carbamate |
| 65 | 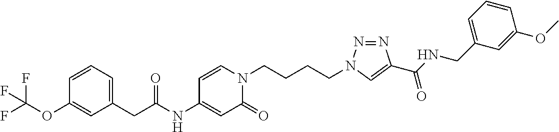 [SMILES: COc1cccc(CNC(=O)c2cn(CCCCn3ccc(NC(=O)Cc4cccc(OC(F)(F)F)c4)cc3=O)nn2)c1] | N-[(3-methoxyphenyl)methyl]-1-[4-(2- oxo-4-{2-[3- (trifluoromethoxy)phenyl]acetamido}- 1,2-dihydropyridin-1-yl)butyl]-1H- 1,2,3-triazole-4-carboxamide |
| 66 | 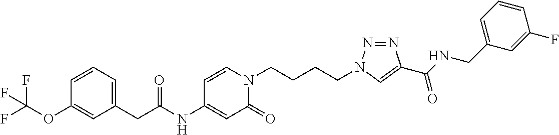 [SMILES: O=C(Cc1cccc(OC(F)(F)F)c1)Nc1ccn(CCCCn2cc(C(=O)NCc3cccc(F)c3)nn2)c(=O)c1] | N-[(3-fluorophenyl)methyl]-1-[4-(2- oxo-4-{2-[3- (trifluoromethoxy)phenyl]acetamido}- 1,2-dihydropyridin-1-yl)butyl]-1H- 1,2,3-triazole-4-carboxamide |
| 67 | 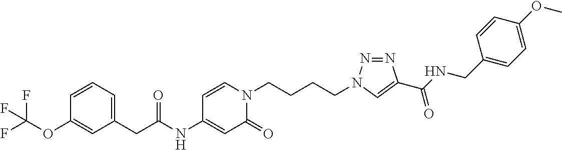 [SMILES: COc1ccc(CNC(=O)c2cn(CCCCn3ccc(NC(=O)Cc4cccc(OC(F)(F)F)c4)cc3=O)nn2)cc1] | N-[(4-methoxyphenyl)methyl]-1-[4-(2- oxo-4-{2-[3- (trifluoromethoxy)phenyl]acetamido}- 1,2-dihydropyridin-1-yl)butyl]-1H- 1,2,3-triazole-4-carboxamide |
| 68 | 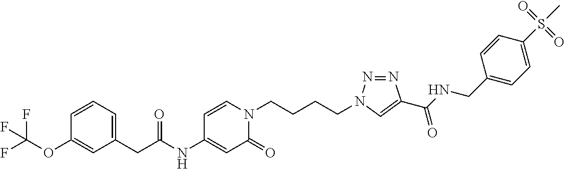 [SMILES: CS(=O)(=O)c1ccc(CNC(=O)c2cn(CCCCn3ccc(NC(=O)Cc4cccc(OC(F)(F)F)c4)cc3=O)nn2)cc1] | N-[(4-methanesulfonylphenyl)methyl]- 1-[4-(2-oxo-4-{2-[3- (trifluoromethoxy)phenyl]acetamido}- 1,2-dihydropyridin-1-yl)butyl]-1H- 1,2,3-triazole-4-carboxamide |
| 69 | 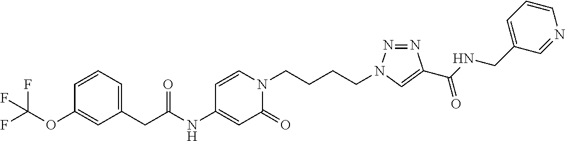 [SMILES: O=C(Cc1cccc(OC(F)(F)F)c1)Nc1ccn(CCCCn2cc(C(=O)NCc3cccnc3)nn2)c(=O)c1] | 1-[4-(2-oxo-4-{2-[3- (trifluoromethoxy)phenyl]acetamido}- 1,2-dihydropyridin-1-yl)butyl]-N- (pyridin-3-ylmethyl)-1H-1,2,3-triazole- 4-carboxamide |
| 70 | 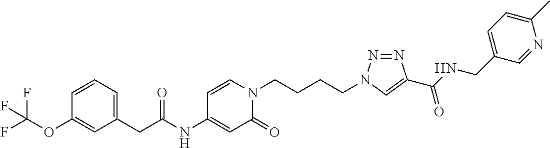 [SMILES: Cc1ccc(CNC(=O)c2cn(CCCCn3ccc(NC(=O)Cc4cccc(OC(F)(F)F)c4)cc3=O)nn2)cn1] | N-[(6-methylpyridin-3-yl)methyl]-1-[4- (2-oxo-4-{2-[3- (trifluoromethoxy)phenyl]acetamido}- 1,2-dihydropyridin-1-yl)butyl]-1H- 1,2,3-triazole-4-carboxamide |
| 71 | 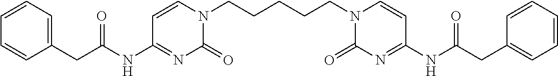 [SMILES: O=C(Cc1ccccc1)Nc1ccn(CCCCCn2ccc(NC(=O)Cc3ccccc3)nc2=O)c(=O)n1] | N-(2-oxo-1-{5-[2-oxo-4-(2-phenyl- acetamido)-1,2-dihydropyrimidin- 1-yl]pentyl}-1,2-dihydropyrimidin- 4-yl)-2-phenylacetamide |
| 72 | 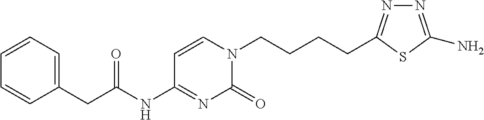 [SMILES: Nc1nnc(CCCCn2ccc(NC(=O)Cc3ccccc3)nc2=O)s1] | N-{1-[4-(5-amino-1,3,4-thiadiazol-2- yl)butyl]-2-oxo-1,2-dihydropyrimidin- 4-yl}-2-phenylacetamide |
| 73 | 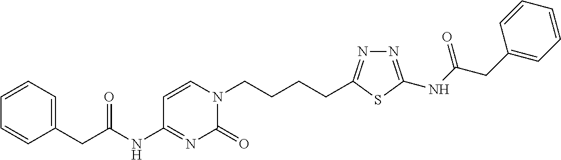 [SMILES: O=C(Cc1ccccc1)Nc1ccn(CCCCc2nnc(NC(=O)Cc3ccccc3)s2)c(=O)n1] | N-(2-oxo-1-{4-[5-(2-phenylacetamido)- 1,3,4-thiadiazol-2-yl]butyl}-1,2- dihydropyrimidin-4-yl)-2- phenylacetamide |
| 74 | 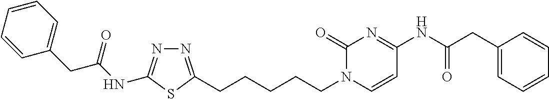 [SMILES: O=C(Cc1ccccc1)Nc1ccn(CCCCCc2nnc(NC(=O)Cc3ccccc3)s2)c(=O)n1] | N-(2-oxo-1-{5-[5-(2-phenylacetamido)- 1,3,4-thiadiazol-2-yl]pentyl}-1,2- dihydropyrimidin-4-yl)-2- phenylacetamide |
| 75 | 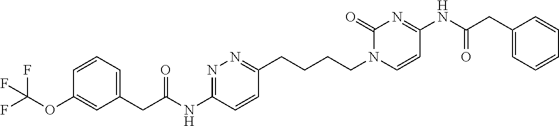 [SMILES: O=C(Cc1cccc(OC(F)(F)F)c1)Nc1ccc(CCCCn2ccc(NC(=O)Cc3ccccc3)nc2=O)nn1] | N-{2-oxo-1-[4-(6-{2-[3-(trifluoro- methoxy)phenyl]acetamido}pyridazin- 3-yl)butyl]-1,2- dihydropyrimidin-4-yl}-2- phenylacetamide |
| 76 | 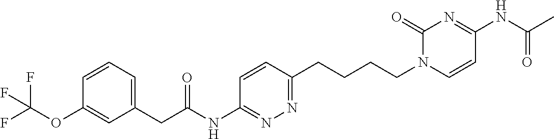 [SMILES: CC(=O)Nc1ccn(CCCCc2ccc(NC(=O)Cc3cccc(OC(F)(F)F)c3)nn2)c(=O)n1] | N-{6-[4-(4-acetamido-2-oxo-1,2- dihydropyrimidin-1-yl)butyl]pyridazin- 3-yl}-2-[3- (trifluoromethoxy)phenyl]acetamide |
| 77 | 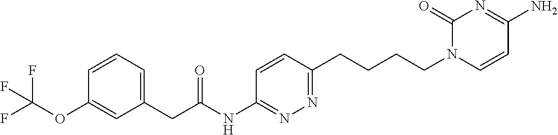 [SMILES: Nc1ccn(CCCCc2ccc(NC(=O)Cc3cccc(OC(F)(F)F)c3)nn2)c(=O)n1] | N-{6-[4-(4-amino-2-oxo-1,2- dihydropyrimidin-1-yl)butyl]pyridazin- 3-yl}-2-[3- (trifluoromethoxy)phenyl]acetamide |
| 78 | 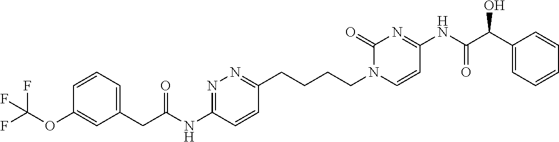 [SMILES: O=C(Cc1cccc(OC(F)(F)F)c1)Nc1ccc(CCCCn2ccc(NC(=O)[C@@H](O)c3ccccc3)nc2=O)nn1] | (2S)-2-hydroxy-N-{2-oxo-1-[4-(6-{2- [3-(trifluoro- methoxy)phenyl]acetamido} pyridazin-3-yl)butyl]-1,2- dihydropyrimidin-4-yl}-2- phenylacetamide |
| 79 | 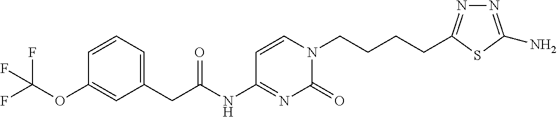 [SMILES: Nc1nnc(CCCCn2ccc(NC(=O)Cc3cccc(OC(F)(F)F)c3)nc2=O)s1] | N-{1-[4-(5-amino-1,3,4-thiadiazol-2- yl)butyl]-2-oxo-1,2-dihydropyrimidin- 4-yl}-2-[3- (trifluoromethoxy)phenyl]acetamide |
| 80 | 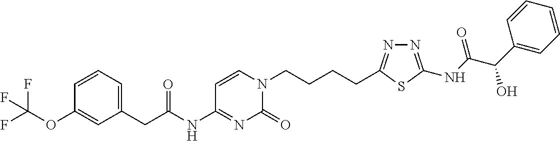 [SMILES: O=C(Cc1cccc(OC(F)(F)F)c1)Nc1ccn(CCCCc2nnc(NC(=O)[C@@H](O)c3ccccc3)s2)c(=O)n1] | (2S)-2-hydroxy-N-{5-[4-(2-oxo-4-{2- [3-(trifluoro- methoxy)phenyl]acetamido}-1,2- dihydropyrimidin-1-yl)butyl]-1,3,4- thiadiazol-2-yl}-2-phenylacetamide |
| 81 | 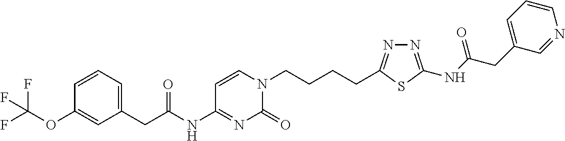 [SMILES: O=C(Cc1cccc(OC(F)(F)F)c1)Nc1ccn(CCCCc2nnc(NC(=O)Cc3cccnc3)s2)c(=O)n1] | N-[2-oxo-1-(4-{5-[2-(pyridin-3- yl)acetamido]-1,3,4-thiadiazol-2- yl]butyl)-1,2-dihydropyrimidin-4-yl]-2- [3-(trifluoromethoxy)phenyl]acetamide |
| 82 | 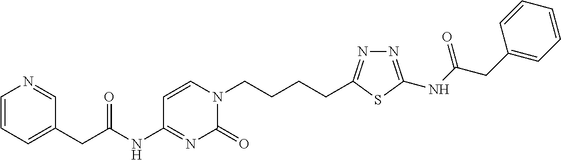 [SMILES: O=C(Cc1cccnc1)Nc1ccn(CCCCc2nnc(NC(=O)Cc3ccccc3)s2)c(=O)n1] | N-[5-(4-{2-oxo-4-[2-(pyridin-3- yl)acetamido]-1,2-dihydropyrimidin-1- yl}butyl)-1,3,4-thiadiazol-2-yl]-2- phenylacetamide |
| 83 | 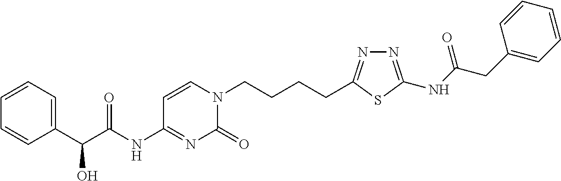 [SMILES: O=C(Cc1ccccc1)Nc1nnc(CCCCn2ccc(NC(=O)[C@@H](O)c3ccccc3)nc2=O)s1] | (2S)-2-hydroxy-N-(2-oxo-1-{4-[5-(2- phenylacetamido)-1,3,4-thiadiazol-2- yl]butyl}-1,2-dihydropyrimidin-4-yl)- 2-phenylacetamide |
| 84 | 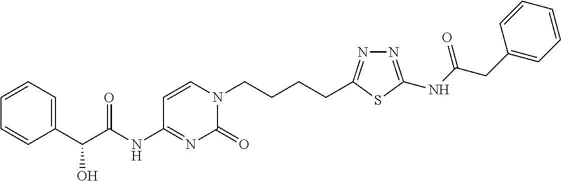 [SMILES: O=C(Cc1ccccc1)Nc1nnc(CCCCn2ccc(NC(=O)[C@H](O)c3ccccc3)nc2=O)s1] | (2R)-2-hydroxy-N-(2-oxo-1-{4-[5-(2- phenylacetamido)-1,3,4-thiadiazol-2- yl]butyl}-1,2-dihydropyrimidin-4-yl)-2- phenylacetamide |
| 85 | 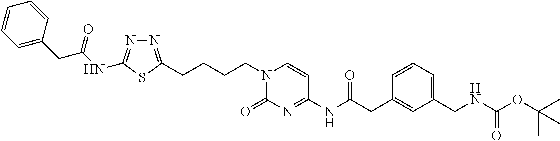 [SMILES: CC(C)(C)OC(=O)NCc1cccc(CC(=O)Nc2ccn(CCCCc3nnc(NC(=O)Cc4ccccc4)s3)c(=O)n2)c1] | tert-butyl N-[(3-{[(2-oxo-1-{4-[5-(2- phenylacetamido)-1,3,4-thiadiazol-2- yl]butyl}-1,2-dihydropyrimidin-4- yl)carbamoyl]methyl}phenyl)methyl] carbamate |
| 86 | 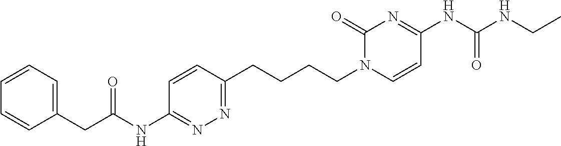 [SMILES: CCNC(=O)Nc1ccn(CCCCc2ccc(NC(=O)Cc3ccccc3)nn2)c(=O)n1] | N-[6-(4-{4-[(ethylcarbamoyl)amino]- 2-oxo-1,2-dihydropyrimidin-1- yl}butyl)pyridazin-3-yl]-2- phenylacetamide |
| 87 | 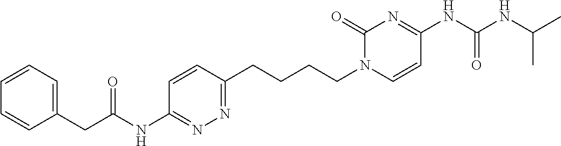 [SMILES: CC(C)NC(=O)Nc1ccn(CCCCc2ccc(NC(=O)Cc3ccccc3)nn2)c(=O)n1] | N-{6-[4-(2-oxo-4-{[(propan-2- yl)carbamoyl]amino}-1,2- dihydropyrimidin-1-yl)butyl]pyridazin- 3-yl}-2-phenylacetamide |
| 88 | 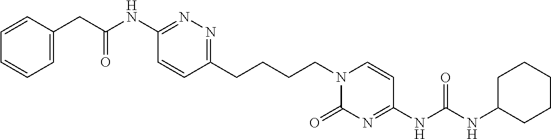 [SMILES: O=C(Cc1ccccc1)Nc1ccc(CCCCn2ccc(NC(=O)NC3CCCCC3)nc2=O)nn1] | N-[6-(4-{4- [(cyclohexylcarbamoyl)amino]-2-oxo- 1,2-dihydropyrimidin-1- yl}butyl)pyridazin-3-yl]-2- phenylacetamide |
| 89 | 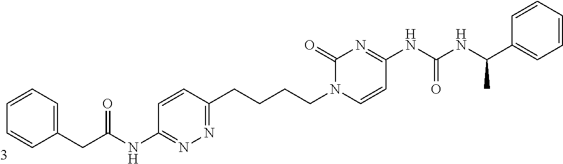 [SMILES: C[C@@H](NC(=O)Nc1ccn(CCCCc2ccc(NC(=O)Cc3ccccc3)nn2)c(=O)n1)c1ccccc1] | N-(6-{4-[2-oxo-4-({[(1R)-1- phenylethyl]carbamoyl}amino)-1,2- dihydropyrimidin-1-yl]butyl}pyridazin- 3-yl)-2-phenylacetamide |
| 90 | 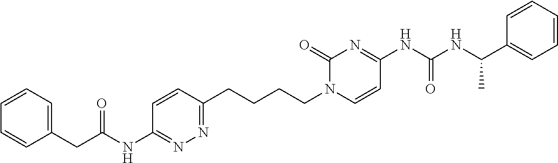 [SMILES: C[C@H](NC(=O)Nc1ccn(CCCCc2ccc(NC(=O)Cc3ccccc3)nn2)c(=O)n1)c1ccccc1] | N-(6-{4-[2-oxo-4-({[(1S)-1- phenylethyl]carbamoyl}amino)-1,2- dihydropyrimidin-1-yl]butyl}pyridazin- 3-yl)-2-phenylacetamide |
| 91 | 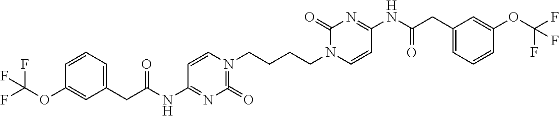 | N-{2-oxo-1-[4-(2-oxo-4-{2-[3- (trifluoromethoxy)phenyl]acetamido}- 1,2-dihydropyrimidin-1-yl)butyl]-1,2- dihydropyrimidin-4-yl}-2-[3- (trifluoromethoxy)phenyl]acetamide |
| 92 | 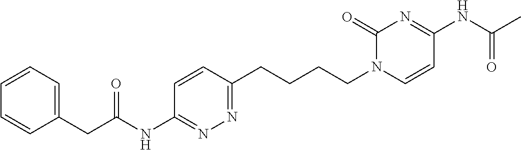 [SMILES: CC(=O)Nc1ccn(CCCCc2ccc(NC(=O)Cc3ccccc3)nn2)c(=O)n1] | N-{6-[4-(4-acetamido-2-oxo-1,2- dihydropyrimidin-1-yl)butyl]pyridazin- 3-yl}-2-phenylacetamide |
| 93 | 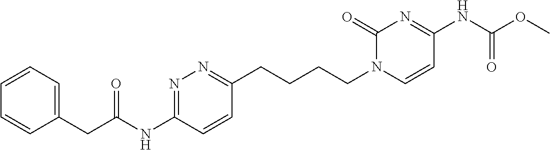 [SMILES: COC(=O)Nc1ccn(CCCCc2ccc(NC(=O)Cc3ccccc3)nn2)c(=O)n1] | methyl N-(2-oxo-1-{4-[6-(2- phenylacetamido)pyridazin-3-yl]butyl}- 1,2-dihydropyrimidin-4-yl)carbamate |
| 94 | 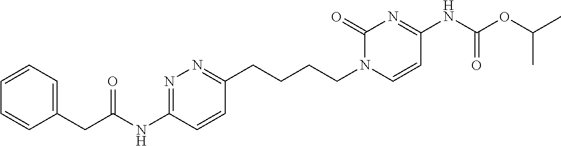 [SMILES: CC(C)OC(=O)Nc1ccn(CCCCc2ccc(NC(=O)Cc3ccccc3)nn2)c(=O)n1] | propan-2-yl N-(2-oxo-1-{4-[6-(2- phenylacetamido)pyridazin-3-yl]butyl}- 1,2-dihydropyrimidin-4-yl)carbamate |
| 95 | 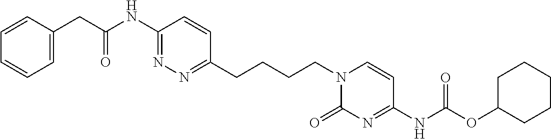 [SMILES: O=C(Cc1ccccc1)Nc1ccc(CCCCn2ccc(NC(=O)OC3CCCCC3)nc2=O)nn1] | cyclohexyl N-(2-oxo-1-{4-[6-(2- phenylacetamido)pyridazin-3-yl]butyl}- 1,2-dihydropyrimidin-4-yl)carbamate |
| 96 | 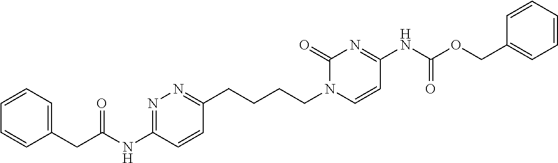 [SMILES: O=C(Cc1ccccc1)Nc1ccc(CCCCn2ccc(NC(=O)OCc3ccccc3)nc2=O)nn1] | benzyl N-(2-oxo-1-{4-[6-(2- phenylacetamido)pyridazin-3-yl]butyl}- 1,2-dihydropyrimidin-4-yl)carbamate |
| 97 | 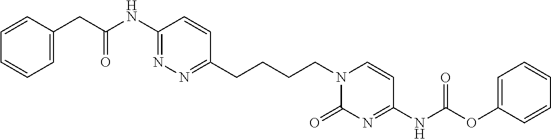 [SMILES: O=C(Cc1ccccc1)Nc1ccc(CCCCn2ccc(NC(=O)Oc3ccccc3)nc2=O)nn1] | phenyl N-(2-oxo-1-{4-[6-(2- phenylacetamido)pyridazin-3-yl]butyl}- 1,2-dihydropyrimidin-4-yl)carbamate |
| 98 | 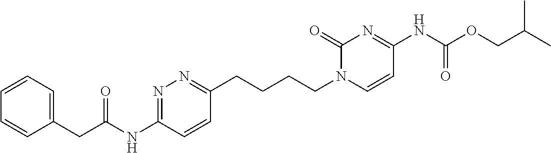 [SMILES: CC(C)COC(=O)Nc1ccn(CCCCc2ccc(NC(=O)Cc3ccccc3)nn2)c(=O)n1] | 2-methylpropyl N-(2-oxo-1-{4-[6-(2- phenylacetamido)pyridazin-3-yl]butyl}- 1,2-dihydropyrimidin-4-yl)carbamate |
| 99 | 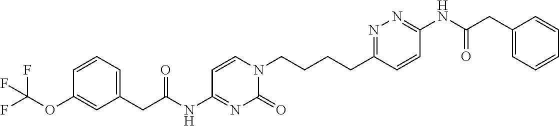 [SMILES: O=C(Cc1ccccc1)Nc1ccc(CCCCn2ccc(NC(=O)Cc3cccc(OC(F)(F)F)c3)nc2=O)nn1] | N-{6-[4-(2-oxo-4-{2-[3- (trifluoromethoxy)phenyl]acetamido}- 1,2-dihydropyrimidin-1- yl)butyl]pyridazin-3-yl}-2- phenylacetamide |
| 100 | 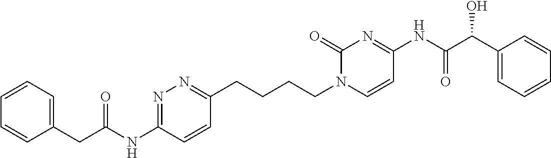 [SMILES: O=C(Cc1ccccc1)Nc1ccc(CCCCn2ccc(NC(=O)[C@H](O)c3ccccc3)nc2=O)nn1] | (2R)-2-hydroxy-N-(2-oxo-1-{4-[6-(2- phenylacetamido)pyridazin-3-yl]butyl}- 1,2-dihydropyrimidin-4-yl)-2- phenylacetamide |
| 101 | 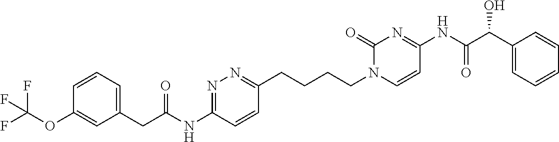 [SMILES: O=C(Cc1cccc(OC(F)(F)F)c1)Nc1ccc(CCCCn2ccc(NC(=O)[C@H](O)c3ccccc3)nc2=O)nn1] | (2R)-2-hydroxy-N-{2-oxo-1-[4-(6-{2- [3-(trifluoro- methoxy)phenyl]acetamido}pyridazin- 3-yl)butyl]-1,2- dihydropyrimidin-4-yl}-2- phenylacetamide |
| 102 | 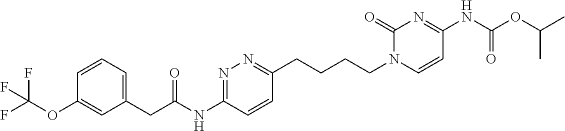 [SMILES: CC(C)OC(=O)Nc1ccn(CCCCc2ccc(NC(=O)Cc3cccc(OC(F)(F)F)c3)nn2)c(=O)n1] | propan-2-yl N-{2-oxo-1-[4-(6-{2-[3- (trifluoromethoxy)phenyl]acetamido} pyridazin-3-yl)butyl]-1,2- dihydropyrimidin-4-yl}carbamate |
| 103 | 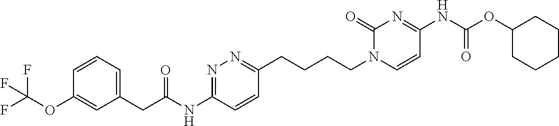 [SMILES: O=C(Cc1cccc(OC(F)(F)F)c1)Nc1ccc(CCCCn2ccc(NC(=O)OC3CCCCC3)nc2=O)nn1] | cyclohexyl N-{2-oxo-1-[4-(6-{2-[3- (trifluoromethoxy)phenyl]acetamido} pyridazin-3-yl)butyl]-1,2- dihydropyrimidin-4-yl}carbamate |
| 104 | 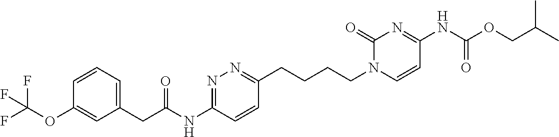 [SMILES: CC(C)COC(=O)Nc1ccn(CCCCc2ccc(NC(=O)Cc3cccc(OC(F)(F)F)c3)nn2)c(=O)n1] | 2-methylpropyl N-{2-oxo-1-[4-(6-{2- [3-(trifluoro- methoxy)phenyl]acetamido}pyridazin- 3-yl)butyl]-1,2- dihydropyrimidin-4-yl}carbamate |
| 105 | 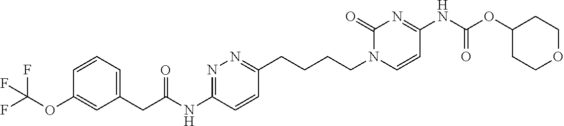 [SMILES: O=C(Cc1cccc(OC(F)(F)F)c1)Nc1ccc(CCCCn2ccc(NC(=O)OC3CCOCC3)nc2=O)nn1] | oxan-4-yl N-{2-oxo-1-[4-(6-{2-[3- (trifluoromethoxy)phenyl]acetamido} pyridazin-3-yl)butyl]-1,2- dihydropyrimidin-4-yl}carbamate |
| 106 | 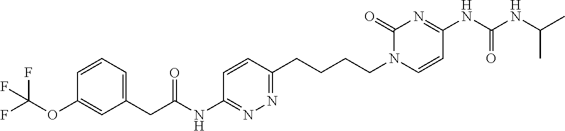 [SMILES: CC(C)NC(=O)Nc1ccn(CCCCc2ccc(NC(=O)Cc3cccc(OC(F)(F)F)c3)nn2)c(=O)n1] | N-{6-[4-(2-oxo-4-{[(propan-2- yl)carbamoyl]amino}-1,2- dihydropyrimidin-1-yl)butyl]pyridazin- 3-yl}-2-[3- (trifluoromethoxy)phenyl]acetamide |
| 107 | 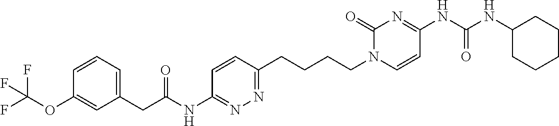 [SMILES: O=C(Cc1cccc(OC(F)(F)F)c1)Nc1ccc(CCCCn2ccc(NC(=O)NC3CCCCC3)nc2=O)nn1] | N-[6-(4-{4- [(cyclohexylcarbamoyl)amino]-2-oxo- 1,2-dihydropyrimidin-1- yl}butyl)pyridazin-3-yl]-2-[3- (trifluoromethoxy)phenyl]acetamide |
| 108 | 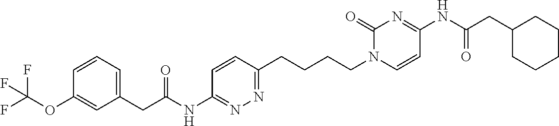 [SMILES: O=C(Cc1cccc(OC(F)(F)F)c1)Nc1ccc(CCCCn2ccc(NC(=O)CC3CCCCC3)nc2=O)nn1] | N-(6-{4-[4-(2-cyclohexylacetamido)-2- oxo-1,2-dihydropyrimidin-1- yl]butyl}pyridazin-3-yl)-2-[3- (trifluoromethoxy)phenyl]acetamide |
| 109 | 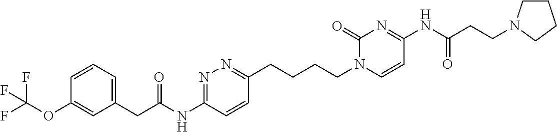 [SMILES: O=C(Cc1cccc(OC(F)(F)F)c1)Nc1ccc(CCCCn2ccc(NC(=O)CCN3CCCC3)nc2=O)nn1] | N-{2-oxo-1-[4-(6-{2-[3- (trifluoromethoxy)phenyl]acetamido} pyridazin-3-yl)butyl]-1,2- dihydropyrimidin-4-yl}-3-(pyrrolidin-1- yl)propanamide |
| 110 | 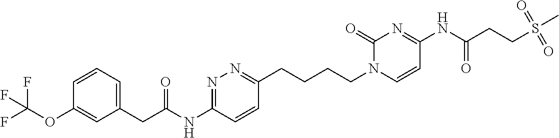 [SMILES: CS(=O)(=O)CCC(=O)Nc1ccn(CCCCc2ccc(NC(=O)Cc3cccc(OC(F)(F)F)c3)nn2)c(=O)n1] | 3-methanesulfonyl-N-{2-oxo-1-[4-(6- {2-[3-(trifluoro- methoxy)phenyl]acetamido}pyridazin- 3-yl)butyl]-1,2- dihydropyrimidin-4-yl}propanamide |
| 111 | 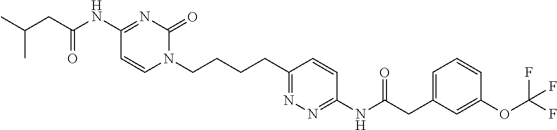 [SMILES: CC(C)CC(=O)Nc1ccn(CCCCc2ccc(NC(=O)Cc3cccc(OC(F)(F)F)c3)nn2)c(=O)n1] | 3-methyl-N-{2-oxo-1-[4-(6-{2-[3- (trifluoromethoxy)phenyl]acetamido} pyridazin-3-yl)butyl]-1,2- dihydropyrimidin-4-yl}butanamide |
| 112 | 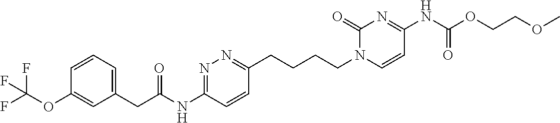 [SMILES: COCCOC(=O)Nc1ccn(CCCCc2ccc(NC(=O)Cc3cccc(OC(F)(F)F)c3)nn2)c(=O)n1] | 2-methoxyethyl N-{2-oxo-1-[4-(6-{2- [3-(trifluoro- methoxy)phenyl]acetamido}pyridazin- 3-yl)butyl]-1,2- dihydropyrimidin-4-yl}carbamate |
| 113 | 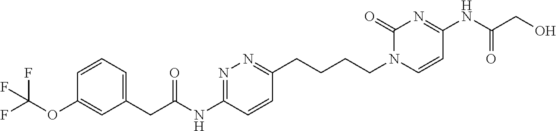 [SMILES: O=C(Cc1cccc(OC(F)(F)F)c1)Nc1ccc(CCCCn2ccc(NC(=O)CO)nc2=O)nn1] | 2-hydroxy-N-{2-oxo-1-[4-(6-{2-[3- (trifluoromethoxy)phenyl]acetamido} pyridazin-3-yl)butyl]-1,2- dihydropyrimidin-4-yl}acetamide |
| 114 | 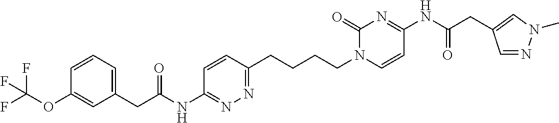 [SMILES: Cn1cc(CC(=O)Nc2ccn(CCCCc3ccc(NC(=O)Cc4cccc(OC(F)(F)F)c4)nn3)c(=O)n2)cn1] | N-[6-(4-{4-[2-(1-methyl-1H-pyrazol-4- yl)acetamido]-2-oxo-1,2- dihydropyrimidin-1-yl}butyl)pyridazin- 3-yl]-2-[3- (trifluoromethoxy)phenyl]acetamide |
| 115 | 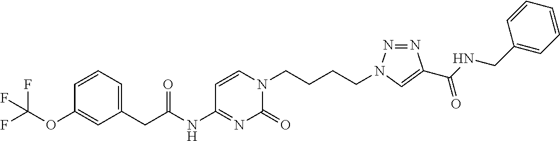 [SMILES: O=C(Cc1cccc(OC(F)(F)F)c1)Nc1ccn(CCCCn2cc(C(=O)NCc3ccccc3)nn2)c(=O)n1] | N-benzyl-1-[4-(2-oxo-4-{2-[3- (trifluoromethoxy)phenyl]acetamido}- 1,2-dihydropyrimidin-1-yl)butyl]-1H- 1,2,3-triazole-4-carboxamide |
| 116 | 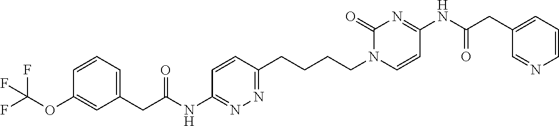 [SMILES: O=C(Cc1cccc(OC(F)(F)F)c1)Nc1ccc(CCCCn2ccc(NC(=O)Cc3cccnc3)nc2=O)nn1] | N-[6-(4-{2-oxo-4-[2-(pyridin-3- yl)acetamido]-1,2-dihydropyrimidin-1- yl}butyl)pyridazin-3-yl]-2-[3- (trifluoromethoxy)phenyl]acetamide |
| 117 | 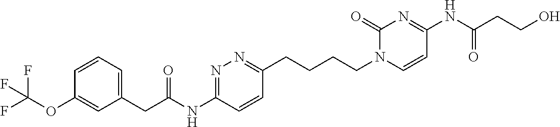 [SMILES: O=C(Cc1cccc(OC(F)(F)F)c1)Nc1ccc(CCCCn2ccc(NC(=O)CCO)nc2=O)nn1] | 3-hydroxy-N-{2-oxo-1-[4-(6-{2-[3- (trifluoromethoxy)phenyl]acetamido} pyridazin-3-yl)butyl]-1,2- dihydropyrimidin-4-yl}propanamide |
| 118 | 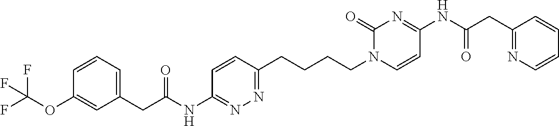 [SMILES: O=C(Cc1cccc(OC(F)(F)F)c1)Nc1ccc(CCCCn2ccc(NC(=O)Cc3ccccn3)nc2=O)nn1] | N-[6-(4-{2-oxo-4-[2-(pyridin-2- yl)acetamido]-1,2-dihydropyrimidin-1- yl}butyl)pyridazin-3-yl]-2-[3- (trifluoromethoxy)phenyl]acetamide |
| 119 | 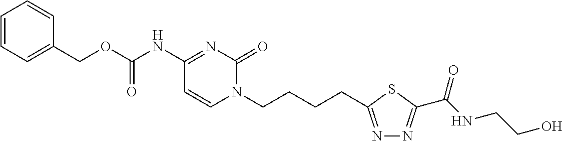 [SMILES: O=C(Nc1ccn(CCCCc2nnc(C(=O)NCCO)s2)c(=O)n1)OCc1ccccc1] | benzyl N-[1-(4-{5-[(2- hydroxyethyl)carbamoyl]-1,3,4- thiadiazol-2-yl}butyl)-2-oxo-1,2- dihydropyrimidin-4-yl]carbamate |
| 120 | 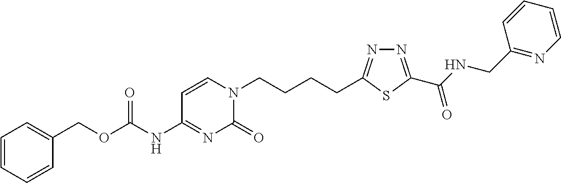 [SMILES: O=C(Nc1ccn(CCCCc2nnc(C(=O)NCc3ccccn3)s2)c(=O)n1)OCc1ccccc1] | benzyl N-[2-oxo-1-(4-{5-[(pyridin-2- ylmethyl)carbamoyl]-1,3,4-thiadiazol-2- yl}butyl)-1,2-dihydropyrimidin-4- yl]carbamate |
| 121 | 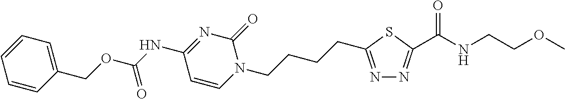 [SMILES: COCCNC(=O)c1nnc(CCCCn2ccc(NC(=O)OCc3ccccc3)nc2=O)s1] | benzyl N-[1-(4-{5-[(2- methoxyethyl)carbamoyl]-1,3,4- thiadiazol-2-yl}butyl)-2-oxo-1,2- dihydropyrimidin-4-yl]carbamate |
| 122 | 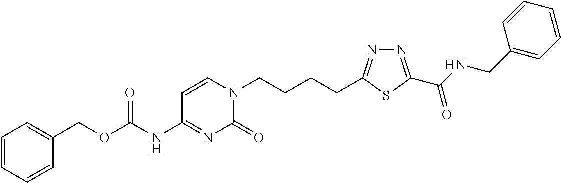 [SMILES: O=C(Nc1ccn(CCCCc2nnc(C(=O)NCc3ccccc3)s2)c(=O)n1)OCc1ccccc1] | benzyl N-(1-{4-[5-(benzylcarbamoyl)- 1,3,4-thiadiazol-2-yl]butyl}-2-oxo-1,2- dihydropyrimidin-4-yl)carbamate |
| 123 | 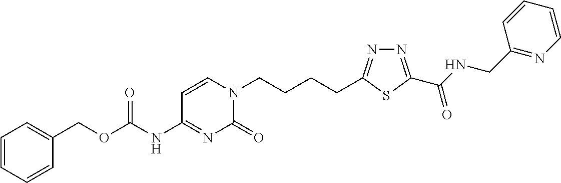 [SMILES: O=C(Nc1ccn(CCCCc2nnc(C(=O)NCc3ccccn3)s2)c(=O)n1)OCc1ccccc1] | benzyl N-[2-oxo-1-(4-{5-[(pyridin-2- ylmethyl)carbamoyl]-1,3,4-thiadiazol-2- yl}butyl)-1,2-dihydropyrimidin-4- yl]carbamate |
| 124 | 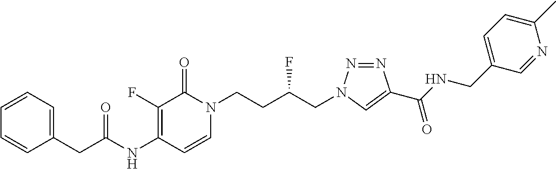 [SMILES: Cc1ccc(CNC(=O)c2cn(C[C@@H](F)CCn3ccc(NC(=O)Cc4ccccc4)c(F)c3=O)nn2)cn1] | (S)-1-(2-fluoro-4-(3-fluoro-2-oxo-4-(2- phenylacetamido)pyridin-1(2H)- yl)butyl)-N-((6-methylpyridin-3- yl)methyl)-1H-1,2,3-triazole-4- carboxamide |
| 125 | 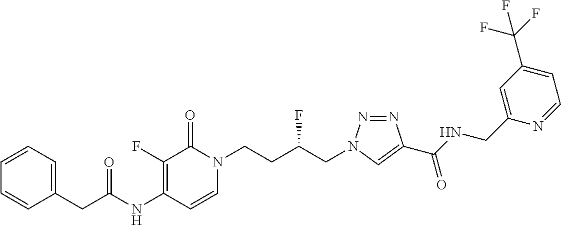 [SMILES: O=C(Cc1ccccc1)Nc1ccn(CC[C@H](F)Cn2cc(C(=O)NCc3cc(C(F)(F)F)ccn3)nn2)c(=O)c1F] | (S)-1-(2-fluoro-4-(3-fluoro-2-oxo-4-(2- phenylacetamido)pyridin-1(2H)- yl)butyl)-N-((4- (trifluoromethyl)pyridin-2-yl)methyl)- 1H-1,2,3-triazole-4-carboxamide |
| 126 | 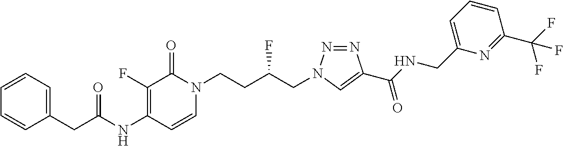 [SMILES: O=C(Cc1ccccc1)Nc1ccn(CC[C@H](F)Cn2cc(C(=O)NCc3cccc(C(F)(F)F)n3)nn2)c(=O)c1F] | (S)-1-(2-fluoro-4-(3-fluoro-2-oxo-4-(2- phenylacetamido)pyridin-1(2H)- yl)butyl)-N-((6- (trifluoromethyl)pyridin-2-yl)methyl)- 1H-1,2,3-triazole-4-carboxamide |
| 127 | 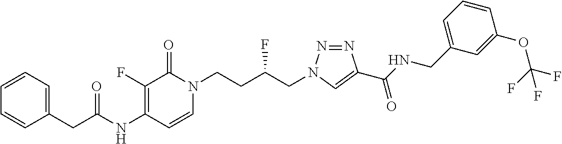 [SMILES: O=C(Cc1ccccc1)Nc1ccn(CC[C@H](F)Cn2cc(C(=O)NCc3cccc(OC(F)(F)F)c3)nn2)c(=O)c1F] | (S)-1-(2-fluoro-4-(3-fluoro-2-oxo-4-(2- phenylacetamido)pyridin-1(2H)- yl)butyl)-N-(3- (trifluoromethoxy)benzyl)-1H-1,2,3- triazole-4-carboxamide |
| 128 | 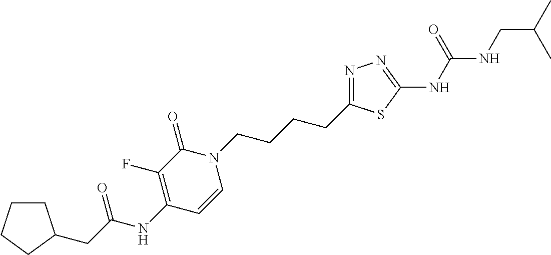 [SMILES: CC(C)CNC(=O)Nc1nnc(CCCCn2ccc(NC(=O)CC3CCCC3)c(F)c2=O)s1] | 2-cyclopentyl-N-(3-fluoro-1-(4-(5-(3- isobutylureido)-1,3,4-thiadiazol-2- yl)butyl)-2-oxo-1,2-dihydropyridin-4- yl)acetamide |
| 129 | 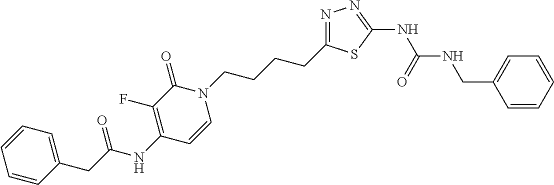 [SMILES: O=C(Cc1ccccc1)Nc1ccn(CCCCc2nnc(NC(=O)NCc3ccccc3)s2)c(=O)c1F] | N-(1-(4-(5-(3-benzylureido)-1,3,4- thiadiazol-2-yl)butyl)-3-fluoro-2-oxo- 1,2-dihydropyridin-4-yl)-2- phenylacetamide |
| 130 | 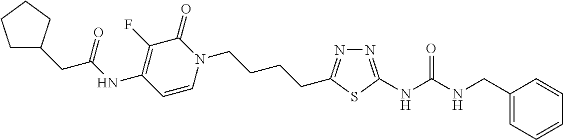 [SMILES: O=C(CC1CCCC1)Nc1ccn(CCCCc2nnc(NC(=O)NCc3ccccc3)s2)c(=O)c1F] | N-(1-(4-(5-(3-benzylureido)-1,3,4- thiadiazol-2-yl)butyl)-3-fluoro-2-oxo- 1,2-dihydropyridin-4-yl)-2- cyclopentylacetamide |
| 131 | 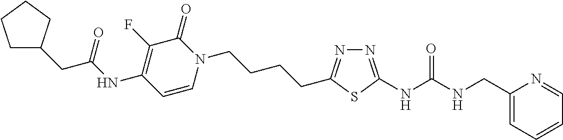 [SMILES: O=C(CC1CCCC1)Nc1ccn(CCCCc2nnc(NC(=O)NCc3ccccn3)s2)c(=O)c1F] | 2-cyclopentyl-N-(3-fluoro-2-oxo-1-(4- (5-(3-(pyridin-2-ylmethyl)ureido)-1,3,4- thiadiazol-2-yl)butyl)-1,2- dihydropyridin-4-yl)acetamide |
| 132 | 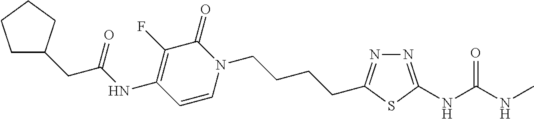 [SMILES: CNC(=O)Nc1nnc(CCCCn2ccc(NC(=O)CC3CCCC3)c(F)c2=O)s1] | 2-cyclopentyl-N-(3-fluoro-1-(4-(5-(3- methylureido)-1,3,4-thiadiazol-2- yl)butyl)-2-oxo-1,2-dihydropyridin-4- yl)acetamide |
| 133 | 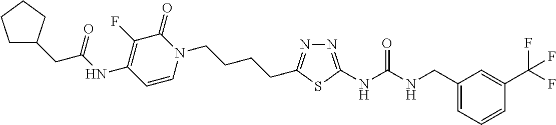 [SMILES: O=C(CC1CCCC1)Nc1ccn(CCCCc2nnc(NC(=O)NCc3cccc(C(F)(F)F)c3)s2)c(=O)c1F] | 2-cyclopentyl-N-(3-fluoro-2-oxo-1-(4- (5-(3-(3- (trifluoromethyl)benzyl)ureido)-1,3,4- thiadiazol-2-yl)butyl)-1,2- dihydropyridin-4-yl)acetamide |
| 134 | 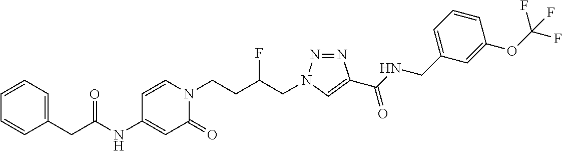 [SMILES: O=C(Cc1ccccc1)Nc1ccn(CCC(F)Cn2cc(C(=O)NCc3cccc(OC(F)(F)F)c3)nn2)c(=O)c1] | 1-{2-fluoro-4-[2-oxo-4-(2- phenylacetamido)-1,2-dihydropyridin-1- yl]butyl}-N-{[3- (trifluoromethoxy)phenyl]methyl}-1H- 1,2,3-triazole-4-carboxamide |
| 135 | 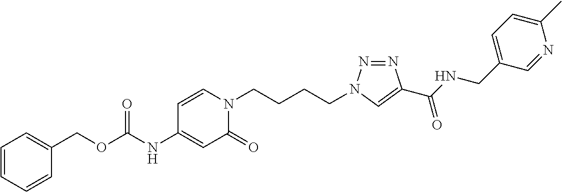 [SMILES: Cc1ccc(CNC(=O)c2cn(CCCCn3ccc(NC(=O)OCc4ccccc4)cc3=O)nn2)cn1] | benzyl N-{1-[4-(4-{[(6-methylpyridin- 3-yl)methyl]carbamoyl}-1H-1,2,3- triazol-1-yl)butyl]-2-oxo-1,2- dihydropyridin-4-yl}carbamate |
| 136 | 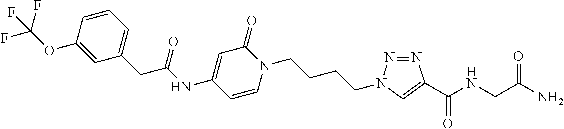 [SMILES: NC(=O)CNC(=O)c1cn(CCCCn2ccc(NC(=O)Cc3cccc(OC(F)(F)F)c3)cc2=O)nn1] | 2-({1-[4-(2-oxo-4-{2-[3- (trifluoromethoxy)phenyl]acetamido}- 1,2-dihydropyridin-1-yl)butyl]-1H- 1,2,3-triazol-4-yl}formamido)acetamide |
| 137 | 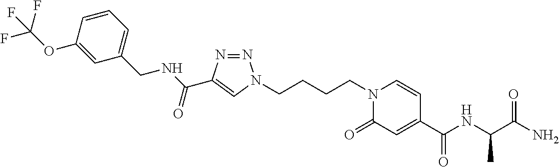 [SMILES: C[C@@H](NC(=O)c1ccn(CCCCn2cc(C(=O)NCc3cccc(OC(F)(F)F)c3)nn2)c(=O)c1)C(N)=O] | 1-[4-(4-{[(1R)-1- carbamoylethyl]carbamoyl}-2-oxo-1,2- dihydropyridin-1-yl)butyl]-N-{[3- (trifluoromethoxy)phenyl]methyl}-1H- 1,2,3-triazole-4-carboxamide |
| 138 | 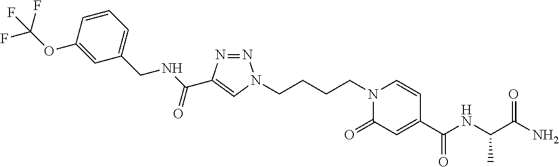 [SMILES: C[C@H](NC(=O)c1ccn(CCCCn2cc(C(=O)NCc3cccc(OC(F)(F)F)c3)nn2)c(=O)c1)C(N)=O] | 1-[4-(4-{[(1S)-1- carbamoylethyl]carbamoyl}-2-oxo-1,2- dihydropyridin-1-yl)butyl]-N-{[3- (trifluoromethoxy)phenyl]methyl}-1H- 1,2,3-triazole-4-carboxamide |
| 139 | 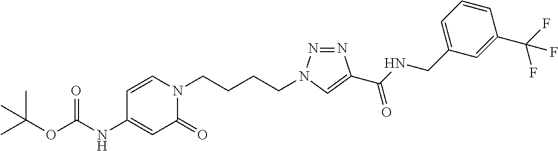 [SMILES: CC(C)(C)OC(=O)Nc1ccn(CCCCn2cc(C(=O)NCc3cccc(C(F)(F)F)c3)nn2)c(=O)c1] | tert-butyl N-(2-oxo-1-{4-[4-({[3- (trifluoromethyl)phenyl]methyl} carbamoyl)-1H-1,2,3-triazol- 1-yl]butyl}-1,2- dihydropyridin-4-yl)carbamate |
| 140 | 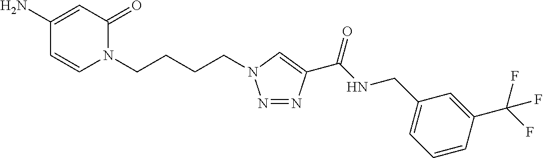 [SMILES: Nc1ccn(CCCCn2cc(C(=O)NCc3cccc(C(F)(F)F)c3)nn2)c(=O)c1] | 1-[4-(4-amino-2-oxo-1,2- dihydropyridin-1-yl)butyl]-N-{[3- (trifluoromethyl)phenyl]methyl}-1H- 1,2,3-triazole-4-carboxamide |
| 141 | 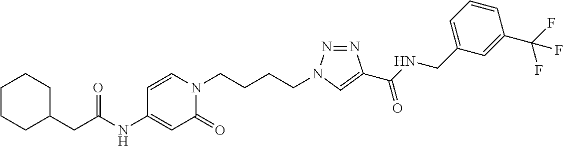 [SMILES: O=C(CC1CCCCC1)Nc1ccn(CCCCn2cc(C(=O)NCc3cccc(C(F)(F)F)c3)nn2)c(=O)c1] | 1-{4-[4-(2-cyclohexylacetamido)-2- oxo-1,2-dihydropyridin-1-yl]butyl}-N- {[3-(trifluoromethyl)phenyl]methyl}- 1H-1,2,3-triazole-4-carboxamide |
| 142 | 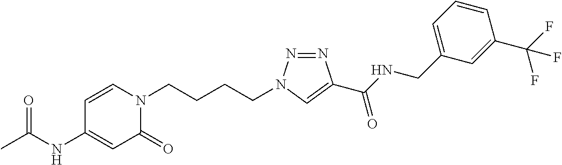 [SMILES: CC(=O)Nc1ccn(CCCCn2cc(C(=O)NCc3cccc(C(F)(F)F)c3)nn2)c(=O)c1] | 1-[4-(4-acetamido-2-oxo-1,2- dihydropyridin-1-yl)butyl]-N-{[3- (trifluoromethyl)phenyl]methyl}-1H- 1,2,3-triazole-4-carboxamide |
| 143 | 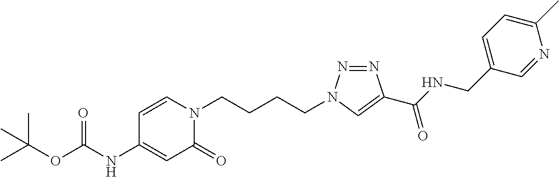 [SMILES: Cc1ccc(CNC(=O)c2cn(CCCCn3ccc(NC(=O)OC(C)(C)C)cc3=O)nn2)cn1] | tert-butyl N-{1-[4-(4-{[(6- methylpyridin-3-yl)methyl]carbamoyl}- 1H-1,2,3-triazol-1-yl)butyl]-2-oxo-1,2- dihydropyridin-4-yl}carbamate |
| 144 | 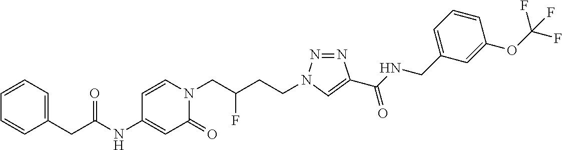 [SMILES: O=C(Cc1ccccc1)Nc1ccn(CC(F)CCn2cc(C(=O)NCc3cccc(OC(F)(F)F)c3)nn2)c(=O)c1] | 1-{3-fluoro-4-[2-oxo-4-(2- phenylacetamido)-1,2-dihydropyridin- 1-yl]butyl}-N-{[3- (trifluoromethoxy)phenyl]methyl}-1H- 1,2,3-triazole-4-carboxamide |
| 145 | 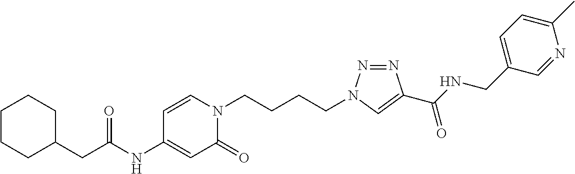 [SMILES: Cc1ccc(CNC(=O)c2cn(CCCCn3ccc(NC(=O)CC4CCCCC4)cc3=O)nn2)cn1] | 1-{4-[4-(2-cyclohexylacetamido)-2- oxo-1,2-dihydropyridin-1-yl]butyl}-N- [(6-methylpyridin-3-yl)methyl]-1H- 1,2,3-triazole-4-carboxamide |
| 146 | 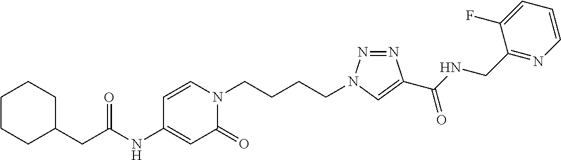 [SMILES: O=C(CC1CCCCC1)Nc1ccn(CCCCn2cc(C(=O)NCc3ncccc3F)nn2)c(=O)c1] | 1-{4-[4-(2-cyclohexylacetamido)-2- oxo-1,2-dihydropyridin-1-yl]butyl}-N- [(3-fluoropyridin-2-yl)methyl]-1H- 1,2,3-triazole-4-carboxamide |
| 147 | 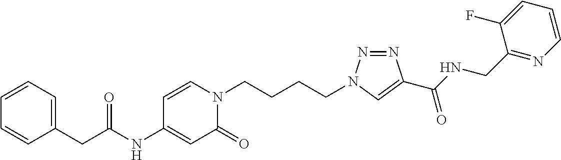 [SMILES: O=C(Cc1ccccc1)Nc1ccn(CCCCn2cc(C(=O)NCc3ncccc3F)nn2)c(=O)c1] | N-[(3-fluoropyridin-2-yl)methyl]-1-{4- [2-oxo-4-(2-phenylacetamido)-1,2- dihydropyridin-1-yl]butyl}-1H-1,2,3- triazole-4-carboxamide |
| 148 | 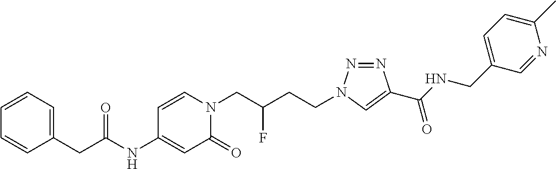 [SMILES: Cc1ccc(CNC(=O)c2cn(CCC(F)Cn3ccc(NC(=O)Cc4ccccc4)cc3=O)nn2)cn1] | 1-{3-fluoro-4-[2-oxo-4-(2- phenylacetamido)-1,2-dihydropyridin-1- yl]butyl}-N-[(6-methylpyridin-3- yl)methyl]-1H-1,2,3-triazole-4- carboxamide |
| 149 | 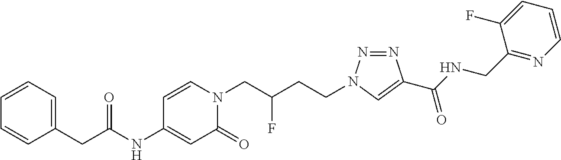 [SMILES: O=C(Cc1ccccc1)Nc1ccn(CC(F)CCn2cc(C(=O)NCc3ncccc3F)nn2)c(=O)c1] | 1-{3-fluoro-4-[2-oxo-4-(2- phenylacetamido)-1,2-dihydropyridin- 1-yl]butyl}-N-[(3-fluoropyridin-2- yl)methyl]-1H-1,2,3-triazole-4- carboxamide |
| 150 | 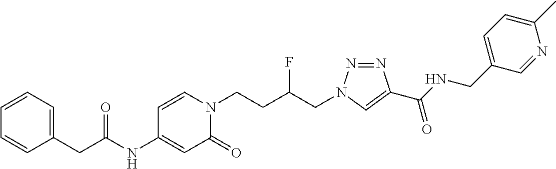 [SMILES: Cc1ccc(CNC(=O)c2cn(CC(F)CCn3ccc(NC(=O)Cc4ccccc4)cc3=O)nn2)cn1] | 1-{2-fluoro-4-[2-oxo-4-(2- phenylacetamido)-1,2-dihydropyridin- 1-yl]butyl}-N-[(6-methylpyridin-3- yl)methyl]-1H-1,2,3-triazole-4- carboxamide |
| 151 | 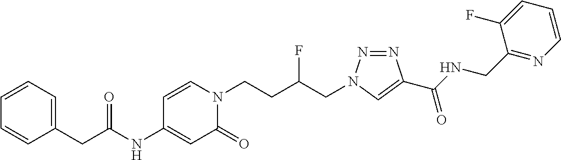 [SMILES: O=C(Cc1ccccc1)Nc1ccn(CCC(F)Cn2cc(C(=O)NCc3ncccc3F)nn2)c(=O)c1] | 1-{2-fluoro-4-[2-oxo-4-(2- phenylacetamido)-1,2-dihydropyridin- 1-yl]butyl}-N-[(3-fluoropyridin-2- yl)methyl]-1H-1,2,3-triazole-4- carboxamide |
| 152 | 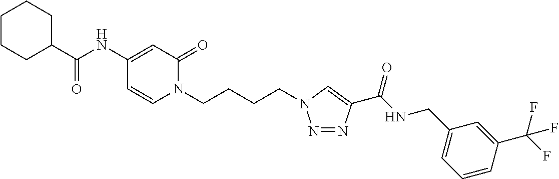 [SMILES: O=C(NCc1cccc(C(F)(F)F)c1)c1cn(CCCCn2ccc(NC(=O)C3CCCCC3)cc2=O)nn1] | 1-[4-(4-cyclohexaneamido-2-oxo-1,2- dihydropyridin-1-yl)butyl]-N-{[3- (trifluoromethyl)phenyl]methyl}-1H- 1,2,3-triazole-4-carboxamide |
| 153 | 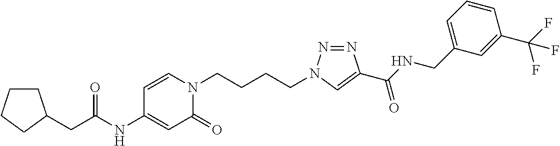 [SMILES: O=C(CC1CCCC1)Nc1ccn(CCCCn2cc(C(=O)NCc3cccc(C(F)(F)F)c3)nn2)c(=O)c1] | 1-{4-[4-(2-cyclopentylacetamido)-2- oxo-1,2-dihydropyridin-1-yl]butyl}-N- {[3-(trifluoromethyl)phenyl]methyl}- 1H-1,2,3-triazole-4-carboxamide |
| 154 | 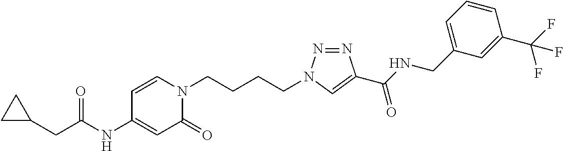 [SMILES: O=C(CC1CC1)Nc1ccn(CCCCn2cc(C(=O)NCc3cccc(C(F)(F)F)c3)nn2)c(=O)c1] | 1-{4-[4-(2-cyclopropylacetamido)-2- oxo-1,2-dihydropyridin-1-yl]butyl}-N- {[3-(trifluoromethyl)phenyl]methyl}- 1H-1,2,3-triazole-4-carboxamide |
| 155 | 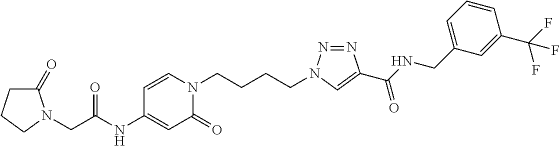 [SMILES: O=C(CN1CCCC1=O)Nc1ccn(CCCCn2cc(C(=O)NCc3cccc(C(F)(F)F)c3)nn2)c(=O)c1] | 1-(4-{2-oxo-4-[2-(2-oxopyrrolidin-1- yl)acetamido]-1,2-dihydropyridin-1- yl}butyl)-N-{[3- (trifluoromethyl)phenyl]methyl}-1H- 1,2,3-triazole-4-carboxamide |
| 156 | 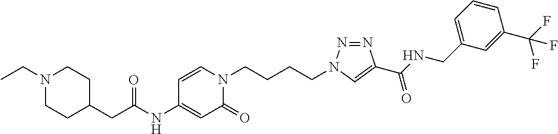 [SMILES: CCN1CCC(CC(=O)Nc2ccn(CCCCn3cc(C(=O)NCc4cccc(C(F)(F)F)c4)nn3)c(=O)c2)CC1] | 1-(4-{4-[2-(1-ethylpiperidin-4- yl)acetamido]-2-oxo-1,2- dihydropyridin-1-yl}butyl)-N-{[3- (trifluoromethyl)phenyl]methyl}-1H- 1,2,3-triazole-4-carboxamide |
| 157 | 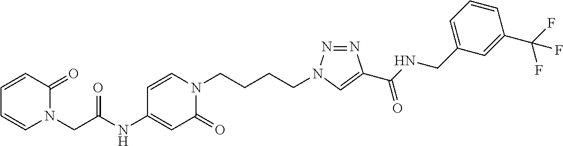 [SMILES: O=C(Cn1ccccc1=O)Nc1ccn(CCCCn2cc(C(=O)NCc3cccc(C(F)(F)F)c3)nn2)c(=O)c1] | 1-(4-{2-oxo-4-[2-(2-oxo-1,2- dihydropyridin-1-yl)acetamido]-1,2- dihydropyridin-1-yl}butyl)-N-{[3- (trifluoromethyl)phenyl]methyl}-1H- 1,2,3-triazole-4-carboxamide |
| 158 | 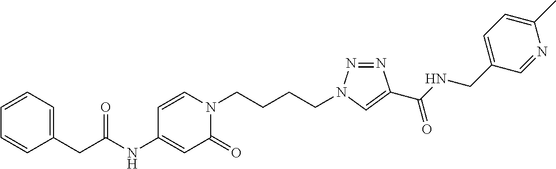 [SMILES: Cc1ccc(CNC(=O)c2cn(CCCCn3ccc(NC(=O)Cc4ccccc4)cc3=O)nn2)cn1] | N-[(6-methylpyridin-3-yl)methyl]-1- {4-[2-oxo-4-(2-phenylacetamido)-1,2- dihydropyridin-1-yl]butyl}-1H-1,2,3- triazole-4-carboxamide |
| 159 | 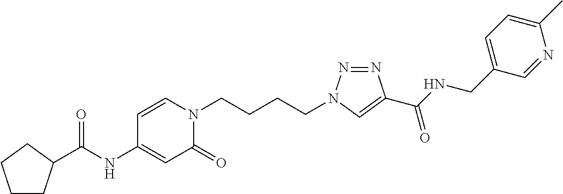 [SMILES: Cc1ccc(CNC(=O)c2cn(CCCCn3ccc(NC(=O)C4CCCC4)cc3=O)nn2)cn1] | 1-[4-(4-cyclopentaneamido-2-oxo-1,2- dihydropyridin-1-yl)butyl]-N-[(6- methylpyridin-3-yl)methyl]-1H-1,2,3- triazole-4-carboxamide |
| 160 | 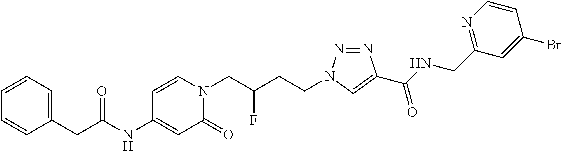 [SMILES: O=C(Cc1ccccc1)Nc1ccn(CC(F)CCn2cc(C(=O)NCc3cc(Br)ccn3)nn2)c(=O)c1] | N-[(4-bromopyridin-2-yl)methyl]-1-{3- fluoro-4-[2-oxo-4-(2-phenylacetamido)- 1,2-dihydropyridin-1-yl]butyl}-1H- 1,2,3-triazole-4-carboxamide |
| 161 | 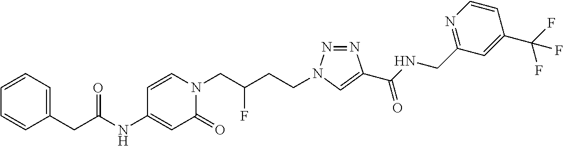 [SMILES: O=C(Cc1ccccc1)Nc1ccn(CC(F)CCn2cc(C(=O)NCc3cc(C(F)(F)F)ccn3)nn2)c(=O)c1] | 1-{3-fluoro-4-[2-oxo-4-(2- phenylacetamido)-1,2-dihydropyridin- 1-yl]butyl}-N-{[4- (trifluoromethyl)pyridin-2-yl]methyl}- 1H-1,2,3-triazole-4-carboxamide |
| 162 | 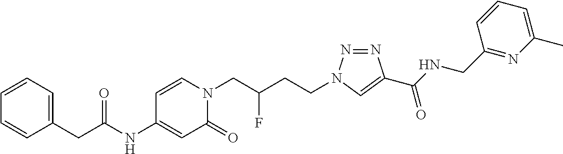 [SMILES: Cc1cccc(CNC(=O)c2cn(CCC(F)Cn3ccc(NC(=O)Cc4ccccc4)cc3=O)nn2)n1] | 1-{3-fluoro-4-[2-oxo-4-(2- phenylacetamido)-1,2-dihydropyridin- 1-yl]butyl}-N-[(6-methylpyridin-2- yl)methyl]-1H-1,2,3-triazole-4- carboxamide |
| 163 | 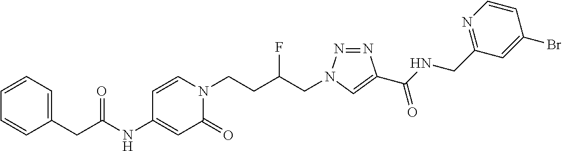 [SMILES: O=C(Cc1ccccc1)Nc1ccn(CCC(F)Cn2cc(C(=O)NCc3cc(Br)ccn3)nn2)c(=O)c1] | N-[(4-bromopyridin-2-yl)methyl]-1-{2- fluoro-4-[2-oxo-4-(2-phenylacetamido)- 1,2-dihydropyridin-1-yl]butyl}-1H- 1,2,3-triazole-4-carboxamide |
| 164 | 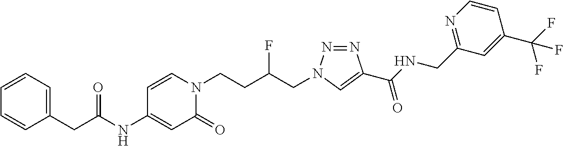 [SMILES: O=C(Cc1ccccc1)Nc1ccn(CCC(F)Cn2cc(C(=O)NCc3cc(C(F)(F)F)ccn3)nn2)c(=O)c1] | 1-{2-fluoro-4-[2-oxo-4-(2- phenylacetamido)-1,2-dihydropyridin- 1-yl]butyl}-N-{[4- (trifluoromethyl)pyridin-2-yl]methyl}- 1H-1,2,3-triazole-4-carboxamide |
| 165 | 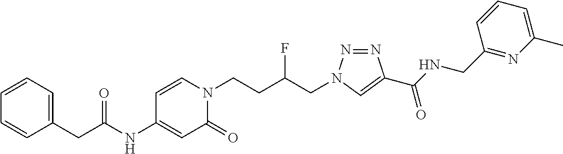 [SMILES: Cc1cccc(CNC(=O)c2cn(CC(F)CCn3ccc(NC(=O)Cc4ccccc4)cc3=O)nn2)n1] | 1-{2-fluoro-4-[2-oxo-4-(2- phenylacetamido)-1,2-dihydropyridin- 1-yl]butyl}-N-[(6-methylpyridin-2- yl)methyl]-1H-1,2,3-triazole-4- carboxamide |
| 166 | 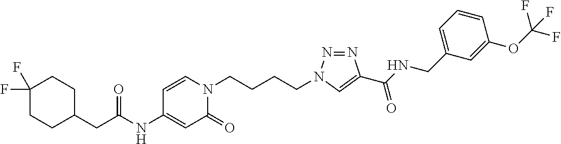 [SMILES: O=C(CC1CCC(F)(F)CC1)Nc1ccn(CCCCn2cc(C(=O)NCc3cccc(OC(F)(F)F)c3)nn2)c(=O)c1] | 1-(4-{4-[2-(4,4- difluorocyclohexyl)acetamido]-2-oxo- 1,2-dihydropyridin-1-yl}butyl)-N-{[3- (trifluoromethoxy)phenyl]methyl}-1H- 1,2,3-triazole-4-carboxamide |
| 167 | 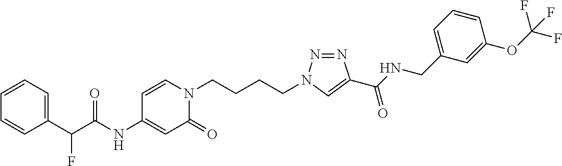 [SMILES: O=C(NCc1cccc(OC(F)(F)F)c1)c1cn(CCCCn2ccc(NC(=O)C(F)c3ccccc3)cc2=O)nn1] | 1-{4-[4-(2-fluoro-2-phenylacetamido)- 2-oxo-1,2-dihydropyridin-1-yl]butyl}- N-{[3- (trifluoromethoxy)phenyl]methyl}-1H- 1,2,3-triazole-4-carboxamide |
| 168 | 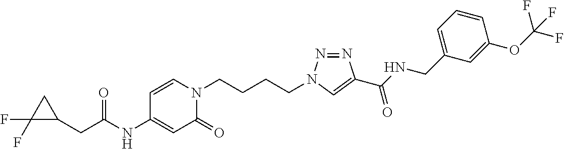 [SMILES: O=C(CC1CC1(F)F)Nc1ccn(CCCCn2cc(C(=O)NCc3cccc(OC(F)(F)F)c3)nn2)c(=O)c1] | 1-(4-{4-[2-(2,2- difluorocyclopropyl)acetamido]-2-oxo- 1,2-dihydropyridin-1-yl}butyl)-N-{[3- (trifluoromethoxy)phenyl]methyl}-1H- 1,2,3-triazole-4-carboxamide |
| 169 | 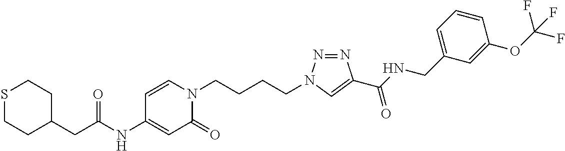 [SMILES: O=C(CC1CCSCC1)Nc1ccn(CCCCn2cc(C(=O)NCc3cccc(OC(F)(F)F)c3)nn2)c(=O)c1] | 1-(4-{2-oxo-4-[2-(thian-4- yl)acetamido]-1,2-dihydropyridin-1- yl}butyl)-N-{[3- (trifluoromethoxy)phenyl]methyl}-1H- 1,2,3-triazole-4-carboxamide |
| 170 | 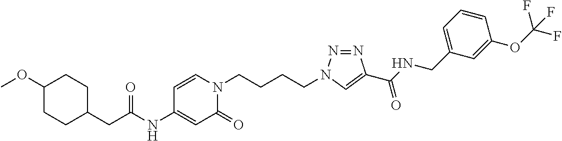 [SMILES: COC1CCC(CC(=O)Nc2ccn(CCCCn3cc(C(=O)NCc4cccc(OC(F)(F)F)c4)nn3)c(=O)c2)CC1] | 1-(4-{4-[2-(4- methoxycyclohexyl)acetamido]-2-oxo- 1,2-dihydropyridin-1-yl}butyl)-N-{[3- (trifluoromethoxy)phenyl]methyl}-1H- 1,2,3-triazole-4-carboxamide |
| 171 | 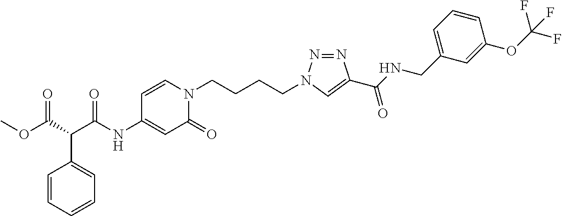 [SMILES: COC(=O)[C@H](C(=O)Nc1ccn(CCCCn2cc(C(=O)NCc3cccc(OC(F)(F)F)c3)nn2)c(=O)c1)c1ccccc1] | (S)-[(2-oxo-1-{4-[4-({[3- (trifluoromethoxy)phenyl]methyl} carbamoyl)-1H-1,2,3-triazol-1- yl]butyl}-1,2-dihydropyridin-4- yl)carbamoyl](phenyl)methyl acetate |
| 172 | 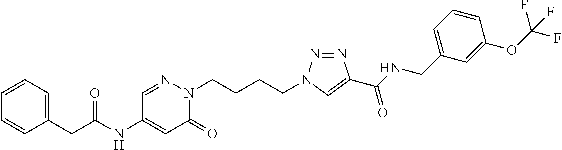 [SMILES: O=C(Cc1ccccc1)Nc1cnn(CCCCn2cc(C(=O)NCc3cccc(OC(F)(F)F)c3)nn2)c(=O)c1] | 1-{4-[6-oxo-4-(2-phenylacetamido)-1,6- dihydropyridazin-1-yl]butyl}-N-{[3- (trifluoromethoxy)phenyl]methyl}-1H- 1,2,3-triazole-4-carboxamide |
| 173 | 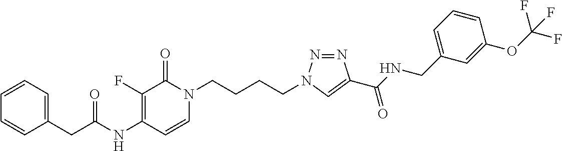 [SMILES: O=C(Cc1ccccc1)Nc1ccn(CCCCn2cc(C(=O)NCc3cccc(OC(F)(F)F)c3)nn2)c(=O)c1F] | 1-{4-[3-fluoro-2-oxo-4-(2- phenylacetamido)-1,2-dihydropyridin- 1-yl]butyl}-N-{[3- (trifluoromethoxy)phenyl]methyl}-1H- 1,2,3-triazole-4-carboxamide |
| 174 | 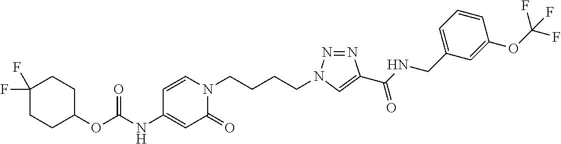 [SMILES: O=C(Nc1ccn(CCCCn2cc(C(=O)NCc3cccc(OC(F)(F)F)c3)nn2)c(=O)c1)OC1CCC(F)(F)CC1] | 4,4-difluorocyclohexyl N-(2-oxo-1-{4- [4-({[3-(trifluoro- methoxy)phenyl]methyl}carbamoyl)- 1H-1,2,3-triazol-1-yl]butyl}-1,2- dihydropyridin-4-yl)carbamate |
| 175 | 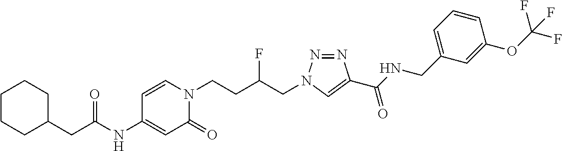 [SMILES: O=C(CC1CCCCC1)Nc1ccn(CCC(F)Cn2cc(C(=O)NCc3cccc(OC(F)(F)F)c3)nn2)c(=O)c1] | 1-{4-[4-(2-cyclohexylacetamido)-2- oxo-1,2-dihydropyridin-1-yl]-2- fluorobutyl}-N-{[3- (trifluoromethoxy)phenyl]methyl}-1H- 1,2,3-triazole-4-carboxamide |
| 176 | 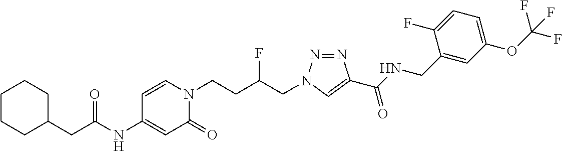 [SMILES: O=C(CC1CCCCC1)Nc1ccn(CCC(F)Cn2cc(C(=O)NCc3cc(OC(F)(F)F)ccc3F)nn2)c(=O)c1] | 1-{4-[4-(2-cyclohexylacetamido)-2- oxo-1,2-dihydropyridin-1-yl]-2- fluorobutyl}-N-{[2-fluoro-5- (trifluoromethoxy)phenyl]methyl}-1H- 1,2,3-triazole-4-carboxamide |
| 177 | 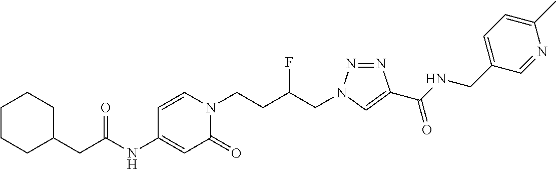 [SMILES: Cc1ccc(CNC(=O)c2cn(CC(F)CCn3ccc(NC(=O)CC4CCCCC4)cc3=O)nn2)cn1] | 1-{4-[4-(2-cyclohexylacetamido)-2- oxo-1,2-dihydropyridin-1-yl]-2- fluorobutyl}-N-[(6-methylpyridin-3- yl)methyl]-1H-1,2,3-triazole-4- carboxamide |
| 178 | 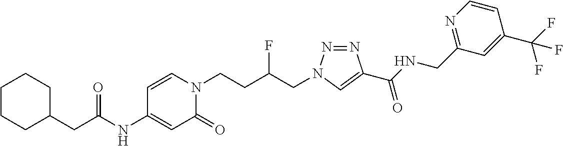 [SMILES: O=C(CC1CCCCC1)Nc1ccn(CCC(F)Cn2cc(C(=O)NCc3cc(C(F)(F)F)ccn3)nn2)c(=O)c1] | 1-{4-[4-(2-cyclohexylacetamido)-2- oxo-1,2-dihydropyridin-1-yl]-2- fluorobutyl}-N-{[4- (trifluoromethyl)pyridin-2-yl]methyl}- 1H-1,2,3-triazole-4-carboxamide |
| 179 | 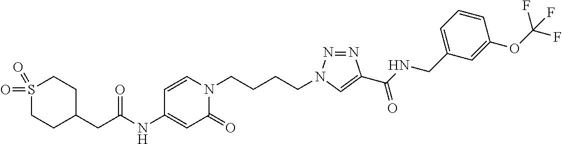 [SMILES: O=C(CC1CCS(=O)(=O)CC1)Nc1ccn(CCCCn2cc(C(=O)NCc3cccc(OC(F)(F)F)c3)nn2)c(=O)c1] | 1-(4-{4-[2-(1,1-dioxo-1$l\hat{ }{6}-thian-4- yl)acetamido]-2-oxo-1,2- dihydropyridin-1-yl}butyl)-N-{[3- (trifluoromethoxy)phenyl]methyl}-1H- 1,2,3-triazole-4-carboxamide |
| 180 | 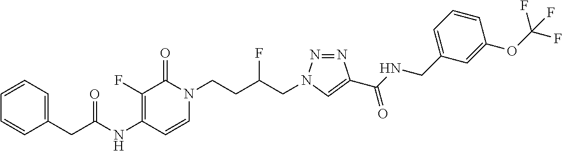 [SMILES: O=C(Cc1ccccc1)Nc1ccn(CCC(F)Cn2cc(C(=O)NCc3cccc(OC(F)(F)F)c3)nn2)c(=O)c1F] | 1-{2-fluoro-4-[3-fluoro-2-oxo-4-(2- phenylacetamido)-1,2-dihydropyridin- 1-yl]butyl}-N-{[3- (trifluoromethoxy)phenyl]methyl}-1H- 1,2,3-triazole-4-carboxamide |
| 181 | 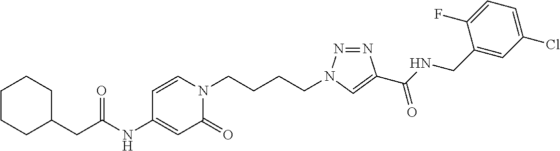 [SMILES: O=C(CC1CCCCC1)Nc1ccn(CCCCn2cc(C(=O)NCc3cc(Cl)ccc3F)nn2)c(=O)c1] | N-[(5-chloro-2-fluorophenyl)methyl]-1- {4-[4-(2-cyclohexylacetamido)-2-oxo- 1,2-dihydropyridin-1-yl]butyl}-1H- 1,2,3-triazole-4-carboxamide |
| 182 | 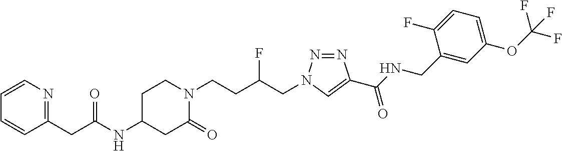 [SMILES: O=C(Cc1ccccn1)NC1CCN(CCC(F)Cn2cc(C(=O)NCc3cc(OC(F)(F)F)ccc3F)nn2)C(=O)C1] | 1-(2-fluoro-4-{2-oxo-4-[2-(pyridin-2- yl)acetamido]-1,2-dihydropyridin-1- yl}butyl)-N-{[2-fluoro-5- (trifluoromethoxy)phenyl]methyl}-1H- 1,2,3-triazole-4-carboxamide |
| 183 | 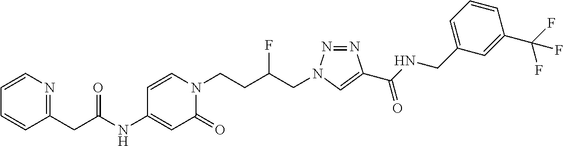 [SMILES: O=C(Cc1ccccn1)Nc1ccn(CCC(F)Cn2cc(C(=O)NCc3cccc(C(F)(F)F)c3)nn2)c(=O)c1] | 1-(2-fluoro-4-{2-oxo-4-[2-(pyridin-2- yl)acetamido]-1,2-dihydropyridin-1- yl}butyl)-N-{[3- (trifluoromethyl)phenyl]methyl}-1H- 1,2,3-triazole-4-carboxamide |
| 184 | 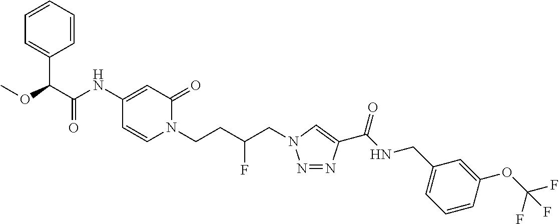 [SMILES: CO[C@H](C(=O)Nc1ccn(CCC(F)Cn2cc(C(=O)NCc3cccc(OC(F)(F)F)c3)nn2)c(=O)c1)c1ccccc1] | 1-(2-fluoro-4-{4-[(2S)-2-methoxy-2- phenylacetamido]-2-oxo-1,2- dihydropyridin-1-yl}butyl)-N-{[3- (trifluoromethoxy)phenyl]methyl}-1H- 1,2,3-triazole-4-carboxamide |
| 185 | 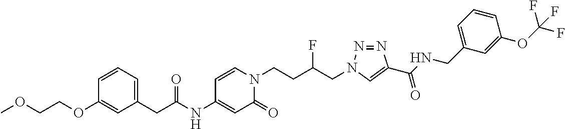 [SMILES: COCCOc1cccc(CC(=O)Nc2ccn(CCC(F)Cn3cc(C(=O)NCc4cccc(OC(F)(F)F)c4)nn3)c(=O)c2)c1] | 1-[2-fluoro-4-(4-{2-[3-(2- methoxyethoxy)phenyl]acetamido}-2- oxo-1,2-dihydropyridin-1-yl)butyl]-N- {[3-(trifluoromethoxy)phenyl]methyl}- 1H-1,2,3-triazole-4-carboxamide |
| 186 | 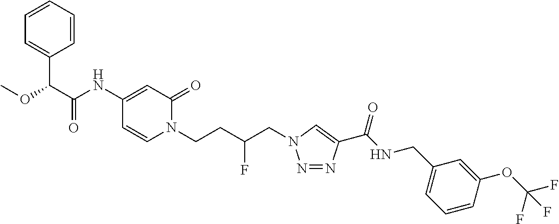 [SMILES: CO[C@@H](C(=O)Nc1ccn(CCC(F)Cn2cc(C(=O)NCc3cccc(OC(F)(F)F)c3)nn2)c(=O)c1)c1ccccc1] | 1-(2-fluoro-4-{4-[(2R)-2-methoxy-2- phenylacetamido]-2-oxo-1,2- dihydropyridin-1-yl}butyl)-N-{[3- (trifluoromethoxy)phenyl]methyl}-1H- 1,2,3-triazole-4-carboxamide |
| 187 | 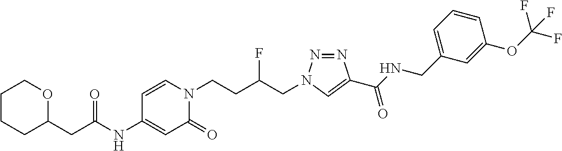 [SMILES: O=C(CC1CCCCO1)Nc1ccn(CCC(F)Cn2cc(C(=O)NCc3cccc(OC(F)(F)F)c3)nn2)c(=O)c1] | 1-(2-fluoro-4-{4-[2-(oxan-2- yl)acetamido]-2-oxo-1,2- dihydropyridin-1-yl}butyl)-N-{[3- (trifluoromethoxy)phenyl]methyl}-1H- 1,2,3-triazole-4-carboxamide |
| 188 | 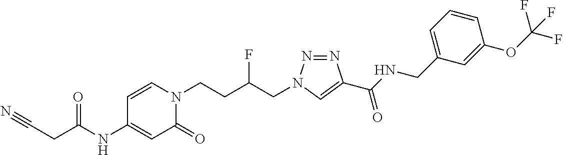 [SMILES: N#CCC(=O)Nc1ccn(CCC(F)Cn2cc(C(=O)NCc3cccc(OC(F)(F)F)c3)nn2)c(=O)c1] | 1-{4-[4-(2-cyanoacetamido)-2-oxo-1,2- dihydropyridin-1-yl]-2-fluorobutyl}-N- {[3-(trifluoromethoxy)phenyl]methyl}- 1H-1,2,3-triazole-4-carboxamide |
| 189 | 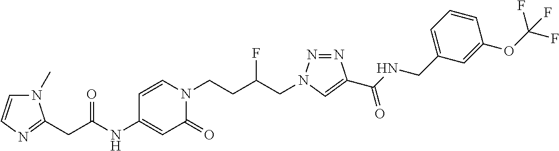 [SMILES: Cn1ccnc1CC(=O)Nc1ccn(CCC(F)Cn2cc(C(=O)NCc3cccc(OC(F)(F)F)c3)nn2)c(=O)c1] | 1-(2-fluoro-4-{4-[2-(1-methyl-1H- imidazol-2-yl)acetamido]-2-oxo-1,2- dihydropyridin-1-yl}butyl)-N-{[3- (trifluoromethoxy)phenyl]methyl}-1H- 1,2,3-triazole-4-carboxamide |
| 190 | 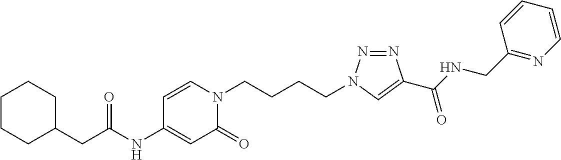 [SMILES: O=C(CC1CCCCC1)Nc1ccn(CCCCn2cc(C(=O)NCc3ccccn3)nn2)c(=O)c1] | 1-{4-[4-(2-cyclohexylacetamido)-2- oxo-1,2-dihydropyridin-1-yl]butyl}-N- (pyridin-2-ylmethyl)-1H-1,2,3-triazole- 4-carboxamide |
| 191 | 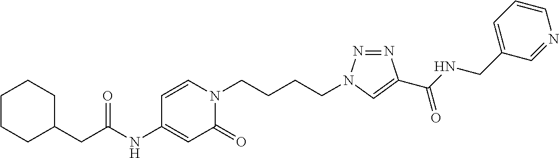 [SMILES: O=C(CC1CCCCC1)Nc1ccn(CCCCn2cc(C(=O)NCc3cccnc3)nn2)c(=O)c1] | 1-{4-[4-(2-cyclohexylacetamido)-2- oxo-1,2-dihydropyridin-1-yl]butyl}-N- (pyridin-3-ylmethyl)-1H-1,2,3-triazole- 4-carboxamide |
| 192 | 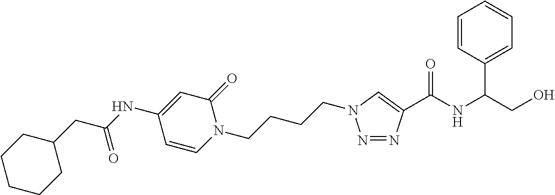 [SMILES: O=C(CC1CCCCC1)Nc1ccn(CCCCn2cc(C(=O)NC(CO)c3ccccc3)nn2)c(=O)c1] | 1-{4-[4-(2-cyclohexylacetamido)-2- oxo-1,2-dihydropyridin-1-yl]butyl}-N- (2-hydroxy-1-phenylethyl)-1H-1,2,3- triazole-4-carboxamide |
| 193 | 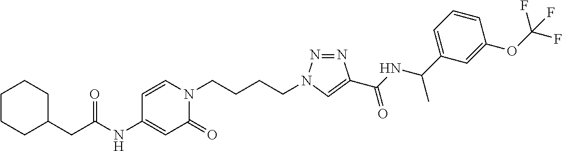 [SMILES: CC(NC(=O)c1cn(CCCCn2ccc(NC(=O)CC3CCCCC3)cc2=O)nn1)c1cccc(OC(F)(F)F)c1] | 1-{4-[4-(2-cyclohexylacetamido)-2- oxo-1,2-dihydropyridin-1-yl]butyl}-N- {1-[3-(trifluoromethoxy)phenyl]ethyl}- 1H-1,2,3-triazole-4-carboxamide |
| 194 | 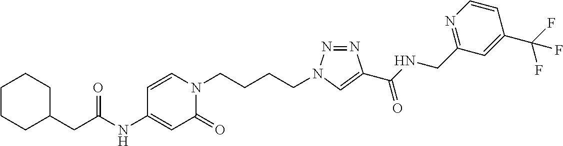 [SMILES: O=C(CC1CCCCC1)Nc1ccn(CCCCn2cc(C(=O)NCc3cc(C(F)(F)F)ccn3)nn2)c(=O)c1] | 1-{4-[4-(2-cyclohexylacetamido)-2- oxo-1,2-dihydropyridin-1-yl]butyl}-N- {[4-(trifluoromethyl)pyridin-2- yl]methyl}-1H-1,2,3-triazole-4- carboxamide |
| 195 | 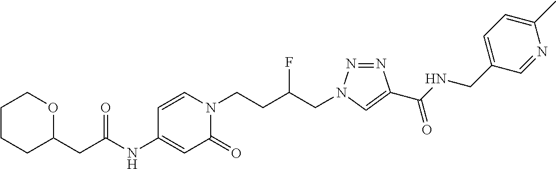 [SMILES: Cc1ccc(CNC(=O)c2cn(CC(F)CCn3ccc(NC(=O)CC4CCCCO4)cc3=O)nn2)cn1] | 1-(2-fluoro-4-{4-[2-(oxan-2- yl)acetamido]-2-oxo-1,2- dihydropyridin-1-yl}butyl)-N-[(6- methylpyridin-3-yl)methyl]-1H-1,2,3- triazole-4-carboxamide |
| 196 | 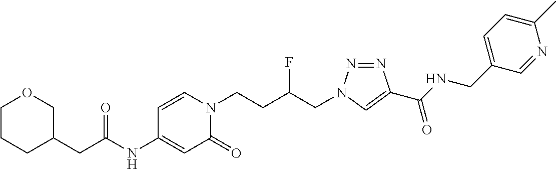 [SMILES: Cc1ccc(CNC(=O)c2cn(CC(F)CCn3ccc(NC(=O)CC4CCCOC4)cc3=O)nn2)cn1] | 1-(2-fluoro-4-{4-[2-(oxan-3- yl)acetamido]-2-oxo-1,2- dihydropyridin-1-yl}butyl)-N-[(6- methylpyridin-3-yl)methyl]-1H-1,2,3- triazole-4-carboxamide |
| 197 | 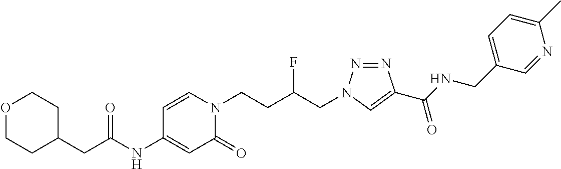 [SMILES: Cc1ccc(CNC(=O)c2cn(CC(F)CCn3ccc(NC(=O)CC4CCOCC4)cc3=O)nn2)cn1] | 1-(2-fluoro-4-{4-[2-(oxan-4- yl)acetamido]-2-oxo-1,2- dihydropyridin-1-yl}butyl)-N-[(6- methylpyridin-3-yl)methyl]-1H-1,2,3- triazole-4-carboxamide |
| 198 | 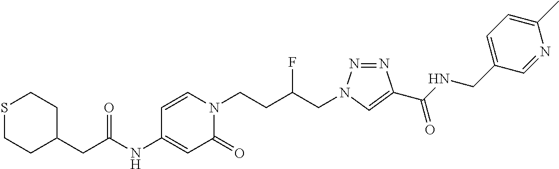 [SMILES: Cc1ccc(CNC(=O)c2cn(CC(F)CCn3ccc(NC(=O)CC4CCSCC4)cc3=O)nn2)cn1] | 1-(2-fluoro-4-{2-oxo-4-[2-(thian-4- yl)acetamido]-1,2-dihydropyridin-1- yl}butyl)-N-[(6-methylpyridin-3- yl)methyl]-1H-1,2,3-triazole-4- carboxamide |
| 199 | 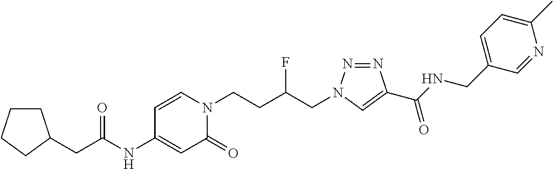 [SMILES: Cc1ccc(CNC(=O)c2cn(CC(F)CCn3ccc(NC(=O)CC4CCCC4)cc3=O)nn2)cn1] | 1-{4-[4-(2-cyclopentylacetamido)-2- oxo-1,2-dihydropyridin-1-yl]-2- fluorobutyl}-N-[(6-methylpyridin-3- yl)methyl]-1H-1,2,3-triazole-4- carboxamide |
| 200 | 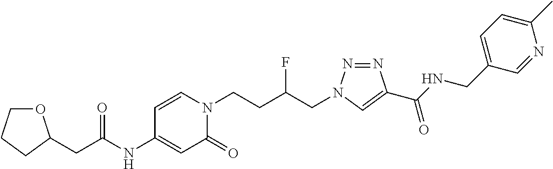 [SMILES: Cc1ccc(CNC(=O)c2cn(CC(F)CCn3ccc(NC(=O)CC4CCCO4)cc3=O)nn2)cn1] | 1-(2-fluoro-4-{2-oxo-4-[2-(oxolan-2- yl)acetamido]-1,2-dihydropyridin-1- yl}butyl)-N-[(6-methylpyridin-3- yl)methyl]-1H-1,2,3-triazole-4- carboxamide |
| 201 | 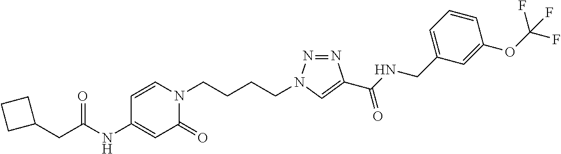 [SMILES: O=C(CC1CCC1)Nc1ccn(CCCCn2cc(C(=O)NCc3cccc(OC(F)(F)F)c3)nn2)c(=O)c1] | 1-{4-[4-(2-cyclobutylacetamido)-2-oxo- 1,2-dihydropyridin-1-yl]butyl}-N-{[3- (trifluoromethoxy)phenyl]methyl}-1H- 1,2,3-triazole-4-carboxamide |
| 202 | 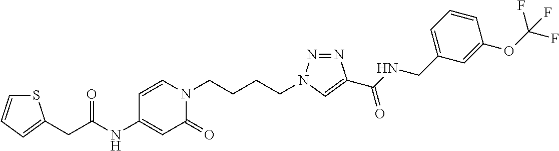 [SMILES: O=C(Cc1cccs1)Nc1ccn(CCCCn2cc(C(=O)NCc3cccc(OC(F)(F)F)c3)nn2)c(=O)c1] | 1-(4-{2-oxo-4-[2-(thiophen-2- yl)acetamido]-1,2-dihydropyridin-1- yl}butyl)-N-{[3- (trifluoromethoxy)phenyl]methyl}-1H- 1,2,3-triazole-4-carboxamide |
| 203 | 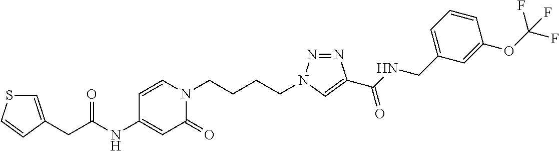 [SMILES: O=C(Cc1ccsc1)Nc1ccn(CCCCn2cc(C(=O)NCc3cccc(OC(F)(F)F)c3)nn2)c(=O)c1] | 1-(4-{2-oxo-4-[2-(thiophen-3- yl)acetamido]-1,2-dihydropyridin-1- yl}butyl)-N-{[3- (trifluoromethoxy)phenyl]methyl}-1H- 1,2,3-triazole-4-carboxamide |
| 204 | 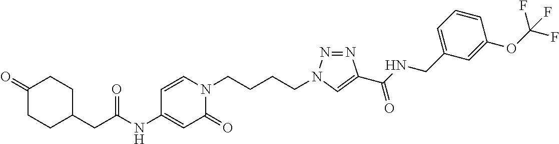 [SMILES: O=C1CCC(CC(=O)Nc2ccn(CCCCn3cc(C(=O)NCc4cccc(OC(F)(F)F)c4)nn3)c(=O)c2)CC1] | 1-(4-{2-oxo-4-[2-(4- oxocyclohexyl)acetamido]-1,2- dihydropyridin-1-yl}butyl)-N-{[3- (trifluoromethoxy)phenyl]methyl}-1H- 1,2,3-triazole-4-carboxamide |
| 205 | 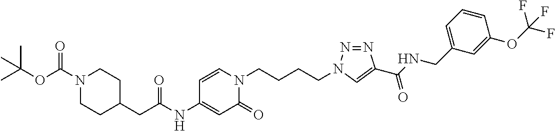 [SMILES: CC(C)(C)OC(=O)N1CCC(CC(=O)Nc2ccn(CCCCn3cc(C(=O)NCc4cccc(OC(F)(F)F)c4)nn3)c(=O)c2)CC1] | tert-butyl 4-{[(2-oxo-1-{4-[4-({[3- (trifluoromethoxy)phenyl]methyl} carbamoyl)-1H-1,2,3-triazol-1-yl]butyl}- 1,2-dihydropyridin-4- yl)carbamoyl]methyl}piperidine-1- carboxylate |
| 206 | 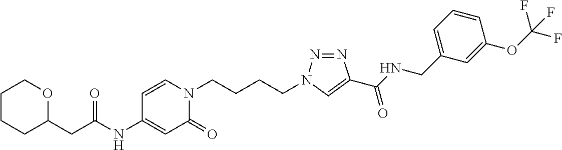 [SMILES: O=C(CC1CCCCO1)Nc1ccn(CCCCn2cc(C(=O)NCc3cccc(OC(F)(F)F)c3)nn2)c(=O)c1] | 1-(4-{4-[2-(oxan-2-yl)acetamido]-2- oxo-1,2-dihydropyridin-1-yl}butyl)-N- {[3-(trifluoromethoxy)phenyl]methyl}- 1H-1,2,3-triazole-4-carboxamide |
| 207 | 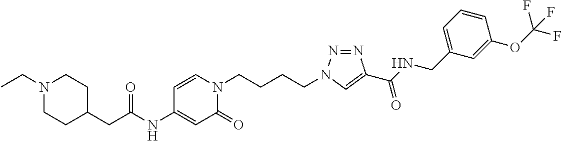 [SMILES: CCN1CCC(CC(=O)Nc2ccn(CCCCn3cc(C(=O)NCc4cccc(OC(F)(F)F)c4)nn3)c(=O)c2)CC1] | 1-(4-{4-[2-(1-ethylpiperidin-4- yl)acetamido]-2-oxo-1,2- dihydropyridin-1-yl}butyl)-N-{[3- (trifluoromethoxy)phenyl]methyl}-1H- 1,2,3-triazole-4-carboxamide |
| 208 | 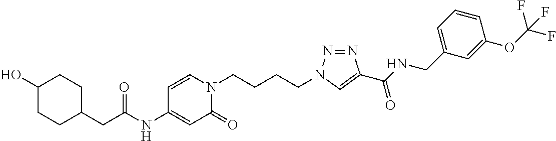 [SMILES: O=C(CC1CCC(O)CC1)Nc1ccn(CCCCn2cc(C(=O)NCc3cccc(OC(F)(F)F)c3)nn2)c(=O)c1] | 1-(4-{4-[2-(4- hydroxycyclohexyl)acetamido]-2-oxo- 1,2-dihydropyridin-1-yl}butyl)-N-{[3- (trifluoromethoxy)phenyl]methyl}-1H- 1,2,3-triazole-4-carboxamide |
| 209 | 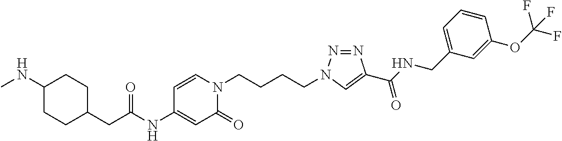 [SMILES: CNC1CCC(CC(=O)Nc2ccn(CCCCn3cc(C(=O)NCc4cccc(OC(F)(F)F)c4)nn3)c(=O)c2)CC1] | 1-[4-(4-{2-[4- (methylamino)cyclohexyl]acetamido}- 2-oxo-1,2-dihydropyridin-1-yl)butyl]- N-{[3- (trifluoromethoxy)phenyl]methyl}-1H- 1,2,3-triazole-4-carboxamide |
| 210 | 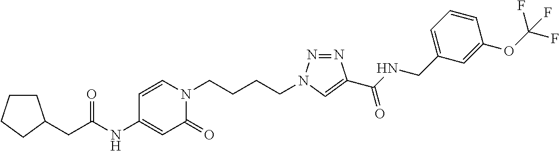 [SMILES: O=C(CC1CCCC1)Nc1ccn(CCCCn2cc(C(=O)NCc3cccc(OC(F)(F)F)c3)nn2)c(=O)c1] | 1-{4-[4-(2-cyclopentylacetamido)-2- oxo-1,2-dihydropyridin-1-yl]butyl}-N- {[3-(trifluoromethoxy)phenyl]methyl}- 1H-1,2,3-triazole-4-carboxamide |
| 211 | 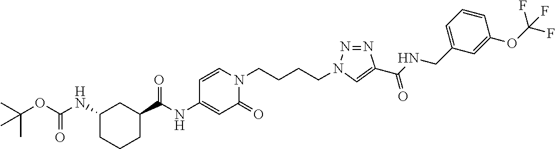 [SMILES: CC(C)(C)OC(=O)N[C@H]1CCC[C@H](C(=O)Nc2ccn(CCCCn3cc(C(=O)NCc4cccc(OC(F)(F)F)c4)nn3)c(=O)c2)C1] | tert-butyl N-[(1S,3S)-3-[(2-oxo-1-{4- [4-({[3-(trifluoro- methoxy)phenyl]methyl}carbamoyl)- 1H-1,2,3-triazol-1-yl]butyl}-1,2- dihydropyridin-4- yl)carbamoyl]cyclohexyl]carbamate |
| 212 | 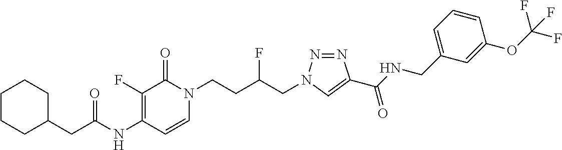 [SMILES: O=C(CC1CCCCC1)Nc1ccn(CCC(F)Cn2cc(C(=O)NCc3cccc(OC(F)(F)F)c3)nn2)c(=O)c1F] | 1-{4-[4-(2-cyclohexylacetamido)-3- fluoro-2-oxo-1,2-dihydropyridin-1-yl]- 2-fluorobutyl}-N-{[3- (trifluoromethoxy)phenyl]methyl}-1H- 1,2,3-triazole-4-carboxamide |
| 213 | 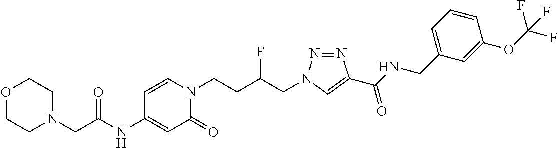 [SMILES: O=C(CN1CCOCC1)Nc1ccn(CCC(F)Cn2cc(C(=O)NCc3cccc(OC(F)(F)F)c3)nn2)c(=O)c1] | 1-(2-fluoro-4-{4-[2-(morpholin-4- yl)acetamido]-2-oxo-1,2- dihydropyridin-1-yl}butyl)-N-{[3- (trifluoromethoxy)phenyl]methyl}-1H- 1,2,3-triazole-4-carboxamide |
| 214 | 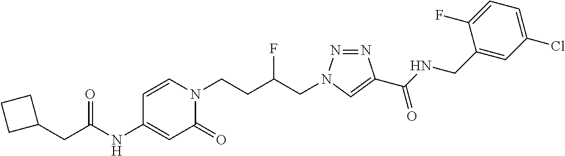 [SMILES: O=C(CC1CCC1)Nc1ccn(CCC(F)Cn2cc(C(=O)NCc3cc(Cl)ccc3F)nn2)c(=O)c1] | N-[(5-chloro-2-fluorophenyl)methyl]-1- {4-[4-(2-cyclobutylacetamido)-2-oxo- 1,2-dihydropyridin-1-yl]-2- fluorobutyl}-1H-1,2,3-triazole-4- carboxamide |
| 215 | 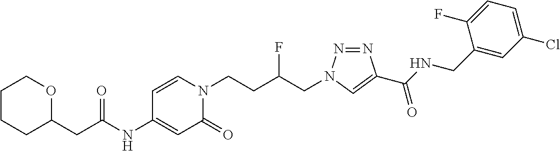 [SMILES: O=C(CC1CCCCO1)Nc1ccn(CCC(F)Cn2cc(C(=O)NCc3cc(Cl)ccc3F)nn2)c(=O)c1] | N-[(5-chloro-2-fluorophenyl)methyl]-1- (2-fluoro-4-{4-[2-(oxan-2- yl)acetamido]-2-oxo-1,2- dihydropyridin-1-yl}butyl)-1H-1,2,3- triazole-4-carboxamide |
| 216 | 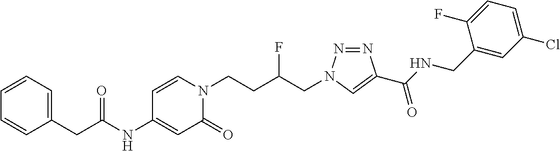 [SMILES: O=C(Cc1ccccc1)Nc1ccn(CCC(F)Cn2cc(C(=O)NCc3cc(Cl)ccc3F)nn2)c(=O)c1] | N-[(5-chloro-2-fluorophenyl)methyl]-1- {2-fluoro-4-[2-oxo-4-(2- phenylacetamido)-1,2-dihydropyridin-1- yl]butyl}-1H-1,2,3-triazole-4- carboxamide |
| 217 | 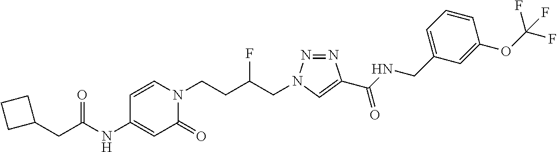 [SMILES: O=C(CC1CCC1)Nc1ccn(CCC(F)Cn2cc(C(=O)NCc3cccc(OC(F)(F)F)c3)nn2)c(=O)c1] | l-{4-[4-(2-cyclobutylacetamido)-2-oxo- 1,2-dihydropyridin-1-yl]-2- fluorobutyl}-N-{[3- (trifluoromethoxy)phenyl]methyl}-1H- 1,2,3-triazole-4-carboxamide |
| 218 | 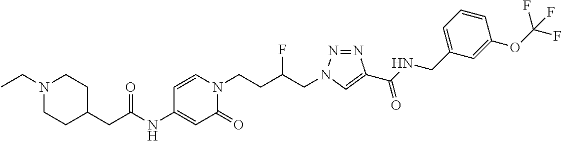 [SMILES: CCN1CCC(CC(=O)Nc2ccn(CCC(F)Cn3cc(C(=O)NCc4cccc(OC(F)(F)F)c4)nn3)c(=O)c2)CC1] | 1-(4-{4-[2-(1-ethylpiperidin-4- yl)acetamido]-2-oxo-1,2- dihydropyridin-1-yl}-2-fluorobutyl)-N- {[3-(trifluoromethoxy)phenyl]methyl}- 1H-1,2,3-triazole-4-carboxamide |
| 219 | 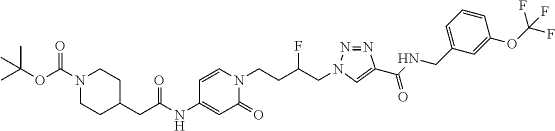 [SMILES: CC(C)(C)OC(=O)N1CCC(CC(=O)Nc2ccn(CCC(F)Cn3cc(C(=O)NCc4cccc(OC(F)(F)F)c4)nn3)c(=O)c2)CC1] | tert-butyl 4-{[(1-{3-fluoro-4-[4-({[3- (trifluoromethoxy)phenyl]methyl} carbamoyl)-1H-1,2,3-triazol-1- yl]butyl}-2-oxo-1,2-dihydropyridin- 4-yl)carbamoyl]methyl}piperidine- 1-carboxylate |
| 220 | 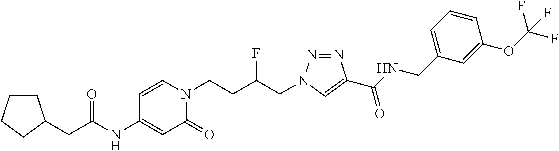 [SMILES: O=C(CC1CCCC1)Nc1ccn(CCC(F)Cn2cc(C(=O)NCc3cccc(OC(F)(F)F)c3)nn2)c(=O)c1] | 1-{4-[4-(2-cyclopentylacetamido)-2- oxo-1,2-dihydropyridin-1-yl]-2- fluorobutyl}-N-{[3- (trifluoromethoxy)phenyl]methyl}-1H- 1,2,3-triazole-4-carboxamide |
| 221 | 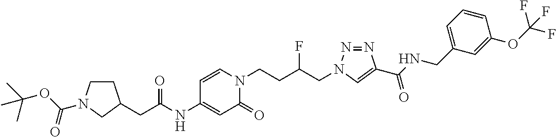 [SMILES: CC(C)(C)OC(=O)N1CCC(CC(=O)Nc2ccn(CCC(F)Cn3cc(C(=O)NCc4cccc(OC(F)(F)F)c4)nn3)c(=O)c2)C1] | tert-butyl 3-{[(1-{3-fluoro-4-[4-({[3- (trifluoromethoxy)phenyl]methyl} carbamoyl)-1H-1,2,3-triazol-1- yl]butyl}-2-oxo-1,2-dihydropyridin-4- yl)carbamoyl]methyl}pyrrolidine-1- carboxylate |
| 222 | 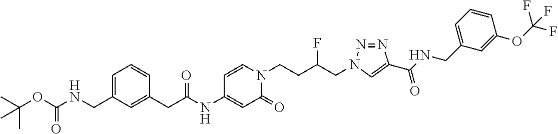 [SMILES: CC(C)(C)OC(=O)NCc1cccc(CC(=O)Nc2ccn(CCC(F)Cn3cc(C(=O)NCc4cccc(OC(F)(F)F)c4)nn3)c(=O)c2)c1] | tert-butyl N-[(3-{[(1-{3-fluoro-4-[4- ({[3-(trifluoro- methoxy)phenyl]methyl}carbamoyl)- 1H-1,2,3-triazol-1-yl]butyl}-2- oxo-1,2-dihydropyridin-4- yl)carbamoyl]methyl}phenyl)methyl] carbamate |
| 223 | 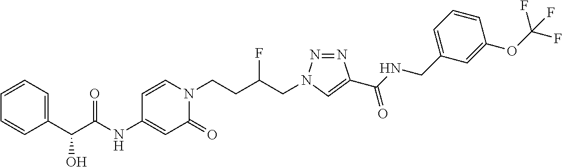 [SMILES: O=C(NCc1cccc(OC(F)(F)F)c1)c1cn(CC(F)CCn2ccc(NC(=O)[C@H](O)c3ccccc3)cc2=O)nn1] | 1-(2-fluoro-4-{4-[(2R)-2-hydroxy-2- phenylacetamido]-2-oxo-1,2- dihydropyridin-1-yl}butyl)-N-{[3- (trifluoromethoxy)phenyl]methyl}-1H- 1,2,3-triazole-4-carboxamide |
| 224 | 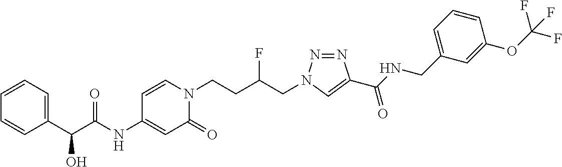 [SMILES: O=C(NCc1cccc(OC(F)(F)F)c1)c1cn(CC(F)CCn2ccc(NC(=O)[C@@H](O)c3ccccc3)cc2=O)nn1] | 1-(2-fluoro-4-{4-[(2S)-2-hydroxy-2- phenylacetamido]-2-oxo-1,2- dihydropyridin-1-yl}butyl)-N-{[3- (trifluoromethoxy)phenyl]methyl}-1H- 1,2,3-triazole-4-carboxamide |
| 225 | 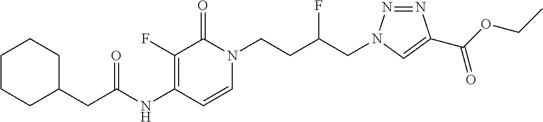 [SMILES: CCOC(=O)c1cn(CC(F)CCn2ccc(NC(=O)CC3CCCCC3)c(F)c2=O)nn1] | ethyl 1-{4-[4-(2-cyclohexylacetamido)- 3-fluoro-2-oxo-1,2-dihydropyridin-1- yl]-2-fluorobutyl}-1H-1,2,3-triazole-4- carboxylate |
| 226 | 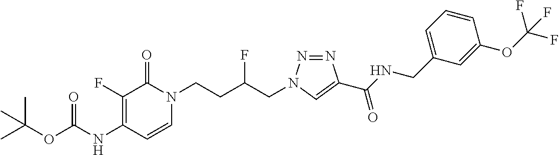 [SMILES: CC(C)(C)OC(=O)Nc1ccn(CCC(F)Cn2cc(C(=O)NCc3cccc(OC(F)(F)F)c3)nn2)c(=O)c1F] | tert-butyl N-(3-fluoro-1-{3-fluoro-4-[4- ({[3-(trifluoro- methoxy)phenyl]methyl}carbamoyl)- 1H-1,2,3-triazol-1-yl]butyl}-2- oxo-1,2-dihydropyridin-4-yl)carbamate |
| 227 | 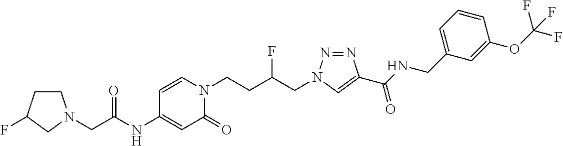 [SMILES: O=C(CN1CCC(F)C1)Nc1ccn(CCC(F)Cn2cc(C(=O)NCc3cccc(OC(F)(F)F)c3)nn2)c(=O)c1] | 1-(2-fluoro-4-{4-[2-(3-fluoropyrrolidin- 1-yl)acetamido]-2-oxo-1,2- dihydropyridin-1-yl}butyl)-N-{[3- (trifluoromethoxy)phenyl]methyl}-1H- 1,2,3-triazole-4-carboxamide |
| 228 | 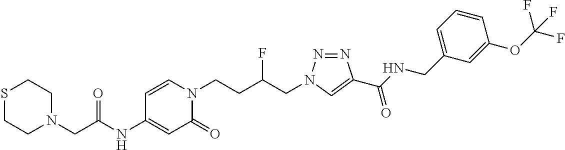 [SMILES: O=C(CN1CCSCC1)Nc1ccn(CCC(F)Cn2cc(C(=O)NCc3cccc(OC(F)(F)F)c3)nn2)c(=O)c1] | 1-(2-fluoro-4-{2-oxo-4-[2- (thiomorpholin-4-yl)acetamido]-1,2- dihydropyridin-1-yl}butyl)-N-{[3- (trifluoromethoxy)phenyl]methyl}-1H- 1,2,3-triazole-4-carboxamide |
| 229 | 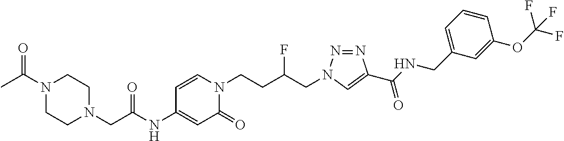 [SMILES: CC(=O)N1CCN(CC(=O)Nc2ccn(CCC(F)Cn3cc(C(=O)NCc4cccc(OC(F)(F)F)c4)nn3)c(=O)c2)CC1] | 1-(4-{4-[2-(4-acetylpiperazin-1- yl)acetamido]-2-oxo-1,2- dihydropyridin-1-yl}-2-fluorobutyl)-N- {[3-(trifluoromethoxy)phenyl]methyl}- 1H-1,2,3-triazole-4-carboxamide |
| 230 | 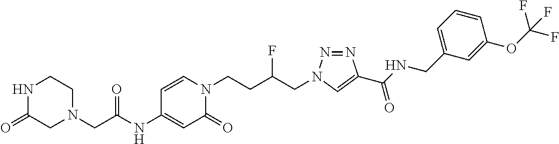 [SMILES: O=C1CN(CC(=O)Nc2ccn(CCC(F)Cn3cc(C(=O)NCc4cccc(OC(F)(F)F)c4)nn3)c(=O)c2)CCN1] | 1-(2-fluoro-4-{2-oxo-4-[2-(3- oxopiperazin-1-yl)acetamido]-1,2- dihydropyridin-1-yl}butyl)-N-{[3- (trifluoromethoxy)phenyl]methyl}-1H- 1,2,3-triazole-4-carboxamide |
| 231 | 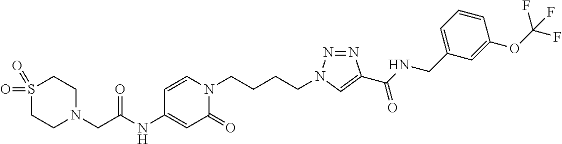 [SMILES: O=C(CN1CCS(=O)(=O)CC1)Nc1ccn(CCCCn2cc(C(=O)NCc3cccc(OC(F)(F)F)c3)nn2)c(=O)c1] | 1-(4-{4-[2-(1,1-dioxo-1$l\hat{ }{6},4- thiomorpholin-4-yl)acetamido]-2-oxo- 1,2-dihydropyridin-1-yl}-2- fluorobutyl)-N-{[3- (trifluoromethoxy)phenyl]methyl}-1H- 1,2,3-triazole-4-carboxamide |
| 232 | 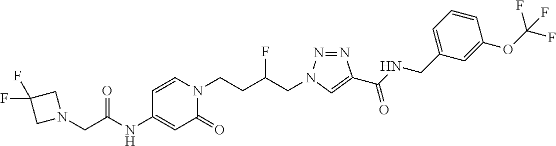 [SMILES: O=C(CN1CC(F)(F)C1)Nc1ccn(CCC(F)Cn2cc(C(=O)NCc3cccc(OC(F)(F)F)c3)nn2)c(=O)c1] | 1-(4-{4-[2-(3,3-difluoroazetidin-1- yl)acetamido]-2-oxo-1,2- dihydropyridin-1-yl}-2-fluorobutyl)-N- {[3-(trifluoromethoxy)phenyl]methyl}- 1H-1,2,3-triazole-4-carboxamide |
| 233 | 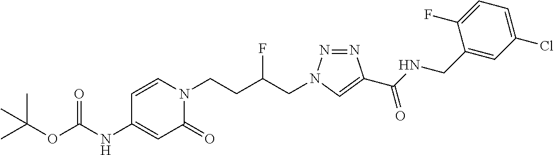 [SMILES: CC(C)(C)OC(=O)Nc1ccn(CCC(F)Cn2cc(C(=O)NCc3cc(Cl)ccc3F)nn2)c(=O)c1] | tert-butyl N-{1-[4-(4-{[(5-chloro-2- fluorophenyl)methyl]carbamoyl}-1H- 1,2,3-triazol-1-yl)-3-fluorobutyl]-2-oxo- 1,2-dihydropyridin-4-yl}carbamate |
| 234 | 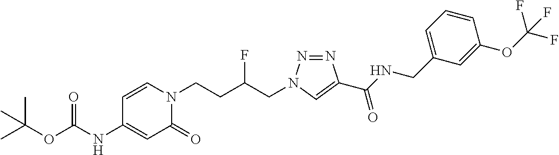 [SMILES: CC(C)(C)OC(=O)Nc1ccn(CCC(F)Cn2cc(C(=O)NCc3cccc(OC(F)(F)F)c3)nn2)c(=O)c1] | tert-butyl N-(1-{3-fluoro-4-[4-({[3- (trifluoromethoxy)phenyl]methyl] carbamoyl)-1H-1,2,3-triazol- 1-yl]butyl}-2-oxo-1,2- dihydropyridin-4-yl)carbamate |
| 235 | 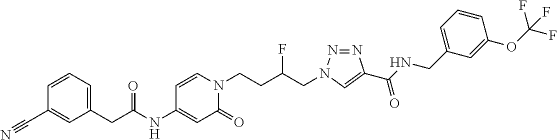 [SMILES: N#Cc1cccc(CC(=O)Nc2ccn(CCC(F)Cn3cc(C(=O)NCc4cccc(OC(F)(F)F)c4)nn3)c(=O)c2)c1] | 1-(4-{4-[2-(3-cyanophenyl)acetamido]- 2-oxo-1,2-dihydropyridin-1-yl}-2- fluorobutyl)-N-{[3- (trifluoromethoxy)phenyl]methyl}-1H- 1,2,3-triazole-4-carboxamide |
| 236 | 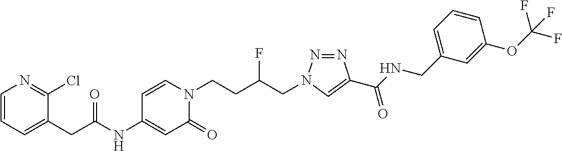 [SMILES: O=C(Cc1cccnc1Cl)Nc1ccn(CCC(F)Cn2cc(C(=O)NCc3cccc(OC(F)(F)F)c3)nn2)c(=O)c1] | 1-(4-{4-[2-(2-chloropyridin-3- yl)acetamido]-2-oxo-1,2- dihydropyridin-1-yl}-2-fluorobutyl)-N- {[3-(trifluoromethoxy)phenyl]methyl}- 1H-1,2,3-triazole-4-carboxamide |
| 237 | 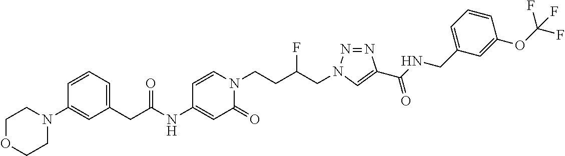 [SMILES: O=C(Cc1cccc(N2CCOCC2)c1)Nc1ccn(CCC(F)Cn2cc(C(=O)NCc3cccc(OC(F)(F)F)c3)nn2)c(=O)c1] | 1-[2-fluoro-4-(4-{2-[3-(morpholin-4- yl)phenyl]acetamido}-2-oxo-1,2- dihydropyridin-1-yl)butyl]-N-{[3- (trifluoromethoxy)phenyl]methyl}-1H- 1,2,3-triazole-4-carboxamide |
| 238 | 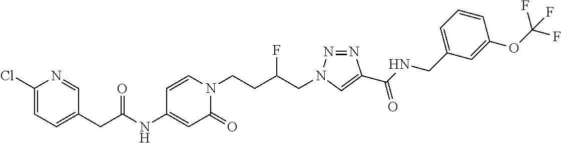 [SMILES: O=C(Cc1ccc(Cl)nc1)Nc1ccn(CCC(F)Cn2cc(C(=O)NCc3cccc(OC(F)(F)F)c3)nn2)c(=O)c1] | 1-(4-{4-[2-(6-chloropyridin-3- yl)acetamido]-2-oxo-1,2- dihydropyridin-1-yl}-2-fluorobutyl)-N- {[3-(trifluoromethoxy)phenyl]methyl}- 1H-1,2,3-triazole-4-carboxamide |
| 239 | 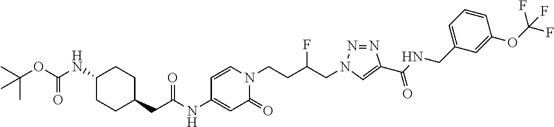 [SMILES: CC(C)(C)OC(=O)N[C@H]1CC[C@H](CC(=O)Nc2ccn(CCC(F)Cn3cc(C(=O)NCc4cccc(OC(F)(F)F)c4)nn3)c(=O)c2)CC1] | tert-butyl N-[(1r,4r)-4-{[(1-{3-fluoro-4- [4-({[3- (trifluoromethoxy)phenyl]methyl} carbamoyl)-1H-1,2,3-triazol- 1-yl]butyl}-2- oxo-1,2-dihydropyridin-4- yl)carbamoyl]methyl}cyclo- hexyl]carbamate |
| 240 | 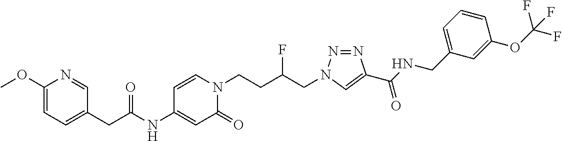 [SMILES: COc1ccc(CC(=O)Nc2ccn(CCC(F)Cn3cc(C(=O)NCc4cccc(OC(F)(F)F)c4)nn3)c(=O)c2)cn1] | 1-(2-fluoro-4-{4-[2-(6-methoxypyridin- 3-yl)acetamido]-2-oxo-1,2- dihydropyridin-1-yl}butyl)-N-{[3- (trifluoromethoxy)phenyl]methyl}-1H- 1,2,3-triazole-4-carboxamide |
| 241 | 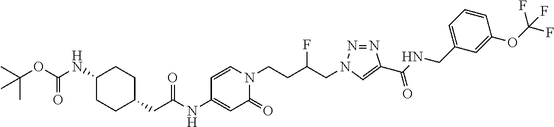 [SMILES: CC(C)(C)OC(=O)N[C@H]1CC[C@@H](CC(=O)Nc2ccn(CCC(F)Cn3cc(C(=O)NCc4cccc(OC(F)(F)F)c4)nn3)c(=O)c2)CC1] | tert-butyl N-[(1s,4s)-4-{[(1-{3-fluoro- 4-[4-({[3-(trifluoromethoxy)phenyl] methyl}carbamoyl)-1H-1,2,3-triazol- 1-yl]butyl}-2-oxo-1,2- dihydropyridin-4-yl)carbamoyl] methyl}cyclohexyl]carbamate |
| 242 | 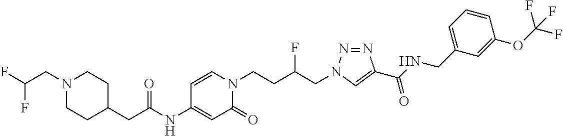 [SMILES: O=C(CC1CCN(CC(F)F)CC1)Nc1ccn(CCC(F)Cn2cc(C(=O)NCc3cccc(OC(F)(F)F)c3)nn2)c(=O)c1] | 1-[4-(4-{2-[1-(2,2- difluoroethyl)piperidin-4- yl]acetamido}-2-oxo-1,2- dihydropyridin-1-yl)-2-fluorobutyl]-N- {[3-(trifluoromethoxy)phenyl]methyl}- 1H-1,2,3-triazole-4-carboxamide |
| 243 | 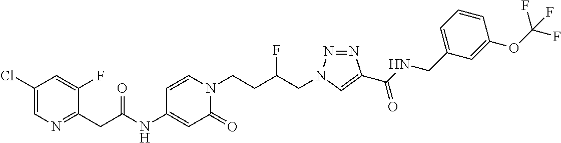 [SMILES: O=C(Cc1ncc(Cl)cc1F)Nc1ccn(CCC(F)Cn2cc(C(=O)NCc3cccc(OC(F)(F)F)c3)nn2)c(=O)c1] | 1-(4-{4-[2-(5-chloro-3-fluoropyridin-2- yl)acetamido]-2-oxo-1,2- dihydropyridin-1-yl}-2-fluorobutyl)-N- {[3-(trifluoromethoxy)phenyl]methyl}- 1H-1,2,3-triazole-4-carboxamide |
| 244 | 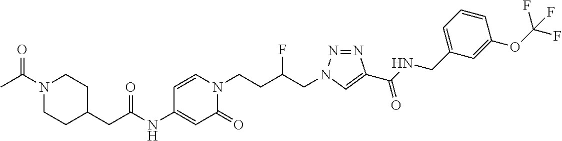 [SMILES: CC(=O)N1CCC(CC(=O)Nc2ccn(CCC(F)Cn3cc(C(=O)NCc4cccc(OC(F)(F)F)c4)nn3)c(=O)c2)CC1] | 1-(4-{4-[2-(1-acetylpiperidin-4- yl)acetamido]-2-oxo-1,2- dihydropyridin-1-yl}-2-fluorobutyl)-N- {[3-(trifluoromethoxy)phenyl]methyl}- 1H-1,2,3-triazole-4-carboxamide |
| 245 | 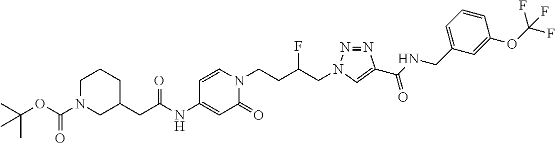 [SMILES: CC(C)(C)OC(=O)N1CCCC(CC(=O)Nc2ccn(CCC(F)Cn3cc(C(=O)NCc4cccc(OC(F)(F)F)c4)nn3)c(=O)c2)C1] | tert-butyl 3-{[(1-{3-fluoro-4-[4-({[3- (trifluoromethoxy)phenyl]methyl} carbamoyl)-1H-1,2,3-triazol- 1-yl]butyl}-2- oxo-1,2-dihydropyridin-4- yl)carbamoyl]methyl}piperidine-1- carboxylate |
| 246 | 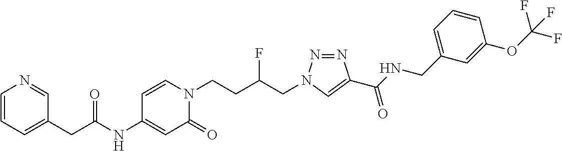 [SMILES: O=C(Cc1cccnc1)Nc1ccn(CCC(F)Cn2cc(C(=O)NCc3cccc(OC(F)(F)F)c3)nn2)c(=O)c1] | 1-(2-fluoro-4-{2-oxo-4-[2-(pyridin-3- yl)acetamido]-1,2-dihydropyridin-1- yl}butyl)-N-{[3- (trifluoromethoxy)phenyl]methyl}-1H- 1,2,3-triazole-4-carboxamide |
| 247 | 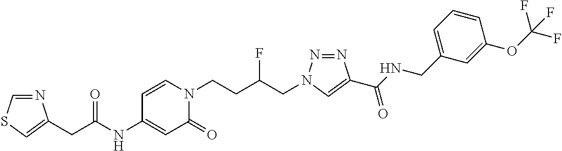 [SMILES: O=C(Cc1cscn1)Nc1ccn(CCC(F)Cn2cc(C(=O)NCc3cccc(OC(F)(F)F)c3)nn2)c(=O)c1] | 1-(2-fluoro-4-{2-oxo-4-[2-(1,3-thiazol- 4-yl)acetamido]-1,2-dihydropyridin-1- yl}butyl)-N-{[3- (trifluoromethoxy)phenyl]methyl}-1H- 1,2,3-triazole-4-carboxamide |
| 248 | 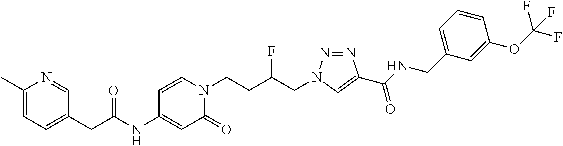 [SMILES: Cc1ccc(CC(=O)Nc2ccn(CCC(F)Cn3cc(C(=O)NCc4cccc(OC(F)(F)F)c4)nn3)c(=O)c2)cn1] | 1-(2-fluoro-4-{4-[2-(6-methylpyridin-3- yl)acetamido]-2-oxo-1,2- dihydropyridin-1-yl}butyl)-N-{[3- (trifluoromethoxy)phenyl]methyl}-1H- 1,2,3-triazole-4-carboxamide |
| 249 | 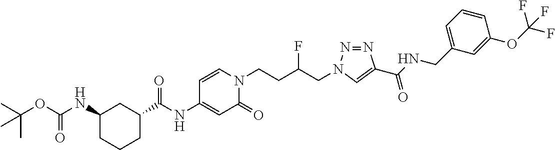 [SMILES: CC(C)(C)OC(=O)N[C@@H]1CCC[C@@H](C(=O)Nc2ccn(CCC(F)Cn3cc(C(=O)NCc4cccc(OC(F)(F)F)c4)nn3)c(=O)c2)C1] | tert-butyl N-[(1R,3R)-3-[(1-{3-fluoro-4- [4-({[3- (trifluoromethoxy)phenyl]methyl} carbamoyl)-1H-1,2,3-triazol- 1-yl]butyl}-2- oxo-1,2-dihydropyridin-4- yl)carbamoyl]cyclohexyl]carbamate |
| 250 | 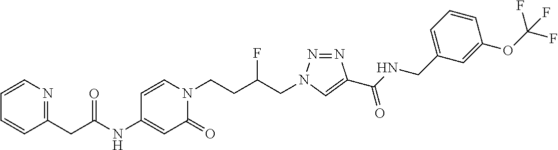 [SMILES: O=C(Cc1ccccn1)Nc1ccn(CCC(F)Cn2cc(C(=O)NCc3cccc(OC(F)(F)F)c3)nn2)c(=O)c1] | 1-(2-fluoro-4-{2-oxo-4-[2-(pyridin-2- yl)acetamido]-1,2-dihydropyridin-1- yl}butyl)-N-{[3- (trifluoromethoxy)phenyl]methyl}-1H- 1,2,3-triazole-4-carboxamide |
| 251 | 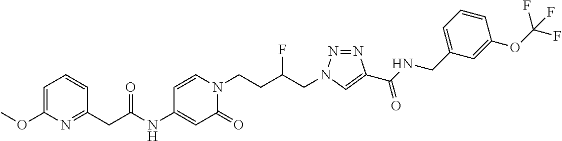 [SMILES: COc1cccc(CC(=O)Nc2ccn(CCC(F)Cn3cc(C(=O)NCc4cccc(OC(F)(F)F)c4)nn3)c(=O)c2)n1] | 1-(2-fluoro-4-{4-[2-(6-methoxypyridin- 2-yl)acetamido]-2-oxo-1,2- dihydropyridin-1-yl}butyl)-N-{[3- (trifluoromethoxy)phenyl]methyl}-1H- 1,2,3-triazole-4-carboxamide |
| 252 | 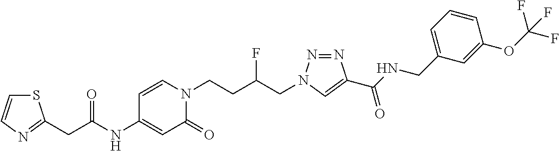 [SMILES: O=C(Cc1nccs1)Nc1ccn(CCC(F)Cn2cc(C(=O)NCc3cccc(OC(F)(F)F)c3)nn2)c(=O)c1] | 1-(2-fluoro-4-{2-oxo-4-[2-(1,3-thiazol- 2-yl)acetamido]-1,2-dihydropyridin-1- yl}butyl)-N-{[3- (trifluoromethoxy)phenyl]methyl}-1H- 1,2,3-triazole-4-carboxamide |
| 253 | 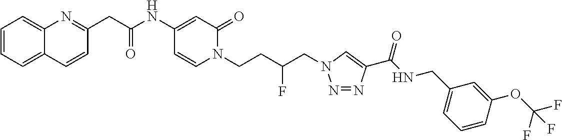 [SMILES: O=C(Cc1ccc2ccccc2n1)Nc1ccn(CCC(F)Cn2cc(C(=O)NCc3cccc(OC(F)(F)F)c3)nn2)c(=O)c1] | 1-(2-fluoro-4-{2-oxo-4-[2-(quinolin-2- yl)acetamido]-1,2-dihydropyridin-1- yl}butyl)-N-{[3- (trifluoromethoxy)phenyl]methyl}-1H- 1,2,3-triazole-4-carboxamide |
| 254 | 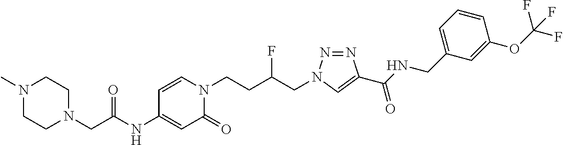 [SMILES: CN1CCN(CC(=O)Nc2ccn(CCC(F)Cn3cc(C(=O)NCc4cccc(OC(F)(F)F)c4)nn3)c(=O)c2)CC1] | 1-(2-fluoro-4-{4-[2-(4-methylpiperazin- 1-yl)acetamido]-2-oxo-1,2- dihydropyridin-1-yl}butyl)-N-{[3- (trifluoromethoxy)phenyl]methyl}-1H- 1,2,3-triazole-4-carboxamide |
| 255 | 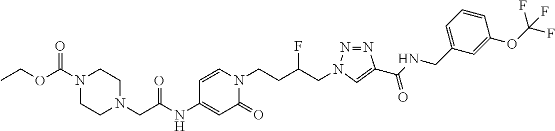 [SMILES: CCOC(=O)N1CCN(CC(=O)Nc2ccn(CCC(F)Cn3cc(C(=O)NCc4cccc(OC(F)(F)F)c4)nn3)c(=O)c2)CC1] | ethyl 4-{[(1-{3-fluoro-4-[4-({[3- (trifluoromethoxy)phenyl]methyl} carbamoyl)-1H-1,2,3-triazol- 1-yl]butyl}-2- oxo-1,2-dihydropyridin-4- yl)carbamoyl]methyl}piperazine-1- carboxylate |
| 256 | 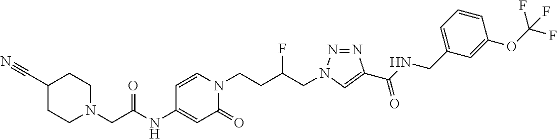 [SMILES: N#CC1CCN(CC(=O)Nc2ccn(CCC(F)Cn3cc(C(=O)NCc4cccc(OC(F)(F)F)c4)nn3)c(=O)c2)CC1] | 1-(4-{4-[2-(4-cyanopiperidin-1- yl)acetamido]-2-oxo-1,2- dihydropyridin-1-yl}-2-fluorobutyl)-N- {[3-(trifluoromethoxy)phenyl]methyl}- 1H-1,2,3-triazole-4-carboxamide |
| 257 | 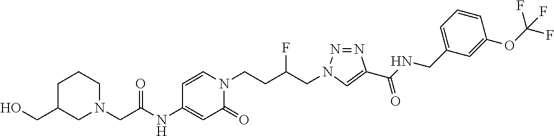 [SMILES: O=C(CN1CCCC(CO)C1)Nc1ccn(CCC(F)Cn2cc(C(=O)NCc3cccc(OC(F)(F)F)c3)nn2)c(=O)c1] | 1-[2-fluoro-4-(4-{2-[3- (hydroxymethyl)piperidin-1- yl]acetamido}-2-oxo-1,2- dihydropyridin-1-yl)butyl]-N-{[3- (trifluoromethoxy)phenyl]methyl}-1H- 1,2,3-triazole-4-carboxamide |
| 258 | 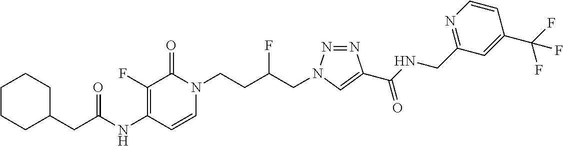 [SMILES: O=C(CC1CCCCC1)Nc1ccn(CCC(F)Cn2cc(C(=O)NCc3cc(C(F)(F)F)ccn3)nn2)c(=O)c1F] | 1-{4-[4-(2-cyclohexylacetamido)-3- fluoro-2-oxo-1,2-dihydropyridin-1-yl]- 2-fluorobutyl}-N-{[4- (trifluoromethyl)pyridin-2-yl]methyl}- 1H-1,2,3-triazole-4-carboxamide |
| 259 | 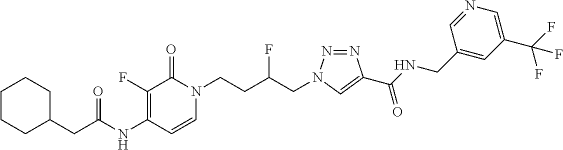 [SMILES: O=C(CC1CCCCC1)Nc1ccn(CCC(F)Cn2cc(C(=O)NCc3cncc(C(F)(F)F)c3)nn2)c(=O)c1F] | 1-{4-[4-(2-cyclohexylacetamido)-3- fluoro-2-oxo-1,2-dihydropyridin-1-yl]- 2-fluorobutyl}-N-{[5- (trifluoromethyl)pyridin-3-yl]methyl}- 1H-1,2,3-triazole-4-carboxamide |
| 260 | 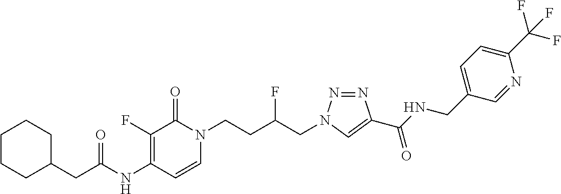 [SMILES: O=C(CC1CCCCC1)Nc1ccn(CCC(F)Cn2cc(C(=O)NCc3ccc(C(F)(F)F)nc3)nn2)c(=O)c1F] | 1-{4-[4-(2-cyclohexylacetamido)-3- fluoro-2-oxo-1,2-dihydropyridin-1-yl]- 2-fluorobutyl}-N-{[6- (trifluoromethyl)pyridin-3-yl]methyl}- 1H-1,2,3-triazole-4-carboxamide |
| 261 | 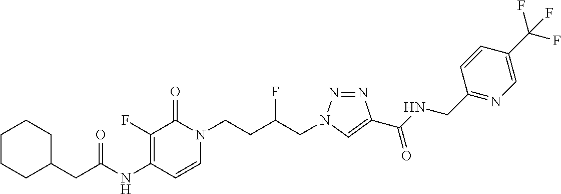 [SMILES: O=C(CC1CCCCC1)Nc1ccn(CCC(F)Cn2cc(C(=O)NCc3ccc(C(F)(F)F)cn3)nn2)c(=O)c1F] | 1-{4-[4-(2-cyclohexylacetamido)-3- fluoro-2-oxo-1,2-dihydropyridin-1-yl]- 2-fluorobutyl}-N-{[5- (trifluoromethyl)pyridin-2-yl]methyl}- 1H-1,2,3-triazole-4-carboxamide |
| 262 | 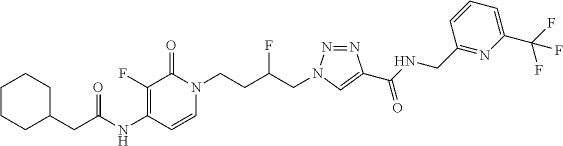 [SMILES: O=C(CC1CCCCC1)Nc1ccn(CCC(F)Cn2cc(C(=O)NCc3cccc(C(F)(F)F)n3)nn2)c(=O)c1F] | 1-{4-[4-(2-cyclohexylacetamido)-3- fluoro-2-oxo-1,2-dihydropyridin-1-yl]- 2-fluorobutyl}-N-{[6- (trifluoromethyl)pyridin-2-yl]methyl}- 1H-1,2,3-triazole-4-carboxamide |
| 263 | 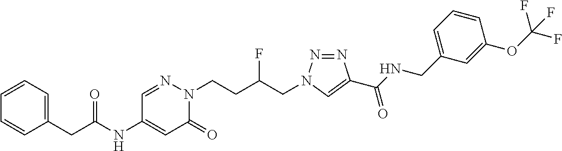 [SMILES: O=C(Cc1ccccc1)Nc1cnn(CCC(F)Cn2cc(C(=O)NCc3cccc(OC(F)(F)F)c3)nn2)c(=O)c1] | 1-{2-fluoro-4-[6-oxo-4-(2- phenylacetamido)-1,6- dihydropyridazin-1-yl]butyl}-N-{[3- (trifluoromethoxy)phenyl]methyl}-1H- 1,2,3-triazole-4-carboxamide |
| 264 | 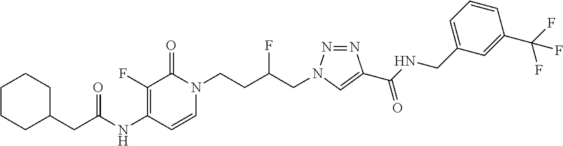 [SMILES: O=C(CC1CCCCC1)Nc1ccn(CCC(F)Cn2cc(C(=O)NCc3cccc(C(F)(F)F)c3)nn2)c(=O)c1F] | 1-{4-[4-(2-cyclohexylacetamido)-3- fluoro-2-oxo-1,2-dihydropyridin-1-yl]- 2-fluorobutyl}-N-{[3- (trifluoromethyl)phenyl]methyl}-1H- 1,2,3-triazole-4-carboxamide |
| 265 | 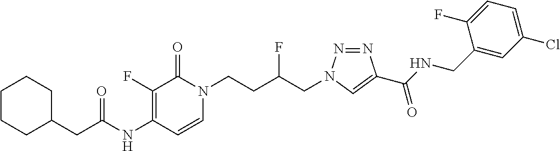 [SMILES: O=C(CC1CCCCC1)Nc1ccn(CCC(F)Cn2cc(C(=O)NCc3cc(Cl)ccc3F)nn2)c(=O)c1F] | N-[(5-chloro-2-fluorophenyl)methyl]-1- {4-[4-(2-cyclohexylacetamido)-3- fluoro-2-oxo-1,2-dihydropyridin-1-yl]- 2-fluorobutyl}-1H-1,2,3-triazole-4- carboxamide |
| 266 | 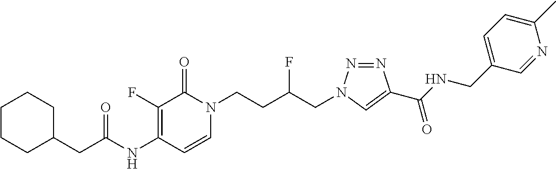 [SMILES: Cc1ccc(CNC(=O)c2cn(CC(F)CCn3ccc(NC(=O)CC4CCCCC4)c(F)c3=O)nn2)cn1] | 1-{4-[4-(2-cyclohexylacetamido)-3- fluoro-2-oxo-1,2-dihydropyridin-1-yl]- 2-fluorobutyl}-N-[(6-methylpyridin-3- yl)methyl]-1H-1,2,3-triazole-4- carboxamide |
| 267 | 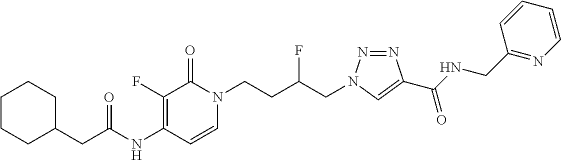 [SMILES: O=C(CC1CCCCC1)Nc1ccn(CCC(F)Cn2cc(C(=O)NCc3ccccn3)nn2)c(=O)c1F] | 1-{4-[4-(2-cyclohexylacetamido)-3- fluoro-2-oxo-1,2-dihydropyridin-1-yl]- 2-fluorobutyl}-N-(pyridin-2-ylmethyl)- 1H-1,2,3-triazole-4-carboxamide |
| 268 | 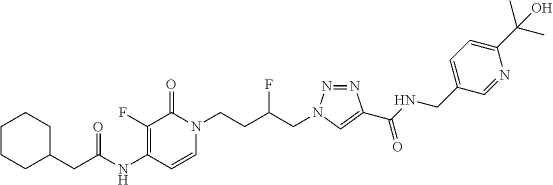 [SMILES: CC(C)(O)c1ccc(CNC(=O)c2cn(CC(F)CCn3ccc(NC(=O)CC4CCCCC4)c(F)c3=O)nn2)cn1] | 1-{4-[4-(2-cyclohexylacetamido)-3- fluoro-2-oxo-1,2-dihydropyridin-1-yl]- 2-fluorobutyl}-N-{[6-(2- hydroxypropan-2-yl)pyridin-3- yl]methyl}-1H-1,2,3-triazole-4- carboxamide |
| 269 | 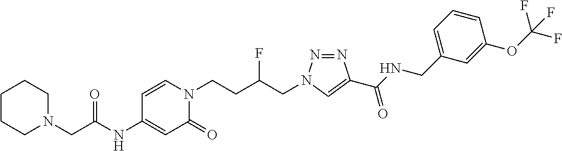 [SMILES: O=C(CN1CCCCC1)Nc1ccn(CCC(F)Cn2cc(C(=O)NCc3cccc(OC(F)(F)F)c3)nn2)c(=O)c1] | 1-(2-fluoro-4-{2-oxo-4-[2-(piperidin-1- yl)acetamido]-1,2-dihydropyridin-1- yl}butyl)-N-{[3- (trifluoromethoxy)phenyl]methyl}-1H- 1,2,3-triazole-4-carboxamide |
| 270 | 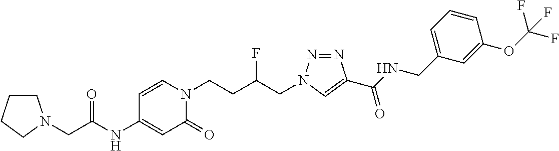 [SMILES: O=C(CN1CCCC1)Nc1ccn(CCC(F)Cn2cc(C(=O)NCc3cccc(OC(F)(F)F)c3)nn2)c(=O)c1] | 1-(2-fluoro-4-{2-oxo-4-[2-(pyrrolidin-1- yl)acetamido]-1,2-dihydropyridin-1- yl}butyl)-N-{[3- (trifluoromethoxy)phenyl]methyl}-1H- 1,2,3-triazole-4-carboxamide |
| 271 | 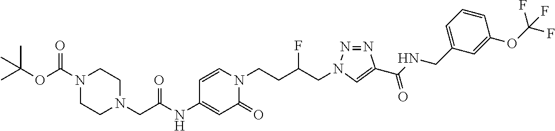 [SMILES: CC(C)(C)OC(=O)N1CCN(CC(=O)Nc2ccn(CCC(F)Cn3cc(C(=O)NCc4cccc(OC(F)(F)F)c4)nn3)c(=O)c2)CC1] | tert-butyl 4-{[(1-{3-fluoro-4-[4-({[3- (trifluoromethoxy)phenyl]methyl} carbamoyl)-1H-1,2,3-triazol- 1-yl]butyl}-2- oxo-1,2-dihydropyridin-4- yl)carbamoyl]methyl}piperazine-1- carboxylate |
| 272 | 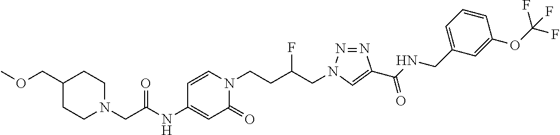 [SMILES: COCC1CCN(CC(=O)Nc2ccn(CCC(F)Cn3cc(C(=O)NCc4cccc(OC(F)(F)F)c4)nn3)c(=O)c2)CC1] | 1-[2-fluoro-4-(4-{2-[4- (methoxymethyl)piperidin-1- yl]acetamido}-2-oxo-1,2- dihydropyridin-1-yl)butyl]-N-{[3- (trifluoromethoxy)phenyl]methyl}-1H- 1,2,3-triazole-4-carboxamide |
| 273 | 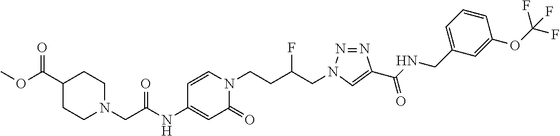 [SMILES: COC(=O)C1CCN(CC(=O)Nc2ccn(CCC(F)Cn3cc(C(=O)NCc4cccc(OC(F)(F)F)c4)nn3)c(=O)c2)CC1] | methyl 1-{[(1-{3-fluoro-4-[4-({[3- (trifluoromethoxy)phenyl]methyl} carbamoyl)-1H-1,2,3-triazol- 1-yl]butyl}-2- oxo-1,2-dihydropyridin-4- yl)carbamoyl]methyl}piperidine-4- carboxylate |
| 274 | 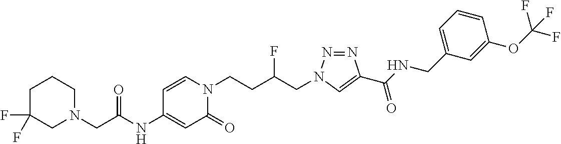 [SMILES: O=C(CN1CCCC(F)(F)C1)Nc1ccn(CCC(F)Cn2cc(C(=O)NCc3cccc(OC(F)(F)F)c3)nn2)c(=O)c1] | 1-(4-{4-[2-(3,3-difluoropiperidin-1- yl)acetamido]-2-oxo-1,2- dihydropyridin-1-yl}-2-fluorobutyl)-N- {[3-(trifluoromethoxy)phenyl]methyl}- 1H-1,2,3-triazole-4-carboxamide |
| 275 | 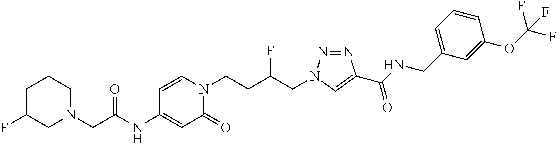 [SMILES: O=C(CN1CCCC(F)C1)Nc1ccn(CCC(F)Cn2cc(C(=O)NCc3cccc(OC(F)(F)F)c3)nn2)c(=O)c1] | 1-(2-fluoro-4-{4-[2-(3-fluoropiperidin- 1-yl)acetamido]-2-oxo-1,2- dihydropyridin-1-yl}butyl)-N-{[3- (trifluoromethoxy)phenyl]methyl}-1H- 1,2,3-triazole-4-carboxamide |
| 276 | 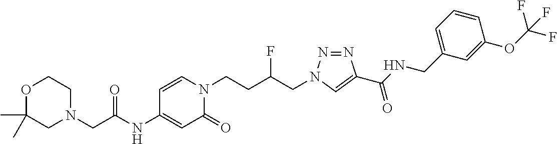 [SMILES: CC1(C)CN(CC(=O)Nc2ccn(CCC(F)Cn3cc(C(=O)NCc4cccc(OC(F)(F)F)c4)nn3)c(=O)c2)CCO1] | 1-(4-{4-[2-(2,2-dimethylmorpholin-4- yl)acetamido]-2-oxo-1,2- dihydropyridin-1-yl}-2-fluorobutyl)-N- {[3-(trifluoromethoxy)phenyl]methyl}- 1H-1,2,3-triazole-4-carboxamide |
| 277 | 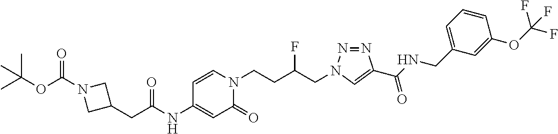 [SMILES: CC(C)(C)OC(=O)N1CC(CC(=O)Nc2ccn(CCC(F)Cn3cc(C(=O)NCc4cccc(OC(F)(F)F)c4)nn3)c(=O)c2)C1] | tert-butyl 3-{[(1-{3-fluoro-4-[4-({[3- (trifluoromethoxy)phenyl]methyl} carbamoyl)-1H-1,2,3-triazol- 1-yl]butyl}-2- oxo-1,2-dihydropyridin-4- yl)carbamoyl]methyl}azetidine-1- carboxylate |
| 278 | 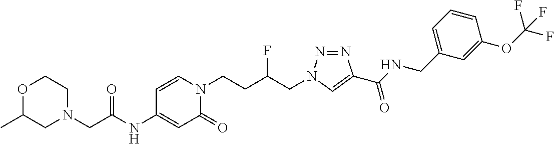 [SMILES: CC1CN(CC(=O)Nc2ccn(CCC(F)Cn3cc(C(=O)NCc4cccc(OC(F)(F)F)c4)nn3)c(=O)c2)CCO1] | 1-(2-fluoro-4-{4-[2-(2- methylmorpholin-4-yl)acetamido]-2- oxo-1,2-dihydropyridin-1-yl}butyl)-N- {[3-(trifluoromethoxy)phenyl]methyl}- 1H-1,2,3-triazole-4-carboxamide |
| 279 | 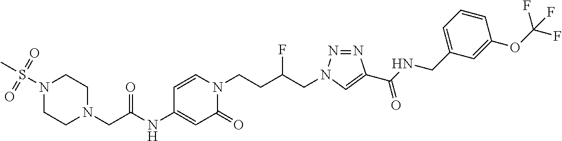 [SMILES: CS(=O)(=O)N1CCN(CC(=O)Nc2ccn(CCC(F)Cn3cc(C(=O)NCc4cccc(OC(F)(F)F)c4)nn3)c(=O)c2)CC1] | 1-(2-fluoro-4-{4-[2-(4- methanesulfonylpiperazin-1- yl)acetamido]-2-oxo-1,2- dihydropyridin-1-yl}butyl)-N-{[3- (trifluoromethoxy)phenyl]methyl}-1H- 1,2,3-triazole-4-carboxamide |
| 280 | 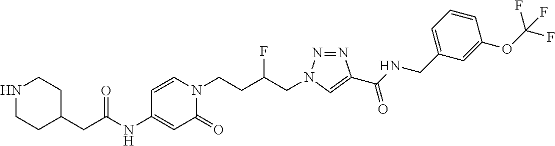 [SMILES: O=C(CC1CCNCC1)Nc1ccn(CCC(F)Cn2cc(C(=O)NCc3cccc(OC(F)(F)F)c3)nn2)c(=O)c1] | 1-(2-fluoro-4-{2-oxo-4-[2-(piperidin-4- yl)acetamido]-1,2-dihydropyridin-1- yl}butyl)-N-{[3- (trifluoromethoxy)phenyl]methyl}-1H- 1,2,3-triazole-4-carboxamide |
| 281 | 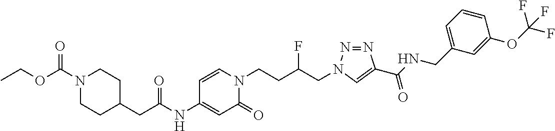 [SMILES: CCOC(=O)N1CCC(CC(=O)Nc2ccn(CCC(F)Cn3cc(C(=O)NCc4cccc(OC(F)(F)F)c4)nn3)c(=O)c2)CC1] | ethyl 4-{[(1-{3-fluoro-4-[4-({[3- (trifluoromethoxy)phenyl]methyl} carbamoyl)-1H-1,2,3-triazol-1- yl]butyl}-2-oxo-1,2- dihydropyridin-4-yl)carbamoyl] methyl}piperidine-1-carboxylate |
| 282 | 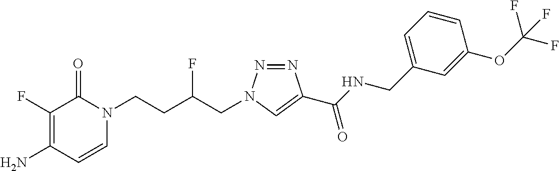 [SMILES: Nc1ccn(CCC(F)Cn2cc(C(=O)NCc3cccc(OC(F)(F)F)c3)nn2)c(=O)c1F] | 1-[4-(4-amino-3-fluoro-2-oxo-1,2- dihydropyridin-1-yl)-2-fluorobutyl]-N- {[3-(trifluoromethoxy)phenyl]methyl}- 1H-1,2,3-triazole-4-carboxamide |
| 283 | 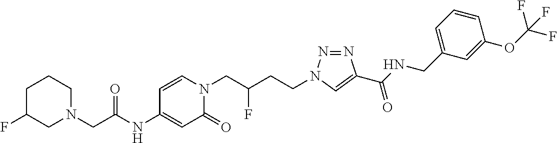 [SMILES: O=C(CN1CCCC(F)C1)Nc1ccn(CC(F)CCn2cc(C(=O)NCc3cccc(OC(F)(F)F)c3)nn2)c(=O)c1] | 1-(3-fluoro-4-{4-[2-(3-fluoropiperidin- 1-yl)acetamido]-2-oxo-1,2- dihydropyridin-1-yl}butyl)-N-{[3- (trifluoromethoxy)phenyl]methyl}-1H- 1,2,3-triazole-4-carboxamide |
| 284 | 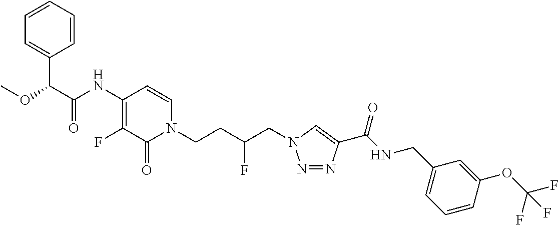 [SMILES: CO[C@@H](C(=O)Nc1ccn(CCC(F)Cn2cc(C(=O)NCc3cccc(OC(F)(F)F)c3)nn2)c(=O)c1F)c1ccccc1] | 1-(2-fluoro-4-{3-fluoro-4-[(2R)-2- methoxy-2-phenylacetamido]-2-oxo- 1,2-dihydropyridin-1-yl}butyl)-N-{[3- (trifluoromethoxy)phenyl]methyl}-1H- 1,2,3-triazole-4-carboxamide |
| 285 | 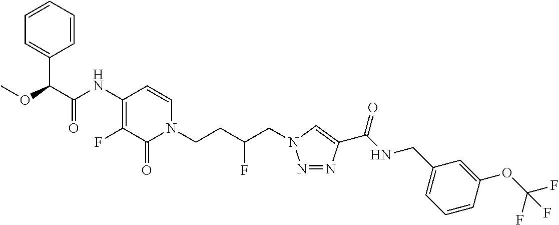 [SMILES: CO[C@H](C(=O)Nc1ccn(CCC(F)Cn2cc(C(=O)NCc3cccc(OC(F)(F)F)c3)nn2)c(=O)c1F)c1ccccc1] | 1-(2-fluoro-4-{3-fluoro-4-[(2S)-2- methoxy-2-phenylacetamido]-2-oxo- 1,2-dihydropyridin-1-yl}butyl)-N-{[3- (trifluoromethoxy)phenyl]methyl}-1H- 1,2,3-triazole-4-carboxamide |
| 286 | 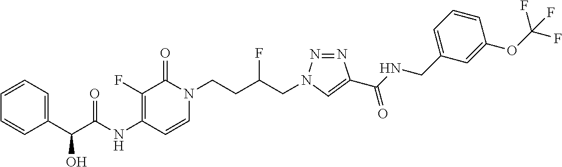 [SMILES: O=C(NCc1cccc(OC(F)(F)F)c1)c1cn(CC(F)CCn2ccc(NC(=O)[C@@H](O)c3ccccc3)c(F)c2=O)nn1] | 1-(2-fluoro-4-{3-fluoro-4-[(2S)-2- hydroxy-2-phenylacetamido]-2-oxo-1,2- dihydropyridin-1-yl}butyl)-N-{[3- (trifluoromethoxy)phenyl]methyl}-1H- 1,2,3-triazole-4-carboxamide |
| 287 | 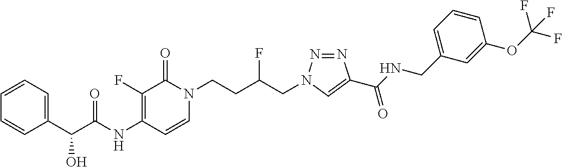 [SMILES: O=C(NCc1cccc(OC(F)(F)F)c1)c1cn(CC(F)CCn2ccc(NC(=O)[C@H](O)c3ccccc3)c(F)c2=O)nn1] | 1-(2-fluoro-4-{3-fluoro-4-[(2R)-2- hydroxy-2-phenylacetamido]-2-oxo-1,2- dihydropyridin-1-yl}butyl)-N-{[3- (trifluoromethoxy)phenyl]methyl}-1H- 1,2,3-triazole-4-carboxamide |
| 288 | 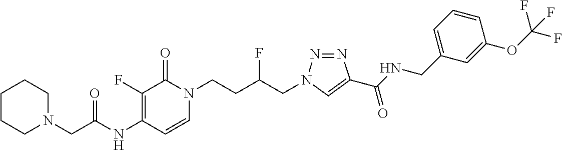 [SMILES: O=C(CN1CCCCC1)Nc1ccn(CCC(F)Cn2cc(C(=O)NCc3cccc(OC(F)(F)F)c3)nn2)c(=O)c1F] | 1-(2-fluoro-4-{3-fluoro-2-oxo-4-[2- (piperidin-1-yl)acetamido]-1,2- dihydropyridin-1-yl}butyl)-N-{[3- (trifluoromethoxy)phenyl]methyl}-1H- 1,2,3-triazole-4-carboxamide |
| 289 | 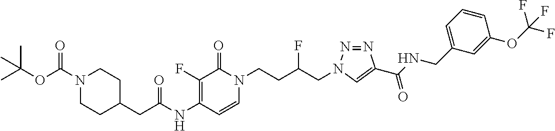 [SMILES: CC(C)(C)OC(=O)N1CCC(CC(=O)Nc2ccn(CCC(F)Cn3cc(C(=O)NCc4cccc(OC(F)(F)F)c4)nn3)c(=O)c2F)CC1] | tert-butyl 4-{[(3-fluoro-1-{3-fluoro-4- [4-({[3-(trifluoro- methoxy)phenyl]methyl}carbamoyl)- 1H-1,2,3-triazol-1-yl]butyl}-2- oxo-1,2-dihydropyridin-4- yl)carbamoyl]methyl}piperidine-1- carboxylate |
| 290 | 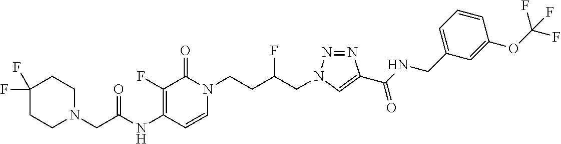 [SMILES: O=C(CN1CCC(F)(F)CC1)Nc1ccn(CCC(F)Cn2cc(C(=O)NCc3cccc(OC(F)(F)F)c3)nn2)c(=O)c1F] | 1-(4-{4-[2-(4,4-difluoropiperidin-1- yl)acetamido]-3-fluoro-2-oxo-1,2- dihydropyridin-1-yl}-2-fluorobutyl)-N- {[3-(trifluoromethoxy)phenyl]methyl}- 1H-1,2,3-triazole-4-carboxamide |
| 291 | 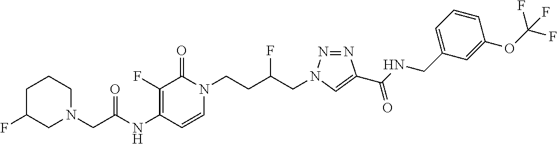 [SMILES: O=C(CN1CCCC(F)C1)Nc1ccn(CCC(F)Cn2cc(C(=O)NCc3cccc(OC(F)(F)F)c3)nn2)c(=O)c1F] | 1-(2-fluoro-4-{3-fluoro-4-[2-(3- fluoropiperidin-1-yl)acetamido]-2-oxo- 1,2-dihydropyridin-1-yl}butyl)-N-{[3- (trifluoromethoxy)phenyl]methyl}-1H- 1,2,3-triazole-4-carboxamide |
| 292 | 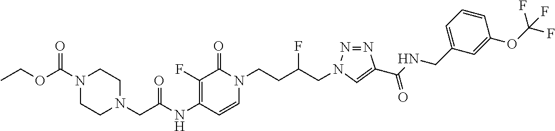 [SMILES: CCOC(=O)N1CCN(CC(=O)Nc2ccn(CCC(F)Cn3cc(C(=O)NCc4cccc(OC(F)(F)F)c4)nn3)c(=O)c2F)CC1] | ethyl 4-{[(3-fluoro-1-{3-fluoro-4-[4- ({[3-(trifluoro- methoxy)phenyl]methyl}carbamoyl)- 1H-1,2,3-triazol-1-yl]butyl}-2- oxo-1,2-dihydropyridin-4- yl)carbamoyl]methyl}piperazine-1- carboxylate |
| 293 | 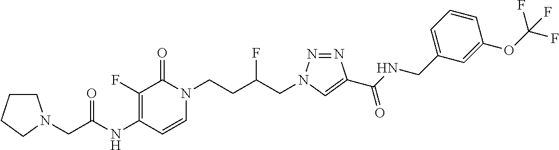 [SMILES: O=C(CN1CCCC1)Nc1ccn(CCC(F)Cn2cc(C(=O)NCc3cccc(OC(F)(F)F)c3)nn2)c(=O)c1F] | 1-(2-fluoro-4-{3-fluoro-2-oxo-4-[2- (pyrrolidin-1-yl)acetamido]-1,2- dihydropyridin-1-yl}butyl)-N-{[3- (trifluoromethoxy)phenyl]methyl}-1H- 1,2,3-triazole-4-carboxamide |
| 294 | 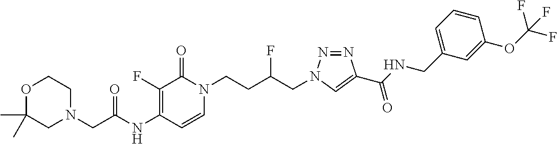 [SMILES: CC1(C)CN(CC(=O)Nc2ccn(CCC(F)Cn3cc(C(=O)NCc4cccc(OC(F)(F)F)c4)nn3)c(=O)c2F)CCO1] | 1-(4-{4-[2-(2,2-dimethylmorpholin-4- yl)acetamido]-3-fluoro-2-oxo-1,2- dihydropyridin-1-yl}-2-fluorobutyl)-N- {[3-(trifluoromethoxy)phenyl]methyl}- 1H-1,2,3-triazole-4-carboxamide |
| 295 | 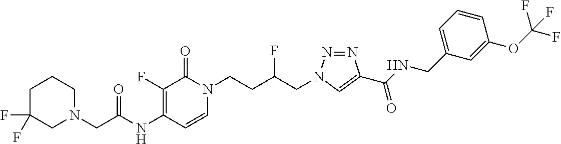 [SMILES: O=C(CN1CCCC(F)(F)C1)Nc1ccn(CCC(F)Cn2cc(C(=O)NCc3cccc(OC(F)(F)F)c3)nn2)c(=O)c1F] | 1-(4-{4-[2-(3,3-difluoropiperidin-1- yl)acetamido]-3-fluoro-2-oxo-1,2- dihydropyridin-1-yl}-2-fluorobutyl)-N- {[3-(trifluoromethoxy)phenyl]methyl}- 1H-1,2,3-triazole-4-carboxamide |
| 296 | 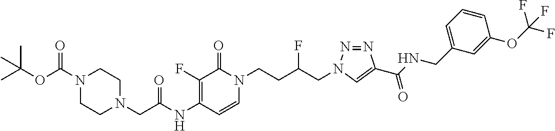 [SMILES: CC(C)(C)OC(=O)N1CCN(CC(=O)Nc2ccn(CCC(F)Cn3cc(C(=O)NCc4cccc(OC(F)(F)F)c4)nn3)c(=O)c2F)CC1] | tert-butyl 4-{[(3-fluoro-1-{3-fluoro-4- [4-({[3-(trifluoro- methoxy)phenyl]methyl}carbamoyl)- 1H-1,2,3-triazol-1-yl]butyl}-2- oxo-1,2-dihydropyridin-4- yl)carbamoyl]methyl}piperazine-1- carboxylate |
| 297 | 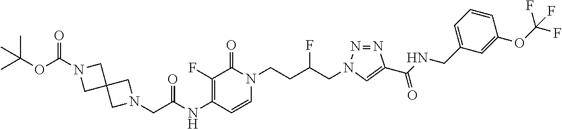 [SMILES: CC(C)(C)OC(=O)N1CC2(CN(CC(=O)Nc3ccn(CCC(F)Cn4cc(C(=O)NCc5cccc(OC(F)(F)F)c5)nn4)c(=O)c3F)C2)C1] | tert-butyl 6-{[(3-fluoro-1-{3-fluoro-4- [4-({[3-(trifluoro- methoxy)phenyl]methyl}carbamoyl)- 1H-1,2,3-triazol-1-yl]butyl}-2- oxo-1,2-dihydropyridin-4- yl)carbamoyl]methyl}-2,6- diazaspiro[3.3]heptane-2-carboxylate |
| 298 | 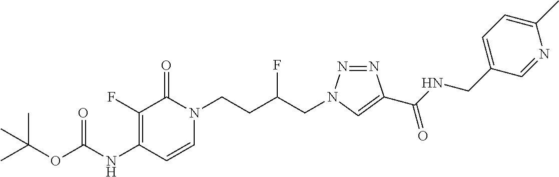 [SMILES: Cc1ccc(CNC(=O)c2cn(CC(F)CCn3ccc(NC(=O)OC(C)(C)C)c(F)c3=O)nn2)cn1] | tert-butyl N-{3-fluoro-1-[3-fluoro-4-(4- {[(6-methylpyridin-3- yl)methyl]carbamoyl}-1H-1,2,3-triazol- 1-yl)butyl]-2-oxo-1,2-dihydropyridin-4- yl}carbamate |
| 299 | 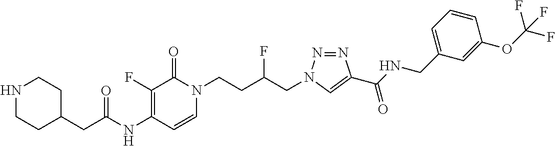 [SMILES: O=C(CC1CCNCC1)Nc1ccn(CCC(F)Cn2cc(C(=O)NCc3cccc(OC(F)(F)F)c3)nn2)c(=O)c1F] | 1-(2-fluoro-4-{3-fluoro-2-oxo-4-[2- (piperidin-4-yl)acetamido]-1,2- dihydropyridin-1-yl}butyl)-N-{[3- (trifluoromethoxy)phenyl]methyl}-1H- 1,2,3-triazole-4-carboxamide |
| 300 | 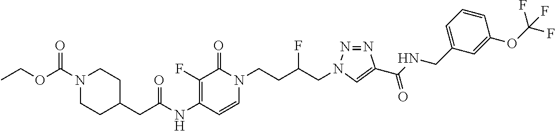 [SMILES: CCOC(=O)N1CCC(CC(=O)Nc2ccn(CCC(F)Cn3cc(C(=O)NCc4cccc(OC(F)(F)F)c4)nn3)c(=O)c2F)CC1] | ethyl 4-{[(3-fluoro-1-{3-fluoro-4-[4- ({[3-(trifluoro- methoxy)phenyl]methyl}carbamoyl)- 1H-1,2,3-triazol-1-yl]butyl}-2- oxo-1,2-dihydropyridin-4- yl)carbamoyl]methyl}piperidine-1- carboxylate |
| 301 | 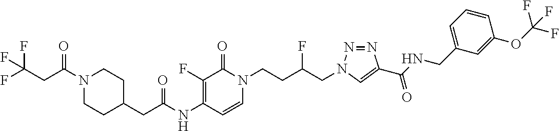 [SMILES: O=C(CC1CCN(C(=O)CC(F)(F)F)CC1)Nc1ccn(CCC(F)Cn2cc(C(=O)NCc3cccc(OC(F)(F)F)c3)nn2)c(=O)c1F] | 1-[2-fluoro-4-(3-fluoro-2-oxo-4-{2-[1- (3,3,3-trifluoropropanoyl)piperidin-4- yl]acetamido}-1,2-dihydropyridin-1- yl)butyl]-N-{[3- (trifluoromethoxy)phenyl]methyl}-1H- 1,2,3-triazole-4-carboxamide |
| 302 | 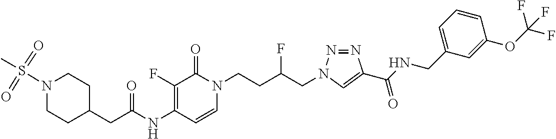 [SMILES: CS(=O)(=O)N1CCC(CC(=O)Nc2ccn(CCC(F)Cn3cc(C(=O)NCc4cccc(OC(F)(F)F)c4)nn3)c(=O)c2F)CC1] | 1-(2-fluoro-4-{3-fluoro-4-[2-(1- methanesulfonylpiperidin-4- yl)acetamido]-2-oxo-1,2- dihydropyridin-1-yl}butyl)-N-{[3- (trifluoromethoxy)phenyl]methyl}-1H- 1,2,3-triazole-4-carboxamide |
| 303 | 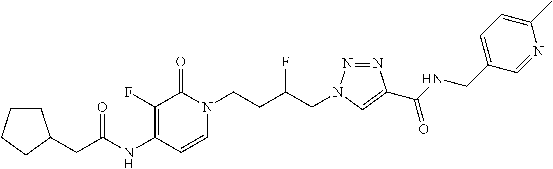 [SMILES: Cc1ccc(CNC(=O)c2cn(CC(F)CCn3ccc(NC(=O)CC4CCCC4)c(F)c3=O)nn2)cn1] | 1-{4-[4-(2-cyclopentylacetamido)-3- fluoro-2-oxo-1,2-dihydropyridin-1-yl]- 2-fluorobutyl}-N-[(6-methylpyridin-3- yl)methyl]-1H-1,2,3-triazole-4- carboxamide |
| 304 | 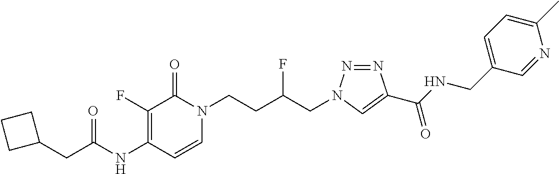 [SMILES: Cc1ccc(CNC(=O)c2cn(CC(F)CCn3ccc(NC(=O)CC4CCC4)c(F)c3=O)nn2)cn1] | 1-{4-[4-(2-cyclobutylacetamido)-3- fluoro-2-oxo-1,2-dihydropyridin-1-yl]- 2-fluorobutyl}-N-[(6-methylpyridin-3- yl)methyl]-1H-1,2,3-triazole-4- carboxamide |
| 305 | 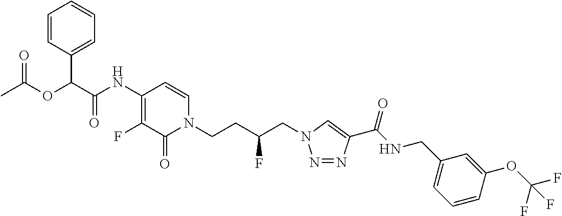 [SMILES: CC(=O)OC(C(=O)Nc1ccn(CC[C@H](F)Cn2cc(C(=O)NCc3cccc(OC(F)(F)F)c3)nn2)c(=O)c1F)c1ccccc1] | (S)-[(3-fluoro-1-{3-fluoro-4-[4-({[3- (trifluoromethoxy)phenyl]methyl} carbamoyl)-1H-1,2,3-triazol- 1-yl]butyl}-2- oxo-1,2-dihydropyridin-4- yl)carbamoyl](phenyl)methyl acetate |
| 306 | 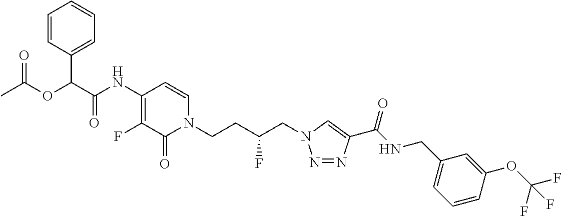 [SMILES: CC(=O)OC(C(=O)Nc1ccn(CC[C@@H](F)Cn2cc(C(=O)NCc3cccc(OC(F)(F)F)c3)nn2)c(=O)c1F)c1ccccc1] | (R)-[(3-fluoro-1-{3-fluoro-4-[4-({[3- (trifluoromethoxy)phenyl]methyl} carbamoyl)-1H-1,2,3-triazol- 1-yl]butyl}-2- oxo-1,2-dihydropyridin-4- yl)carbamoyl](phenyl)methyl acetate |
| 307 | 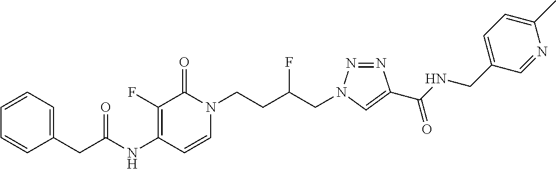 [SMILES: Cc1ccc(CNC(=O)c2cn(CC(F)CCn3ccc(NC(=O)Cc4ccccc4)c(F)c3=O)nn2)cn1] | 1-{2-fluoro-4-[3-fluoro-2-oxo-4-(2- phenylacetamido)-1,2-dihydropyridin- 1-yl]butyl}-N-[(6-methylpyridin-3- yl)methyl]-1H-1,2,3-triazole-4- carboxamide |
| 308 | 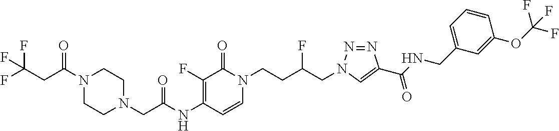 [SMILES: O=C(CN1CCN(C(=O)CC(F)(F)F)CC1)Nc1ccn(CCC(F)Cn2cc(C(=O)NCc3cccc(OC(F)(F)F)c3)nn2)c(=O)c1F] | 1-[2-fluoro-4-(3-fluoro-2-oxo-4-{2-[4- (3,3,3-trifluoropropanoyl)piperazin-1- yl]acetamido}-1,2-dihydropyridin-1- yl)butyl]-N-{[3- (trifluoromethoxy)phenyl]methyl}-1H- 1,2,3-triazole-4-carboxamide |
| 309 | 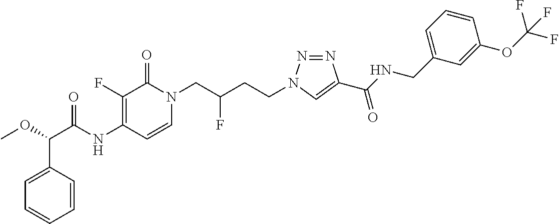 [SMILES: CO[C@H](C(=O)Nc1ccn(CC(F)CCn2cc(C(=O)NCc3cccc(OC(F)(F)F)c3)nn2)c(=O)c1F)c1ccccc1] | 1-(3-fluoro-4-{3-fluoro-4-[(2S)-2- methoxy-2-phenylacetamido]-2-oxo- 1,2-dihydropyridin-1-yl}butyl)-N-{[3- (trifluoromethoxy)phenyl]methyl}-1H- 1,2,3-triazole-4-carboxamide |
| 310 | 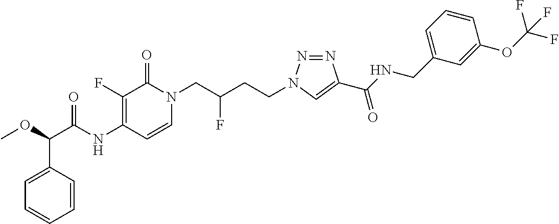 [SMILES: CO[C@@H](C(=O)Nc1ccn(CC(F)CCn2cc(C(=O)NCc3cccc(OC(F)(F)F)c3)nn2)c(=O)c1F)c1ccccc1] | 1-(3-fluoro-4-{3-fluoro-4-[(2R)-2- methoxy-2-phenylacetamido]-2-oxo- 1,2-dihydropyridin-1-yl}butyl)-N-{[3- (trifluoromethoxy)phenyl]methyl}-1H- 1,2,3-triazole-4-carboxamide |
| 311 | 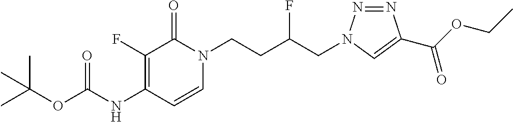 [SMILES: CCOC(=O)c1cn(CC(F)CCn2ccc(NC(=O)OC(C)(C)C)c(F)c2=O)nn1] | ethyl 1-[4-(4-{[(tert- butoxy)carbonyl]amino}-3-fluoro-2- oxo-1,2-dihydropyridin-1-yl)-2- fluorobutyl]-1H-1,2,3-triazole-4- carboxylate |
| 312 | 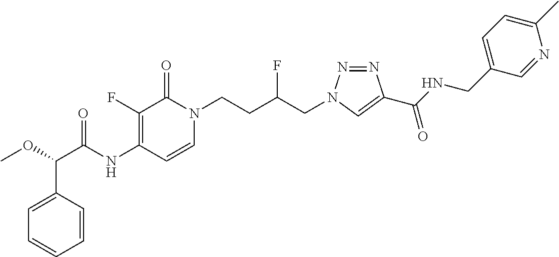 | 1-(2-fluoro-4-{3-fluoro-4-[(2S)-2- methoxy-2-phenylacetamido]-2-oxo- 1,2-dihydropyridin-1-yl}butyl)-N-[(6- methylpyridin-3-yl)methyl]-1H-1,2,3- triazole-4-carboxamide |
| 313 | 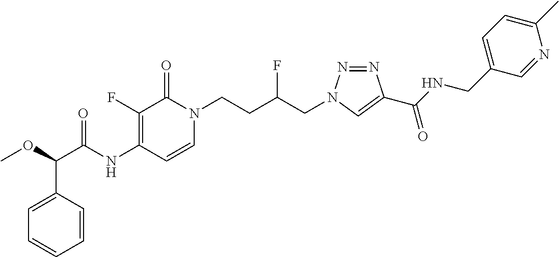 | 1-(2-fluoro-4-{3-fluoro-4-[(2R)-2- methoxy-2-phenylacetamido]-2-oxo- 1,2-dihydropyridin-1-yl}butyl)-N-[(6- methylpyridin-3-yl)methyl]-1H-1,2,3- triazole-4-carboxamide |
| 314 | 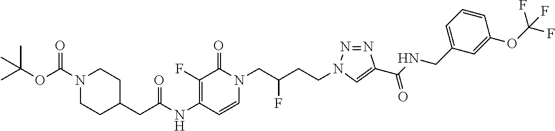 [SMILES: CC(C)(C)OC(=O)N1CCC(CC(=O)Nc2ccn(CC(F)CCn3cc(C(=O)NCc4cccc(OC(F)(F)F)c4)nn3)c(=O)c2F)CC1] | tert-butyl 4-{[(3-fluoro-1-{2-fluoro-4- [4-({[3-(trifluoro- methoxy)phenyl]methyl}carbamoyl)- 1H-1,2,3-triazol-1-yl]butyl}-2- oxo-1,2-dihydropyridin-4- yl)carbamoyl]methyl}piperidine-1- carboxylate |
| 315 | 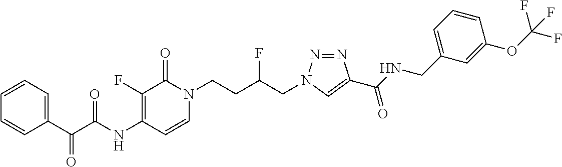 [SMILES: O=C(Nc1ccn(CCC(F)Cn2cc(C(=O)NCc3cccc(OC(F)(F)F)c3)nn2)c(=O)c1F)C(=O)c1ccccc1] | 1-{2-fluoro-4-[3-fluoro-2-oxo-4-(2-oxo- 2-phenylacetamido)-1,2-dihydropyridin- 1-yl]butyl}-N-{[3- (trifluoromethoxy)phenyl]methyl}-1H- 1,2,3-triazole-4-carboxamide |
| 316 | 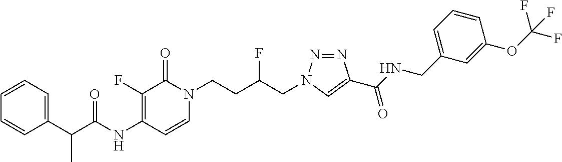 [SMILES: CC(C(=O)Nc1ccn(CCC(F)Cn2cc(C(=O)NCc3cccc(OC(F)(F)F)c3)nn2)c(=O)c1F)c1ccccc1] | 1-{2-fluoro-4-[3-fluoro-2-oxo-4-(2- phenylpropanamido)-1,2- dihydropyridin-1-yl]butyl}-N-{[3- (trifluoromethoxy)phenyl]methyl}-1H- 1,2,3-triazole-4-carboxamide |
| 317 | 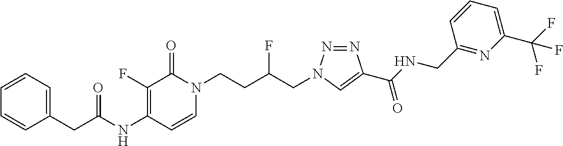 [SMILES: O=C(Cc1ccccc1)Nc1ccn(CCC(F)Cn2cc(C(=O)NCc3cccc(C(F)(F)F)n3)nn2)c(=O)c1F] | 1-{2-fluoro-4-[3-fluoro-2-oxo-4-(2- phenylacetamido)-1,2-dihydropyridin- 1-yl]butyl}-N-{[6- (trifluoromethyl)pyridin-2-yl]methyl}- 1H-1,2,3-triazole-4-carboxamide |
| 318 | 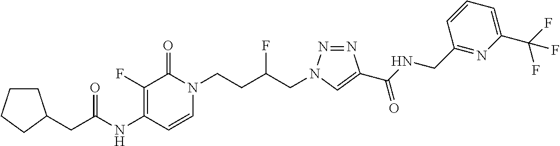 [SMILES: O=C(CC1CCCC1)Nc1ccn(CCC(F)Cn2cc(C(=O)NCc3cccc(C(F)(F)F)n3)nn2)c(=O)c1F] | 1-{4-[4-(2-cyclopentylacetamido)-3- fluoro-2-oxo-1,2-dihydropyridin-1-yl]- 2-fluorobutyl}-N-{[6- (trifluoromethyl)pyridin-2-yl]methyl}- 1H-1,2,3-triazole-4-carboxamide |
| 319 | 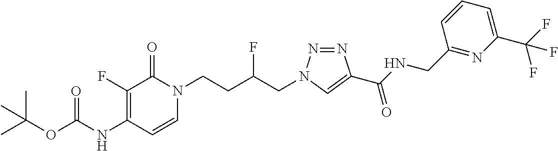 [SMILES: CC(C)(C)OC(=O)Nc1ccn(CCC(F)Cn2cc(C(=O)NCc3cccc(C(F)(F)F)n3)nn2)c(=O)c1F] | tert-butyl N-(3-fluoro-1-{3-fluoro-4-[4- ({[6-(trifluoromethyl)pyridin-2- yl]methyl}carbamoyl)-1H-1,2,3-triazol- 1-yl]butyl}-2-oxo-1,2-dihydropyridin- 4-yl)carbamate |
| 320 | 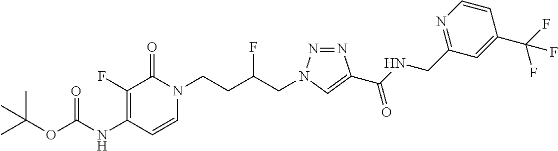 [SMILES: CC(C)(C)OC(=O)Nc1ccn(CCC(F)Cn2cc(C(=O)NCc3cc(C(F)(F)F)ccn3)nn2)c(=O)c1F] | tert-butyl N-(3-fluoro-1-{3-fluoro-4-[4- ({[4-(trifluoromethyl)pyridin-2- yl]methyl}carbamoyl)-1H-1,2,3-triazol- 1-yl]butyl}-2-oxo-1,2-dihydropyridin- 4-yl)carbamate |
| 321 | 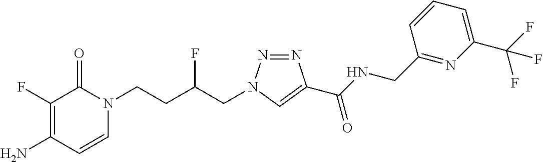 [SMILES: Nc1ccn(CCC(F)Cn2cc(C(=O)NCc3cccc(C(F)(F)F)n3)nn2)c(=O)c1F] | 1-[4-(4-amino-3-fluoro-2-oxo-1,2- dihydropyridin-1-yl)-2-fluorobutyl]-N- {[6-(trifluoromethyl)pyridin-2- yl]methyl}-1H-1,2,3-triazole-4- carboxamide |
| 322 | 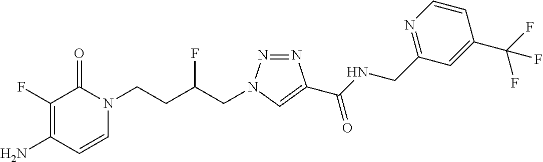 [SMILES: Nc1ccn(CCC(F)Cn2cc(C(=O)NCc3cc(C(F)(F)F)ccn3)nn2)c(=O)c1F] | 1-[4-(4-amino-3-fluoro-2-oxo-1,2- dihydropyridin-1-yl)-2-fluorobutyl]-N- {[4-(trifluoromethyl)pyridin-2- yl]methyl}-1H-1,2,3-triazole-4- carboxamide |
| 323 | 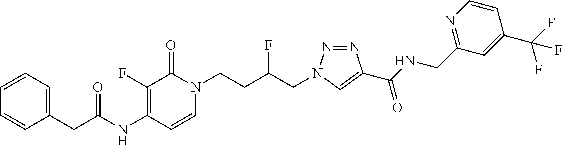 [SMILES: O=C(Cc1ccccc1)Nc1ccn(CCC(F)Cn2cc(C(=O)NCc3cc(C(F)(F)F)ccn3)nn2)c(=O)c1F] | 1-{2-fluoro-4-[3-fluoro-2-oxo-4-(2- phenylacetamido)-1,2-dihydropyridin- 1-yl]butyl}-N-{[4- (trifluoromethyl)pyridin-2-yl]methyl}- 1H-1,2,3-triazole-4-carboxamide |
| 324 | 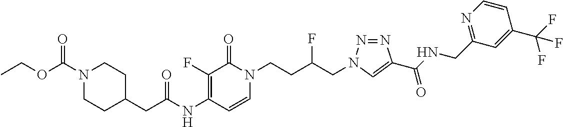 [SMILES: CCOC(=O)N1CCC(CC(=O)Nc2ccn(CCC(F)Cn3cc(C(=O)NCc4cc(C(F)(F)F)ccn4)nn3)c(=O)c2F)CC1] | ethyl 4-{[(3-fluoro-1-{3-fluoro-4-[4- ({[4-(trifluoromethyl)pyridin-2- yl]methyl}carbamoyl)-1H-1,2,3-triazol- 1-yl]butyl}-2-oxo-1,2-dihydropyridin-4- yl)carbamoyl]methyl}piperidine-1- carboxylate |
| 325 | 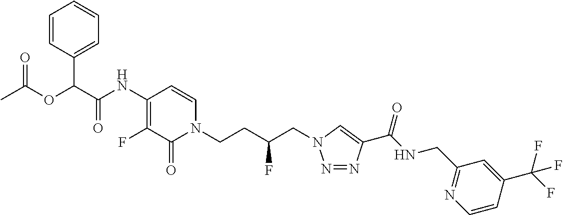 [SMILES: CC(=O)OC(C(=O)Nc1ccn(CC[C@H](F)Cn2cc(C(=O)NCc3cc(C(F)(F)F)ccn3)nn2)c(=O)c1F)c1ccccc1] | (S)-[(3-fluoro-1-{3-fluoro-4-[4-({[4- (trifluoromethyl)pyridin-2- yl]methyl}carbamoyl)-1H-1,2,3-triazol- 1-yl]butyl}-2-oxo-1,2-dihydropyridin-4- yl)carbamoyl](phenyl)methyl acetate |
| 326 | 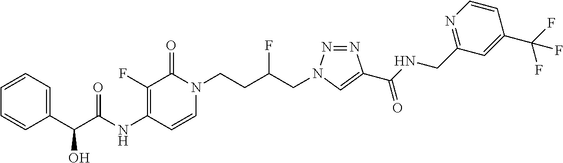 [SMILES: O=C(NCc1cc(C(F)(F)F)ccn1)c1cn(CC(F)CCn2ccc(NC(=O)[C@@H](O)c3ccccc3)c(F)c2=O)nn1] | 1-(2-fluoro-4-{3-fluoro-4-[(2S)-2- hydroxy-2-phenylacetamido]-2-oxo-1,2- dihydropyridin-1-yl}butyl)-N-{[4- (trifluoromethyl)pyridin-2-yl]methyl}- 1H-1,2,3-triazole-4-carboxamide |
| 327 | 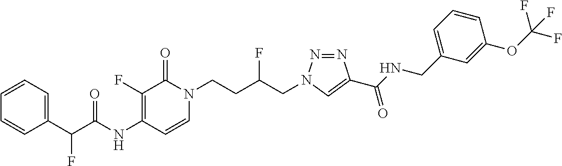 [SMILES: O=C(NCc1cccc(OC(F)(F)F)c1)c1cn(CC(F)CCn2ccc(NC(=O)C(F)c3ccccc3)c(F)c2=O)nn1] | 1-{2-fluoro-4-[3-fluoro-4-(2-fluoro-2- phenylacetamido)-2-oxo-1,2- dihydropyridin-1-yl]butyl}-N-{[3- (trifluoromethoxy)phenyl]methyl}-1H- 1,2,3-triazole-4-carboxamide |
| 328 | 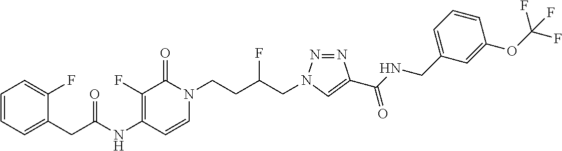 [SMILES: O=C(Cc1ccccc1F)Nc1ccn(CCC(F)Cn2cc(C(=O)NCc3cccc(OC(F)(F)F)c3)nn2)c(=O)c1F] | 1-(2-fluoro-4-{3-fluoro-4-[2-(2- fluorophenyl)acetamido]-2-oxo-1,2- dihydropyridin-1-yl}butyl)-N-{[3- (trifluoromethoxy)phenyl]methyl}-1H- 1,2,3-triazole-4-carboxamide |
| 329 | 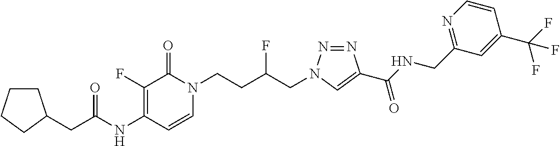 [SMILES: O=C(CC1CCCC1)Nc1ccn(CCC(F)Cn2cc(C(=O)NCc3cc(C(F)(F)F)ccn3)nn2)c(=O)c1F] | 1-{4-[4-(2-cyclopentylacetamido)-3- fluoro-2-oxo-1,2-dihydropyridin-1-yl]- 2-fluorobutyl}-N-{[4- (trifluoromethyl)pyridin-2-yl]methyl}- 1H-1,2,3-triazole-4-carboxamide |
| 330 | 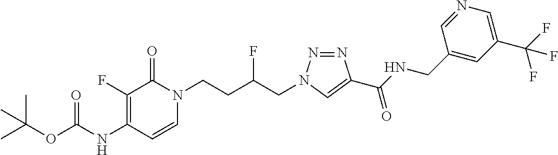 [SMILES: CC(C)(C)OC(=O)Nc1ccn(CCC(F)Cn2cc(C(=O)NCc3cncc(C(F)(F)F)c3)nn2)c(=O)c1F] | tert-butyl N-(3-fluoro-1-{3-fluoro-4-[4- ({[5-(trifluoromethyl)pyridin-3- yl]methyl}carbamoyl)-1H-1,2,3-triazol- 1-yl]butyl}-2-oxo-1,2-dihydropyridin-4- yl)carbamate |
| 331 | 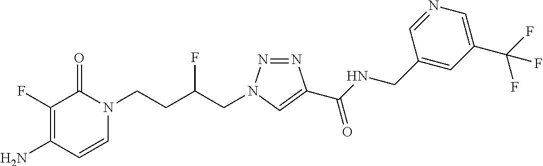 [SMILES: Nc1ccn(CCC(F)Cn2cc(C(=O)NCc3cncc(C(F)(F)F)c3)nn2)c(=O)c1F] | 1-[4-(4-amino-3-fluoro-2-oxo-1,2- dihydropyridin-1-yl)-2-fluorobutyl]-N- {[5-(trifluoromethyl)pyridin-3- yl]methyl}-1H-1,2,3-triazole-4- carboxamide |
| 332 | 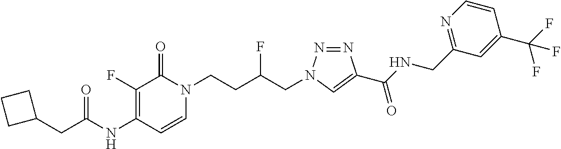 [SMILES: O=C(CC1CCC1)Nc1ccn(CCC(F)Cn2cc(C(=O)NCc3cc(C(F)(F)F)ccn3)nn2)c(=O)c1F] | 1-{4-[4-(2-cyclobutylacetamido)-3- fluoro-2-oxo-1,2-dihydropyridin-1-yl]- 2-fluorobutyl}-N-{[4- (trifluoromethyl)pyridin-2-yl]methyl}- 1H-1,2,3-triazole-4-carboxamide |
| 333 | 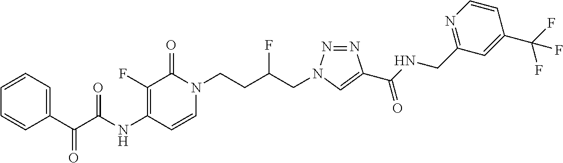 [SMILES: O=C(Nc1ccn(CCC(F)Cn2cc(C(=O)NCc3cc(C(F)(F)F)ccn3)nn2)c(=O)c1F)C(=O)c1ccccc1] | 1-{2-fluoro-4-[3-fluoro-2-oxo-4-(2-oxo- 2-phenylacetamido)-1,2-dihydropyridin- 1-yl]butyl}-N-{[4- (trifluoromethyl)pyridin-2-yl]methyl}- 1H-1,2,3-triazole-4-carboxamide |
| 334 | 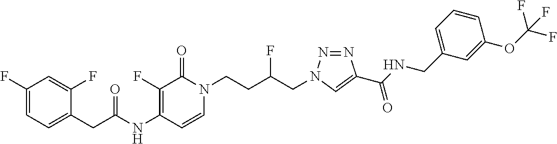 [SMILES: O=C(Cc1ccc(F)cc1F)Nc1ccn(CCC(F)Cn2cc(C(=O)NCc3cccc(OC(F)(F)F)c3)nn2)c(=O)c1F] | 1-(4-{4-[2-(2,4- difluorophenyl)acetamido]-3-fluoro-2- oxo-1,2-dihydropyridin-1-yl}-2- fluorobutyl)-N-{[3- (trifluoromethoxy)phenyl]methyl}-1H- 1,2,3-triazole-4-carboxamide |
| 335 | 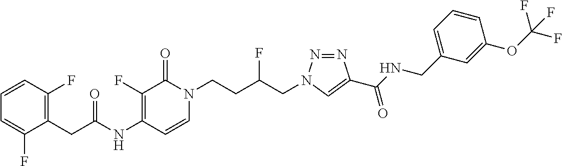 [SMILES: O=C(Cc1c(F)cccc1F)Nc1ccn(CCC(F)Cn2cc(C(=O)NCc3cccc(OC(F)(F)F)c3)nn2)c(=O)c1F] | l-(4-{4-[2-(2,6- difluorophenyl)acetamido]-3-fluoro-2- oxo-1,2-dihydropyridin-1-yl}-2- fluorobutyl)-N-{[3- (trifluoromethoxy)phenyl]methyl}-1H- 1,2,3-triazole-4-carboxamide |
| 336 | 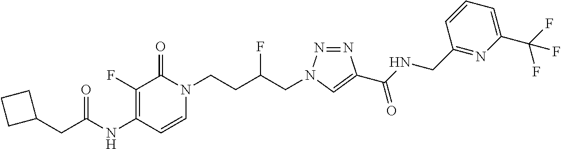 [SMILES: O=C(CC1CCC1)Nc1ccn(CCC(F)Cn2cc(C(=O)NCc3cccc(C(F)(F)F)n3)nn2)c(=O)c1F] | 1-{4-[4-(2-cyclobutylacetamido)-3- fluoro-2-oxo-1,2-dihydropyridin-1-yl]- 2-fluorobutyl}-N-{[6- (trifluoromethyl)pyridin-2-yl]methyl}- 1H-1,2,3-triazole-4-carboxamide |
| 337 | 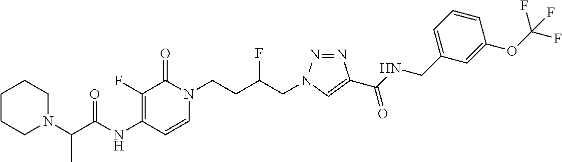 [SMILES: CC(C(=O)Nc1ccn(CCC(F)Cn2cc(C(=O)NCc3cccc(OC(F)(F)F)c3)nn2)c(=O)c1F)N1CCCCC1] | 1-(2-fluoro-4-{3-fluoro-2-oxo-4-[2- (piperidin-1-yl)propanamido]-1,2- dihydropyridin-1-yl}butyl)-N-{[3- (trifluoromethoxy)phenyl]methyl}-1H- 1,2,3-triazole-4-carboxamide |
| 338 | 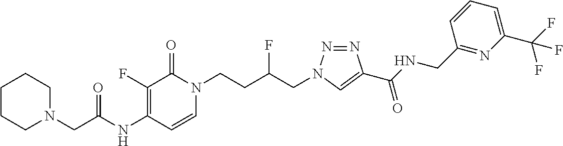 [SMILES: O=C(CN1CCCCC1)Nc1ccn(CCC(F)Cn2cc(C(=O)NCc3cccc(C(F)(F)F)n3)nn2)c(=O)c1F] | 1-(2-fluoro-4-{3-fluoro-2-oxo-4-[2- (piperidin-1-yl)acetamido]-1,2- dihydropyridin-1-yl}butyl)-N-{[6- (trifluoromethyl)pyridin-2-yl]methyl}- 1H-1,2,3-triazole-4-carboxamide |
| 339 | 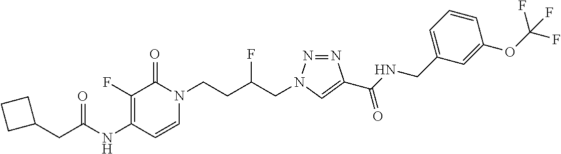 [SMILES: O=C(CC1CCC1)Nc1ccn(CCC(F)Cn2cc(C(=O)NCc3cccc(OC(F)(F)F)c3)nn2)c(=O)c1F] | 1-{4-[4-(2-cyclobutylacetamido)-3- fluoro-2-oxo-1,2-dihydropyridin-1-yl]- 2-fluorobutyl}-N-{[3- (trifluoromethoxy)phenyl]methyl}-1H- 1,2,3-triazole-4-carboxamide |
| 340 | 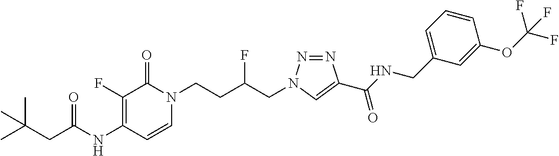 [SMILES: CC(C)(C)CC(=O)Nc1ccn(CCC(F)Cn2cc(C(=O)NCc3cccc(OC(F)(F)F)c3)nn2)c(=O)c1F] | 1-{4-[4-(3,3-dimethylbutanamido)-3- fluoro-2-oxo-1,2-dihydropyridin-1-yl]- 2-fluorobutyl}-N-{[3- (trifluoromethoxy)phenyl]methyl}-1H- 1,2,3-triazole-4-carboxamide |
| 341 | 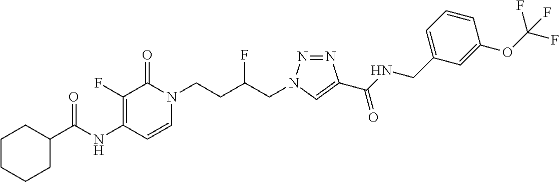 [SMILES: O=C(NCc1cccc(OC(F)(F)F)c1)c1cn(CC(F)CCn2ccc(NC(=O)C3CCCCC3)c(F)c2=O)nn1] | 1-[4-(4-cyclohexaneamido-3-fluoro-2- oxo-1,2-dihydropyridin-1-yl)-2- fluorobutyl]-N-{[3- (trifluoromethoxy)phenyl]methyl}-1H- 1,2,3-triazole-4-carboxamide |
| 342 | 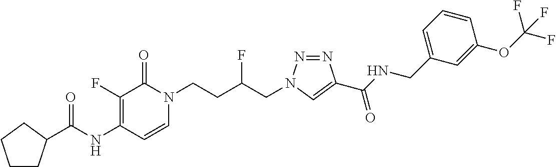 [SMILES: O=C(NCc1cccc(OC(F)(F)F)c1)c1cn(CC(F)CCn2ccc(NC(=O)C3CCCC3)c(F)c2=O)nn1] | 1-[4-(4-cyclopentaneamido-3-fluoro-2- oxo-1,2-dihydropyridin-1-yl)-2- fluorobutyl]-N-{[3- (trifluoromethoxy)phenyl]methyl}-1H- 1,2,3-triazole-4-carboxamide |
| 343 | 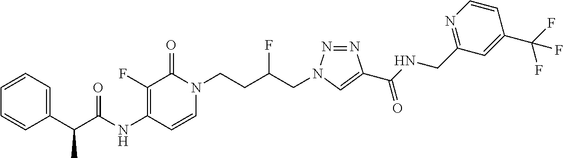 [SMILES: C[C@H](C(=O)Nc1ccn(CCC(F)Cn2cc(C(=O)NCc3cc(C(F)(F)F)ccn3)nn2)c(=O)c1F)c1ccccc1] | 1-(2-fluoro-4-{3-fluoro-2-oxo-4-[(2S) 2-phenylpropanamido]-1,2- dihydropyridin-1-yl}butyl)-N-{[4- (trifluoromethyl)pyridin-2-yl]methyl}- 1H-1,2,3-triazole-4-carboxamide |
| 344 | 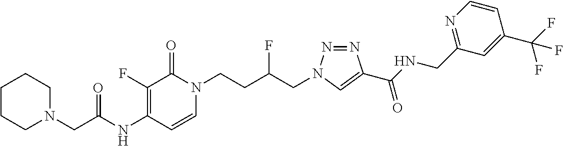 [SMILES: O=C(CN1CCCCC1)Nc1ccn(CCC(F)Cn2cc(C(=O)NCc3cc(C(F)(F)F)ccn3)nn2)c(=O)c1F] | 1-(2-fluoro-4-{3-fluoro-2-oxo-4-[2- (piperidin-1-yl)acetamido]-1,2- dihydropyridin-1-yl}butyl)-N-{[4- (trifluoromethyl)pyridin-2-yl]methyl}- 1H-1,2,3-triazole-4-carboxamide |
| 345 | 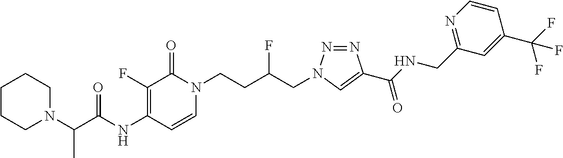 [SMILES: CC(C(=O)Nc1ccn(CCC(F)Cn2cc(C(=O)NCc3cc(C(F)(F)F)ccn3)nn2)c(=O)c1F)N1CCCCC1] | 1-(2-fluoro-4-{3-fluoro-2-oxo-4-[2- (piperidin-1-yl)propanamido]-1,2- dihydropyridin-1-yl}butyl)-N-{[4- (trifluoromethyl)pyridin-2-yl]methyl}- 1H-1,2,3-triazole-4-carboxamide |
| 346 | 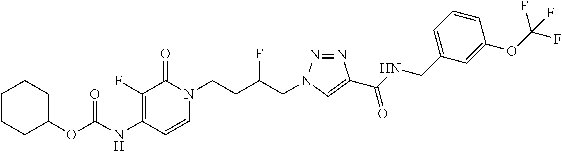 [SMILES: O=C(Nc1ccn(CCC(F)Cn2cc(C(=O)NCc3cccc(OC(F)(F)F)c3)nn2)c(=O)c1F)OC1CCCCC1] | cyclohexyl N-(3-fluoro-1-{3-fluoro-4- [4-({[3-(trifluoro- methoxy)phenyl]methyl}carbamoyl)- 1H-1,2,3-triazol-1-yl]butyl}-2- oxo-1,2-dihydropyridin-4-yl)carbamate |
| 347 | 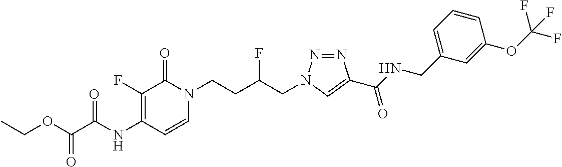 [SMILES: CCOC(=O)C(=O)Nc1ccn(CCC(F)Cn2cc(C(=O)NCc3cccc(OC(F)(F)F)c3)nn2)c(=O)c1F] | ethyl [(3-fluoro-1-{3-fluoro-4-[4-({[3- (trifluoromethoxy)phenyl]methyl} carbamoyl)-1H-1,2,3-triazol- 1-yl]butyl}-2- oxo-1,2-dihydropyridin-4- yl)carbamoyl]formate |
| 348 | 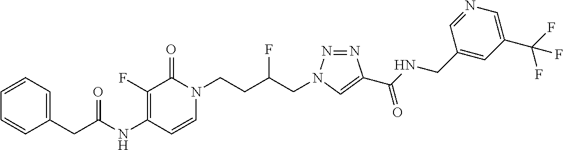 [SMILES: O=C(Cc1ccccc1)Nc1ccn(CCC(F)Cn2cc(C(=O)NCc3cncc(C(F)(F)F)c3)nn2)c(=O)c1F] | 1-{2-fluoro-4-[3-fluoro-2-oxo-4-(2- phenylacetamido)-1,2-dihydropyridin- 1-yl]butyl}-N-{[5- (trifluoromethyl)pyridin-3-yl]methyl}- 1H-1,2,3-triazole-4-carboxamide |
| 349 | 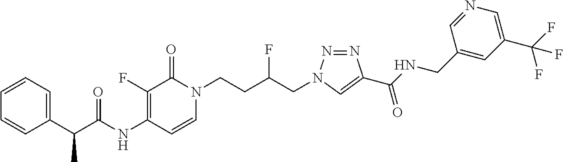 [SMILES: C[C@H](C(=O)Nc1ccn(CCC(F)Cn2cc(C(=O)NCc3cncc(C(F)(F)F)c3)nn2)c(=O)c1F)c1ccccc1] | 1-(2-fluoro-4-{3-fluoro-2-oxo-4-[(2S)- 2-phenylpropanamido]-1,2- dihydropyridin-1-yl}butyl)-N-{[5- (trifluoromethy])pyridin-3-yl]methyl}- 1H-1,2,3-triazole-4-carboxamide |
| 350 | 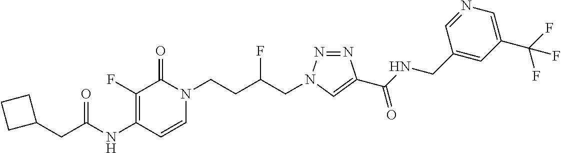 [SMILES: O=C(CC1CCC1)Nc1ccn(CCC(F)Cn2cc(C(=O)NCc3cncc(C(F)(F)F)c3)nn2)c(=O)c1F] | 1-{4-[4-(2-cyclobutylacetamido)-3- fluoro-2-oxo-1,2-dihydropyridin-1-yl]- 2-fluorobutyl}-N-{[5- (trifluoromethyl)pyridin-3-yl]methyl}- 1H-1,2,3-triazole-4-carboxamide |
| 351 | 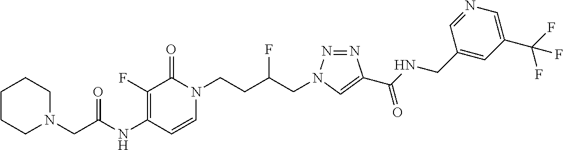 [SMILES: O=C(CN1CCCCC1)Nc1ccn(CCC(F)Cn2cc(C(=O)NCc3cncc(C(F)(F)F)c3)nn2)c(=O)c1F] | 1-(2-fluoro-4-{3-fluoro-2-oxo-4-[2- (piperidin-1-yl)acetamido]-1,2- dihydropyridin-1-yl}butyl)-N-{[5- (trifluoromethyl)pyridin-3-yl]methyl}- 1H-1,2,3-triazole-4-carboxamide |
| 352 | 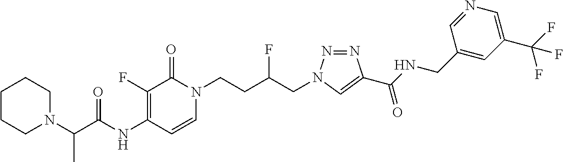 [SMILES: CC(C(=O)Nc1ccn(CCC(F)Cn2cc(C(=O)NCc3cncc(C(F)(F)F)c3)nn2)c(=O)c1F)N1CCCCC1] | 1-(2-fluoro-4-{3-fluoro-2-oxo-4-[2- (piperidin-1-yl)propanamido]-1,2- dihydropyridin-1-yl}butyl)-N-{[5- (trifluoromethyl)pyridin-3-yl]methyl}- 1H-1,2,3-triazole-4-carboxamide |
| 353 | 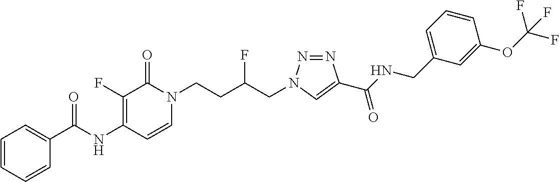 [SMILES: O=C(Nc1ccn(CCC(F)Cn2cc(C(=O)NCc3cccc(OC(F)(F)F)c3)nn2)c(=O)c1F)c1ccccc1] | 1-[4-(4-benzamido-3-fluoro-2-oxo-1,2- dihydropyridin-1-yl)-2-fluorobutyl]-N- {[3-(trifluoromethoxy)phenyl]methyl}- 1H-1,2,3-triazole-4-carboxamide |
| 354 | 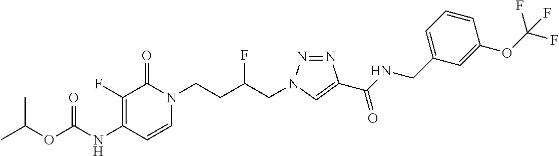 [SMILES: CC(C)OC(=O)Nc1ccn(CCC(F)Cn2cc(C(=O)NCc3cccc(OC(F)(F)F)c3)nn2)c(=O)c1F] | propan-2-yl N-(3-fluoro-1-{3-fluoro-4- [4-({[3-(trifluoro- methoxy)phenyl]methyl}carbamoy])- 1H-1,2,3-triazol-1-yl]butyl}-2- oxo-1,2-dihydropyridin-4-yl)carbamate |
| 355 | 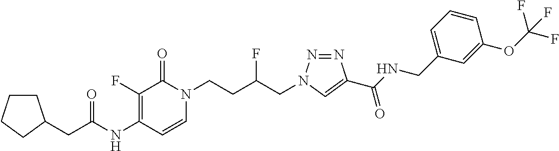 [SMILES: O=C(CC1CCCC1)Nc1ccn(CCC(F)Cn2cc(C(=O)NCc3cccc(OC(F)(F)F)c3)nn2)c(=O)c1F] | 1-{4-[4-(2-cyclopentylacetamido)-3- fluoro-2-oxo-1,2-dihydropyridin-1-yl]- 2-fluorobutyl}-N-{[3- (trifluoromethoxy)phenyl]methyl}-1H- 1,2,3-triazole-4-carboxamide |
| 356 | 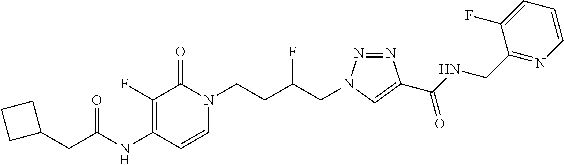 [SMILES: O=C(CC1CCC1)Nc1ccn(CCC(F)Cn2cc(C(=O)NCc3ncccc3F)nn2)c(=O)c1F] | 1-{4-[4-(2-cyclobutylacetamido)-3- fluoro-2-oxo-1,2-dihydropyridin-1-yl]- 2-fluorobutyl}-N-[(3-fluoropyridin-2- yl)methyl]-1H-1,2,3-triazole-4- carboxamide |
| 357 | 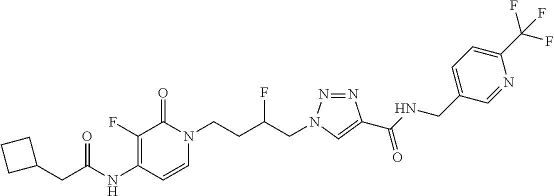 [SMILES: O=C(CC1CCC1)Nc1ccn(CCC(F)Cn2cc(C(=O)NCc3ccc(C(F)(F)F)nc3)nn2)c(=O)c1F] | 1-{4-[4-(2-cyclobutylacetamido)-3- fluoro-2-oxo-1,2-dihydropyridin-1-yl]- 2-fluorobutyl}-N-{[6- (trifluoromethyl)pyridin-3-yl]methyl}- 1H-1,2,3-triazole-4-carboxamide |
| 358 | 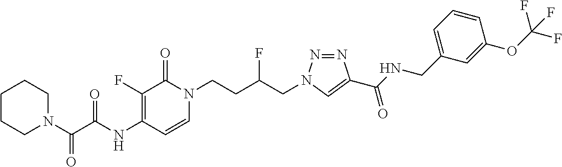 [SMILES: O=C(Nc1ccn(CCC(F)Cn2cc(C(=O)NCc3cccc(OC(F)(F)F)c3)nn2)c(=O)c1F)C(=O)N1CCCCC1] | 1-(2-fluoro-4-{3-fluoro-2-oxo-4-[2-oxo- 2-(piperidin-1-yl)acetamido]-1,2- dihydropyridin-1-yl}butyl)-N-{[3- (trifluoromethoxy)phenyl]methyl}-1H- 1,2,3-triazole-4-carboxamide |
| 359 | 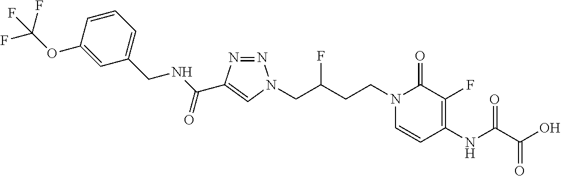 [SMILES: O=C(O)C(=O)Nc1ccn(CCC(F)Cn2cc(C(=O)NCc3cccc(OC(F)(F)F)c3)nn2)c(=O)c1F] | [(3-fluoro-1-{3-fluoro-4-[4-({[3- (trifluoromethoxy)phenyl]methyl} carbamoyl)-1H-1,2,3-triazol- 1-yl]butyl}-2- oxo-1,2-dihydropyridin-4- yl)carbamoyl]formic acid |
| 360 | 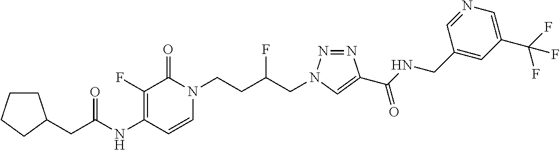 [SMILES: O=C(CC1CCCC1)Nc1ccn(CCC(F)Cn2cc(C(=O)NCc3cncc(C(F)(F)F)c3)nn2)c(=O)c1F] | 1-{4-[4-(2-cyclopentylacetamido)-3- fluoro-2-oxo-1,2-dihydropyridin-1-yl]- 2-fluorobutyl}-N-{[5- (trifluoromethyl)pyridin-3-yl]methyl}- 1H-1,2,3-triazole-4-carboxamide |
| 361 | 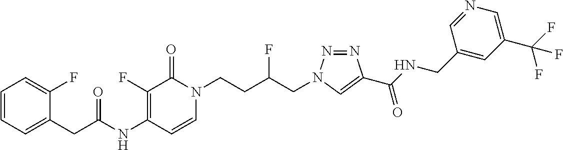 [SMILES: O=C(Cc1ccccc1F)Nc1ccn(CCC(F)Cn2cc(C(=O)NCc3cncc(C(F)(F)F)c3)nn2)c(=O)c1F] | 1-(2-fluoro-4-{3-fluoro-4-[2-(2- fluorophenyl)acetamido]-2-oxo-1,2- dihydropyridin-1-yl}butyl)-N-{[5- (trifluoromethyl)pyridin-3-yl]methyl}- 1H-1,2,3-triazole-4-carboxamide |
| 362 | 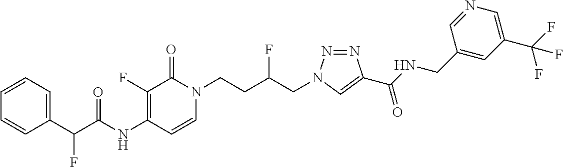 [SMILES: O=C(NCc1cncc(C(F)(F)F)c1)c1cn(CC(F)CCn2ccc(NC(=O)C(F)c3ccccc3)c(F)c2=O)nn1] | 1-{2-fluoro-4-[3-fluoro-4-(2-fluoro-2- phenylacetamido)-2-oxo-1,2- dihydropyridin-1-yl]butyl}-N-{[5- (trifluoromethyl)pyridin-3-yl]methyl}- 1H-1,2,3-triazole-4-carboxamide |
| 363 | 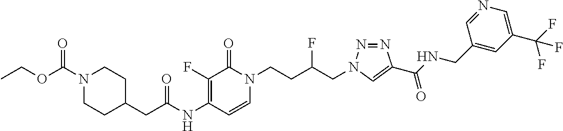 [SMILES: CCOC(=O)N1CCC(CC(=O)Nc2ccn(CCC(F)Cn3cc(C(=O)NCc4cncc(C(F)(F)F)c4)nn3)c(=O)c2F)CC1] | ethyl 4-{[(3-fluoro-1-{3-fluoro-4-[4- ({[5-(trifluoromethyl)pyridin-3- yl]methyl}carbamoyl)-1H-1,2,3-triazol- 1-yl]butyl}-2-oxo-1,2-dihydropyridin-4- yl)carbamoyl]methyl}piperidine-1- carboxylate |
| 364 | 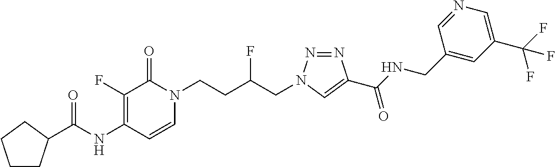 [SMILES: O=C(NCc1cncc(C(F)(F)F)c1)c1cn(CC(F)CCn2ccc(NC(=O)C3CCCC3)c(F)c2=O)nn1] | 1-[4-(4-cyclopentaneamido-3-fluoro-2- oxo-1,2-dihydropyridin-1-yl)-2- fluorobutyl]-N-{[5- (trifluoromethyl)pyridin-3-yl]methyl}- 1H-1,2,3-triazole-4-carboxamide |
| 365 | 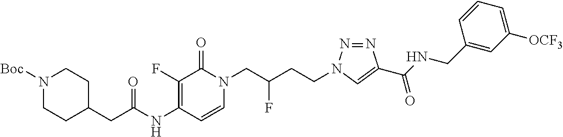 [SMILES: CC(C)(C)OC(=O)N1CCC(CC(=O)Nc2ccn(CC(F)CCn3cc(C(=O)NCc4cccc(OC(F)(F)F)c4)nn3)c(=O)c2F)CC1] | tert-butyl 4-(2-((3-fluoro-1-(2-fluoro-4- (4-((3- (trifluoromethoxy)benzyl)carbamoyl)- 1H-1,2,3-triazol-1-yl)butyl)-2-oxo-1,2- dihydropyridin-4-yl)amino)-2- oxoethyl)piperidine-1-carboxylate |
| 366 | 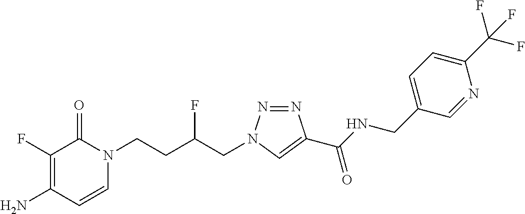 [SMILES: Nc1ccn(CCC(F)Cn2cc(C(=O)NCc3ccc(C(F)(F)F)nc3)nn2)c(=O)c1F] | 1-[4-(4-amino-3-fluoro-2-oxo-1,2- dihydropyridin-1-yl)-2-fluorobutyl]-N- {[6-(trifluoromethyl)pyridin-3- yl]methyl}-1H-1,2,3-triazole-4- carboxamide |
| 367 | 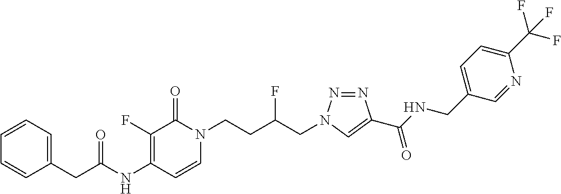 [SMILES: O=C(Cc1ccccc1)Nc1ccn(CCC(F)Cn2cc(C(=O)NCc3ccc(C(F)(F)F)nc3)nn2)c(=O)c1F] | 1-{2-fluoro-4-[3-fluoro-2-oxo-4-(2- phenylacetamido)-1,2-dihydropyridin- 1-yl]butyl}-N-{[6- (trifluoromethyl)pyridin-3-yl]methyl}- 1H-1,2,3-triazole-4-carboxamide |
| 368 | 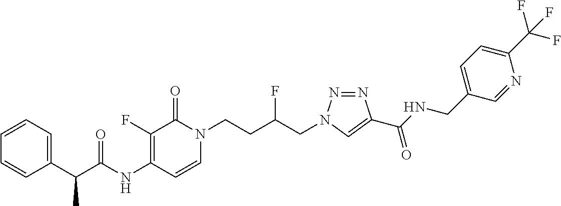 [SMILES: C[C@H](C(=O)Nc1ccn(CCC(F)Cn2cc(C(=O)NCc3ccc(C(F)(F)F)nc3)nn2)c(=O)c1F)c1ccccc1] | 1-(2-fluoro-4-{3-fluoro-2-oxo-4-[(2S)- 2-phenylpropanamido]-1,2- dihydropyridin-1-yl}butyl)-N-{[6- (trifluoromethyl)pyridin-3-yl]methyl}- 1H-1,2,3-triazole-4-carboxamide |
| 369 | 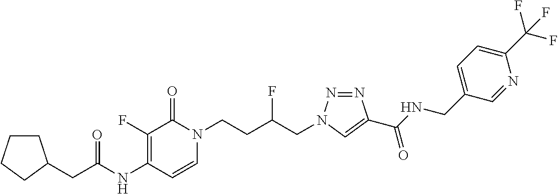 [SMILES: O=C(CC1CCCC1)Nc1ccn(CCC(F)Cn2cc(C(=O)NCc3ccc(C(F)(F)F)nc3)nn2)c(=O)c1F] | 1-{4-[4-(2-cyclopentylacetamido)-3- fluoro-2-oxo-1,2-dihydropyridin-1-yl]- 2-fluorobutyl}-N-{[6- (trifluoromethyl)pyridin-3-yl]methyl}- 1H-1,2,3-triazole-4-carboxamide |
| 370 | 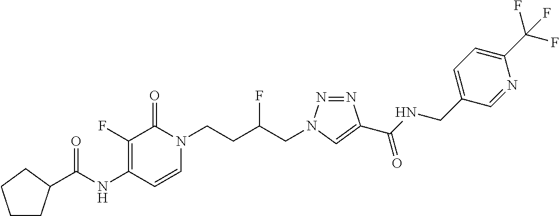 [SMILES: O=C(NCc1ccc(C(F)(F)F)nc1)c1cn(CC(F)CCn2ccc(NC(=O)C3CCCC3)c(F)c2=O)nn1] | 1-[4-(4-cyclopentaneamido-3-fluoro-2- oxo-1,2-dihydropyridin-1-yl)-2- fluorobutyl]-N-{[6- (trifluoromethyl)pyridin-3-yl]methyl}- 1H-1,2,3-triazole-4-carboxamide |
| 371 | 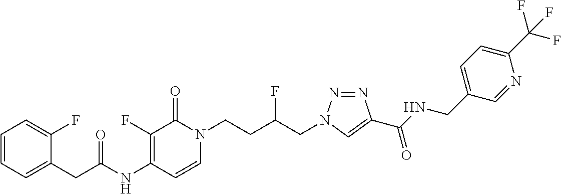 [SMILES: O=C(Cc1ccccc1F)Nc1ccn(CCC(F)Cn2cc(C(=O)NCc3ccc(C(F)(F)F)nc3)nn2)c(=O)c1F] | 1-(2-fluoro-4-{3-fluoro-4-[2-(2- fluorophenyl)acetamido]-2-oxo-1,2- dihydropyridin-1-yl}butyl)-N-{[6- (trifluoromethyl)pyridin-3-yl]methyl}- 1H-1,2,3-triazole-4-carboxamide |
| 372 | 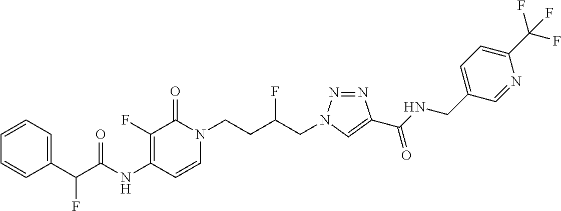 [SMILES: O=C(NCc1ccc(C(F)(F)F)nc1)c1cn(CC(F)CCn2ccc(NC(=O)C(F)c3ccccc3)c(F)c2=O)nn1] | l-{2-fluoro-4-[3-fluoro-4-(2-fluoro-2- phenylacetamido)-2-oxo-1,2- dihydropyridin-1-yl]butyl}-N-{[6- (trifluoromethyl)pyridin-3-yl]methyl}- 1H-1,2,3-triazole-4-carboxamide |
| 373 | 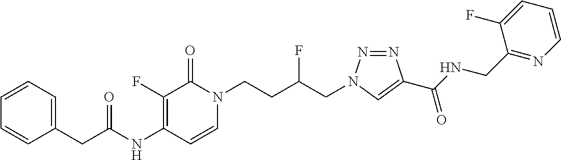 [SMILES: O=C(Cc1ccccc1)Nc1ccn(CCC(F)Cn2cc(C(=O)NCc3ncccc3F)nn2)c(=O)c1F] | 1-{2-fluoro-4-[3-fluoro-2-oxo-4-(2- phenylacetamido)-1,2-dihydropyridin- 1-yl]butyl}-N-[(3-fluoropyridin-2- yl)methyl]-1H-1,2,3-triazole-4- carboxamide |
| 374 | 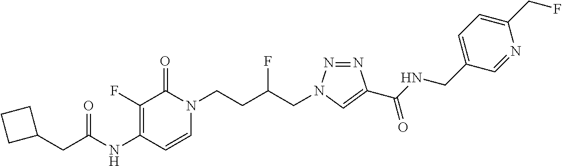 [SMILES: O=C(CC1CCC1)Nc1ccn(CCC(F)Cn2cc(C(=O)NCc3ccc(CF)nc3)nn2)c(=O)c1F] | 1-{4-[4-(2-cyclobutylacetamido)-3- fluoro-2-oxo-1,2-dihydropyridin-1-yl]- 2-fluorobutyl}-N-{[6- (fluoromethyl)pyridin-3-yl]methyl}- 1H-1,2,3-triazole-4-carboxamide |
| 375 | 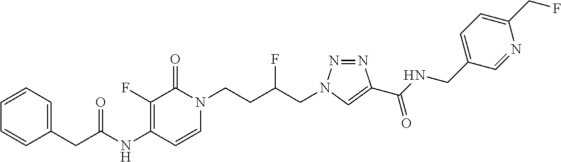 [SMILES: O=C(Cc1ccccc1)Nc1ccn(CCC(F)Cn2cc(C(=O)NCc3ccc(CF)nc3)nn2)c(=O)c1F] | 1-{2-fluoro-4-[3-fluoro-2-oxo-4-(2- phenylacetamido)-1,2-dihydropyridin- 1-yl]butyl}-N-{[6-(fluoro- methyl)pyridin-3-yl]methyl}- 1H-1,2,3-triazole-4-carboxamide |
| 376 | 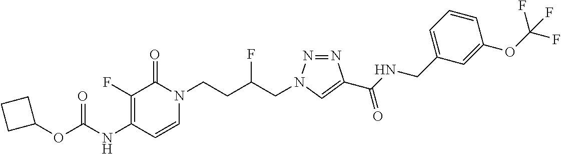 [SMILES: O=C(Nc1ccn(CCC(F)Cn2cc(C(=O)NCc3cccc(OC(F)(F)F)c3)nn2)c(=O)c1F)OC1CCC1] | cyclobutyl N-(3-fluoro-1-{3-fluoro-4- [4-({[3-(trifluoro- methoxy)phenyl]methyl}carbamoyl)- 1H-1,2,3-triazol-1-yl]butyl}-2- oxo-1,2-dihydropyridin-4-yl)carbamate |
| 377 | 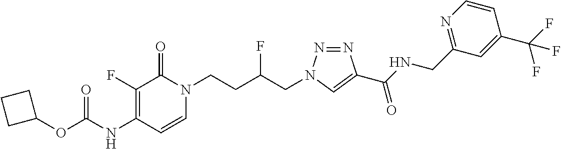 [SMILES: O=C(Nc1ccn(CCC(F)Cn2cc(C(=O)NCc3cc(C(F)(F)F)ccn3)nn2)c(=O)c1F)OC1CCC1] | cyclobutyl N-(3-fluoro-1-{3-fluoro-4- [4-({[4-(trifluoromethyl)pyridin-2- yl]methyl}carbamoyl)-1H-1,2,3-triazol- 1-yl]butyl}-2-oxo-1,2-dihydropyridin-4- yl)carbamate |
| 378 | 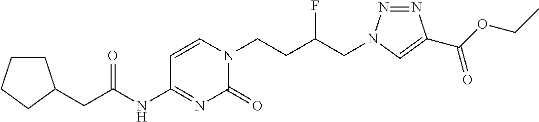 [SMILES: CCOC(=O)c1cn(CC(F)CCn2ccc(NC(=O)CC3CCCC3)nc2=O)nn1] | ethyl 1-{4-[4-(2-cyclopentylacetamido)- 2-oxo-1,2-dihydropyrimidin-1-yl]-2- fluorobutyl}-1H-1,2,3-triazole-4- carboxylate |
| 379 | 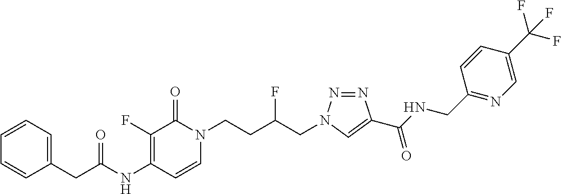 [SMILES: O=C(Cc1ccccc1)Nc1ccn(CCC(F)Cn2cc(C(=O)NCc3ccc(C(F)(F)F)cn3)nn2)c(=O)c1F] | 1-{2-fluoro-4-[3-fluoro-2-oxo-4-(2- phenylacetamido)-1,2-dihydropyridin- 1-yl]butyl}-N-{[5- (trifluoromethyl)pyridin-2-yl]methyl}- 1H-1,2,3-triazole-4-carboxamide |
| 380 | 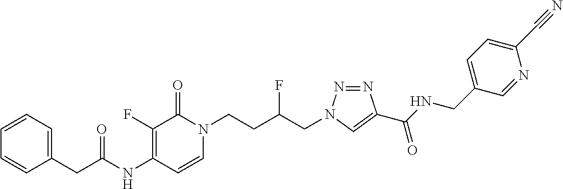 [SMILES: N#Cc1ccc(CNC(=O)c2cn(CC(F)CCn3ccc(NC(=O)Cc4ccccc4)c(F)c3=O)nn2)cn1] | N-[(6-cyanopyridin-3-yl)methyl]-1-{2- fluoro-4-[3-fluoro-2-oxo-4-(2- phenylacetamido)-1,2-dihydropyridin- 1-yl]butyl}-1H-1,2,3-triazole-4- carboxamide |
| 381 | 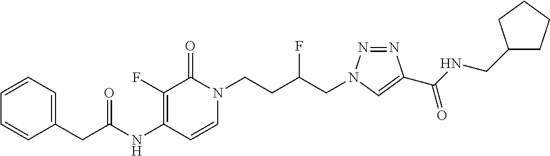 [SMILES: O=C(Cc1ccccc1)Nc1ccn(CCC(F)Cn2cc(C(=O)NCC3CCCC3)nn2)c(=O)c1F] | N-(cyclopentylmethyl)-1-{2-fluoro-4- [3-fluoro-2-oxo-4-(2-phenylacetamido)- 1,2-dihydropyridin-1-yl]butyl}-1H- 1,2,3-triazole-4-carboxamide |
| 382 | 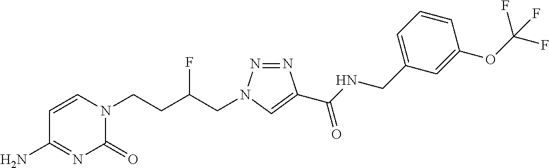 [SMILES: Nc1ccn(CCC(F)Cn2cc(C(=O)NCc3cccc(OC(F)(F)F)c3)nn2)c(=O)n1] | 1-[4-(4-amino-2-oxo-1,2- dihydropyrimidin-1-yl)-2-fluorobutyl]- N-{[3- (trifluoromethoxy)phenyl]methyl}-1H- 1,2,3-triazole-4-carboxamide |
| 383 | 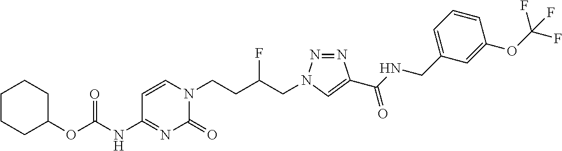 [SMILES: O=C(Nc1ccn(CCC(F)Cn2cc(C(=O)NCc3cccc(OC(F)(F)F)c3)nn2)c(=O)n1)OC1CCCCC1] | cyclohexyl N-(1-{3-fluoro-4-[4-({[3- (trifluoromethoxy)phenyl]methyl} carbamoyl)-1H-1,2,3-triazol- 1-yl]butyl}-2- oxo-1,2-dihydropyrimidin-4- yl)carbamate |
| 384 | 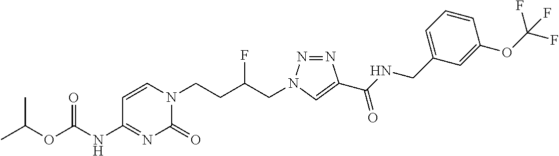 [SMILES: CC(C)OC(=O)Nc1ccn(CCC(F)Cn2cc(C(=O)NCc3cccc(OC(F)(F)F)c3)nn2)c(=O)n1] | propan-2-yl N-(1-{3-fluoro-4-[4-({[3- (trifluoromethoxy)phenyl]methyl} carbamoyl)-1H-1,2,3-triazol- 1-yl]butyl}-2-oxo-1,2-dihydro- pyrimidin-4-yl)carbamate |
| 385 | 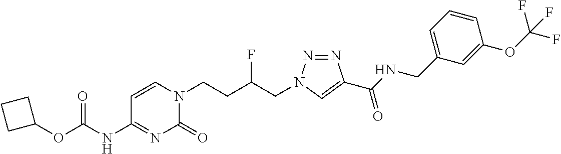 [SMILES: O=C(Nc1ccn(CCC(F)Cn2cc(C(=O)NCc3cccc(OC(F)(F)F)c3)nn2)c(=O)n1)OC1CCC1] | cyclobutyl N-(1-{3-fluoro-4-[4-({[3- (trifluoromethoxy)phenyl]methyl} carbamoyl)-1H-1,2,3-triazol- 1-yl]butyl}-2-oxo-1,2-dihydro- pyrimidin-4-yl)carbamate |
| 386 | 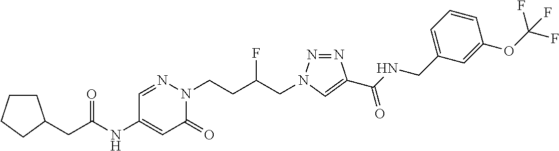 [SMILES: O=C(CC1CCCC1)Nc1cnn(CCC(F)Cn2cc(C(=O)NCc3cccc(OC(F)(F)F)c3)nn2)c(=O)c1] | 1-{4-[4-(2-cyclopentylacetamido)-6- oxo-1,6-dihydropyridazin-1-yl]-2- fluorobutyl}-N-{[3- (trifluoromethoxy)phenyl]methyl}-1H- 1,2,3-triazole-4-carboxamide |
| 387 | 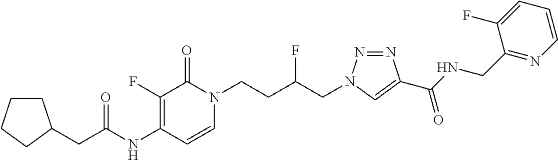 [SMILES: O=C(CC1CCCC1)Nc1ccn(CCC(F)Cn2cc(C(=O)NCc3ncccc3F)nn2)c(=O)c1F] | 1-{4-[4-(2-cyclopentylacetamido)-3- fluoro-2-oxo-1,2-dihydropyridin-1-yl]- 2-fluorobutyl}-N-[(3-fluoropyridin-2- yl)methyl]-1H-1,2,3-triazole-4- carboxamide |
| 388 | 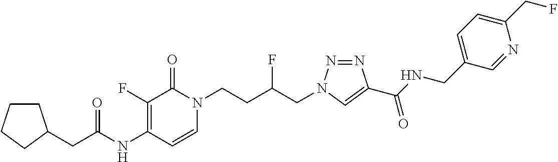 [SMILES: O=C(CC1CCCC1)Nc1ccn(CCC(F)Cn2cc(C(=O)NCc3ccc(CF)nc3)nn2)c(=O)c1F] | 1-{4-[4-(2-cyclopentylacetamido)-3- fluoro-2-oxo-1,2-dihydropyridin-1-yl]- 2-fluorobutyl}-N-{[6- (fluoromethyl)pyridin-3-yl]methyl}- 1H-1,2,3-triazole-4-carboxamide |
| 389 | 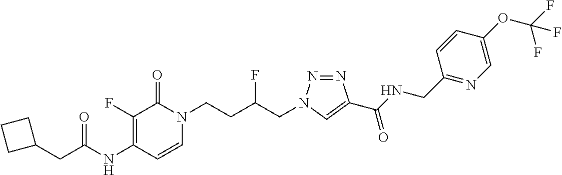 [SMILES: O=C(CC1CCC1)Nc1ccn(CCC(F)Cn2cc(C(=O)NCc3ccc(OC(F)(F)F)cn3)nn2)c(=O)c1F] | 1-{4-[4-(2-cyclobutylacetamido)-3- fluoro-2-oxo-1,2-dihydropyridin-1-yl]- 2-fluorobutyl}-N-{[5- (trifluoromethoxy)pyridin-2-yl]methyl}- 1H-1,2,3-triazole-4-carboxamide |
| 390 | 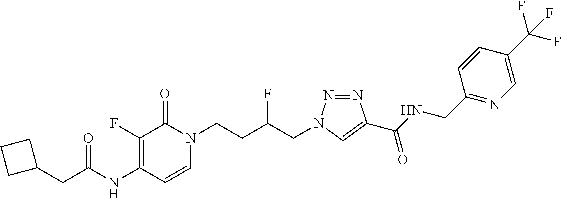 [SMILES: O=C(CC1CCC1)Nc1ccn(CCC(F)Cn2cc(C(=O)NCc3ccc(C(F)(F)F)cn3)nn2)c(=O)c1F] | 1-{4-[4-(2-cyclobutylacetamido)-3- fluoro-2-oxo-1,2-dihydropyridin-1-yl]- 2-fluorobutyl}-N-{[5- (trifluoromethyl)pyridin-2-yl]methyl}- 1H-1,2,3-triazole-4-carboxamide |
| 391 | 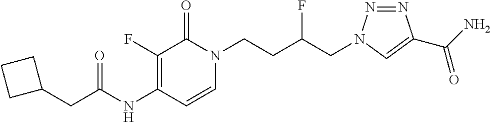 [SMILES: NC(=O)c1cn(CC(F)CCn2ccc(NC(=O)CC3CCC3)c(F)c2=O)nn1] | 1-{4-[4-(2-cyclobutylacetamido)-3- fluoro-2-oxo-1,2-dihydropyridin-1-yl]- 2-fluorobutyl}-1H-1,2,3-triazole-4- carboxamide |
| 392 | 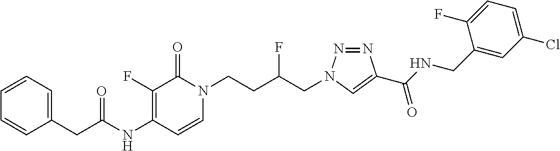 [SMILES: O=C(Cc1ccccc1)Nc1ccn(CCC(F)Cn2cc(C(=O)NCc3cc(Cl)ccc3F)nn2)c(=O)c1F] | N-[(5-chloro-2-fluorophenyl)methyl]-1- {2-fluoro-4-[3-fluoro-2-oxo-4-(2- phenylacetamido)-1,2-dihydropyridin- 1-yl]butyl}-1H-1,2,3-triazole-4- carboxamide |
| 393 | 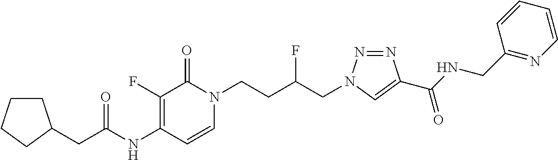 [SMILES: O=C(CC1CCCC1)Nc1ccn(CCC(F)Cn2cc(C(=O)NCc3ccccn3)nn2)c(=O)c1F] | 1-{4-[4-(2-cyclopentylacetamido)-3- fluoro-2-oxo-1,2-dihydropyridin-1-yl]- 2-fluorobutyl}-N-(pyridin-2-ylmethyl)- 1H-1,2,3-triazole-4-carboxamide |
| 394 | 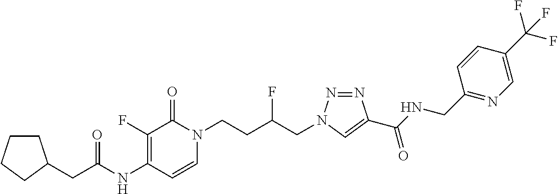 [SMILES: O=C(CC1CCCC1)Nc1ccn(CCC(F)Cn2cc(C(=O)NCc3ccc(C(F)(F)F)cn3)nn2)c(=O)c1F] | 1-{4-[4-(2-cyclopentylacetamido)-3- fluoro-2-oxo-1,2-dihydropyridin-1-yl]- 2-fluorobutyl}-N-{[5- (trifluoromethyl)pyridin-2-yl]methyl}- 1H-1,2,3-triazole-4-carboxamide |
| 395 | 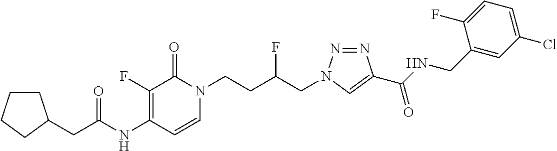 [SMILES: O=C(CC1CCCC1)Nc1ccn(CCC(F)Cn2cc(C(=O)NCc3cc(Cl)ccc3F)nn2)c(=O)c1F] | N-[(5-chloro-2-fluorophenyl)methyl]-1- {4-[4-(2-cyclopentylacetamido)-3- fluoro-2-oxo-1,2-dihydropyridin-1-yl]- 2-fluorobutyl}-1H-1,2,3-triazole-4- carboxamide |
| 396 | 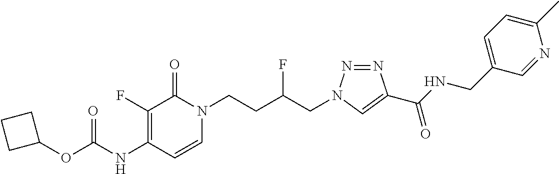 [SMILES: Cc1ccc(CNC(=O)c2cn(CC(F)CCn3ccc(NC(=O)OC4CCC4)c(F)c3=O)nn2)cn1] | cyclobutyl N-{3-fluoro-1-[3-fluoro-4- (4-{[(6-methylpyridin-3- yl)methyl]carbamoyl}-1H-1,2,3-triazol- 1-yl)butyl]-2-oxo-1,2-dihydropyridin-4- yl}carbamate |
| 397 | 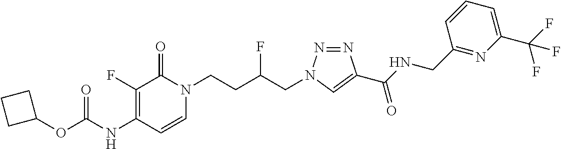 [SMILES: O=C(Nc1ccn(CCC(F)Cn2cc(C(=O)NCc3cccc(C(F)(F)F)n3)nn2)c(=O)c1F)OC1CCC1] | cyclobutyl N-(3-fluoro-1-{3-fluoro-4- [4-({[6-(trifluoromethyl)pyridin-2- yl]methyl}carbamoyl)-1H-1,2,3-triazol- 1-yl]butyl}-2-oxo-1,2-dihydropyridin-4- yl)carbamate |
| 398 | 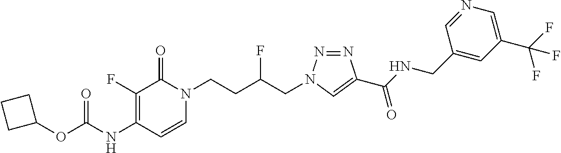 [SMILES: O=C(Nc1ccn(CCC(F)Cn2cc(C(=O)NCc3cncc(C(F)(F)F)c3)nn2)c(=O)c1F)OC1CCC1] | cyclobutyl N-(3-fluoro-1-{3-fluoro-4- [4-({[5-(trifluoromethyl)pyridin-3- yl]methyl}carbamoyl)-1H-1,2,3-triazol- 1-yl]butyl}-2-oxo-1,2-dihydropyridin-4- yl)carbamate |
| 399 | 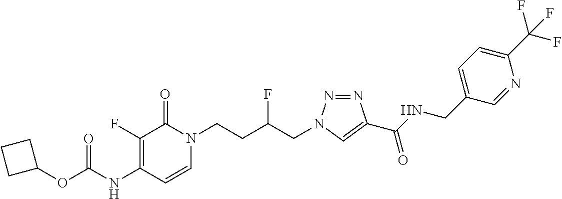 [SMILES: O=C(Nc1ccn(CCC(F)Cn2cc(C(=O)NCc3ccc(C(F)(F)F)nc3)nn2)c(=O)c1F)OC1CCC1] | cyclobutyl N-(3-fluoro-1-{3-fluoro-4- [4-({[6-(trifluoromethyl)pyridin-3- yl]methyl}carbamoyl)-1H-1,2,3-triazol- 1-yl]butyl}-2-oxo-1,2-dihydropyridin-4- yl)carbamate |
| 400 | 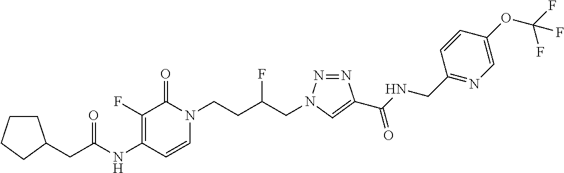 [SMILES: O=C(CC1CCCC1)Nc1ccn(CCC(F)Cn2cc(C(=O)NCc3ccc(OC(F)(F)F)cn3)nn2)c(=O)c1F] | 1-{4-[4-(2-cyclopentylacetamido)-3- fluoro-2-oxo-1,2-dihydropyridin-1-yl]- 2-fluorobutyl}-N-{[5- (trifluoromethoxy)pyridin-2-yl]methyl}- 1H-1,2,3-triazole-4-carboxamide |
| 401 | 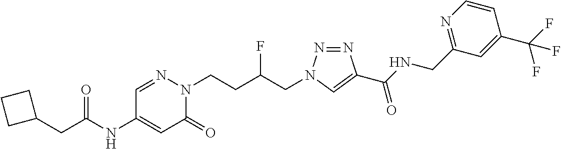 [SMILES: O=C(CC1CCC1)Nc1cnn(CCC(F)Cn2cc(C(=O)NCc3cc(C(F)(F)F)ccn3)nn2)c(=O)c1] | 1-{4-[4-(2-cyclobutylacetamido)-6-oxo- 1,6-dihydropyridazin-1-yl]-2- fluorobutyl}-N-{[4- (trifluoromethyl)pyridin-2-yl]methyl}- 1H-1,2,3-triazole-4-carboxamide |
| 402 | 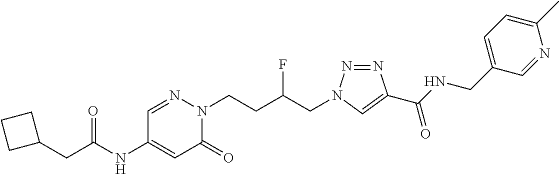 [SMILES: Cc1ccc(CNC(=O)c2cn(CC(F)CCn3ncc(NC(=O)CC4CCC4)cc3=O)nn2)cn1] | 1-{4-[4-(2-cyclobutylacetamido)-6-oxo- 1,6-dihydropyridazin-1-yl]-2- fluorobutyl}-N-[(6-methylpyridin-3- yl)methyl]-1H-1,2,3-triazole-4- carboxamide |
| 403 | 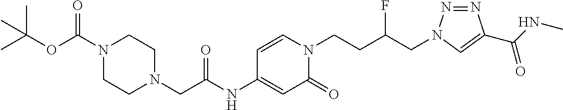 [SMILES: CNC(=O)c1cn(CC(F)CCn2ccc(NC(=O)CN3CCN(C(=O)OC(C)(C)C)CC3)cc2=O)nn1] | tert-butyl 4-{[(1-{3-fluoro-4-[4- (methylcarbamoyl)-1H-1,2,3-triazol-1- yl]butyl}-2-oxo-1,2-dihydropyridin-4- yl)carbamoyl]methyl}piperazine-1- carboxylate |
| 404 | 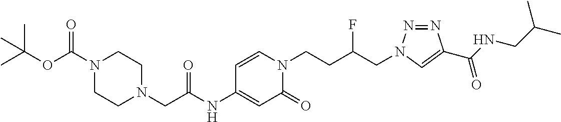 [SMILES: CC(C)CNC(=O)c1cn(CC(F)CCn2ccc(NC(=O)CN3CCN(C(=O)OC(C)(C)C)CC3)cc2=O)nn1] | tert-butyl 4-({[1-(3-fluoro-4-{4-[(2- methylpropyl)carbamoyl]-lH-1,2,3- triazol-1-yl}butyl)-2-oxo-1,2- dihydropyridin-4- yl]carbamoyl}methyl)piperazine-1- carboxylate |
| 405 | 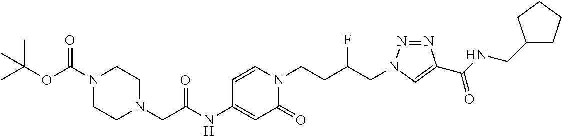 [SMILES: CC(C)(C)OC(=O)N1CCN(CC(=O)Nc2ccn(CCC(F)Cn3cc(C(=O)NCC4CCCC4)nn3)c(=O)c2)CC1] | tert-butyl 4-({[1-(4-{4- [(cyclopentylmethyl)carbamoyl]-1H- 1,2,3-triazol-1-yl}-3-fluorobutyl)-2- oxo-1,2-dihydropyridin-4- yl]carbamoyl}methyl)piperazine-1- carboxylate |
| 406 | 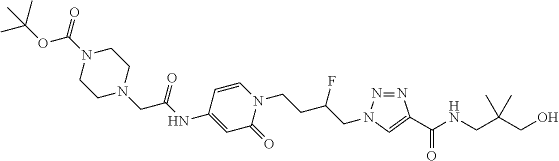 [SMILES: CC(C)(CO)CNC(=O)c1cn(CC(F)CCn2ccc(NC(=O)CN3CCN(C(=O)OC(C)(C)C)CC3)cc2=O)nn1] | tert-butyl 4-({[1-(3-fluoro-4-{4-[(3- hydroxy-2,2- dimethylpropyl)carbamoyl]-1H-1,2,3- triazol-1-yl}butyl)-2-oxo-1,2- dihydropyridin-4- yl]carbamoyl}methyl)piperazine-1- carboxylate |
| 407 | 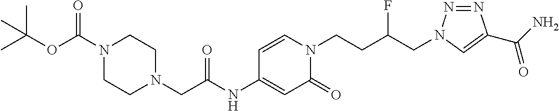 [SMILES: CC(C)(C)OC(=O)N1CCN(CC(=O)Nc2ccn(CCC(F)Cn3cc(C(N)=O)nn3)c(=O)c2)CC1] | tert-butyl 4-[({1-[4-(4-carbamoyl-1H- 1,2,3-triazol-1-yl)-3-fluorobutyl]-2-oxo- 1,2-dihydropyridin-4- yl}carbamoyl)methyl]piperazine-1- carboxylate |
| 408 | 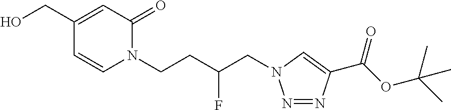 [SMILES: CC(C)(C)OC(=O)c1cn(CC(F)CCn2ccc(CO)cc2=O)nn1] | tert-butyl 1-{2-fluoro-4-[4- (hydroxymethyl)-2-oxo-1,2- dihydropyridin-1-yl]butyl}-1H-1,2,3- triazole-4-carboxylate |
| 409 | 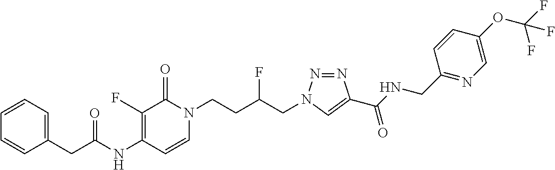 [SMILES: O=C(Cc1ccccc1)Nc1ccn(CCC(F)Cn2cc(C(=O)NCc3ccc(OC(F)(F)F)cn3)nn2)c(=O)c1F] | 1-{2-fluoro-4-[3-fluoro-2-oxo-4-(2- phenylacetamido)-1,2-dihydropyridin- 1-yl]butyl}-N-{[5- (trifluoromethoxy)pyridin-2-yl]methyl}- 1H-1,2,3-triazole-4-carboxamide |
| 410 | 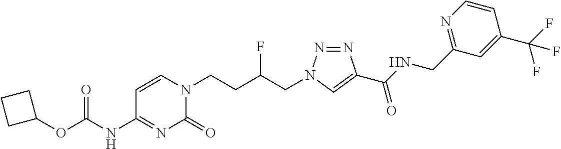 [SMILES: O=C(Nc1ccn(CCC(F)Cn2cc(C(=O)NCc3cc(C(F)(F)F)ccn3)nn2)c(=O)n1)OC1CCC1] | cyclobutyl N-(1-{3-fluoro-4-[4-({[4- (trifluoromethyl)pyridin-2- yl]methyl}carbamoyl)-1H-1,2,3-triazol- 1-yl]butyl}-2-oxo-1,2- dihydropyrimidin-4-yl)carbamate |
| 411 | 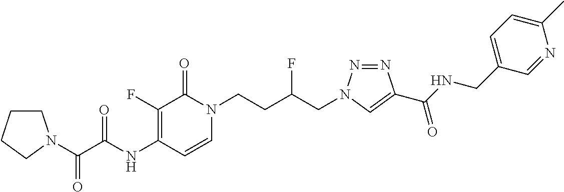 [SMILES: Cc1ccc(CNC(=O)c2cn(CC(F)CCn3ccc(NC(=O)C(=O)N4CCCC4)c(F)c3=O)nn2)cn1] | 1-(2-fluoro-4-{3-fluoro-2-oxo-4-[2-oxo- 2-(pyrrolidin-1-yl)acetamido]-1,2- dihydropyridin-1-yl}butyl)-N-[(6- methylpyridin-3-yl)methyl]-1H-1,2,3- triazole-4-carboxamide |
| 412 | 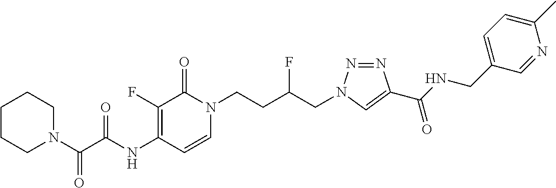 [SMILES: Cc1ccc(CNC(=O)c2cn(CC(F)CCn3ccc(NC(=O)C(=O)N4CCCCC4)c(F)c3=O)nn2)cn1] | 1-(2-fluoro-4-{3-fluoro-2-oxo-4-[2-oxo- 2-(piperidin-1-yl)acetamido]-1,2- dihydropyridin-1-yl}butyl)-N-[(6- methylpyridin-3-yl)methyl]-1H-1,2,3- triazole-4-carboxamide |
| 413 | 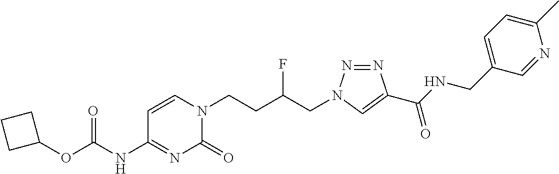 [SMILES: Cc1ccc(CNC(=O)c2cn(CC(F)CCn3ccc(NC(=O)OC4CCC4)nc3=O)nn2)cn1] | cyclobutyl N-{1-[3-fluoro-4-(4-{[(6- methylpyridin-3-yl)methyl]carbamoyl}- 1H-1,2,3-triazol-1-yl)butyl]-2-oxo-1,2- dihydropyrimidin-4-yl}carbamate |
| 414 | 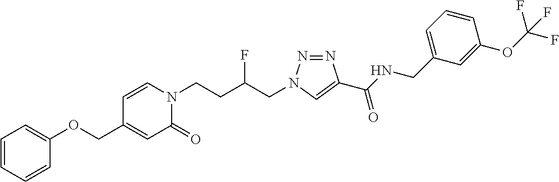 [SMILES: O=C(NCc1cccc(OC(F)(F)F)c1)c1cn(CC(F)CCn2ccc(COc3ccccc3)cc2=O)nn1] | 1-{2-fluoro-4-[2-oxo-4- (phenoxymethyl)-1,2-dihydropyridin-1- yl]butyl}-N-{[3- (trifluoromethoxy)phenyl]methyl}-1H- 1,2,3-triazole-4-carboxamide |
| 415 | 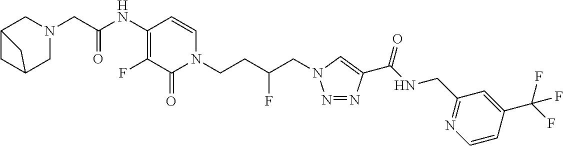 [SMILES: O=C(CN1CC2CC(C2)C1)Nc1ccn(CCC(F)Cn2cc(C(=O)NCc3cc(C(F)(F)F)ccn3)nn2)c(=O)c1F] | 1-{4-[4-(2-{3-azabicyclo[3.1.1]heptan- 3-yl}acetamido)-3-fluoro-2-oxo-1,2- dihydropyridin-1-yl]-2-fluorobutyl}-N- {[4-(trifluoromethyl)pyridin-2- yl]methyl}-1H-1,2,3-triazole-4- carboxamide |
| 416 | 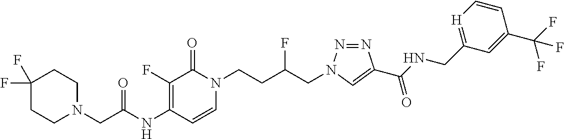 | 1-(4-{4-[2-(4,4-difluoropiperidin-1- yl)acetamido]-3-fluoro-2-oxo-1,2- dihydropyridin-1-yl}-2-fluorobutyl)-N- {[4-(trifluoromethyl)pyridin-2- yl]methyl}-1H-1,2,3-triazole-4- carboxamide |
| 417 | 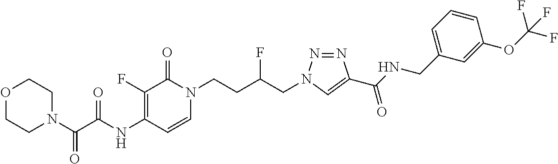 [SMILES: O=C(Nc1ccn(CCC(F)Cn2cc(C(=O)NCc3cccc(OC(F)(F)F)c3)nn2)c(=O)c1F)C(=O)N1CCOCC1] | 1-(2-fluoro-4-{3-fluoro-4-[2- (morpholin-4-yl)-2-oxoacetamido]-2- oxo-1,2-dihydropyridin-1-yl}butyl)-N- {[3-(trifluoromethoxy)phenyl]methyl}- 1H-1,2,3-triazole-4-carboxamide |
| 418 | 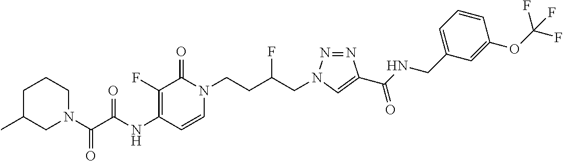 [SMILES: CC1CCCN(C(=O)C(=O)Nc2ccn(CCC(F)Cn3cc(C(=O)NCc4cccc(OC(F)(F)F)c4)nn3)c(=O)c2F)C1] | 1-(2-fluoro-4-{3-fluoro-4-[2-(3- methylpiperidin-1-yl)-2-oxoacetamido]- 2-oxo-1,2-dihydropyridin-1-yl}butyl)- N-{[3- (trifluoromethoxy)phenyl]methyl}-1H- 1,2,3-triazole-4-carboxamide |
| 419 | 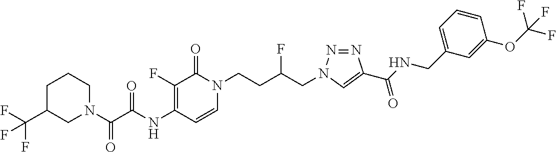 [SMILES: O=C(Nc1ccn(CCC(F)Cn2cc(C(=O)NCc3cccc(OC(F)(F)F)c3)nn2)c(=O)c1F)C(=O)N1CCCC(C(F)(F)F)C1] | 1-[2-fluoro-4-(3-fluoro-2-oxo-4-{2-oxo- 2-[3-(trifluoromethyl)piperidin-1- yl]acetamido}-1,2-dihydropyridin-1- yl)butyl]-N-{[3- (trifluoromethoxy)phenyl]methyl}-1H- 1,2,3-triazole-4-carboxamide |
| 420 | 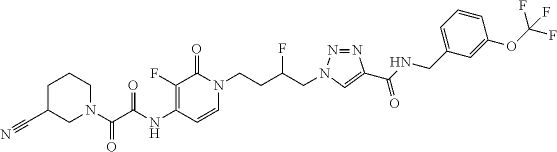 [SMILES: N#CC1CCCN(C(=O)C(=O)Nc2ccn(CCC(F)Cn3cc(C(=O)NCc4cccc(OC(F)(F)F)c4)nn3)c(=O)c2F)C1] | 1-(4-{4-[2-(3-cyanopiperidin-1-yl)-2- oxoacetamido]-3-fluoro-2-oxo-1,2- dihydropyridin-1-yl}-2-fluorobutyl)-N- {[3-(trifluoromethoxy)phenyl]methyl}- 1H-1,2,3-triazole-4-carboxamide |
| 421 | 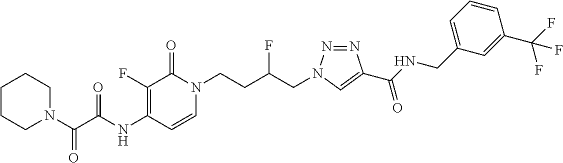 [SMILES: O=C(Nc1ccn(CCC(F)Cn2cc(C(=O)NCc3cccc(C(F)(F)F)c3)nn2)c(=O)c1F)C(=O)N1CCCCC1] | 1-(2-fluoro-4-{3-fluoro-2-oxo-4-[2-oxo- 2-(piperidin-1-yl)acetamido]-1,2- dihydropyridin-1-yl}butyl)-N-{[3- (trifluoromethyl)phenyl]methyl}-1H- 1,2,3-triazole-4-carboxamide |
| 422 | 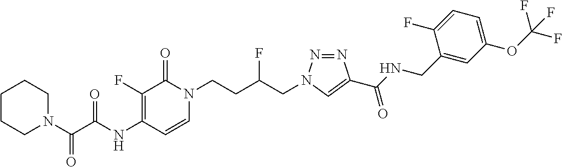 [SMILES: O=C(Nc1ccn(CCC(F)Cn2cc(C(=O)NCc3cc(OC(F)(F)F)ccc3F)nn2)c(=O)c1F)C(=O)N1CCCCC1] | 1-(2-fluoro-4-{3-fluoro-2-oxo-4-[2-oxo- 2-(piperidin-1-yl)acetamido]-1,2- dihydropyridin-1-yl}butyl)-N-{[2- fluoro-5- (trifluoromethoxy)phenyl]methyl}-1H- 1,2,3-triazole-4-carboxamide |
| 423 | 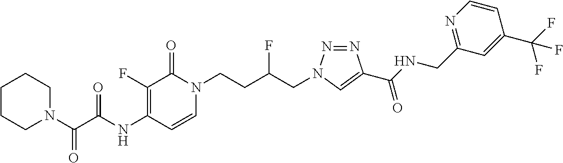 [SMILES: O=C(Nc1ccn(CCC(F)Cn2cc(C(=O)NCc3cc(C(F)(F)F)ccn3)nn2)c(=O)c1F)C(=O)N1CCCCC1] | 1-(2-fluoro-4-{3-fluoro-2-oxo-4-[2-oxo- 2-(piperidin-1-yl)acetamido]-1,2- dihydropyridin-1-yl}butyl)-N-{[4- (trifluoromethyl)pyridin-2-yl]methyl}- 1H-1,2,3-triazole-4-carboxamide |
| 424 | 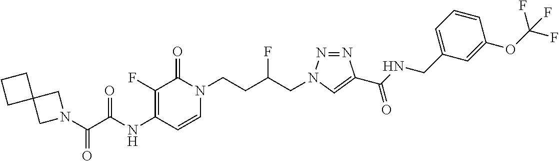 [SMILES: O=C(Nc1ccn(CCC(F)Cn2cc(C(=O)NCc3cccc(OC(F)(F)F)c3)nn2)c(=O)c1F)C(=O)N1CC2(CCC2)C1] | 1-{4-[4-(2-{2-azaspiro[3.3]heptan-2- yl}-2-oxoacetamido)-3-fluoro-2-oxo- 1,2-dihydropyridin-1-yl]-2- fluorobutyl}-N-{[3- (trifluoromethoxy)phenyl]methyl}-1H- 1,2,3-triazole-4-carboxamide |
| 425 | 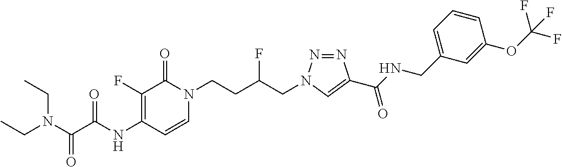 [SMILES: CCN(CC)C(=O)C(=O)Nc1ccn(CCC(F)Cn2cc(C(=O)NCc3cccc(OC(F)(F)F)c3)nn2)c(=O)c1F] | N,N-diethyl-N′-(3-fluoro-1-{3-fluoro- 4-[4-({[3-(trifluoro- methoxy)phenyl]methyl}carbamoyl)- 1H-1,2,3-triazol-1-yl]butyl}-2- oxo-1,2-dihydropyridin-4- yl)ethanediamide |
| 426 | 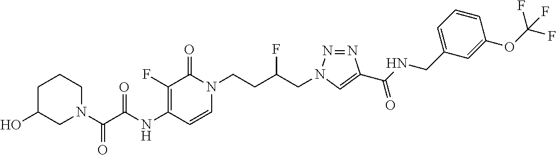 [SMILES: O=C(Nc1ccn(CCC(F)Cn2cc(C(=O)NCc3cccc(OC(F)(F)F)c3)nn2)c(=O)c1F)C(=O)N1CCCC(O)C1] | 1-(2-fluoro-4-{3-fluoro-4-[2-(3- hydroxypiperidin-1-yl)-2- oxoacetamido]-2-oxo-1,2- dihydropyridin-1-yl}butyl)-N-{[3- (trifluoromethoxy)phenyl]methyl}-1H- 1,2,3-triazole-4-carboxamide |
| 427 | 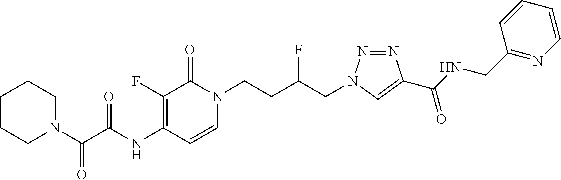 [SMILES: O=C(Nc1ccn(CCC(F)Cn2cc(C(=O)NCc3ccccn3)nn2)c(=O)c1F)C(=O)N1CCCCC1] | 1-(2-fluoro-4-{3-fluoro-2-oxo-4-[2-oxo- 2-(piperidin-1-yl)acetamido]-1,2- dihydropyridin-1-yl}butyl)-N-(pyridin- 2-ylmethyl)-1H-1,2,3-triazole-4- carboxamide |
| 428 | 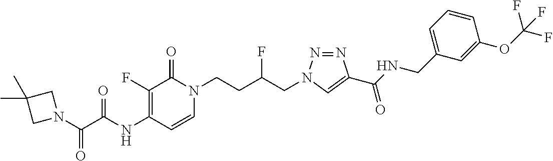 [SMILES: CC1(C)CN(C(=O)C(=O)Nc2ccn(CCC(F)Cn3cc(C(=O)NCc4cccc(OC(F)(F)F)c4)nn3)c(=O)c2F)C1] | 1-(4-{4-[2-(3,3-dimethylazetidin-1-yl)- 2-oxoacetamido]-3-fluoro-2-oxo-1,2- dihydropyridin-1-yl}-2-fluorobutyl)-N- {[3-(trifluoromethoxy)phenyl]methyl}- 1H-1,2,3-triazole-4-carboxamide |
| 429 | 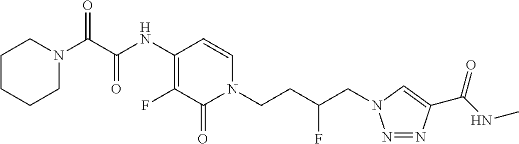 [SMILES: CNC(=O)c1cn(CC(F)CCn2ccc(NC(=O)C(=O)N3CCCCC3)c(F)c2=O)nn1] | 1-(2-fluoro-4-{3-fluoro-2-oxo-4-[2-oxo- 2-(piperidin-1-yl)acetamido]-1,2- dihydropyridin-1-yl}butyl)-N-methyl- 1H-1,2,3-triazole-4-carboxamide |
| 430 | 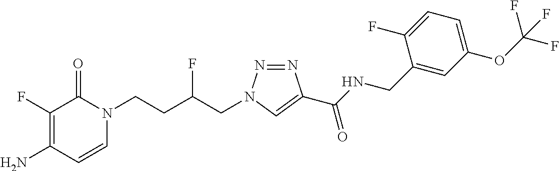 [SMILES: Nc1ccn(CCC(F)Cn2cc(C(=O)NCc3cc(OC(F)(F)F)ccc3F)nn2)c(=O)c1F] | 1-[4-(4-amino-3-fluoro-2-oxo-1,2- dihydropyridin-1-yl)-2-fluorobutyl]-N- {[2-fluoro-5- (trifluoromethoxy)phenyl]methyl}-1H- 1,2,3-triazole-4-carboxamide |
| 431 | 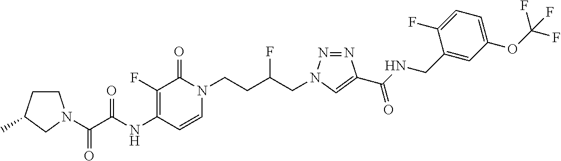 [SMILES: C[C@@H]1CCN(C(=O)C(=O)Nc2ccn(CCC(F)Cn3cc(C(=O)NCc4cc(OC(F)(F)F)ccc4F)nn3)c(=O)c2F)C1] | 1-[2-fluoro-4-(3-fluoro-4-{2-[(3R)-3- methylpyrrolidin-1-yl]-2- oxoacetamido}-2-oxo-1,2- dihydropyridin-1-yl)butyl]-N-{[2- fluoro-5- (trifluoromethoxy)phenyl]methyl}-1H- 1,2,3-triazole-4-carboxamide |
| 432 | 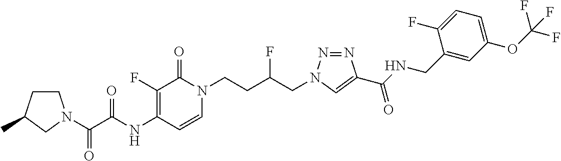 [SMILES: C[C@H]1CCN(C(=O)C(=O)Nc2ccn(CCC(F)Cn3cc(C(=O)NCc4cc(OC(F)(F)F)ccc4F)nn3)c(=O)c2F)C1] | 1-[2-fluoro-4-(3-fluoro-4-{2-[(3S)-3- methylpyrrolidin-1-yl]-2- oxoacetamido}-2-oxo-1,2- dihydropyridin-1-yl)butyl]-N-{[2- fluoro-5- (trifluoromethoxy)phenyl]methyl}-1H- 1,2,3-triazole-4-carboxamide |
| 433 | 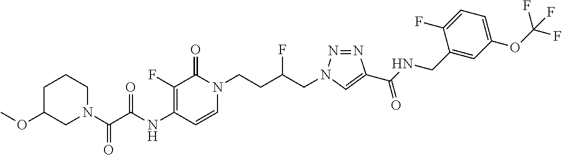 [SMILES: COC1CCCN(C(=O)C(=O)Nc2ccn(CCC(F)Cn3cc(C(=O)NCc4cc(OC(F)(F)F)ccc4F)nn3)c(=O)c2F)C1] | 1-(2-fluoro-4-{3-fluoro-4-[2-(3- methoxypiperidin-1-yl)-2- oxoacetamido]-2-oxo-1,2- dihydropyridin-1-yl}butyl)-N-{[2- fluoro-5- (trifluoromethoxy)phenyl]methyl}-1H- 1,2,3-triazole-4-carboxamide |
| 434 | 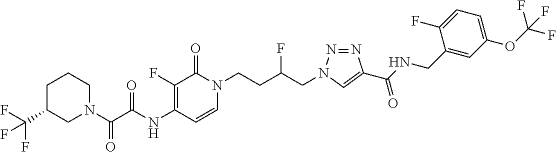 [SMILES: O=C(Nc1ccn(CCC(F)Cn2cc(C(=O)NCc3cc(OC(F)(F)F)ccc3F)nn2)c(=O)c1F)C(=O)N1CCC[C@@H](C(F)(F)F)C1] | 1-[2-fluoro-4-(3-fluoro-2-oxo-4-{2-oxo- 2-[(3R)-3-(trifluoromethyl)piperidin-1- yl]acetamido)-1,2-dihydropyridin-1- yl)butyl]-N-{[2-fluoro-5- (trifluoromethoxy)phenyl]methyl}-1H- 1,2,3-triazole-4-carboxamide |
| 435 | 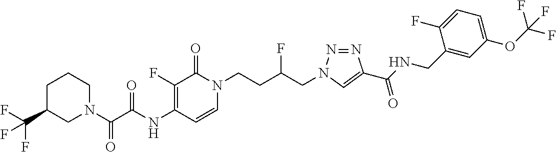 [SMILES: O=C(Nc1ccn(CCC(F)Cn2cc(C(=O)NCc3cc(OC(F)(F)F)ccc3F)nn2)c(=O)c1F)C(=O)N1CCC[C@H](C(F)(F)F)C1] | 1-[2-fluoro-4-(3-fluoro-2-oxo-4-{2-oxo- 2-[(3S)-3-(trifluoromethyl)piperidin-1- yl]acetamido}-1,2-dihydropyridin-1- yl)butyl]-N-{[2-fluoro-5- (trifluoromethoxy)phenyl]methyl}-1H- 1,2,3-triazole-4-carboxamide |
| 436 | 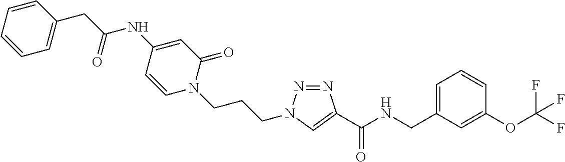 [SMILES: O=C(Cc1ccccc1)Nc1ccn(CCCn2cc(C(=O)NCc3cccc(OC(F)(F)F)c3)nn2)c(=O)c1] | 1-{3-[2-oxo-4-(2-phenylacetamido)-1,2- dihydropyridin-1-yl]propyl}-N-{[3- (trifluoromethoxy)phenyl]methyl}-1H- 1,2,3-triazole-4-carboxamide |
| 437 | 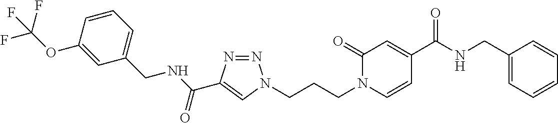 [SMILES: O=C(NCc1ccccc1)c1ccn(CCCn2cc(C(=O)NCc3cccc(OC(F)(F)F)c3)nn2)c(=O)c1] | N-benzyl-2-oxo-1-{ 3-[4-({[3- (trifluoromethoxy)phenyl]methyl} carbamoyl)-1H-1,2,3-triazol- 1-yl]propyl}- 1,2-dihydropyridine-4-carboxamide |
| 438 | 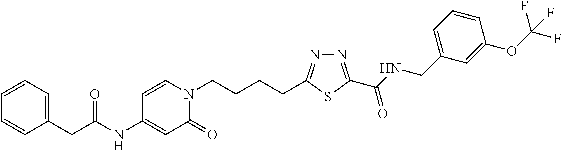 [SMILES: O=C(Cc1ccccc1)Nc1ccn(CCCCc2nnc(C(=O)NCc3cccc(OC(F)(F)F)c3)s2)c(=O)c1] | 5-{4-[2-oxo-4-(2-phenylacetamido)- 1,2-dihydropyridin-1-yl]butyl}-N-{[3- (trifluoromethoxy)phenyl]methyl}- 1,3,4-thiadiazole-2-carboxamide |
| 439 | 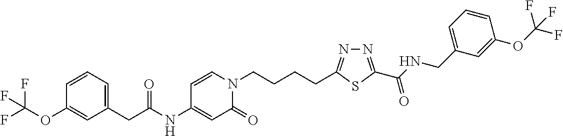 [SMILES: O=C(Cc1cccc(OC(F)(F)F)c1)Nc1ccn(CCCCc2nnc(C(=O)NCc3cccc(OC(F)(F)F)c3)s2)c(=O)c1] | 5-[4-(2-oxo-4-{2-[3- (trifluoromethoxy)phenyl]acetamido}- 1,2-dihydropyridin-1-yl)butyl]-N-{[3- (trifluoromethoxy)phenyl]methyl}- 1,3,4-thiadiazole-2-carboxamide |
| 440 | 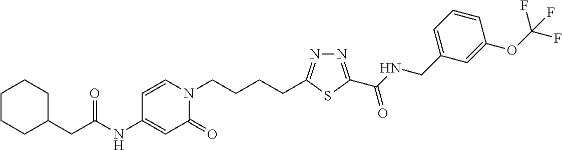 [SMILES: O=C(CC1CCCCC1)Nc1ccn(CCCCc2nnc(C(=O)NCc3cccc(OC(F)(F)F)c3)s2)c(=O)c1] | 5-{4-[4-(2-cyclohexylacetamido)-2- oxo-1,2-dihydropyridin-1-yl]butyl}-N- {[3-(trifluoromethoxy)phenyl]methyl]- 1,3,4-thiadiazole-2-carboxamide |
| 441 | 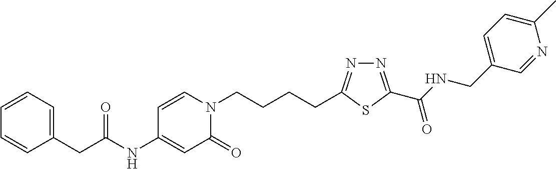 [SMILES: Cc1ccc(CNC(=O)c2nnc(CCCCn3ccc(NC(=O)Cc4ccccc4)cc3=O)s2)cn1] | N-[(6-methylpyridin-3-yl)methyl]-5-{4- [2-oxo-4-(2-phenylacetamido)-1,2- dihydropyridin-1-yl]butyl}-1,3,4- thiadiazole-2-carboxamide |
| 442 | 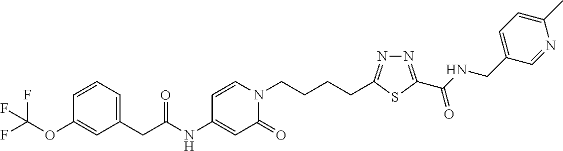 [SMILES: Cc1ccc(CNC(=O)c2nnc(CCCCn3ccc(NC(=O)Cc4cccc(OC(F)(F)F)c4)cc3=O)s2)cn1] | N-[(6-methylpyridin-3-yl)methyl]-5-[4- (2-oxo-4-{2-[3- (trifluoromethoxy)phenyl]acetamido}- 1,2-dihydropyridin-1-yl)butyl]-1,3,4- thiadiazole-2-carboxamide |
| 443 | 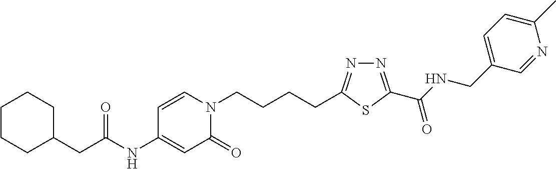 [SMILES: Cc1ccc(CNC(=O)c2nnc(CCCCn3ccc(NC(=O)CC4CCCCC4)cc3=O)s2)cn1] | 5-{4-[4-(2-cyclohexylacetamido)-2- oxo-1,2-dihydropyridin-1-yl]butyl}-N- [(6-methylpyridin-3-yl)methyl]-1,3,4- thiadiazole-2-carboxamide |
| 444 | 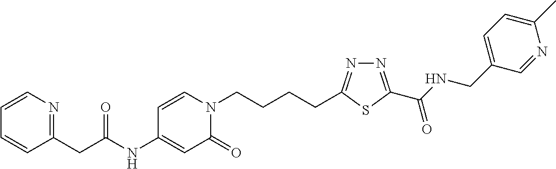 [SMILES: Cc1ccc(CNC(=O)c2nnc(CCCCn3ccc(NC(=O)Cc4ccccn4)cc3=O)s2)cn1] | N-[(6-methylpyridin-3-yl)methyl]-5-(4- {2-oxo-4-[2-(pyridin-2-yl)acetamido]- 1,2-dihydropyridin-1-yl}butyl)-1,3,4- thiadiazole-2-carboxamide |
| 445 | 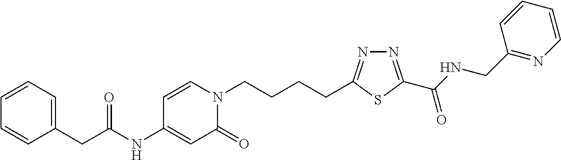 [SMILES: O=C(Cc1ccccc1)Nc1ccn(CCCCc2nnc(C(=O)NCc3ccccn3)s2)c(=O)c1] | 5-{4-[2-oxo-4-(2-phenylacetamido)-1,2- dihydropyridin-1-yl]butyl}-N-(pyridin- 2-ylmethyl)-1,3,4-thiadiazole-2- carboxamide |
| 446 | 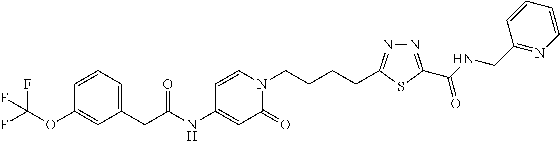 [SMILES: O=C(Cc1cccc(OC(F)(F)F)c1)Nc1ccn(CCCCc2nnc(C(=O)NCc3ccccn3)s2)c(=O)c1] | 5-[4-(2-oxo-4-{2-[3- (trifluoromethoxy)phenyl]acetamido}- 1,2-dihydropyridin-1-yl)butyl]-N- (pyridin-2-ylmethyl)-1,3,4-thiadiazole- 2-carboxamide |
| 447 | 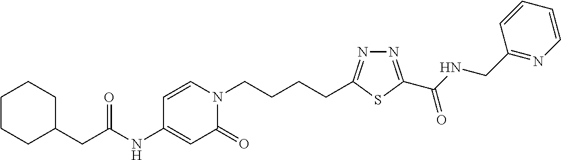 [SMILES: O=C(CC1CCCCC1)Nc1ccn(CCCCc2nnc(C(=O)NCc3ccccn3)s2)c(=O)c1] | 5-{4-[4-(2-cyclohexylacetamido)-2- oxo-1,2-dihydropyridin-1-yl]butyl}-N- (pyridin-2-ylmethyl)-1,3,4-thiadiazole- 2-carboxamide |
| 448 | 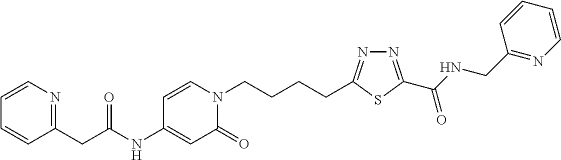 [SMILES: O=C(Cc1ccccn1)Nc1ccn(CCCCc2nnc(C(=O)NCc3ccccn3)s2)c(=O)c1] | 5-(4-{2-oxo-4-[2-(pyridin-2- yl)acetamido]-1,2-dihydropyridin-1- yl}butyl)-N-(pyridin-2-ylmethyl)-1,3,4- thiadiazole-2-carboxamide |
| 449 | 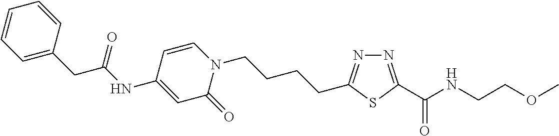 [SMILES: COCCNC(=O)c1nnc(CCCCn2ccc(NC(=O)Cc3ccccc3)cc2=O)s1] | N-(2-methoxyethyl)-5-{4-[2-oxo-4-(2- phenylacetamido)-1,2-dihydropyridin- 1-yl]butyl}-1,3,4-thiadiazole-2- carboxamide |
| 450 | 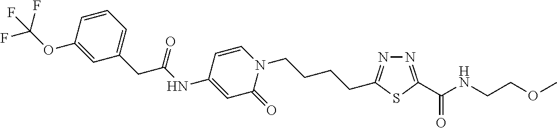 [SMILES: COCCNC(=O)c1nnc(CCCCn2ccc(NC(=O)Cc3cccc(OC(F)(F)F)c3)cc2=O)s1] | N-(2-methoxyethyl)-5-[4-(2-oxo-4-{2- [3-(trifluoro- methoxy)phenyl]acetamido}- 1,2-dihydropyridin-1-yl)butyl]-1,3,4- thiadiazole-2-carboxamide |
| 451 | 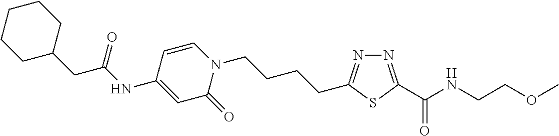 [SMILES: COCCNC(=O)c1nnc(CCCCn2ccc(NC(=O)CC3CCCCC3)cc2=O)s1] | 5-{4-[4-(2-cyclohexylacetamido)-2- oxo-1,2-dihydropyridin-1-yl]butyl)-N- (2-methoxyethyl)-1,3,4-thiadiazole-2- carboxamide |
| 452 | 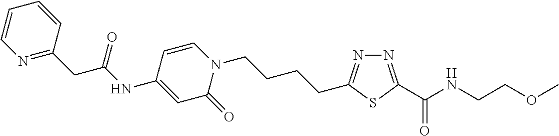 [SMILES: COCCNC(=O)c1nnc(CCCCn2ccc(NC(=O)Cc3ccccn3)cc2=O)s1] | N-(2-methoxyethyl)-5-(4-{2-oxo-4-[2- (pyridin-2-yl)acetamido]-1,2- dihydropyridin-1-yl]butyl)-1,3,4- thiadiazole-2-carboxamide |
| 453 | 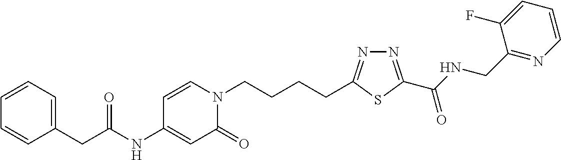 [SMILES: O=C(Cc1ccccc1)Nc1ccn(CCCCc2nnc(C(=O)NCc3ncccc3F)s2)c(=O)c1] | N-[(3-fluoropyridin-2-yl)methyl]-5-{4- [2-oxo-4-(2-phenylacetamido)-1,2- dihydropyridin-1-yl]butyl}-1,3,4- thiadiazole-2-carboxamide |
| 454 | 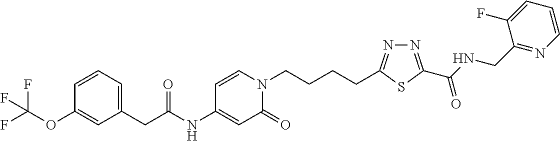 [SMILES: O=C(Cc1cccc(OC(F)(F)F)c1)Nc1ccn(CCCCc2nnc(C(=O)NCc3ncccc3F)s2)c(=O)c1] | N-[(3-fluoropyridin-2-yl)methyl]-5-[4- (2-oxo-4-{2-[3- (trifluoromethoxy)phenyl]acetamido}- 1,2-dihydropyridin-1-yl)butyl]-1,3,4- thiadiazole-2-carboxamide |
| 455 | 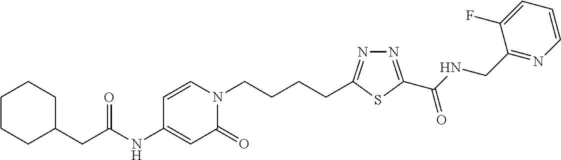 [SMILES: O=C(CC1CCCCC1)Nc1ccn(CCCCc2nnc(C(=O)NCc3ncccc3F)s2)c(=O)c1] | 5-{4-[4-(2-cyclohexylacetamido)-2- oxo-1,2-dihydropyridin-1-yl]butyl}-N- [(3-fluoropyridin-2-yl)methyl]-1,3,4- thiadiazole-2-carboxamide |
| 456 | 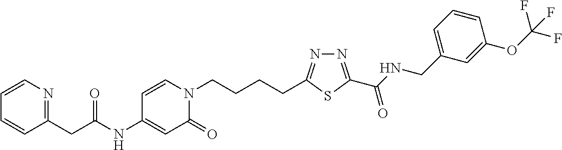 [SMILES: O=C(Cc1ccccn1)Nc1ccn(CCCCc2nnc(C(=O)NCc3cccc(OC(F)(F)F)c3)s2)c(=O)c1] | 5-(4-{2-oxo-4-[2-(pyridin-2- yl)acetamido]-1,2-dihydropyridin-1- yl}butyl)-N-{[3- (trifluoromethoxy)phenyl]methyl}- 1,3,4-thiadiazole-2-carboxamide |
| 457 | 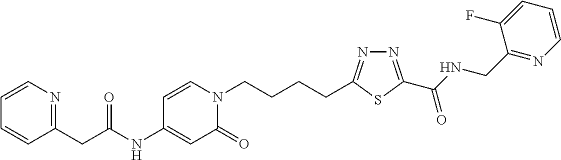 [SMILES: O=C(Cc1ccccn1)Nc1ccn(CCCCc2nnc(C(=O)NCc3ncccc3F)s2)c(=O)c1] | N-[(3-fluoropyridin-2-yl)methyl]-5-(4- {2-oxo-4-[2-(pyridin-2-yl)acetamido]- 1,2-dihydropyridin-1-yl}butyl)-1,3,4- thiadiazole-2-carboxamide |
| 458 | 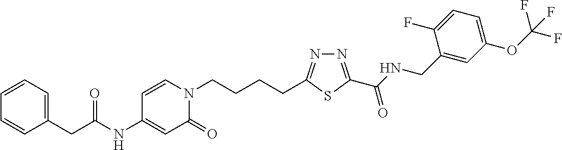 [SMILES: O=C(Cc1ccccc1)Nc1ccn(CCCCc2nnc(C(=O)NCc3cc(OC(F)(F)F)ccc3F)s2)c(=O)c1] | N-{[2-fluoro-5- (trifluoromethoxy)phenyl]methyl}-5- {4-[2-oxo-4-(2-phenylacetamido)-1,2- dihydropyridin-1-yl]butyl}-1,3,4- thiadiazole-2-carboxamide |
| 459 | 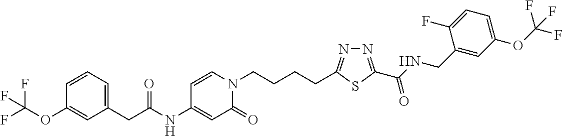 [SMILES: O=C(Cc1cccc(OC(F)(F)F)c1)Nc1ccn(CCCCc2nnc(C(=O)NCc3cc(OC(F)(F)F)ccc3F)s2)c(=O)c1] | N-{[2-fluoro-5- (trifluoromethoxy)phenyl]methyl}-5-[4- (2-oxo-4-{2-[3- (trifluoromethoxy)phenyl]acetamido}- 1,2-dihydropyridin-1-yl)butyl]-1,3,4- thiadiazole-2-carboxamide |
| 460 | 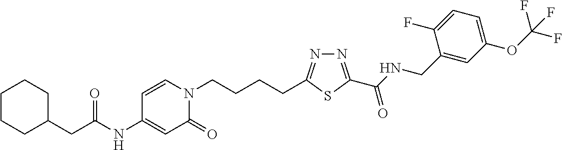 [SMILES: O=C(CC1CCCCC1)Nc1ccn(CCCCc2nnc(C(=O)NCc3cc(OC(F)(F)F)ccc3F)s2)c(=O)c1] | 5-{4-[4-(2-cyclohexylacetamido)-2- oxo-1,2-dihydropyridin-1-yl]butyl}-N- {[2-fluoro-5- (trifluoromethoxy)phenyl]methyl}- 1,3,4-thiadiazole-2-carboxamide |
| 461 | 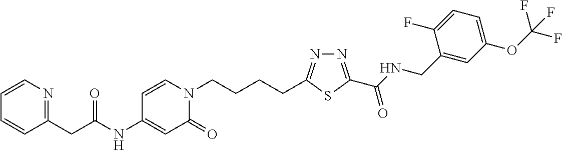 [SMILES: O=C(Cc1ccccn1)Nc1ccn(CCCCc2nnc(C(=O)NCc3cc(OC(F)(F)F)ccc3F)s2)c(=O)c1] | N-{[2-fluoro-5- (trifluoromethoxy)phenyl]methyl}-5-(4- {2-oxo-4-[2-(pyridin-2-yl)acetamido]- 1,2-dihydropyridin-1-yl}butyl)-1,3,4- thiadiazole-2-carboxamide |
| 462 | 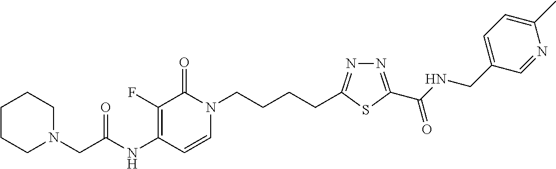 [SMILES: Cc1ccc(CNC(=O)c2nnc(CCCCn3ccc(NC(=O)CN4CCCCC4)c(F)c3=O)s2)cn1] | 5-(4-{3-fluoro-2-oxo-4-[2-(piperidin-1- yl)acetamido]-1,2-dihydropyridin-1- yl}butyl)-N-[(6-methylpyridin-3- yl)methyl]-1,3,4-thiadiazole-2- carboxamide |
| 463 | 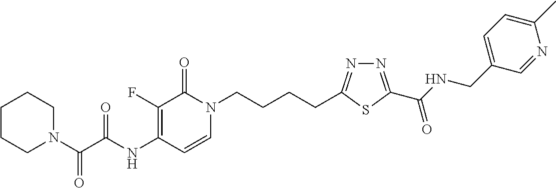 [SMILES: Cc1ccc(CNC(=O)c2nnc(CCCCn3ccc(NC(=O)C(=O)N4CCCCC4)c(F)c3=O)s2)cn1] | 5-(4-{3-fluoro-2-oxo-4-[2-oxo-2- (piperidin-1-yl)acetamido]-1,2- dihydropyridin-1-yl}butyl)-N-[(6- methylpyridin-3-yl)methyl]-1,3,4- thiadiazole-2-carboxamide |
| 464 | 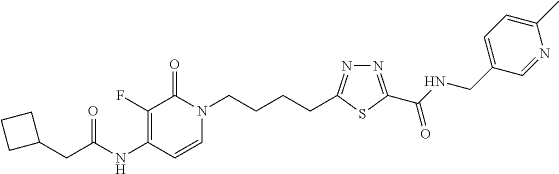 [SMILES: Cc1ccc(CNC(=O)c2nnc(CCCCn3ccc(NC(=O)CC4CCC4)c(F)c3=O)s2)cn1] | 5-{4-[4-(2-cyclobutylacetamido)-3- fluoro-2-oxo-1,2-dihydropyridin-1- yl]butyl}-N-[(6-methylpyridin-3- yl)methyl]-1,3,4-thiadiazole-2- carboxamide |
| 465 | 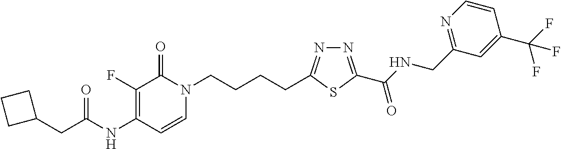 [SMILES: O=C(CC1CCC1)Nc1ccn(CCCCc2nnc(C(=O)NCc3cc(C(F)(F)F)ccn3)s2)c(=O)c1F] | 5-{4-[4-(2-cyclobutylacetamido)-3- fluoro-2-oxo-1,2-dihydropyridin-1- yl]butyl}-N-{[4- (trifluoromethyl)pyridin-2-yl]methyl}- 1,3,4-thiadiazole-2-carboxamide |
| 466 | 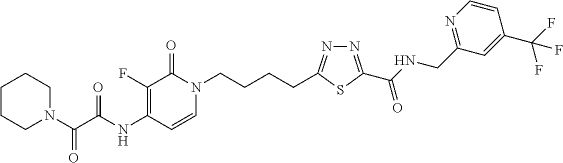 [SMILES: O=C(Nc1ccn(CCCCc2nnc(C(=O)NCc3cc(C(F)(F)F)ccn3)s2)c(=O)c1F)C(=O)N1CCCCC1] | 5-(4-{3-fluoro-2-oxo-4-[2-oxo-2- (piperidin-1-yl)acetamido]-1,2- dihydropyridin-1-yl)butyl)-N-{[4- (trifluoromethyl)pyridin-2-yl]methyl}- 1,3,4-thiadiazole-2-carboxamide |
| 467 | 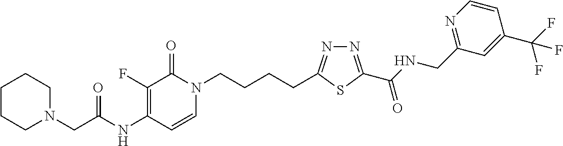 [SMILES: O=C(CN1CCCCC1)Nc1ccn(CCCCc2nnc(C(=O)NCc3cc(C(F)(F)F)ccn3)s2)c(=O)c1F] | 5-(4-{3-fluoro-2-oxo-4-[2-(piperidin-1- yl)acetamido]-1,2-dihydropyridin-1- yl}butyl)-N-{[4- (trifluoromethyl)pyridin-2-yl]methyl}- 1,3,4-thiadiazole-2-carboxamide |
| 468 | 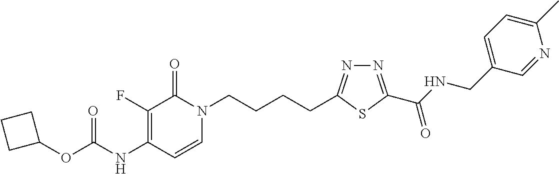 [SMILES: Cc1ccc(CNC(=O)c2nnc(CCCCn3ccc(NC(=O)OC4CCC4)c(F)c3=O)s2)cn1] | cyclobutyl N-{3-fluoro-1-[4-(5-{[(6- methylpyridin-3-yl)methyl]carbamoyl}- 1,3,4-thiadiazol-2-yl)butyl]-2-oxo-1,2- dihydropyridin-4-yl}carbamate |
| 469 | 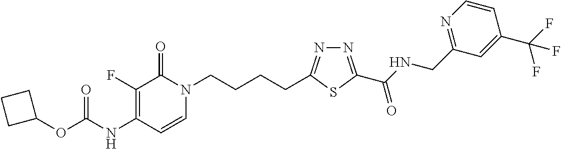 [SMILES: O=C(Nc1ccn(CCCCc2nnc(C(=O)NCc3cc(C(F)(F)F)ccn3)s2)c(=O)c1F)OC1CCC1] | cyclobutyl N-(3-fluoro-2-oxo-1-{4-[5- ({[4-(trifluoromethyl)pyridin-2- yl]methyl}carbamoyl)-1,3,4-thiadiazol- 2-yl]butyl}-1,2-dihydropyridin-4- yl)carbamate |
| 470 | 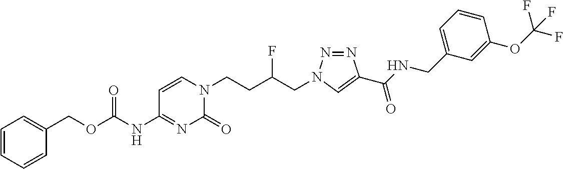 [SMILES: O=C(Nc1ccn(CCC(F)Cn2cc(C(=O)NCc3cccc(OC(F)(F)F)c3)nn2)c(=O)n1)OCc1ccccc1] | benzyl N-(1-{3-fluoro-4-[4-({[3- (trifluoromethoxy)phenyl]methyl} carbamoyl)-1H-1,2,3-triazol- 1-yl]butyl}-2- oxo-1,2-dihydropyrimidin-4- yl)carbamate |
| 471 | 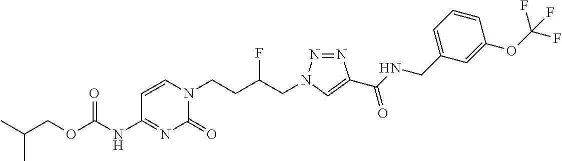 [SMILES: CC(C)COC(=O)Nc1ccn(CCC(F)Cn2cc(C(=O)NCc3cccc(OC(F)(F)F)c3)nn2)c(=O)n1] | 2-methylpropyl N-(1-{3-fluoro-4-[4- ({[3-(trifluoro- methoxy)phenyl]methyl}carbamoyl)- 1H-1,2,3-triazol-1-yl]butyl}-2- oxo-1,2-dihydropyrimidin-4- yl)carbamate |
| 472 | 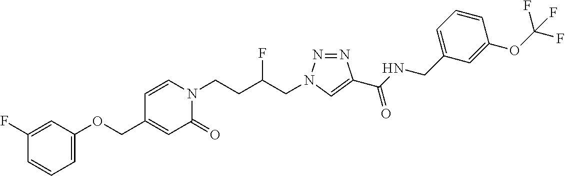 [SMILES: O=C(NCc1cccc(OC(F)(F)F)c1)c1cn(CC(F)CCn2ccc(COc3cccc(F)c3)cc2=O)nn1] | 1-{2-fluoro-4-[4-(3- fluorophenoxymethyl)-2-oxo-1,2- dihydropyridin-1-yl]butyl}-N-{[3- (trifluoromethoxy)phenyl]methyl}-1H- 1,2,3-triazole-4-carboxamide |
| 473 | 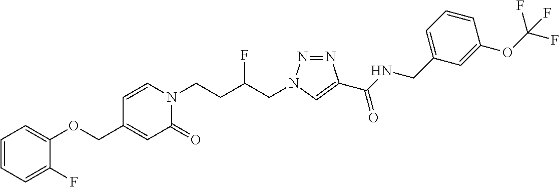 [SMILES: O=C(NCc1cccc(OC(F)(F)F)c1)c1cn(CC(F)CCn2ccc(COc3ccccc3F)cc2=O)nn1] | 1-{2-fluoro-4-[4-(2- fluorophenoxymethyl)-2-oxo-1,2- dihydropyridin-1-yl]butyl}-N-{[3- (trifluoromethoxy)phenyl]methyl}-1H- 1,2,3-triazole-4-carboxamide |
| 474 | 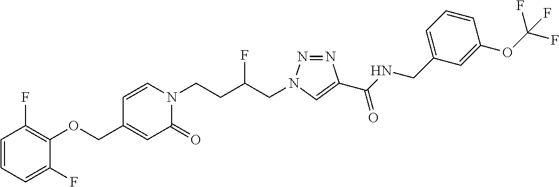 [SMILES: O=C(NCc1cccc(OC(F)(F)F)c1)c1cn(CC(F)CCn2ccc(COc3c(F)cccc3F)cc2=O)nn1] | 1-{4-[4-(2,6-difluorophenoxymethyl)-2- oxo-1,2-dihydropyridin-1-yl]-2- fluorobutyl}-N-{[3- (trifluoromethoxy)phenyl]methyl}-1H- 1,2,3-triazole-4-carboxamide |
| 475 | 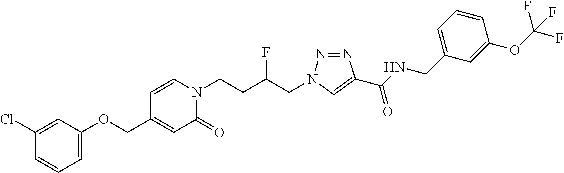 [SMILES: O=C(NCc1cccc(OC(F)(F)F)c1)c1cn(CC(F)CCn2ccc(COc3cccc(Cl)c3)cc2=O)nn1] | 1-{4-[4-(3-chlorophenoxymethyl)-2- oxo-1,2-dihydropyridin-1-yl]-2- fluorobutyl}-N-{[3- (trifluoromethoxy)phenyl]methyl}-1H- 1,2,3-triazole-4-carboxamide |
| 476 | 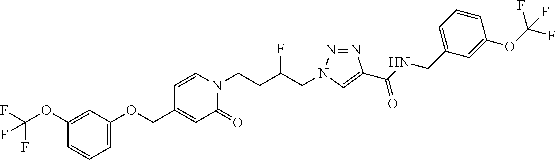 [SMILES: O=C(NCc1cccc(OC(F)(F)F)c1)c1cn(CC(F)CCn2ccc(COc3cccc(OC(F)(F)F)c3)cc2=O)nn1] | 1-(2-fluoro-4-{2-oxo-4-[3- (trifluoromethoxy)phenoxymethyl]-1,2- dihydropyridin-1-yl}butyl)-N-{[3- (trifluoromethoxy)phenyl]methyl}-1H- 1,2,3-triazole-4-carboxamide |
| 477 | 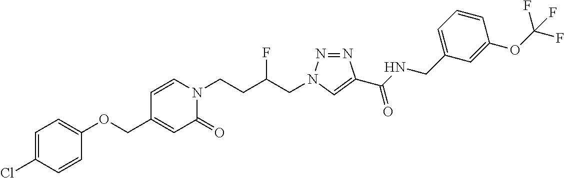 [SMILES: O=C(NCc1cccc(OC(F)(F)F)c1)c1cn(CC(F)CCn2ccc(COc3ccc(Cl)cc3)cc2=O)nn1] | 1-{4-[4-(4-chlorophenoxymethyl)-2- oxo-1,2-dihydropyridin-1-yl]-2- fluorobutyl}-N-{[3- (trifluoromethoxy)phenyl]methyl}-1H- 1,2,3-triazole-4-carboxamide |
| 478 | 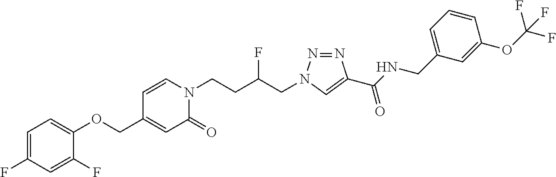 [SMILES: O=C(NCc1cccc(OC(F)(F)F)c1)c1cn(CC(F)CCn2ccc(COc3ccc(F)cc3F)cc2=O)nn1] | 1-{4-[4-(2,4-difluorophenoxymethyl)-2- oxo-1,2-dihydropyridin-1-yl]-2- fluorobutyl}-N-{[3- (trifluoromethoxy)phenyl]methyl}-1H- 1,2,3-triazole-4-carboxamide |
| 479 | 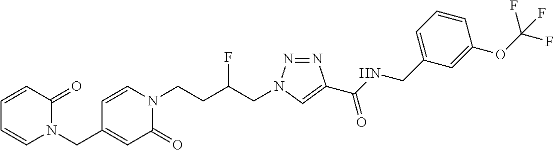 [SMILES: O=C(NCc1cccc(OC(F)(F)F)c1)c1cn(CC(F)CCn2ccc(Cn3ccccc3=O)cc2=O)nn1] | 1-(2-fluoro-4-{2-oxo-4-[(2-oxo-1,2- dihydropyridin-1-yl)methyl]-1,2- dihydropyridin-1-yl}butyl)-N-{[3- (trifluoromethoxy)phenyl]methyl}-1H- 1,2,3-triazole-4-carboxamide |
| 480 | 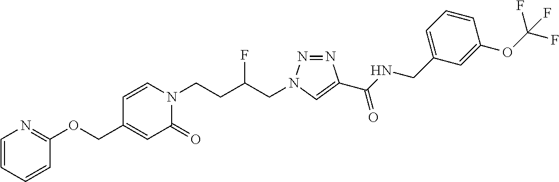 [SMILES: O=C(NCc1cccc(OC(F)(F)F)c1)c1cn(CC(F)CCn2ccc(COc3ccccn3)cc2=O)nn1] | 1-(2-fluoro-4-{2-oxo-4-[(pyridin-2- yloxy)methyl]-1,2-dihydropyridin-1- yl}butyl)-N-{[3- (trifluoromethoxy)phenyl]methyl}-1H- 1,2,3-triazole-4-carboxamide |
| 481 | 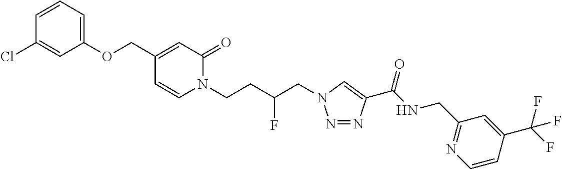 [SMILES: O=C(NCc1cc(C(F)(F)F)ccn1)c1cn(CC(F)CCn2ccc(COc3cccc(Cl)c3)cc2=O)nn1] | 1-{4-[4-(3-chlorophenoxymethyl)-2- oxo-1,2-dihydropyridin-1-yl]-2- fluorobutyl}-N-{[4- (trifluoromethyl)pyridin-2-yl]methyl}- 1H-1,2,3-triazole-4-carboxamide |
| 482 | 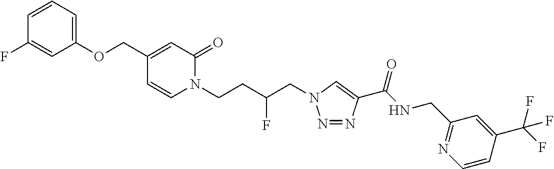 [SMILES: O=C(NCc1cc(C(F)(F)F)ccn1)c1cn(CC(F)CCn2ccc(COc3cccc(F)c3)cc2=O)nn1] | 1-{2-fluoro-4-[4-(3- fluorophenoxymethyl)-2-oxo-1,2- dihydropyridin-1-yl]butyl}-N-{[4- (trifluoromethyl)pyridin-2-yl]methyl}- 1H-1,2,3-triazole-4-carboxamide |
| 483 | 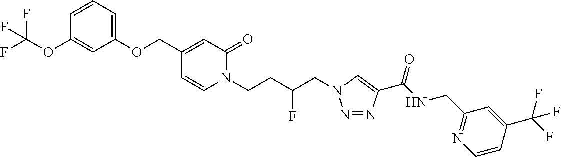 [SMILES: O=C(NCc1cc(C(F)(F)F)ccn1)c1cn(CC(F)CCn2ccc(COc3cccc(OC(F)(F)F)c3)cc2=O)nn1] | 1-(2-fluoro-4-{2-oxo-4-[3- (trifluoromethoxy)phenoxymethyl]-1,2- dihydropyridin-1-yl}butyl)-N-{[4- (trifluoromethyl)pyridin-2-yl]methyl}- 1H-1,2,3-triazole-4-carboxamide |
| 484 | 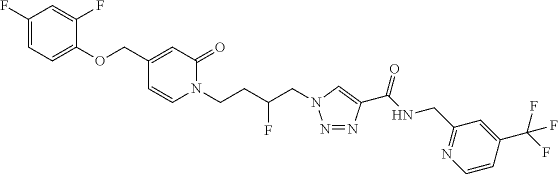 [SMILES: O=C(NCc1cc(C(F)(F)F)ccn1)c1cn(CC(F)CCn2ccc(COc3ccc(F)cc3F)cc2=O)nn1] | 1-{4-[4-(2,4-difluorophenoxymethyl)-2- oxo-1,2-dihydropyridin-1-yl]-2- fluorobutyl}-N-{[4- (trifluoromethyl)pyridin-2-yl]methyl}- 1H-1,2,3-triazole-4-carboxamide |
| 485 | 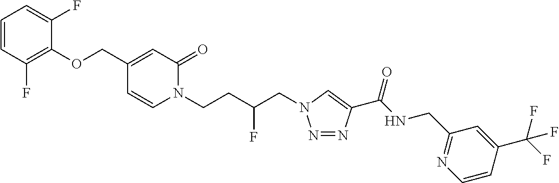 [SMILES: O=C(NCc1cc(C(F)(F)F)ccn1)c1cn(CC(F)CCn2ccc(COc3c(F)cccc3F)cc2=O)nn1] | 1-{4-[4-(2,6-difluorophenoxymethyl)-2- oxo-1,2-dihydropyridin-1-yl]-2- fluorobutyl}-N-{[4- (trifluoromethyl)pyridin-2-yl]methyl}- 1H-1,2,3-triazole-4-carboxamide |
| 486 | 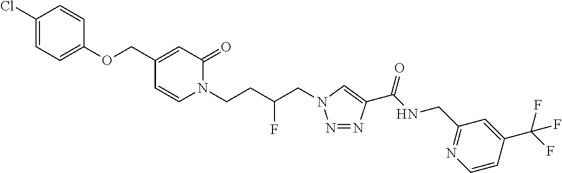 [SMILES: O=C(NCc1cc(C(F)(F)F)ccn1)c1cn(CC(F)CCn2ccc(COc3ccc(Cl)cc3)cc2=O)nn1] | 1-{4-[4-(4-chlorophenoxymethyl)-2- oxo-1,2-dihydropyridin-1-yl]-2- fluorobutyl}-N-{[4- (trifluoromethyl)pyridin-2-yl]methyl}- 1H-1,2,3-triazole-4-carboxamide |
| 487 | 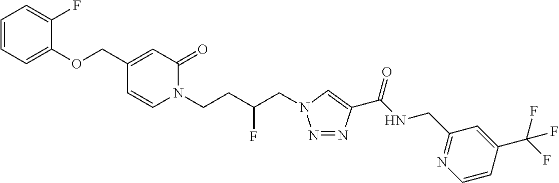 [SMILES: O=C(NCc1cc(C(F)(F)F)ccn1)c1cn(CC(F)CCn2ccc(COc3ccccc3F)cc2=O)nn1] | 1-{2-fluoro-4-[4-(2- fluorophenoxymethyl)-2-oxo-1,2- dihydropyridin-1-yl]butyl}-N-{[4- (trifluoromethyl)pyridin-2-yl]methyl}- 1H-1,2,3-triazole-4-carboxamide |
| 488 | 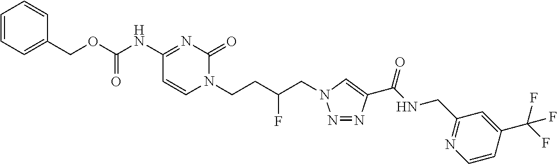 [SMILES: O=C(Nc1ccn(CCC(F)Cn2cc(C(=O)NCc3cc(C(F)(F)F)ccn3)nn2)c(=O)n1)OCc1ccccc1] | benzyl N-(1-{3-fluoro-4-[4-({[4- (trifluoromethyl)pyridin-2- yl]methyl}carbamoyl)-1H-1,2,3-triazol- 1-yl]butyl}-2-oxo-1,2- dihydropyrimidin-4-yl)carbamate |
| 489 | 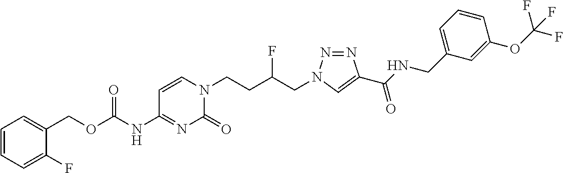 [SMILES: O=C(Nc1ccn(CCC(F)Cn2cc(C(=O)NCc3cccc(OC(F)(F)F)c3)nn2)c(=O)n1)OCc1ccccc1F] | (2-fluorophenyl)methyl N-(1-{3-fluoro- 4-[4-({[3-(trifluoro- methoxy)phenyl]methyl}carbamoyl)- 1H-1,2,3-triazol-1-yl]butyl}-2- oxo-1,2-dihydropyrimidin-4- yl)carbamate |
| 490 | 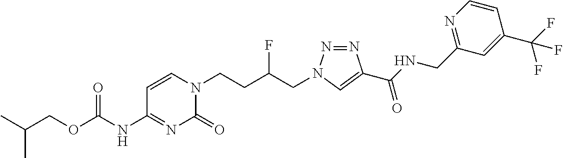 [SMILES: CC(C)COC(=O)Nc1ccn(CCC(F)Cn2cc(C(=O)NCc3cc(C(F)(F)F)ccn3)nn2)c(=O)n1] | 2-methylpropyl N-(1-{3-fluoro-4-[4- ({[4-(trifluoromethyl)pyridin-2- yl]methyl}carbamoyl)-1H-1,2,3-triazol- 1-yl]butyl}-2-oxo-1,2- dihydropyrimidin-4-yl)carbamate |
| 491 | 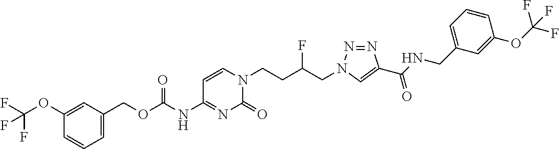 [SMILES: O=C(Nc1ccn(CCC(F)Cn2cc(C(=O)NCc3cccc(OC(F)(F)F)c3)nn2)c(=O)n1)OCc1cccc(OC(F)(F)F)c1] | [3-(trifluoromethoxy)phenyl]methyl N- (1-{3-fluoro-4-[4-({[3-(trifluoro- methoxy)phenyl]methyl}carbamoyl)- 1H-1,2,3-triazol-1-yl]butyl}-2- oxo-1,2-dihydropyrimidin-4- yl)carbamate |
| 492 | 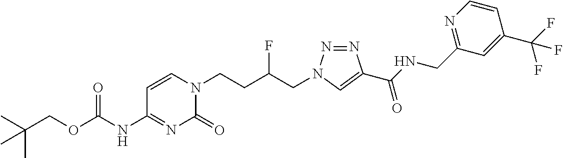 [SMILES: CC(C)(C)COC(=O)Nc1ccn(CCC(F)Cn2cc(C(=O)NCc3cc(C(F)(F)F)ccn3)nn2)c(=O)n1] | 2,2-dimethylpropyl N-(1-{3-fluoro-4- [4-({[4-(trifluoromethyl)pyridin-2- yl]methyl}carbamoyl)-1H-1,2,3-triazol- 1-yl]butyl}-2-oxo-1,2- dihydropyrimidin-4-yl)carbamate |
| 493 | 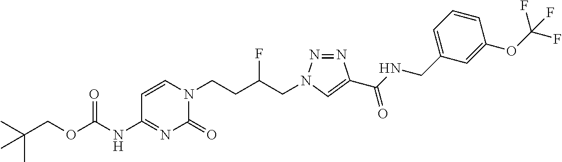 [SMILES: CC(C)(C)COC(=O)Nc1ccn(CCC(F)Cn2cc(C(=O)NCc3cccc(OC(F)(F)F)c3)nn2)c(=O)n1] | 2,2-dimethylpropyl N-(1-{3-fluoro-4- [4-({[3-(trifluoro- methoxy)phenyl]methyl}carbamoyl)- 1H-1,2,3-triazol-1-yl]butyl}-2- oxo-1,2-dihydropyrimidin-4- yl)carbamate |
| 494 | 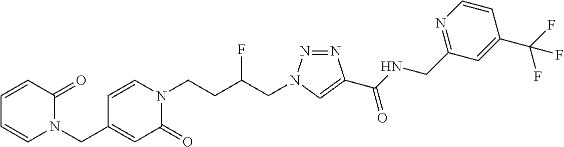 [SMILES: O=C(NCc1cc(C(F)(F)F)ccn1)c1cn(CC(F)CCn2ccc(Cn3ccccc3=O)cc2=O)nn1] | 1-(2-fluoro-4-{2-oxo-4-[(2-oxo-1,2- dihydropyridin-1-yl)methyl]-1,2- dihydropyridin-1-yl}butyl)-N-{[4- (trifluoromethyl)pyridin-2-yl]methyl}- 1H-1,2,3-triazole-4-carboxamide |
| 495 | 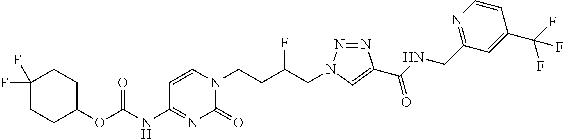 [SMILES: O=C(Nc1ccn(CCC(F)Cn2cc(C(=O)NCc3cc(C(F)(F)F)ccn3)nn2)c(=O)n1)OC1CCC(F)(F)CC1] | 4,4-difluorocyclohexyl N-(1-{3-fluoro- 4-[4-({[4-(trifluoromethyl)pyridin-2- yl]methyl}carbamoyl)-1H-1,2,3-triazol- 1-yl]butyl}-2-oxo-1,2- dihydropyrimidin-4-yl)carbamate |
| 496 | 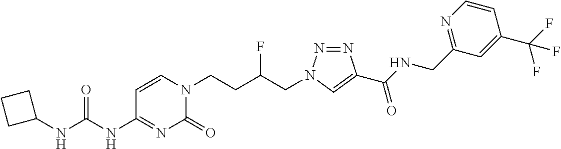 [SMILES: O=C(Nc1ccn(CCC(F)Cn2cc(C(=O)NCc3cc(C(F)(F)F)ccn3)nn2)c(=O)n1)NC1CCC1] | 1-(4-{4-[(cyclobutylcarbamoyl)amino]- 2-oxo-1,2-dihydropyrimidin-1-yl}-2- fluorobutyl)-N-{[4- (trifluoromethyl)pyridin-2-yl]methyl}- 1H-1,2,3-triazole-4-carboxamide |
| 497 | 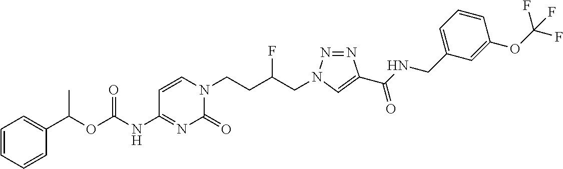 [SMILES: CC(OC(=O)Nc1ccn(CCC(F)Cn2cc(C(=O)NCc3cccc(OC(F)(F)F)c3)nn2)c(=O)n1)c1ccccc1] | 1-phenylethyl N-(1-{3-fluoro-4-[4-({[3- (trifluoromethoxy)phenyl]methyl} carbamoyl)-1H-1,2,3-triazol- 1-yl]butyl}-2-oxo-1,2- dihydropyrimidin-4-yl)carbamate |
| 498 | 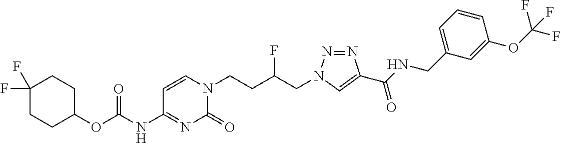 [SMILES: O=C(Nc1ccn(CCC(F)Cn2cc(C(=O)NCc3cccc(OC(F)(F)F)c3)nn2)c(=O)n1)OC1CCC(F)(F)CC1] | 4,4-difluorocyclohexyl N-(1-{3-fluoro- 4-[4-({[3-(trifluoro- methoxy)phenyl]methyl}carbamoyl)- 1H-1,2,3-triazol-1-yl]butyl}-2- oxo-1,2-dihydropyrimidin-4- yl)carbamate |
| 499 | 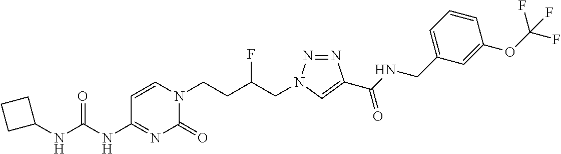 [SMILES: O=C(Nc1ccn(CCC(F)Cn2cc(C(=O)NCc3cccc(OC(F)(F)F)c3)nn2)c(=O)n1)NC1CCC1] | 1-(4-{4-[(cyclobutylcarbamoyl)amino]- 2-oxo-1,2-dihydropyrimidin-1-yl}-2- fluorobutyl)-N-{[3- (trifluoromethoxy)phenyl]methyl}-1H- 1,2,3-triazole-4-carboxamide |
| 500 | 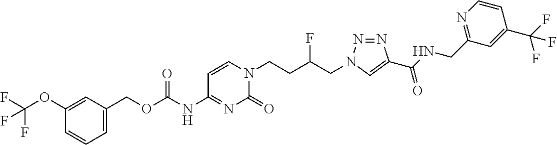 [SMILES: O=C(Nc1ccn(CCC(F)Cn2cc(C(=O)NCc3cc(C(F)(F)F)ccn3)nn2)c(=O)n1)OCc1cccc(OC(F)(F)F)c1] | [3-(trifluoromethoxy)phenyl]methyl N- (1-{3-fluoro-4-[4-({[4- (trifluoromethyl)pyridin-2- yl]methyl}carbamoyl)-1H-1,2,3-triazol- 1-yl]butyl}-2-oxo-1,2- dihydropyrimidin-4-yl)carbamate |
| 501 | 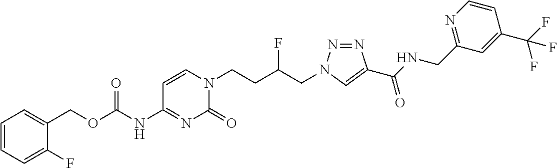 [SMILES: O=C(Nc1ccn(CCC(F)Cn2cc(C(=O)NCc3cc(C(F)(F)F)ccn3)nn2)c(=O)n1)OCc1ccccc1F] | (2-fluorophenyl)methyl N-(1-{3-fluoro- 4-[4-({[4-(trifluoromethyl)pyridin-2- yl]methyl}carbamoyl)-1H-1,2,3-triazol- 1-yl]butyl}-2-oxo-1,2- dihydropyrimidin-4-yl)carbamate |
| 502 | 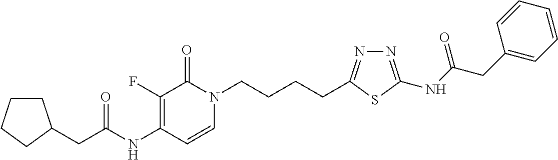 [SMILES: O=C(Cc1ccccc1)Nc1nnc(CCCCn2ccc(NC(=O)CC3CCCC3)c(F)c2=O)s1] | 2-cyclopentyl-N-(3-fluoro-2-oxo-1-{4- [5-(2-phenylacetamido)-1,3,4- thiadiazol-2-yl]butyl}-1,2- dihydropyridin-4-yl)acetamide |
| 503 | 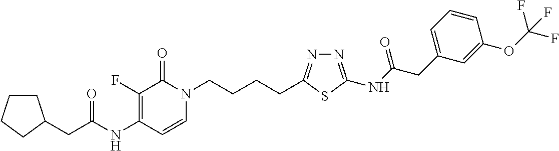 [SMILES: O=C(Cc1cccc(OC(F)(F)F)c1)Nc1nnc(CCCCn2ccc(NC(=O)CC3CCCC3)c(F)c2=O)s1] | 2-cyclopentyl-N-{3-fluoro-2-oxo-1-[4- (5-{2-[3- (trifluoromethoxy)phenyl]acetamido}- 1,3,4-thiadiazol-2-yl)butyl]-1,2- dihydropyridin-4-yl}acetamide |
| 504 | 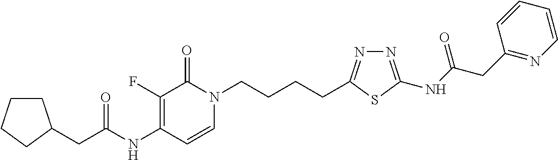 [SMILES: O=C(Cc1ccccn1)Nc1nnc(CCCCn2ccc(NC(=O)CC3CCCC3)c(F)c2=O)s1] | 2-cyclopentyl-N-[3-fluoro-2-oxo-1-(4- {5-[2-(pyridin-2-yl)acetamido]-1,3,4- thiadiazol-2-yl}butyl)-1,2- dihydropyridin-4-yl]acetamide |
| 505 | 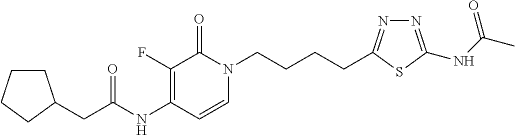 [SMILES: CC(=O)Nc1nnc(CCCCn2ccc(NC(=O)CC3CCCC3)c(F)c2=O)s1] | 2-cyclopentyl-N-{1-[4-(5-acetamido- 1,3,4-thiadiazol-2-yl)butyl]-3-fluoro-2- oxo-1,2-dihydropyridin-4-yl}acetamide |

Table 2 below reports the observed molecular ion (ES⁺) (Mass Spec) [M+H]⁺ of each Example, as well as the method by which each compound may be made by reference to each Example whose synthesis is substantially similar that one skilled in the art could produce the compound using, if necessary, variations know in the art.

TABLE 2
Observed Molecular Weight and Synthesis for Examples

| Ex. | Exact | Obs. | Synthesis, |
|---|---|---|---|
| No. | mass | mass | as in Ex. |
| 30 | 383 | 384 | 1 |
| 31 | 501 | 502 | 1 |
| 32 | 409 | 409 | 2 |
| 33 | 569 | 569 | 2 |
| 34 | 485 | 486 | 2 |
| 35 | 450 | 451 | 2 |
| 36 | 490 | 491 | 2 |
| 37 | 451 | 452 | 2 |
| 38 | 451 | 452 | 2 |
| 39 | 456 | 457 | 2 |
| 40 | 492 | 493 | 10 |
| 41 | 574 | 575 | 10 |
| 42 | 584 | 585 | 7 |
| 43 | 536 | 537 | 7 |
| 44 | 576 | 577 | 7 |
| 45 | 574 | 575 | 6 |
| 46 | 572 | 573 | 6 |
| 47 | 562 | 563 | 5 |
| 48 | 576 | 577 | 5 |
| 49 | 569 | 570 | 8 |
| 50 | 492 | 493 | 5 |
| 51 | 568 | 569 | 10 |
| 52 | 652 | 653 | 10 |
| 53 | 646 | 647 | 10 |
| 54 | 520 | 521 | 5 |
| 55 | 586 | 587 | 5 |
| 56 | 576 | 577 | 5 |
| 57 | 501 | 502 | 7 |
| 58 | 492 | 493 | 8 |
| 59 | 652 | 653 | 8 |
| 60 | 569 | 570 | 8 |
| 61 | 628 | 629 | 8 |
| 62 | 534 | 535 | 8 |
| 63 | 574 | 575 | 8 |
| 64 | 668 | 669 | 7 |
| 65 | 598 | 599 | 10 |
| 66 | 586 | 587 | 10 |
| 67 | 598 | 599 | 10 |
| 68 | 646 | 647 | 10 |
| 69 | 569 | 570 | 10 |
| 70 | 583 | 584 | 10 |
| 71 | 526 | 527 | 15 |
| 72 | 384 | 385 | 16 |
| 73 | 504 | 503 | 16 |
| 74 | 516 | 517 | 16 |
| 75 | 580 | 581 | 20 |
| 76 | 504 | 505 | 20 |
| 77 | 462 | 463 | 21 |
| 78 | 596 | 597 | 20 |
| 79 | 468 | 469 | 16 |
| 80 | 602 | 603 | 19 |
| 81 | 587 | 588 | 19 |
| 82 | 503 | 504 | 19 |
| 83 | 518 | 519 | 19 |
| 84 | 518 | 519 | 19 |
| 85 | 631 | 632 | 19 |
| 86 | 449 | 450 | 23 |
| 87 | 463 | 464 | 23 |
| 88 | 503 | 504 | 23 |
| 89 | 525 | 526 | 23 |
| 90 | 525 | 526 | 23 |
| 91 | 680 | 681 | 15 |
| 92 | 420 | 421 | 15 |
| 93 | 436 | 437 | 24 |
| 94 | 464 | 465 | 24 |
| 95 | 504 | 505 | 24 |
| 96 | 512 | 513 | 24 |
| 97 | 498 | 499 | 24 |
| 98 | 478 | 479 | 24 |
| 99 | 580 | 581 | 22 |
| 100 | 512 | 513 | 22 |
| 101 | 596 | 597 | 22 |
| 102 | 548 | 549 | 24 |
| 103 | 588 | 589 | 24 |
| 104 | 562 | 563 | 24 |
| 105 | 590 | 591 | 24 |
| 106 | 547 | 548 | 23 |
| 107 | 587 | 588 | 23 |
| 108 | 586 | 587 | 22 |
| 109 | 587 | 588 | 22 |
| 110 | 596 | 597 | 22 |
| 111 | 546 | 547 | 22 |
| 112 | 564 | 565 | 24 |
| 113 | 520 | 521 | 22 |
| 114 | 584 | 585 | 22 |
| 115 | 569 | 570 | 10 |
| 116 | 581 | 582 | 22 |
| 117 | 534 | 535 | 22 |
| 118 | 581 | 582 | 22 |
| 119 | 472 | 473 | 28 |
| 120 | 519 | 520 | 28 |
| 120 | 519 | 520 | 28 |
| 121 | 486 | 487 | 28 |
| 122 | 518 | 519 | 28 |
| 124 | 535 | 536 | 127 |
| 125 | 589 | 590 | 127 |
| 126 | 589 | 590 | 127 |
| 127 | Exemplified | | |
| 128 | 492 | 493 | 1 |
| 129 | 519 | 520 | 1 |
| 130 | 526 | 527 | 1 |
| 131 | 527 | 528 | 1 |
| 132 | 450 | 451 | 1 |
| 133 | 594 | 595 | 1 |
| 134 | 586 | 587 | 12 |
| 135 | 515 | 516 | 7 |
| 136 | 535 | 536 | 6 |
| 137 | 549 | 550 | 8 |
| 138 | 549 | 550 | 8 |
| 139 | 534 | 535 | 180 |
| 140 | 434 | 435 | 4 |
| 141 | 558 | 559 | 5 |
| 142 | 476 | 477 | 5 |
| 143 | 481 | 482 | 10 |
| 144 | 586 | 587 | 13 |
| 145 | 505 | 506 | 10 |
| 146 | 509 | 510 | 10 |
| 147 | 503 | 504 | 2 |
| 148 | 517 | 518 | 13 |
| 149 | 521 | 522 | 13 |
| 150 | 517 | 518 | 12 |
| 151 | 521 | 522 | 12 |
| 152 | 544 | 545 | 10 |
| 153 | 544 | 545 | 10 |
| 154 | 516 | 517 | 10 |
| 155 | 559 | 560 | 10 |
| 156 | 587 | 588 | 10 |
| 157 | 569 | 570 | 10 |
| 158 | 499 | 500 | 7 |
| 159 | 477 | 478 | 7 |
| 160 | 581 | 582 | 13 |
| 161 | 571 | 572 | 3 |
| 162 | 517 | 518 | 13 |
| 163 | 581 | 582 | 12 |
| 164 | 571 | 572 | 12 |
| 165 | 517 | 518 | 12 |
| 166 | 610 | 611 | 10 |
| 167 | 586 | 587 | 10 |
| 168 | 568 | 569 | 10 |
| 169 | 592 | 593 | 10 |
| 170 | 604 | 605 | 10 |
| 171 | 626 | 627 | 10 |
| 172 | 569 | 570 | 386 |
| 173 | 586 | 587 | 180 |
| 174 | 612 | 613 | 7 |
| 175 | 592 | 593 | 12 |
| 176 | 610 | 611 | 12 |
| 177 | 523 | 524 | 12 |
| 178 | 577 | 578 | 12 |
| 179 | 624 | 625 | 10 |
| 180 | Exemplified | | |
| 181 | 542 | 543 | 10 |
| 182 | 605 | 606 | 12 |
| 183 | 571 | 572 | 12 |
| 184 | 616 | 617 | 12 |
| 185 | 660 | 661 | 12 |
| 186 | 616 | 617 | 12 |
| 187 | 594 | 595 | 12 |
| 188 | 535 | 536 | 12 |
| 189 | 590 | 591 | 12 |
| 190 | 491 | 492 | 10 |
| 191 | 491 | 492 | 10 |
| 192 | 520 | 521 | 10 |
| 193 | 588 | 589 | 10 |
| 194 | 559 | 560 | 10 |
| 195 | 525 | 526 | 12 |
| 196 | 525 | 526 | 12 |
| 197 | 525 | 526 | 12 |
| 198 | 541 | 542 | 12 |
| 199 | 509 | 510 | 12 |
| 200 | 511 | 512 | 12 |
| 201 | 546 | 547 | 10 |
| 202 | 574 | 575 | 10 |
| 203 | 574 | 575 | 10 |
| 204 | 588 | 589 | 10 |
| 205 | 675 | 676 | 10 |
| 206 | 576 | 577 | 10 |
| 207 | 603 | 604 | 10 |
| 208 | 590 | 591 | 10 |
| 209 | 603 | 604 | 10 |
| 210 | 560 | 561 | 10 |
| 211 | 675 | 676 | 10 |
| 212 | 610 | 611 | 180 |
| 213 | 595 | 596 | 337 |
| 214 | 532 | 533 | 12 |
| 215 | 562 | 563 | 12 |
| 216 | 554 | 555 | 12 |
| 217 | 564 | 565 | 12 |
| 218 | 621 | 622 | 12 |
| 219 | 693 | 694 | 12 |
| 220 | 578 | 579 | 12 |
| 221 | 679 | 680 | 12 |
| 222 | 715 | 716 | 12 |
| 223 | 602 | 603 | 12 |
| 224 | 602 | 603 | 12 |
| 225 | 465 | 466 | 180 |
| 226 | 586 | 587 | 180 |
| 227 | 597 | 598 | 337 |
| 228 | 611 | 612 | 337 |
| 229 | 636 | 637 | 337 |
| 230 | 608 | 609 | 337 |
| 231 | 643 | 644 | 337 |
| 232 | 601 | 602 | 337 |
| 233 | 536 | 537 | 337 |
| 234 | 568 | 569 | 12 |
| 235 | 611 | 612 | 12 |
| 236 | 621 | 622 | 12 |
| 237 | 671 | 672 | 12 |
| 238 | 621 | 622 | 12 |
| 239 | 707 | 708 | 12 |
| 240 | 617 | 618 | 12 |
| 241 | 707 | 708 | 12 |
| 242 | 657 | 658 | 12 |
| 243 | 639 | 640 | 12 |
| 244 | 635 | 636 | 12 |
| 245 | 693 | 694 | 12 |
| 246 | 587 | 588 | 12 |
| 247 | 593 | 594 | 12 |
| 248 | 601 | 602 | 12 |
| 249 | 693 | 694 | 12 |
| 250 | 587 | 588 | 12 |
| 251 | 617 | 618 | 12 |
| 252 | 593 | 594 | 12 |
| 253 | 637 | 638 | 12 |
| 254 | 608 | 609 | 337 |
| 255 | 666 | 667 | 12 |
| 256 | 618 | 619 | 337 |
| 257 | 623 | 624 | 337 |
| 258 | 595 | 596 | 180 |
| 259 | 595 | 596 | 180 |
| 260 | 595 | 596 | 180 |
| 261 | 595 | 596 | 180 |
| 262 | 595 | 596 | 180 |
| 263 | 587 | 588 | 386 |
| 264 | 594 | 595 | 180 |
| 265 | 578 | 579 | 180 |
| 266 | 541 | 542 | 180 |
| 267 | 527 | 528 | 180 |
| 268 | 585 | 586 | 180 |
| 269 | 593 | 594 | 337 |
| 270 | 579 | 580 | 337 |
| 271 | 694 | 695 | 337 |
| 272 | 637 | 638 | 12 |
| 273 | 651 | 652 | 12 |
| 274 | 629 | 630 | 337 |
| 275 | 611 | 612 | 337 |
| 276 | 623 | 624 | 337 |
| 277 | 665 | 666 | 337 |
| 278 | 609 | 610 | 337 |
| 279 | 672 | 673 | 337 |
| 280 | 593 | 594 | 337 |
| 281 | 665 | 666 | 12 |
| 282 | 486 | 487 | 180 |
| 283 | 611 | 612 | 365 |
| 284 | 634 | 635 | 180 |
| 285 | 634 | 635 | 180 |
| 286 | 620 | 621 | 180 |
| 287 | 620 | 621 | 180 |
| 288 | 611 | 612 | 337 |
| 289 | 711 | 712 | 180 |
| 290 | 647 | 648 | 337 |
| 291 | 629 | 630 | 337 |
| 292 | 684 | 685 | 337 |
| 293 | 597 | 598 | 337 |
| 294 | 641 | 642 | 337 |
| 295 | 647 | 648 | 337 |
| 296 | 712 | 713 | 337 |
| 297 | 724 | 725 | 337 |
| 298 | 517 | 518 | 180 |
| 299 | 611 | 612 | 180 |
| 300 | 683 | 684 | 180 |
| 301 | 721 | 722 | 180 |
| 302 | 689 | 690 | 180 |
| 303 | 527 | 528 | 180 |
| 304 | Exemplified | | |
| 305 | 662 | 663 | 180 |
| 306 | 662 | 663 | 180 |
| 307 | 535 | 536 | 180 |
| 308 | 722 | 723 | 337 |
| 309 | 634 | 635 | 365 |
| 310 | 634 | 635 | 365 |
| 311 | 441 | 442 | 180 |
| 312 | 565 | 566 | 180 |
| 313 | 565 | 566 | 180 |
| 314 | Exemplified | | |
| 315 | 618 | 619 | 180 |
| 316 | 618 | 619 | 180 |
| 317 | 589 | 589 | 180 |
| 318 | 581 | 582 | 180 |
| 319 | 571 | 572 | 180 |
| 320 | 571 | 572 | 180 |
| 321 | 471 | 472 | 180 |
| 322 | 471 | 472 | 180 |
| 323 | 589 | 590 | 180 |
| 324 | 668 | 669 | 180 |
| 325 | 647 | 648 | 180 |
| 326 | 605 | 606 | 180 |
| 327 | 622 | 623 | 180 |
| 328 | 622 | 623 | 180 |
| 329 | 581 | 582 | 180 |
| 330 | 571 | 572 | 180 |
| 331 | 471 | 472 | 180 |
| 332 | 567 | 568 | 180 |
| 333 | 603 | 604 | 180 |
| 334 | 640 | 641 | 180 |
| 335 | 640 | 641 | 180 |
| 336 | 567 | 568 | 180 |
| 337 | Exemplified | | |
| 338 | 596 | 597 | 337 |
| 339 | 582 | 583 | 180 |
| 340 | 584 | 585 | 180 |
| 341 | 596 | 597 | 180 |
| 342 | 582 | 583 | 180 |
| 343 | 603 | 604 | 180 |
| 344 | 596 | 597 | 180 |
| 345 | 610 | 611 | 180 |
| 346 | 612 | 613 | 180 |
| 347 | 586 | 587 | 358 |
| 348 | 589 | 590 | 180 |
| 349 | 603 | 604 | 180 |
| 350 | 567 | 568 | 180 |
| 351 | 596 | 597 | 337 |
| 352 | 610 | 611 | 337 |
| 353 | 590 | 591 | 180 |
| 354 | 572 | 573 | 180 |
| 355 | 596 | 597 | 180 |
| 356 | 517 | 518 | 180 |
| 357 | 567 | 568 | 180 |
| 358 | Exemplified | | |
| 359 | 558 | 559 | 358 |
| 360 | 581 | 582 | 180 |
| 361 | 607 | 608 | 180 |
| 362 | 607 | 608 | 180 |
| 363 | 668 | 669 | 180 |
| 364 | 567 | 568 | 180 |
| 365 | Exemplified | | |
| 366 | 471 | 472 | 180 |
| 367 | 589 | 590 | 180 |
| 368 | 603 | 604 | 180 |
| 369 | 581 | 582 | 180 |
| 370 | 567 | 568 | 180 |
| 371 | 607 | 608 | 180 |
| 372 | 607 | 608 | 180 |
| 373 | 539 | 540 | 180 |
| 374 | 531 | 532 | 180 |
| 375 | 553 | 554 | 180 |
| 376 | 584 | 585 | 180 |
| 377 | 569 | 570 | 180 |
| 379 | 589 | 590 | 180 |
| 380 | 546 | 547 | 180 |
| 381 | 512 | 513 | 180 |
| 382 | 469 | 470 | 491 |
| 383 | 595 | 596 | 491 |
| 384 | 555 | 556 | 491 |
| 385 | 567 | 568 | 491 |
| 386 | Exemplified | | |
| 387 | 531 | 532 | 180 |
| 388 | 545 | 546 | 180 |
| 389 | 583 | 584 | 180 |
| 390 | 567 | 568 | 180 |
| 391 | 408 | 409 | 180 |
| 392 | 572 | 573 | 180 |
| 393 | 513 | 514 | 180 |
| 394 | 581 | 582 | 180 |
| 395 | 564 | 565 | 180 |
| 396 | 515 | 516 | 180 |
| 397 | 569 | 570 | 180 |
| 398 | 569 | 570 | 180 |
| 399 | 569 | 570 | 180 |
| 400 | 597 | 598 | 180 |
| 401 | 550 | 551 | 386 |
| 402 | 496 | 497 | 386 |
| 403 | 534 | 535 | 337 |
| 404 | 576 | 577 | 10 |
| 405 | 602 | 603 | 10 |
| 406 | 606 | 607 | 10 |
| 407 | 520 | 521 | 337 |
| 408 | 366 | 367 | 414 |
| 409 | 605 | 606 | 180 |
| 410 | 552 | 553 | 491 |
| 411 | 542 | 543 | 358 |
| 412 | 556 | 557 | 358 |
| 413 | 498 | 499 | 491 |
| 414 | | | |
| 415 | 608 | 609 | 337 |
| 416 | 632 | 633 | 337 |
| 417 | 627 | 628 | 422 |
| 418 | 639 | 640 | 435 |
| 419 | 693 | 694 | 435 |
| 420 | 650 | 651 | 435 |
| 421 | 609 | 610 | 422 |
| 422 | Exemplified | | |
| 423 | 610 | 611 | 422 |
| 424 | 637 | 638 | 435 |
| 425 | 613 | 614 | 435 |
| 426 | 641 | 642 | 435 |
| 427 | 542 | 543 | 422 |
| 428 | 625 | 626 | 358 |
| 429 | 465 | 466 | 422 |
| 430 | 504 | 505 | 180 |
| 431 | 643 | 644 | 435 |
| 432 | 643 | 644 | 435 |
| 433 | 673 | 674 | 435 |
| 434 | 711 | 712 | 435 |
| 435 | Exemplified | | |
| 436 | 554 | 555 | 8 |
| 437 | 554 | 555 | 8 |
| 438 | 585 | 586 | 464 |
| 439 | 669 | 670 | 464 |
| 440 | 591 | 592 | 464 |
| 441 | 516 | 517 | 464 |
| 442 | 600 | 601 | 464 |
| 443 | 522 | 523 | 464 |
| 444 | 517 | 518 | 464 |
| 445 | 502 | 503 | 464 |
| 446 | 586 | 587 | 464 |
| 447 | 508 | 509 | 464 |
| 448 | 503 | 504 | 464 |
| 449 | 469 | 470 | 464 |
| 450 | 553 | 554 | 464 |
| 451 | 475 | 476 | 464 |
| 452 | 470 | 471 | 464 |
| 453 | 520 | 521 | 464 |
| 454 | 604 | 605 | 464 |
| 455 | 526 | 527 | 464 |
| 456 | 586 | 587 | 464 |
| 457 | 521 | 522 | 464 |
| 458 | 603 | 604 | 464 |
| 459 | 687 | 688 | 464 |
| 460 | 609 | 610 | 464 |
| 461 | 604 | 605 | 464 |
| 462 | 541 | 542 | 464 |
| 463 | 555 | 556 | 464 |
| 464 | Exemplified | | |
| 465 | 566 | 567 | 464 |
| 466 | 609 | 610 | 464 |
| 467 | 595 | 596 | 464 |
| 468 | Exemplified | | |
| 469 | 568 | 569 | 468 |
| 470 | 603 | 604 | 491 |
| 471 | 569 | 570 | 491 |
| 472 | 577 | 578 | 414 |
| 473 | 577 | 578 | 414 |
| 474 | 595 | 596 | 414 |
| 475 | 593 | 594 | 414 |
| 476 | 643 | 644 | 414 |
| 477 | 593 | 594 | 414 |
| 478 | 595 | 596 | 414 |
| 479 | 560 | 561 | 414 |
| 480 | 560 | 561 | 414 |
| 481 | 578 | 579 | 414 |
| 482 | 562 | 563 | 414 |
| 483 | 628 | 629 | 414 |
| 484 | 580 | 581 | 414 |
| 485 | 580 | 581 | 414 |
| 486 | 578 | 579 | 414 |
| 487 | 562 | 563 | 414 |
| 488 | 588 | 589 | 491 |
| 489 | 621 | 622 | 491 |
| 490 | 554 | 555 | 491 |
| 491 | Exemplified | | |
| 492 | 568 | 569 | 491 |
| 493 | 583 | 584 | 491 |
| 494 | 545 | 546 | 414 |
| 495 | 616 | 617 | 491 |
| 496 | 551 | 552 | 491 |
| 497 | 617 | 618 | 491 |
| 498 | 631 | 632 | 491 |
| 499 | 566 | 567 | 491 |
| 500 | 672 | 673 | 491 |
| 501 | 606 | 607 | 491 |
| 502 | 511 | 512 | 1 |
| 503 | 595 | 596 | 1 |
| 504 | 512 | 513 | 1 |
| 505 | 435 | 436 | 1 |

### Biological Activity Assays

The following are assays that may be used to evaluate the biological efficacy of compounds of Formula (I).

### GLS1 Enzymatic Activity Assay

The inhibition of purified recombinant human GAC by varying concentrations of inhibitors is assessed via a dual-coupled enzymatic assay. The glutamate produced by the glutaminase reaction is used by glutamate oxidase to produce α-ketoglutarate, ammonia, and hydrogen peroxide, with this hydrogen peroxide subsequently being used by horseradish peroxidase to produce resorufin in the presence of Amplex UltraRed. The assay buffer consisted of 50 mM Hepes (pH 7.4), 0.25 mM EDTA and 0.1 mM Triton X-100. GAC was incubated with potassium phosphate (10 minutes at room temperature) prior to incubation with inhibitor (10 minutes at room temperature). The final reaction conditions were as follows: 2 nM GAC, 50 mM potassium phosphate, 100 mU/mL glutamate oxidase (Sigma), 1 mM glutamine (Sigma), 100 mU/mL horseradish peroxidase (Sigma), 75 μM Amplex UltraRed (Life Technologies), and 1% (v/v) DMSO. The production of resorufin was monitored on a Perkin Elmer Envision plate reader (excitation 530 nm, emission 590 nm) either in a kinetics or endpoint mode (at 20 minutes). IC₅₀ values were calculated using a four-parameter logistic curve fit.

### Proliferation Assay

A549 cells were routinely maintained in RPMI 1640 media (Gibco catalog number 11875-093) supplemented with 10% dialyzed fetal bovine serum using a humidified incubator (37° C., 5% CO₂ and ambient O₂). In preparation for the viability assay, cells were inoculated into 384-well black CulturPlates (Perkin Elmer) at a density of 1000 cells/well in a volume of 40 uL. Following a 24-hour incubation at 37° C., 5% CO₂ and ambient O₂, cells were treated with compound (10 uL) in a final DMSO concentration of 0.5% (v/v). The microplates were then incubated for 72 hours (37° C., 5% CO₂ and ambient O₂). Cell Titer Fluor (Promega) was subsequently added (10 uL of 6× reagent) and mixed for 15 minutes at room temperature. The plates were then incubated for 30 minutes (37° C., 5% CO₂ and ambient O₂) and fluorescence was subsequently read on the Perkin Elmer Envision plate reader. EC₅₀ values were calculated using a four-parameter logistic curve fit.

Non-limiting examples include the following compounds and pharmaceutically acceptable salts thereof.

Table 3 below reports the IC₅₀ against GLS1 and the EC₅₀ against A549 cell proliferation, both in nanomolar, and both wherein A=<100 nM, B=100-500 nM, C>500-5000 nM, and D>5000 nM. “ND” indicates no data.

TABLE 3
GLS1 IC₅₀ Data and A549 EC₅₀ Data

| | GLS1 | A549 |
|---|---|---|
| Ex. | IC₅₀ | EC₅₀ |
| 1 | A | C |
| 2 | B | C |
| 3 | A | B |
| 4 | C | D |
| 5 | A | B |
| 6 | A | C |
| 7 | A | B |
| 8 | A | B |
| 9 | C | N.D. |
| 10 | A | C |
| 11 | C | D |
| 12 | A | A |
| 13 | A | A |
| 14 | A | A |
| 15 | A | D |
| 16 | A | C |
| 17 | A | D |
| 18 | B | D |
| 19 | A | B |
| 20 | A | A |
| 21 | C | N.D. |
| 22 | A | C |
| 23 | A | C |
| 24 | A | A |
| 25 | C | N.D. |
| 26 | C | N.D. |
| 27 | C | N.D. |
| 28 | A | B |
| 29 | A | D |
| 30 | C | N.D. |
| 31 | A | C |
| 32 | C | D |
| 33 | A | B |
| 34 | B | D |
| 35 | C | D |
| 36 | B | C |
| 37 | C | D |
| 38 | C | D |
| 39 | B | C |
| 40 | B | C |
| 41 | A | C |
| 42 | A | B |
| 43 | A | B |
| 44 | A | A |
| 45 | A | A |
| 46 | B | D |
| 47 | A | C |
| 48 | A | C |
| 49 | B | C |
| 50 | C | D |
| 51 | A | C |
| 52 | A | B |
| 53 | A | C |
| 54 | B | C |
| 55 | A | B |
| 56 | A | C |
| 57 | B | C |
| 58 | C | D |
| 59 | A | B |
| 60 | B | D |
| 61 | B | C |
| 62 | B | C |
| 63 | A | B |
| 64 | A | B |
| 65 | A | C |
| 66 | A | C |
| 67 | A | C |
| 68 | A | D |
| 69 | A | C |
| 70 | A | C |
| 71 | C | D |
| 72 | C | D |
| 73 | A | C |
| 74 | C | D |
| 75 | A | A |
| 76 | A | B |
| 77 | C | D |
| 78 | A | A |
| 79 | C | C |
| 80 | A | B |
| 81 | A | C |
| 82 | A | D |
| 83 | A | D |
| 84 | A | D |
| 85 | A | B |
| 86 | C | D |
| 87 | C | D |
| 88 | B | C |
| 89 | B | D |
| 90 | B | D |
| 91 | A | B |
| 92 | A | C |
| 93 | B | C |
| 94 | A | B |
| 95 | A | A |
| 96 | A | B |
| 97 | B | D |
| 98 | A | B |
| 99 | A | A |
| 100 | A | C |
| 101 | A | B |
| 102 | A | B |
| 103 | A | A |
| 104 | A | A |
| 105 | A | B |
| 106 | B | C |
| 107 | A | C |
| 108 | A | A |
| 109 | B | C |
| 110 | A | B |
| 111 | A | A |
| 112 | A | B |
| 113 | A | C |
| 114 | A | A |
| 115 | C | N.D. |
| 116 | A | A |
| 117 | A | C |
| 118 | A | A |
| 119 | C | D |
| 120 | C | C |
| 120 | B | C |
| 121 | C | D |
| 122 | B | B |
| 124 | B | C |
| 125 | A | A |
| 126 | A | B |
| 127 | A | A |
| 128 | B | D |
| 129 | A | C |
| 130 | A | C |
| 131 | A | C |
| 132 | B | D |
| 133 | A | B |
| 134 | A | A |
| 135 | B | C |
| 136 | B | D |
| 137 | C | N.D. |
| 138 | C | N.D. |
| 139 | A | B |
| 140 | C | N.D. |
| 141 | A | A |
| 142 | C | N.D. |
| 143 | B | C |
| 144 | A | A |
| 145 | A | B |
| 146 | A | B |
| 147 | B | D |
| 148 | A | C |
| 149 | B | C |
| 150 | B | D |
| 151 | B | C |
| 152 | A | B |
| 153 | A | A |
| 154 | A | C |
| 155 | B | D |
| 156 | B | C |
| 157 | C | D |
| 158 | A | C |
| 159 | B | C |
| 160 | A | C |
| 161 | A | B |
| 162 | A | C |
| 163 | A | B |
| 164 | A | B |
| 165 | A | C |
| 166 | A | A |
| 167 | A | A |
| 168 | A | C |
| 169 | A | B |
| 170 | A | B |
| 171 | A | B |
| 172 | B | C |
| 173 | A | A |
| 174 | A | B |
| 175 | A | A |
| 176 | A | A |
| 177 | A | A |
| 178 | A | A |
| 179 | A | C |
| 180 | A | A |
| 181 | A | B |
| 182 | A | B |
| 183 | A | C |
| 184 | A | A |
| 185 | A | C |
| 186 | A | A |
| 187 | A | A |
| 188 | C | D |
| 189 | B | D |
| 190 | A | C |
| 191 | A | B |
| 192 | B | D |
| 193 | B | C |
| 194 | A | A |
| 195 | B | C |
| 196 | A | C |
| 197 | A | C |
| 198 | A | B |
| 199 | A | A |
| 200 | B | C |
| 201 | A | B |
| 202 | A | B |
| 203 | A | B |
| 204 | A | C |
| 205 | A | A |
| 206 | A | A |
| 207 | C | N.D. |
| 208 | A | C |
| 209 | A | C |
| 210 | A | A |
| 211 | A | B |
| 212 | A | A |
| 213 | B | C |
| 214 | A | B |
| 215 | A | B |
| 216 | A | B |
| 217 | A | A |
| 218 | B | N.D. |
| 219 | A | A |
| 220 | A | A |
| 221 | A | A |
| 222 | A | B |
| 223 | A | A |
| 224 | A | A |
| 225 | C | N.D. |
| 226 | A | A |
| 227 | A | B |
| 228 | A | A |
| 229 | B | C |
| 230 | B | N.D. |
| 231 | B | D |
| 232 | A | B |
| 233 | A | C |
| 234 | A | B |
| 235 | A | C |
| 236 | A | C |
| 237 | A | B |
| 238 | A | B |
| 239 | A | A |
| 240 | A | C |
| 241 | A | A |
| 242 | A | B |
| 243 | A | B |
| 244 | A | C |
| 245 | A | A |
| 246 | A | C |
| 247 | A | C |
| 248 | A | C |
| 249 | A | A |
| 250 | A | B |
| 251 | A | B |
| 252 | A | B |
| 253 | A | C |
| 254 | B | N.D. |
| 255 | A | A |
| 256 | A | C |
| 257 | A | C |
| 258 | A | A |
| 259 | A | A |
| 260 | A | A |
| 261 | A | A |
| 262 | A | A |
| 263 | A | B |
| 264 | A | A |
| 265 | A | A |
| 266 | A | A |
| 267 | A | A |
| 268 | A | B |
| 269 | A | A |
| 270 | B | C |
| 271 | A | A |
| 272 | A | B |
| 273 | A | C |
| 274 | A | A |
| 275 | A | A |
| 276 | A | B |
| 277 | A | B |
| 278 | A | B |
| 279 | A | D |
| 280 | B | C |
| 281 | A | A |
| 282 | C | D |
| 283 | A | A |
| 284 | A | A |
| 285 | A | A |
| 286 | A | A |
| 287 | A | A |
| 288 | A | A |
| 289 | A | A |
| 290 | A | A |
| 291 | A | A |
| 292 | A | A |
| 293 | A | B |
| 294 | A | A |
| 295 | A | A |
| 296 | A | A |
| 297 | A | A |
| 298 | B | C |
| 299 | B | C |
| 300 | A | A |
| 301 | A | A |
| 302 | A | B |
| 303 | A | A |
| 304 | A | A |
| 305 | A | A |
| 306 | A | A |
| 307 | A | B |
| 308 | A | B |
| 309 | C | C |
| 310 | C | C |
| 311 | C | D |
| 312 | A | B |
| 313 | A | B |
| 314 | C | B |
| 315 | A | A |
| 316 | A | A |
| 317 | A | A |
| 318 | A | A |
| 319 | B | N.D. |
| 320 | B | B |
| 321 | D | N.D. |
| 322 | D | N.D. |
| 323 | A | A |
| 324 | A | A |
| 325 | A | A |
| 326 | A | A |
| 327 | A | A |
| 328 | A | A |
| 329 | A | A |
| 330 | A | B |
| 331 | C | D |
| 332 | A | A |
| 333 | A | A |
| 334 | A | A |
| 335 | A | B |
| 336 | A | A |
| 337 | A | A |
| 338 | B | B |
| 339 | A | A |
| 340 | A | A |
| 341 | A | A |
| 342 | A | A |
| 343 | A | A |
| 344 | B | A |
| 345 | B | A |
| 346 | A | A |
| 347 | B | D |
| 348 | A | A |
| 349 | A | A |
| 350 | A | A |
| 351 | A | A |
| 352 | A | A |
| 353 | A | B |
| 354 | A | A |
| 355 | A | A |
| 356 | B | B |
| 357 | A | B |
| 358 | A | A |
| 359 | B | C |
| 360 | A | A |
| 361 | A | A |
| 362 | A | A |
| 363 | A | A |
| 364 | A | A |
| 365 | C | B |
| 366 | D | D |
| 367 | A | B |
| 368 | A | A |
| 369 | A | A |
| 370 | B | B |
| 371 | A | B |
| 372 | A | B |
| 373 | B | C |
| 374 | A | B |
| 375 | B | C |
| 376 | A | A |
| 377 | A | A |
| 379 | A | B |
| 380 | B | C |
| 381 | B | C |
| 382 | C | D |
| 383 | A | A |
| 384 | A | B |
| 385 | A | B |
| 386 | A | B |
| 387 | A | A |
| 388 | A | A |
| 389 | A | A |
| 390 | B | A |
| 391 | C | D |
| 392 | A | A |
| 393 | A | A |
| 394 | A | A |
| 395 | A | A |
| 396 | B | A |
| 397 | B | B |
| 398 | A | A |
| 399 | B | B |
| 400 | A | A |
| 401 | A | B |
| 402 | B | C |
| 403 | B | C |
| 404 | B | C |
| 405 | B | B |
| 406 | C | D |
| 407 | C | D |
| 408 | D | D |
| 409 | A | A |
| 410 | B | C |
| 411 | C | C |
| 412 | B | C |
| 413 | B | C |
| 414 | A | C |
| 415 | A | A |
| 416 | A | A |
| 417 | B | C |
| 418 | A | A |
| 419 | A | A |
| 420 | A | C |
| 421 | A | A |
| 422 | A | A |
| 423 | A | B |
| 424 | A | B |
| 425 | A | B |
| 426 | A | C |
| 427 | C | C |
| 428 | A | A |
| 429 | C | C |
| 430 | C | D |
| 431 | A | A |
| 432 | A | A |
| 433 | A | B |
| 434 | A | A |
| 435 | A | A |
| 436 | D | N.D. |
| 437 | D | N.D. |
| 438 | A | A |
| 439 | A | A |
| 440 | A | A |
| 441 | A | C |
| 442 | A | A |
| 443 | A | A |
| 444 | B | N.D. |
| 445 | B | C |
| 446 | A | B |
| 447 | A | A |
| 448 | C | N.D. |
| 449 | C | N.D. |
| 450 | B | C |
| 451 | A | B |
| 452 | C | N.D. |
| 453 | B | C |
| 454 | A | B |
| 455 | A | A |
| 456 | A | B |
| 457 | C | N.D. |
| 458 | A | A |
| 459 | A | A |
| 460 | A | A |
| 461 | A | C |
| 462 | B | B |
| 463 | B | B |
| 464 | A | A |
| 465 | A | A |
| 466 | A | B |
| 467 | A | B |
| 468 | B | B |
| 469 | A | B |
| 470 | A | A |
| 471 | A | A |
| 472 | A | B |
| 473 | A | B |
| 474 | A | B |
| 475 | A | C |
| 476 | A | C |
| 477 | A | B |
| 478 | A | B |
| 479 | C | C |
| 480 | B | C |
| 481 | A | B |
| 482 | A | B |
| 483 | A | B |
| 484 | A | B |
| 485 | B | B |
| 486 | A | A |
| 487 | B | B |
| 488 | A | A |
| 489 | A | A |
| 490 | A | B |
| 491 | A | A |
| 492 | A | A |
| 493 | A | A |
| 494 | C | D |
| 495 | A | C |
| 496 | C | D |
| 497 | A | B |
| 498 | A | A |
| 499 | C | D |
| 500 | A | A |
| 501 | A | B |
| 502 | A | B |
| 503 | A | A |
| 504 | A | B |
| 505 | A | C |

### Other Embodiments

The detailed description set-forth above is provided to aid those skilled in the art in practicing the present disclosure. However, the disclosure described and claimed herein is not to be limited in scope by the specific embodiments herein disclosed because these embodiments are intended as illustration of several aspects of the disclosure. Any equivalent embodiments are intended to be within the scope of this disclosure. Indeed, various modifications of the disclosure in addition to those shown and described herein will become apparent to those skilled in the art from the foregoing description, which do not depart from the spirit or scope of the present inventive discovery. Such modifications are also intended to fall within the scope of the appended claims.

What is claimed is:

1. A compound of structural Formula I 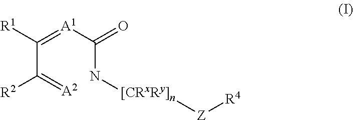
or a salt thereof, wherein:
n is chosen from 3, 4, and 5;
each R^{x} and R^{y} is independently chosen from alkyl, cyano, H, and halo, wherein two R^{x} groups together with the atoms to which they are attached optionally form a cycloalkyl ring;
A¹ and A² are independently chosen from C—H, C—F, and N;
R¹ and R⁴ are independently chosen from alkenyl, alkoxy, alkyl, aryl, arylalkyl, cyano, cycloalkyl, cycloalkylalkyl, H, halo, haloalkyl, heteroaryl, heteroarylalkyl, heterocycloalkyl, heterocycloalkylalkyl, hydroxyl, C(R³)₂C(O)R³, C(R³)₂C(O)N(R³)₂, C(R³)₂N(R³)₂, C(R³)₂NR³C(O)R³, C(R³)₂NR³C(O)OR³, C(R³)₂NR³C(O)N(R³)₂, C(R³)₂NR³S(O)R³, C(R³)₂NR³S(O)₂R³, N(R³)₂, NR³C(O)R³, NR³C(O)OR³, NR³C(O)N(R³)₂, NR³S(O)R³, NR³S(O)₂R³, C(O)N(R³)₂, S(O)N(R³)₂, S(O)₂N(R³)₂, C(O)R³, SR³, S(O)R³, and S(O)₂R³, wherein each R¹ and R⁴ may be optionally substituted with between 0 and 3 R^{z} groups;
R² is chosen from alkyl, heterocycloalkyl, cyano, cycloalkyl, H, halo, and haloalkyl, wherein R¹ and R² together with the atoms to which they are attached optionally form an form an aryl, cycloalkyl, heteroaryl, or heterocycloalkyl ring, which may be optionally substituted with between 0 and 3 R^{z} groups;
each R³ is independently chosen from alkenyl, alkoxy, alkyl, aryl, arylalkyl, cyano, cycloalkyl, cycloalkylalkyl, H, halo, haloalkyl, heteroaryl, heteroarylalkyl, heterocycloalkyl, heterocycloalkylalkyl, and hydroxyl, wherein each R³ may be optionally substituted with between 0 and 3 R^{z} groups, wherein two R³ groups together with the atoms to which they are attached optionally form an aryl, cycloalkyl, heteroaryl, or heterocycloalkyl ring, which may be optionally substituted with between 0 and 3 R^{z} groups;
each R^{Z} group is independently chosen from alkenyl, alkoxy, alkoxyalkyl, alkoxyaryl, alkoxyarylalkyl, alkoxycycloalkyl, alkoxycycloalkylalkyl, alkoxyhaloalkyl, alkoxyheteroaryl, alkoxyheteroarylalkyl, alkoxyheterocycloalkyl, alkoxyheterocycloalkylalkyl, alkyl, alkylaryl, alkylarylalkyl, alkylcycloalkyl, alkylcycloalkylalkyl, alkylheteroaryl, alkylheteroarylalkyl, alkylheterocycloalkyl, alkylheterocycloalkylalkyl, aryl, arylalkyl, arylalkyloxy, arylhaloalkyl, aryloxy, cyano, cycloalkyl, cycloalkylalkyl, cycloalkylalkyloxy, cycloalkylhaloalkyl, cycloalkyloxy, H, halo, haloalkoxy, haloalkoxyalkyl, haloalkoxyaryl, haloalkoxyarylalkyl, haloalkoxycycloalkyl, haloalkoxycycloalkylalkyl, haloalkoxyheteroaryl, haloalkoxyheteroarylalkyl, haloalkoxyheterocycloalkyl, haloalkoxyheterocycloalkylalkyl, haloalkyl, haloalkylaryl, haloalkylarylalkyl, haloalkylcycloalkyl, haloalkylcycloalkylalkyl, haloalkylheteroaryl, haloalkylheteroarylalkyl, haloalkylheterocycloalkyl, haloalkylheterocycloalkylalkyl, haloaryl, haloarylalkyl, haloarylalkyloxy, haloaryloxy, halocycloalkyl, halocycloalkylalkyl, halocycloalkylalkyloxy, halocycloalkyloxy, haloheteroaryl, haloheteroarylalkyl, haloheteroarylalkyloxy, haloheteroaryloxy, haloheterocycloalkyl, haloheterocycloalkylalkyl, haloheterocycloalkylalkyloxy, haloheterocycloalkyloxy, heteroaryl, heteroarylalkyl, heteroarylalkyloxy, heteroarylhaloalkyl, heteroaryloxy, heterocycloalkyl, heterocycloalkylalkyl, heterocycloalkylalkyloxy, heterocycloalkylhaloalkyl, heterocycloalkyloxy, hydroxyl, oxo, N(R⁶)₂, NR⁶C(O)C(R⁶)₃, NR⁶C(O)OC(R⁶)₃, NR⁶C(O)N(R⁶)₂, NR⁶S(O)C(R⁶)₃, NR⁶S(O)₂C(R⁶)₃, C(O)N(R⁶)₂, S(O)N(R⁶)₂, S(O)₂N(R⁶)₂, C(O)C(R⁶)₃, SC(R⁶)₃, S(O)C(R⁶)₃, and S(O)₂C(R⁶)₃;
each R⁶ is independently chosen from alkenyl, alkoxy, alkyl, aryl, arylalkyl, cyano, cycloalkyl, cycloalkylalkyl, H, halo, haloalkyl, heteroaryl, heteroarylalkyl, heterocycloalkyl, heterocycloalkylalkyl, and hydroxyl, wherein two R⁶ groups together with the atoms to which they are attached optionally form an aryl, cycloalkyl, heteroaryl, or heterocycloalkyl ring, which may be optionally substituted with between 0 and 3 R^{x} groups; and
Z is heteroaryl, which may be optionally substituted.

2. The compound as recited in claim 1, wherein the compound has structural Formula II: 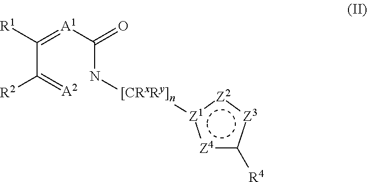
or a salt thereof, wherein:
n is chosen from 3, 4, and 5;
each R^{x} and R^{y} is independently chosen from alkyl, cyano, H, and halo, wherein two R^{x} groups together with the atoms to which they are attached optionally form a cycloalkyl ring;
A¹ and A² are independently chosen from C—H, C—F, and N;
Z¹ is chosen from C and N;
Z², Z³, and Z⁴ are independently chosen from N, O, S, and CH, wherein at least one of Z¹, Z², Z³, and Z⁴ is chosen from N, O, and S;
R¹ and R⁴ are independently chosen from alkenyl, alkoxy, alkyl, aryl, arylalkyl, cyano, cycloalkyl, cycloalkylalkyl, H, halo, haloalkyl, heteroaryl, heteroarylalkyl, heterocycloalkyl, heterocycloalkylalkyl, hydroxyl, C(R³)₂C(O)R³, C(R³)₂C(O)N(R³)₂, C(R³)₂N(R³)₂, C(R³)₂NR³C(O)R³, C(R³)₂NR³C(O)OR³, C(R³)₂NR³C(O)N(R³)₂, C(R³)₂NR³S(O)R³, C(R³)₂NR³S(O)₂R³, N(R³)₂, NR³C(O)R³, NR³C(O)OR³, NR³C(O)N(R³)₂, NR³S(O)R³, NR³S(O)₂R³, C(O)N(R³)₂, S(O)N(R³)₂, S(O)₂N(R³)₂, C(O)R³, SR³, S(O)R³, and S(O)₂R³, wherein each R¹ and R⁴ may be optionally substituted with between 0 and 3 R^{z} groups;
R² is chosen from alkyl, heterocycloalkyl, cyano, cycloalkyl, H, halo, and haloalkyl, wherein R¹ and R² together with the atoms to which they are attached optionally form an form an aryl, cycloalkyl, heteroaryl, or heterocycloalkyl ring, which may be optionally substituted with between 0 and 3 R^{z} groups;
each R³ is independently chosen from alkenyl, alkoxy, alkyl, aryl, arylalkyl, cyano, cycloalkyl, cycloalkylalkyl, H, halo, haloalkyl, heteroaryl, heteroarylalkyl, heterocycloalkyl, heterocycloalkylalkyl, and hydroxyl, wherein each R³ may be optionally substituted with between 0 and 3 R^{z} groups, wherein two R³ groups together with the atoms to which they are attached optionally form an aryl, cycloalkyl, heteroaryl, or heterocycloalkyl ring, which may be optionally substituted with between 0 and 3 R^{z} groups;
each R^{Z} group is independently chosen from alkenyl, alkoxy, alkoxyalkyl, alkoxyaryl, alkoxyarylalkyl, alkoxycycloalkyl, alkoxycycloalkylalkyl, alkoxyhaloalkyl, alkoxyheteroaryl, alkoxyheteroarylalkyl, alkoxyheterocycloalkyl, alkoxyheterocycloalkylalkyl, alkyl, alkylaryl, alkylarylalkyl, alkylcycloalkyl, alkylcycloalkylalkyl, alkylheteroaryl, alkylheteroarylalkyl, alkylheterocycloalkyl, alkylheterocycloalkylalkyl, aryl, arylalkyl, arylalkyloxy, arylhaloalkyl, aryloxy, cyano, cycloalkyl, cycloalkylalkyl, cycloalkylalkyloxy, cycloalkylhaloalkyl, cycloalkyloxy, H, halo, haloalkoxy, haloalkoxyalkyl, haloalkoxyaryl, haloalkoxyarylalkyl, haloalkoxycycloalkyl, haloalkoxycycloalkylalkyl, haloalkoxyheteroaryl, haloalkoxyheteroarylalkyl, haloalkoxyheterocycloalkyl, haloalkoxyheterocycloalkylalkyl, haloalkyl, haloalkylaryl, haloalkylarylalkyl, haloalkylcycloalkyl, haloalkylcycloalkylalkyl, haloalkylheteroaryl, haloalkylheteroarylalkyl, haloalkylheterocycloalkyl, haloalkylheterocycloalkylalkyl, haloaryl, haloarylalkyl, haloarylalkyloxy, haloaryloxy, halocycloalkyl, halocycloalkylalkyl, halocycloalkylalkyloxy, halocycloalkyloxy, haloheteroaryl, haloheteroarylalkyl, haloheteroarylalkyloxy, haloheteroaryloxy, haloheterocycloalkyl, haloheterocycloalkylalkyl, haloheterocycloalkylalkyloxy, haloheterocycloalkyloxy, heteroaryl, heteroarylalkyl, heteroarylalkyloxy, heteroarylhaloalkyl, heteroaryloxy, heterocycloalkyl, heterocycloalkylalkyl, heterocycloalkylalkyloxy, heterocycloalkylhaloalkyl, heterocycloalkyloxy, hydroxyl, oxo, N(R⁶)₂, NR⁶C(O)C(R⁶)₃, NR⁶C(O)OC(R⁶)₃, NR⁶C(O)N(R⁶)₂, NR⁶S(O)C(R⁶)₃, NR⁶S(O)₂C(R⁶)₃, C(O)N(R⁶)₂, S(O)N(R⁶)₂, S(O)₂N(R⁶)₂, C(O)C(R⁶)₃, SC(R⁶)₃, S(O)C(R⁶)₃, and S(O)₂C(R⁶)₃; and
each R⁶ is independently chosen from alkenyl, alkoxy, alkyl, aryl, arylalkyl, cyano, cycloalkyl, cycloalkylalkyl, H, halo, haloalkyl, heteroaryl, heteroarylalkyl, heterocycloalkyl, heterocycloalkylalkyl, and hydroxyl, wherein two R⁶ groups together with the atoms to which they are attached optionally form an aryl, cycloalkyl, heteroaryl, or heterocycloalkyl ring, which may be optionally substituted with between 0 and 3 R^{x} groups.

3. The compound as recited in claim 2, wherein:
n is 4; and
A¹ and A² are CH.

4. The compound as recited in claim 2, wherein:
n is 4;
A¹ is N; and
A² is CH.

5. The compound as recited in claim 2, wherein:
Z¹ is C;
Z² and Z³ are N;
Z⁴ is S; and
R⁴ is chosen from N(R³)₂, NR³C(O)C(R³)₃, NR³C(O)OC(R³)₃, and NR³C(O)N(R³)₂.

6. The compound as recited in claim 2, wherein:
n is 4;
A¹ and A² are CH;
Z¹ is C;
Z² and Z³ are N;
Z⁴ is S; and
R⁴ is chosen from N(R³)₂, NR³C(O)C(R³)₃, NR³C(O)OC(R³)₃, and NR³C(O)N(R³)₂.

7. The compound as recited in claim 2, wherein:
Z¹ is C;
Z² and Z³ are N;
Z⁴ is S; and
R⁴ is C(O)N(R³)₂.

8. The compound as recited in claim 2, wherein:
n is 4;
A¹ and A² are CH;
Z¹ is C;
Z² and Z³ are N;
Z⁴ is S; and
R⁴ is C(O)N(R³)₂.

9. The compound as recited in claim 2, wherein:
Z¹, Z², and Z³ are N;
Z⁴ is CH; and
R⁴ is chosen from N(R³)₂, NR³C(O)C(R³)₃, NR³C(O)OC(R³)₃, and NR³C(O)N(R³)₂.

10. The compound as recited in claim 2, wherein:
n is 4;
A¹ and A² are CH;
Z¹, Z², and Z³ are N;
Z⁴ is CH; and
R⁴ is chosen from N(R³)₂, NR³C(O)C(R³)₃, NR³C(O)OC(R³)₃, and NR³C(O)N(R³)₂.

11. The compound as recited in claim 2, wherein:
Z¹, Z², and Z³ are N;
Z⁴ is CH; and
R⁴ is C(O)N(R³)₂.

12. The compound as recited in claim 2, wherein:
n is 4;
A¹ and A² are CH;
Z¹, Z², and Z³ are N;
Z⁴ is CH; and
R⁴ is C(O)N(R³)₂.

13. The compound as recited in claim 1, wherein the compound has structural Formula III: 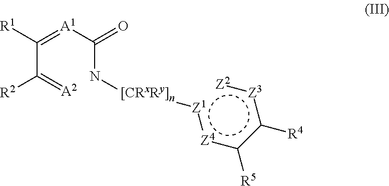
or a salt thereof, wherein:
n is chosen from 3, 4, and 5;
each R^{x} and R^{y} is independently chosen from alkyl, cyano, H, and halo, wherein two R^{x} groups together with the atoms to which they are attached optionally form a cycloalkyl ring;
A¹ and A² are independently chosen from C—H, C—F, and N;
Z¹ is chosen from C and N;
Z² is chosen from N, CH, and C(O);
Z³, and Z⁴ are independently chosen from N and CH, wherein at least one of Z¹, Z², Z³, and Z⁴ is N;
R¹ and R⁴ are independently chosen from alkenyl, alkoxy, alkyl, aryl, arylalkyl, cyano, cycloalkyl, cycloalkylalkyl, H, halo, haloalkyl, heteroaryl, heteroarylalkyl, heterocycloalkyl, heterocycloalkylalkyl, hydroxyl, C(R³)₂C(O)R³, C(R³)₂C(O)N(R³)₂, C(R³)₂N(R³)₂, C(R³)₂NR³C(O)R³, C(R³)₂NR³C(O)OR³, C(R³)₂NR³C(O)N(R³)₂, C(R³)₂NR³S(O)R³, C(R³)₂NR³S(O)₂R³, N(R³)₂, NR³C(O)R³, NR³C(O)OR³, NR³C(O)N(R³)₂, NR³S(O)R³, NR³S(O)₂R³, C(O)N(R³)₂, S(O)N(R³)₂, S(O)₂N(R³)₂, C(O)R³, SR³, S(O)R³, and S(O)₂R³, wherein each R¹ and R⁴ may be optionally substituted with between 0 and 3 R^{z} groups;
R² and R⁵ are chosen from alkyl, heterocycloalkyl, cyano, cycloalkyl, H, halo, and haloalkyl, wherein R¹ and R² together with the atoms to which they are attached optionally form an form an aryl, cycloalkyl, heteroaryl, or heterocycloalkyl ring, which may be optionally substituted with between 0 and 3 R^{z} groups, wherein R⁴ and R⁵ together with the atoms to which they are attached optionally form an aryl, cycloalkyl, heteroaryl, or heterocycloalkyl ring, which may be optionally substituted with between 0 and 3 R^{z} groups;
each R³ is independently chosen from alkenyl, alkoxy, alkyl, aryl, arylalkyl, cyano, cycloalkyl, cycloalkylalkyl, H, halo, haloalkyl, heteroaryl, heteroarylalkyl, heterocycloalkyl, heterocycloalkylalkyl, and hydroxyl, wherein each R³ may be optionally substituted with between 0 and 3 R^{z} groups, wherein two R³ groups together with the atoms to which they are attached optionally form an aryl, cycloalkyl, heteroaryl, or heterocycloalkyl ring, which may be optionally substituted with between 0 and 3 R^{z} groups;
each R^{Z} group is independently chosen from alkenyl, alkoxy, alkoxyalkyl, alkoxyaryl, alkoxyarylalkyl, alkoxycycloalkyl, alkoxycycloalkylalkyl, alkoxyhaloalkyl, alkoxyheteroaryl, alkoxyheteroarylalkyl, alkoxyheterocycloalkyl, alkoxyheterocycloalkylalkyl, alkyl, alkylaryl, alkylarylalkyl, alkylcycloalkyl, alkylcycloalkylalkyl, alkylheteroaryl, alkylheteroarylalkyl, alkylheterocycloalkyl, alkylheterocycloalkylalkyl, aryl, arylalkyl, arylalkyloxy, arylhaloalkyl, aryloxy, cyano, cycloalkyl, cycloalkylalkyl, cycloalkylalkyloxy, cycloalkylhaloalkyl, cycloalkyloxy, H, halo, haloalkoxy, haloalkoxyalkyl, haloalkoxyaryl, haloalkoxyarylalkyl, haloalkoxycycloalkyl, haloalkoxycycloalkylalkyl, haloalkoxyheteroaryl, haloalkoxyheteroarylalkyl, haloalkoxyheterocycloalkyl, haloalkoxyheterocycloalkylalkyl, haloalkyl, haloalkylaryl, haloalkylarylalkyl, haloalkylcycloalkyl, haloalkylcycloalkylalkyl, haloalkylheteroaryl, haloalkylheteroarylalkyl, haloalkylheterocycloalkyl, haloalkylheterocycloalkylalkyl, haloaryl, haloarylalkyl, haloarylalkyloxy, haloaryloxy, halocycloalkyl, halocycloalkylalkyl, halocycloalkylalkyloxy, halocycloalkyloxy, haloheteroaryl, haloheteroarylalkyl, haloheteroarylalkyloxy, haloheteroaryloxy, haloheterocycloalkyl, haloheterocycloalkylalkyl, haloheterocycloalkylalkyloxy, haloheterocycloalkyloxy, heteroaryl, heteroarylalkyl, heteroarylalkyloxy, heteroarylhaloalkyl, heteroaryloxy, heterocycloalkyl, heterocycloalkylalkyl, heterocycloalkylalkyloxy, heterocycloalkylhaloalkyl, heterocycloalkyloxy, hydroxyl, oxo, N(R⁶)₂, NR⁶C(O)C(R⁶)₃, NR⁶C(O)OC(R⁶)₃, NR⁶C(O)N(R⁶)₂, NR⁶S(O)C(R⁶)₃, NR⁶S(O)₂C(R⁶)₃, C(O)N(R⁶)₂, S(O)N(R⁶)₂, S(O)₂N(R⁶)₂, C(O)C(R⁶)₃, SC(R⁶)₃, S(O)C(R⁶)₃, and S(O)₂C(R⁶)₃; and
each R⁶ is independently chosen from alkenyl, alkoxy, alkyl, aryl, arylalkyl, cyano, cycloalkyl, cycloalkylalkyl, H, halo, haloalkyl, heteroaryl, heteroarylalkyl, heterocycloalkyl, heterocycloalkylalkyl, and hydroxyl, wherein two R⁶ groups together with the atoms to which they are attached optionally form an aryl, cycloalkyl, heteroaryl, or heterocycloalkyl ring, which may be optionally substituted with between 0 and 3 R^{x} groups.

14. The compound as recited in claim 13, wherein:
n is 4; and
A¹ and A² is are CH.

15. The compound as recited in claim 13, wherein:
n is 4;
A¹ is N;
and A² is CH.

16. The compound as recited in claim 13, wherein:
Z¹ is C;
Z² and Z³ are N;
Z⁴ is CH;
R⁴ is chosen from N(R³)₂, NR³C(O)C(R³)₃, NR³C(O)OC(R³)₃, and NR³C(O)N(R³)₂; and
R⁵ is H.

17. The compound as recited in claim 13, wherein:
n is 4;
A¹ and A² is are CH;
Z¹ is C;
Z² and Z³ are N;
Z⁴ is CH;
R⁴ is chosen from N(R³)₂, NR³C(O)C(R³)₃, NR³C(O)OC(R³)₃, and NR³C(O)N(R³)₂; and
R⁵ is H.

18. The compound as recited in claim 13, wherein:
Z¹ is C;
Z² and Z³ are N;
Z⁴ is CH;
R⁴ is C(O)N(R³)₂; and
R⁵ is H.

19. The compound as recited in claim 13, wherein:
n is 4;
A¹ and A² is are CH;
Z¹ is C;
Z² and Z³ are N;
Z⁴ is CH;
R⁴ is C(O)N(R³)₂; and
R⁵ is H.

20. The compound as recited in claim 13, wherein:
Z¹ is N;
Z² is C(O);
Z⁴ is CH;
R⁴ is chosen from N(R³)₂, NR³C(O)C(R³)₃, NR³C(O)OC(R³)₃, and NR³C(O)N(R³)₂; and
R⁵ is H.

21. The compound as recited in claim 13, wherein:
n is 4;
A¹ and A² is are CH;
Z¹ is N;
Z² is C(O);
Z⁴ is CH;
R⁴ is chosen from N(R³)₂, NR³C(O)C(R³)₃, NR³C(O)OC(R³)₃, and NR³C(O)N(R³)₂; and
R⁵ is H.

22. The compound as recited in claim 13, wherein:
Z¹ is N;
Z² is C(O);
Z⁴ is CH;
R⁴ is C(O)N(R³)₂; and
R⁵ is H.

23. The compound as recited in claim 13, wherein:
n is 4;
A¹ and A² is are CH;
Z¹ is N;
Z² is C(O);
Z⁴ is CH;
R⁴ is C(O)N(R³)₂; and
R⁵ is H.

24. The compound as recited in claim 1, or a salt thereof, wherein the compound is chosen from Examples 1-505.

25. A pharmaceutical composition comprising a compound as recited in claim 1 and a pharmaceutically acceptable carrier, adjuvant, or vehicle.

26. A method of inhibiting GLS1 activity in a biological sample comprising contacting the biological sample with a compound as recited in claim 1.

27. A method of treating a GLS1-mediated disorder in a subject in need thereof, comprising the step of administering to the subject a compound as recited in claim 1.

28. The method as recited in claim 27, wherein the subject is a human.

29. The method as recited in claim 27, wherein the GLS1-mediated disorder is chosen from cancer, immunological disorders, and neurological disorders.

30. The method as recited in claim 29, wherein the GLS1-mediated disorder is cancer.

31. The method as recited in claim 30, wherein the cancer is chosen from Acute Lymphoblastic Leukemia (ALL), Acute Myeloid Leukemia (AML), Adrenocortical Carcinoma, AIDS-Related Cancers (Kaposi Sarcoma and Lymphoma), Anal Cancer, Appendix Cancer, Atypical Teratoid/Rhabdoid Tumor, Basal Cell Carcinoma, Bile Duct Cancer (including Extrahepatic), Bladder Cancer, Bone Cancer (including Osteosarcoma and Malignant Fibrous Histiocytoma), Brain Tumor (such as Astrocytomas, Brain and Spinal Cord Tumors, Brain Stem Glioma, Central Nervous System Atypical Teratoid/Rhabdoid Tumor, Central Nervous System Embryonal Tumors, Craniopharyngioma, Ependymoblastoma, Ependymoma, Medulloblastoma, Medulloepithelioma, Pineal Parenchymal Tumors of Intermediate Differentiation, Supratentorial Primitive Neuroectodermal Tumors and Pineoblastoma), Breast Cancer, Bronchial Tumors, Burkitt Lymphoma, Basal Cell Carcinoma, Bile Duct Cancer (including Extrahepatic), Bladder Cancer, Bone Cancer (including Osteosarcoma and Malignant Fibrous Histiocytoma), Carcinoid Tumor, Carcinoma of Unknown Primary, Central Nervous System (such as Atypical Teratoid/Rhabdoid Tumor, Embryonal Tumors and Lymphoma), Cervical Cancer, Childhood Cancers, Chordoma, Chronic Lymphocytic Leukemia (CLL), Chronic Myelogenous Leukemia (CML), Chronic Myeloproliferative Disorders, Colon Cancer, Colorectal Cancer, Craniopharyngioma, Cutaneous T-Cell Lymphoma (Mycosis Fungoides and Sézary Syndrome), Duct, Bile (Extrahepatic), Ductal Carcinoma In Situ (DCIS), Embryonal Tumors (Central Nervous System), Endometrial Cancer, Ependymoblastoma, Ependymoma, Esophageal Cancer, Esthesioneuroblastoma, Ewing Sarcoma Family of Tumors, Extracranial Germ Cell Tumor, Extragonadal Germ Cell Tumor, Extrahepatic Bile Duct Cancer, Eye Cancer (like Intraocular Melanoma, Retinoblastoma), Fibrous Histiocytoma of Bone (including Malignant and Osteosarcoma) Gallbladder Cancer, Gastric (Stomach) Cancer, Gastrointestinal Carcinoid Tumor, Gastrointestinal Stromal Tumors (GIST), Germ Cell Tumor (Extracranial, Extragonadal, Ovarian), Gestational Trophoblastic Tumor, Glioma, Hairy Cell Leukemia, Head and Neck Cancer, Heart Cancer, Hepatocellular (Liver) Cancer, Histiocytosis, Langerhans Cell, Hodgkin Lymphoma, Hypopharyngeal Cancer, Intraocular Melanoma, Islet Cell Tumors (Endocrine, Pancreas), Kaposi Sarcoma, Kidney (including Renal Cell), Langerhans Cell Histiocytosis, Laryngeal Cancer, Leukemia (including Acute Lymphoblastic (ALL), Acute Myeloid (AML), Chronic Lymphocytic (CLL), Chronic Myelogenous (CML), Hairy Cell), Lip and Oral Cavity Cancer, Liver Cancer (Primary), Lobular Carcinoma In Situ (LCIS), Lung Cancer (Non-Small Cell and Small Cell), Lymphoma (AIDS-Related, Burkitt, Cutaneous T-Cell (Mycosis Fungoides and Sézary Syndrome), Hodgkin, Non-Hodgkin, Primary Central Nervous System (CNS), Macroglobulinemia, Waldenstrim, Male Breast Cancer, Malignant Fibrous Histiocytoma of Bone and Osteosarcoma, Medulloblastoma, Medulloepithelioma, Melanoma (including Intraocular (Eye)), Merkel Cell Carcinoma, Mesothelioma (Malignant), Metastatic Squamous Neck Cancer with Occult Primary, Midline Tract Carcinoma Involving NUT Gene, Mouth Cancer, Multiple Endocrine Neoplasia Syndromes, Multiple Myeloma/Plasma Cell Neoplasm, Mycosis Fungoides, Myelodysplastic Syndromes, Myelodysplastic/Myeloproliferative Neoplasms, Myelogenous Leukemia, Chronic (CML), Myeloid Leukemia, Acute (AML), Myeloma and Multiple Myeloma, Myeloproliferative Disorders (Chronic), Nasal Cavity and Paranasal Sinus Cancer, Nasopharyngeal Cancer, Neuroblastoma, Non-Hodgkin Lymphoma, Non-Small Cell Lung Cancer, Oral Cancer, Oral Cavity Cancer, Lip and, Oropharyngeal Cancer, Osteosarcoma and Malignant Fibrous Histiocytoma of Bone, Ovarian Cancer (such as Epithelial, Germ Cell Tumor, and Low Malignant Potential Tumor), Pancreatic Cancer (including Islet Cell Tumors), Papillomatosis, Paraganglioma, Paranasal Sinus and Nasal Cavity Cancer, Parathyroid Cancer, Penile Cancer, Pharyngeal Cancer, Pheochromocytoma, Pineal Parenchymal Tumors of Intermediate Differentiation, Pineoblastoma and Supratentorial Primitive Neuroectodermal Tumors, Pituitary Tumor, Plasma Cell Neoplasm/Multiple Myeloma, Pleuropulmonary Blastoma, Pregnancy and Breast Cancer, Primary Central Nervous System (CNS) Lymphoma, Prostate Cancer, Rectal Cancer, Renal Cell (Kidney) Cancer, Renal Pelvis and Ureter, Transitional Cell Cancer, Retinoblastoma, Rhabdomyosarcoma, Salivary Gland Cancer, Sarcoma (like Ewing Sarcoma Family of Tumors, Kaposi, Soft Tissue, Uterine), Sézary Syndrome, Skin Cancer (such as Melanoma, Merkel Cell Carcinoma, Nonmelanoma), Small Cell Lung Cancer, Small Intestine Cancer, Soft Tissue Sarcoma, Squamous Cell Carcinoma, Squamous Neck Cancer with Occult Primary, Metastatic, Stomach (Gastric) Cancer, Supratentorial Primitive Neuroectodermal Tumors, T-Cell Lymphoma (Cutaneous, Mycosis Fungoides and Sézary Syndrome), Testicular Cancer, Throat Cancer, Thymoma and Thymic Carcinoma, Thyroid Cancer, Transitional Cell Cancer of the Renal Pelvis and Ureter, Trophoblastic Tumor (Gestational), Unknown Primary, Unusual Cancers of Childhood, Ureter and Renal Pelvis, Transitional Cell Cancer, Urethral Cancer, Uterine Cancer, Endometrial, Uterine Sarcoma, Waldenstrim Macroglobulinemia and Wilms Tumor, or a variant thereof.

32. A method of treating a GLS1-mediated disorder in a subject in need thereof, comprising the sequential or co-administration of a compound as recited in claim 1 or a pharmaceutically acceptable salt thereof, and another therapeutic agent.

33. The method as recited in claim 32, wherein the therapeutic agent is chosen from a taxane, inhibitor of bcr-abl, inhibitor of EGFR, DNA damaging agent, and antimetabolite.

34. The method as recited in claim 32, wherein the therapeutic agent is chosen from aminoglutethimide, amsacrine, anastrozole, asparaginase, bcg, bicalutamide, bleomycin, buserelin, busulfan, campothecin, capecitabine, carboplatin, carmustine, chlorambucil, chloroquine, cisplatin, cladribine, clodronate, colchicine, cyclophosphamide, cyproterone, cytarabine, dacarbazine, dactinomycin, daunorubicin, demethoxyviridin, dichloroacetate, dienestrol, diethylstilbestrol, docetaxel, doxorubicin, epirubicin, estradiol, estramustine, etoposide, everolimus, exemestane, filgrastim, fludarabine, fludrocortisone, fluorouracil, fluoxymesterone, flutamide, gemcitabine, genistein, goserelin, hydroxyurea, idarubicin, ifosfamide, imatinib, interferon, irinotecan, ironotecan, letrozole, leucovorin, leuprolide, levamisole, lomustine, lonidamine, mechlorethamine, medroxyprogesterone, megestrol, melphalan, mercaptopurine, mesna, metformin, methotrexate, mitomycin, mitotane, mitoxantrone, nilutamide, nocodazole, octreotide, oxaliplatin, paclitaxel, pamidronate, pentostatin, perifosine, plicamycin, porfimer, procarbazine, raltitrexed, rituximab, sorafenib, streptozocin, sunitinib, suramin, tamoxifen, temozolomide, temsirolimus, teniposide, testosterone, thioguanine, thiotepa, titanocene dichloride, topotecan, trastuzumab, tretinoin, vinblastine, vincristine, vindesine, and vinorelbine.

35. The method as recited in claim 30, wherein the method further comprises administering non-chemical methods of cancer treatment.

36. The method as recited in claim 35, wherein the method further comprises administering radiation therapy.

37. The method as recited in claim 36, wherein the method further comprises administering surgery, thermoablation, focused ultrasound therapy, cryotherapy, or any combination thereof.

38. A compound of any as recited in claim 1 for use in human therapy.

39. A compound of any as recited in claim 1 for use in treating a GLS1-mediated disease.

40. Use of a compound as recited in claim 1 for the manufacture of a medicament to treat a GLS1-mediated disease.
